(19) United States
(12) Patent Application Publication
(10) Pub. No.: US 2023/0290452 A1
(43) Pub. Date: Sep. 14, 2023

(54) Electronic Health Record (EHR)-Based Classifier for Acute Respiratory Distress Syndrome (ARDS) Subtyping

(71) Applicant: Endpoint Health Inc., Palo Alto, CA (US)

(72) Inventors: Rachel Elizabeth Kast, Palo Alto, CA (US); Emily Mary Van Ark, Palo Alto, CA (US); Rodrigo Octavio Deliberato, Palo Alto, CA (US); Jeffrey Robert Osborn, Palo Alto, CA (US); Diego Ariel Rey, Palo Alto, CA (US)

(21) Appl. No.: 18/007,608

(22) PCT Filed: Jun. 3, 2021
(86) PCT No.: PCT/US2021/035638

§ 371 Date: Dec. 1, 2022

Related U.S. Application Data
(60) Provisional application No. 63/034,368, filed on Jun. 3, 2020.
(60) Provisional application No. 63/064,054, filed on Aug. 11, 2020.
(60) Provisional application No. 63/180,880, filed on Apr. 28, 2021.

(51) Int. Cl. G16H 10/60; G16B 40/00

(52) U.S. Cl. CPC G16H 10/60; G16B 40/00

(57) ABSTRACT

Disclosed herein are methods, non-transitory computer readable media, and systems for subphenotyping acute respiratory distress syndrome (ARDS) patients by analyzing electronic health data (EHR) using a subphenotype classifier. According to their classification, different treatments can be selected which are likely to be efficacious in treating ARDS. Such methods, non-transitory computer readable media, and systems are useful for rapid classification and guided treatment in critical care settings, such as in hospitals.

### CROSS REFERENCE TO RELATED APPLICATIONS

This application claims the benefit of and priority to U.S. Provisional Pat. Application No. 63/034,368 filed on Jun. 3, 2020, U.S. Provisional Pat. Application No. 63/064,054 filed on Aug. 11, 2020, and U.S. Provisional Pat. Application No. 63/180,880 filed on Apr. 28, 2021, the entire disclosure of each of which is hereby incorporated by reference in its entirety for all purposes.

### BACKGROUND

Acute Respiratory Distress Syndrome (ARDS) is respiratory failure with rapid onset of widespread inflammation in the lungs. In many scenarios, ARDS is not triggered by a single pathology as it can be caused by sepsis, pneumonia, trauma, aspiration, pancreatitis, and/or other insults. Therefore, ARDS patients are often not responsive to certain therapies, given the underlying differences in pathologies. Prior attempts to distinguish ARDS patients have implemented machine learning classifier models that are complex (e.g., they use up to 40 predictor variables). For example, in Calfee C.S. et al (2014) Subphenotypes in acute respiratory distress syndrome: latent class analysis of data from two randomised controlled trials. The Lancet Respiratory Medicine 2:611-620, the authors describe models that use biomarkers and other variables that are not easily and readily available at the bedside, which makes generalizability of these models very limited.

### SUMMARY OF THE INVENTION

Disclosed herein are methods, non-transitory computer readable media, and systems for subphenotyping acute respiratory distress syndrome (ARDS) patients by analyzing corresponding electronic health data (EHR) using a patient subphenotype classifier. For example, using a patient subphenotype classifier, the ARDS subjects can be classified into one out of two or more ARDS subphenotypes, examples of which include an ARDS subphenotype characterized by hyperinflammation and an ARDS subphenotype characterized by hypoinflammation. Depending on the particular ARDS subphenotype determined for a subject, a treatment recommendation can be selected and provided to the subject. Here, the patient subphenotype classifiers analyze EHR data without necessarily analyzing other variables (e.g., biomarker values) that would problematically increase the complexity of the model. Thus, such patient subphenotype classifiers can be rapidly deployed on readily obtainable EHR data, thereby enabling their implementation in settings where time is of the essence (e.g., in hospital intensive care units and/or emergency rooms).

Disclosed herein is a method comprising: obtaining or having obtained electronic health record (EHR) data for a subject exhibiting acute respiratory distress syndrome (ARDS); and determining a classification of the subject selected from two or more subphenotypes by analyzing, using a patient subphenotype classifier, the EHR data for the subject without analyzing biomarker levels of the subject. In various embodiments, the patient subphenotype classifier receives one or more input variables comprising heart rate, mean arterial pressure, and respiratory rate. In various embodiments, the patient subphenotype classifier receives each of the input variables of heart rate, mean arterial pressure, and respiratory rate. In various embodiments, the patient subphenotype classifier further receives one or more input variables comprising arterial pH, partial pressure of oxygen, and bicarbonate. In various embodiments, the patient subphenotype classifier further receives each of the input variables comprising arterial pH, partial pressure of oxygen, and bicarbonate. In various embodiments, the patient subphenotype classifier further receives one or more input variables comprising inspirited fraction of oxygen, creatinine, and bilirubin. In various embodiments, the patient subphenotype classifier further receives each of the input variables comprising inspirited fraction of oxygen, creatinine, and bilirubin. In various embodiments, the patient subphenotype classifier further receives one or more input variables comprising partial pressure of carbon dioxide, PaO₂/FiO₂, platelet count, age, gender, positive end-expiratory pressure, and tidal volume. In various embodiments, the patient subphenotype classifier further receives each of the input variables comprising partial pressure of carbon dioxide, PaO₂/FiO₂, platelet count, age, gender, positive end-expiratory pressure, and tidal volume. In various embodiments, the patient subphenotype classifier further receives one or more input variables comprising body mass index, plateau pressure, minute ventilation, and vasopressor use in prior 24 hours. In various embodiments, the patient subphenotype classifier further receives each of the input variables comprising body mass index, plateau pressure, minute ventilation, and vasopressor use in prior 24 hours.

In various embodiments, the patient subphenotype classifier comprises a subphenotyping submodel that outputs a prediction for an ARDS subphenotype. In various embodiments, the patient subphenotype classifier comprises a mortality submodel that outputs a prediction of an ARDS mortality rate. In various embodiments, the patient subphenotype classifier comprises: (A) a subphenotyping submodel that outputs a prediction for an ARDS subphenotype; and (B) a mortality submodel that outputs a prediction of an ARDS mortality rate. In various embodiments, the prediction for the ARDS subphenotype outputted by the subphenotyping submodel serves as an input to the mortality submodel. In various embodiments, the subphenotyping submodel receives one or more input variables comprising the subject’s arterial pH, bicarbonate, creatinine, fraction of inspired oxygen (FiO₂), heart rate, arterial pressure, respiration rate, and partial pressure of oxygen (PaO₂). In various embodiments, the subphenotyping submodel receives each of the input variables of the subject’s arterial pH, bicarbonate, creatinine, fraction of inspired oxygen (FIO₂), heart rate, arterial pressure, respiration rate, and partial pressure of oxygen (PaO₂).

In various embodiments, implementation of the subphenotyping submodel comprises implementing an unsupervised clustering algorithm. In various embodiments, the mortality submodel receives input variables comprising the subject’s gender and age. In various embodiments, the mortality submodel receives input variables comprising the subject’s bilirubin, partial pressure of carbon dioxide (PaCO₂), PaO₂/FiO₂, positive end expiratory pressure (PEEP), platelet count, and tidal volume. In various embodiments, the mortality submodel receives input variables comprising the subject’s arterial pH, bicarbonate, creatinine, fraction of inspired oxygen (FiO₂), heart rate, arterial pressure, respiration rate, and partial pressure of oxygen (PaO₂). In various embodiments, the mortality submodel receives 10 or more input variables comprising the prediction for the ARDS subphenotype outputted by the subphenotyping submodel, the subject’s gender, age, bilirubin, partial pressure of carbon dioxide (PaCO₂), PaO₂/FiO₂, positive end expiratory pressure (PEEP), platelet count, tidal volume, and BMI. In various embodiments, the patient subphenotype classifier has at least one of an area under receiver-operator curve (AUROC) greater than or equal to 0.689 and an area under the precision-recall curve (AUPRC) greater than or equal to 0.650.

In various embodiments, the mortality submodel receives 9 or more input variables comprising the prediction for the ARDS subphenotype outputted by the subphenotyping submodel, the subject’s gender, age, bilirubin, partial pressure of carbon dioxide (PaCO₂), PaO₂/FiO₂, positive end expiratory pressure (PEEP), platelet count, and tidal volume. In various embodiments, the patient subphenotype classifier has at least one of an area under receiver-operator curve (AUROC) greater than or equal to 0.673 and an area under the precision-recall curve (AUPRC) greater than or equal to 0.668. In various embodiments, the mortality submodel receives 12 or more input variables comprising the prediction for the ARDS subphenotype outputted by the subphenotyping submodel, the subject’s gender, age, bilirubin, arterial pH, bicarbonate, creatinine, fraction of inspired oxygen (FIO₂), heart rate, arterial pressure, respiration rate, and partial pressure of oxygen (PaO₂). In various embodiments, the patient subphenotype classifier has at least one of an area under receiver-operator curve (AUROC) greater than or equal to 0.658 and an area under the precision-recall curve (AUPRC) greater than or equal to 0.597. In various embodiments, the mortality submodel receives 11 or more input variables comprising the prediction for the ARDS subphenotype outputted by the subphenotyping submodel, the subject’s gender, age, arterial pH, bicarbonate, creatinine, fraction of inspired oxygen (FiO₂), heart rate, arterial pressure, respiration rate, and partial pressure of oxygen (PaO₂). In various embodiments, the patient subphenotype classifier has at least one of an area under receiver-operator curve (AUROC) greater than or equal to 0.643 and an area under the precision-recall curve (AUPRC) greater than or equal to 0.532.

In various embodiments, implementation of the mortality submodel comprises implementing a supervised machine learning algorithm. In various embodiments, determining the classification of the subject based on the EHR data using the patient subphenotype classifier comprises: determining that data elements of a higher rank mortality submodel are unavailable in the EHR data; and determining that data elements of the mortality submodel are available in the EHR data. In various embodiments, determining the classification of the subject based on the EHR data using the patient subphenotype classifier comprises implementing the mortality submodel responsive to determining that data elements of the mortality submodel are available in the EHR data.

In various embodiments, the mortality submodel comprises two or more sub-models that each outputs a prediction informative for determining an ARDS mortality rate. In various embodiments, the first sub-model receives input variables comprising a first prediction for the ARDS subphenotype outputted by the subphenotyping submodel and the second sub-model receives input variables comprising a second prediction for the ARDS subphenotype outputted by the subphenotyping submodel. In various embodiments, the first sub-model receives input variables further comprising the subject’s bilirubin. In various embodiments, the second sub-model receives input variables further comprising the subject’s bilirubin, partial pressure of carbon dioxide (PaCO₂), PaO₂/FiO₂, positive end expiratory pressure (PEEP), platelet count, and tidal volume. In various embodiments, the subphenotyping submodel comprises two or more sub-models that each outputs a prediction of an ARDS subphenotype.

In various embodiments, implementation of the two or more sub-models comprises implementing unsupervised clustering algorithms. In various embodiments, the patient subphenotype classifier further comprises a pre-mortality model that outputs a prediction that serves as input to the mortality submodel. In various embodiments, implementation of the pre-mortality model comprises implementing a supervised machine learning algorithm.

In various embodiments, the mortality submodel receives, as input, 8 or more input variables. In various embodiments, the 8 or more input variables comprise at least the subject’s arterial pH, bicarbonate, creatinine, fraction of inspired oxygen (FiO₂), and heart rate. In various embodiments, the 8 or more input variables further comprise at least the subject’s airway pressure, arterial pressure, respiration rate, and partial pressure of oxygen (PaO₂). In various embodiments, the patient subphenotype classifier comprises one of a first model, a second model, a third model, and a fourth model, wherein the first model receives, as input, 13 input variables, wherein the second model receives, as input, 8 input variables, wherein the third model receives, as input, 17 input variables, and wherein the fourth model receives, as input, 13 input variables. In various embodiments, the 13 input variables of the first model comprise the subject’s arterial pH, bicarbonate, creatinine, diastolic blood pressure (BP), FiO₂, heart rate, highest mean arterial pressure, lowest mean arterial pressure, potassium, highest respiratory rate, lowest respiratory rate, SPO₂, and systolic BP. In various embodiments, the 13 input variables of the first model comprise the subject’s most recent arterial pH, lowest bicarbonate, most recent creatinine, most recent diastolic blood pressure (BP), most recent FiO₂, most recent heart rate, highest mean arterial pressure, lowest mean arterial pressure, most recent potassium, highest respiratory rate, lowest respiratory rate, most recent SPO₂, and most recent systolic BP. In various embodiments, the patient subphenotype classifier has at least one of an area under receiver-operator curve (AUROC) greater than or equal to 0.67 and an area under the precision-recall curve (AUPRC) greater than or equal to 0.40.

In various embodiments, the 8 input variables of the second model comprise the subject’s arterial pH, bicarbonate, creatinine, FiO₂, heart rate, PaO₂, mean arterial pressure, and respiratory rate. In various embodiments, the 8 input variables of the second model comprise the subject’s most recent arterial pH, lowest bicarbonate, most recent creatinine, most recent FiO₂, most recent heart rate, most recent PaO₂, most recent mean arterial pressure, and most recent respiratory rate. In various embodiments, the patient subphenotype classifier has at least one of an area under receiver-operator curve (AUROC) greater than or equal to 0.69 and an area under the precision-recall curve (AUPRC) greater than or equal to 0.42.

In various embodiments, the 17 input variables of the third model comprise the subject’s age, arterial pH, bicarbonate, bilirubin, BMI, creatinine, FiO₂, gender, heart rate, PaCO₂, PaO₂/FiO₂, PaO₂, positive end-expiratory pressure (PEEP), platelet count, tidal volume, mean arterial pressure, and respiratory rate. In various embodiments, the 17 input variables of the third model comprise the subject’s age, most recent arterial pH, lowest bicarbonate, highest bilirubin, BMI, most recent creatinine, most recent FiO₂, gender, most recent heart rate, most recent PaCO₂, lowest PaO₂/FiO₂ within 24 hours following ARDS diagnosis, most recent PaO₂, most recent positive end-expiratory pressure (PEEP), lowest platelet count, lowest tidal volume, most recent mean arterial pressure, and most recent respiratory rate. In various embodiments, the patient subphenotype classifier has at least one of an area under receiver-operator curve (AUROC) greater than or equal to 0.71 and an area under the precision-recall curve (AUPRC) greater than or equal to 0.62. In various embodiments, the 13 input variables of the fourth model comprise the subject’s arterial pH, bicarbonate, BMI, creatinine, FiO₂, gender, heart rate, PaCO₂, PaO₂/FiO₂, PEEP, platelet count, mean arterial pressure, and respiratory rate. In various embodiments, the 13 input variables of the fourth model comprise the subject’s most recent arterial pH, most recent bicarbonate, BMI, most recent creatinine, most recent FiO₂, gender, most recent heart rate, most recent PaCO₂, lowest PaO₂/FiO₂ within 24 hours following ARDS diagnosis, most recent PEEP, lowest platelet count, most recent mean arterial pressure, and most recent respiratory rate. In various embodiments, the patient subphenotype classifier has at least one of an area under receiver-operator curve (AUROC) greater than or equal to 0.67 and an area under the precision-recall curve (AUPRC) greater than or equal to 0.46.

In various embodiments, the classification of the subject is selected from three or more subphenotypes. In various embodiments, the three or more subphenotypes comprise a lower risk subphenotype, a medium risk subphenotype, and a high risk subphenotype. In various embodiments, the classification of the subject is selected from three by comparing a score to two threshold values. In various embodiments, the patient subphenotype classifier has at least an area under receiver-operator curve (AUROC) greater than or equal to 0.691.

In various embodiments, the patient subphenotype classifier is trained using a training dataset comprising patient data from one or more clinical trial datasets. In various embodiments, the one or more clinical trial datasets are any of ARMA dataset, KARMA dataset, LARMA dataset, ALVEOLI dataset, EDEN dataset, FACTT dataset, SAILS dataset, ROSE dataset, eICU-CRD dataset, and the Brazillian ART dataset. In various embodiments, the patient data is derived from a sub-cohort of patients of the one or more clinical trial datasets, wherein the sub-cohort of patients are characterized by having a ratio of arterial oxygen concentration to the fraction of inspired oxygen (P/F ratio) of less than or equal to 200. In various embodiments, the patient data is derived from a sub-cohort of patients of the one or more clinical trial datasets, wherein the sub-cohort of patients are characterized by having a ratio of arterial oxygen concentration to the fraction of inspired oxygen (P/F ratio) of less than or equal to 300.

In various embodiments, the two or more subphenotypes comprise subphenotype A and subphenotype B that are characterized by differences in expression levels in one or more biomarkers. In various embodiments, the one or more biomarkers comprise one or more of PAI-1, IL-6, IL-8, IL-10, TNFR-I, TNFR-II, ICAM-1, or von Willebrand factor. In various embodiments, the one or more biomarkers comprise each of PAI-1, IL-6, IL-8, IL-10, TNFR-I, TNFR-II, ICAM-1, or von Willebrand factor.

Additionally disclosed herein is a method for identifying a mortality prognosis for a subject, the method comprising: obtaining a classification of the subject exhibiting acute respiratory distress syndrome (ARDS), the classification of the subject selected from two or more subphenotypes and determined using methods disclosed herein; and identifying a mortality prognosis for the subject based at least in part on the classification, wherein responsive to the classification of the subject comprising subphenotype B from the two or more subphenotypes, the mortality prognosis identified for the subject comprises high mortality risk, and wherein responsive to the classification of the subject comprising subphenotype A from the two or more subphenotypes, the mortality prognosis identified for the subject comprises low mortality risk. In various embodiments, low mortality risk comprises at least one of reduced risk of hospital mortality, reduced risk of ICU mortality, reduced risk of 28-day mortality, reduced risk of 90-day mortality, reduced risk of 180-day mortality, and reduced risk of 6-month mortality relative to high mortality risk. In various embodiments, low mortality risk further comprises positive patient outcome, wherein high mortality risk further comprises negative patient outcome, and wherein positive patient outcome comprises at least one of shorter hospital length of stay, shorter ICU length of stay and more ventilator-free days relative to negative patient outcome.

Additionally disclosed herein is a method for identifying a therapy recommendation for a subject, the method comprising: obtaining a classification of a subject exhibiting acute respiratory distress syndrome (ARDS), the classification of the subject selected from two or more subphenotypes and determined using methods disclosed herein; and identifying a therapy recommendation for the subject based at least in part on the classification, wherein responsive to the classification of the subject comprising subphenotype B from the two or more subphenotypes, the therapy recommendation identified for the subject comprises one or more of neuromuscular blockade (NMB) therapy or no NMB therapy, high PEEP or low PEEP, no treatment or methylprednisolone, dexamethasone, no lisofylline, ketoconazole, catheter and fluid treatment, recruitment maneuver, statins, or full or trophic enteral feeding and wherein responsive to the classification of the subject comprising subphenotype A from the two or more subphenotypes, the therapy recommendation identified for the subject comprises one or more of NMB therapy, low PEEP therapy, no methylprednisolone, no treatment or dexamethasone, no treatment or lisofylline, no treatment or ketoconazole, no combination of catheter and fluid treatment, no recruitment maneuver, statins as a preemptive therapy, or full enteral feeding.

Additionally disclosed herein is a method for identifying candidate subjects to be provided a therapy, the method comprising: for one or more subjects, obtaining a classification of the subject exhibiting acute respiratory distress syndrome (ARDS), the classification of the subject selected from two or more subphenotypes and determined using methods disclosed herein; and determining whether the subject is a candidate subject based at least in part on the classification. In various embodiments, the therapy is a neuromuscular blockade (NMB) therapy, and wherein determining whether the subject is a candidate subject comprises determining that the subject is a likely responder responsive to the classification of the subject comprising subphenotype A from the two or more subphenotypes. In various embodiments, the therapy is a neuromuscular blockade (NMB) therapy, and wherein determining whether the subject is a candidate subject comprises determining that the subject is unlikely to be a responder responsive to the classification of the subject comprising subphenotype B from the two or more subphenotypes. In various embodiments, the therapy is a low positive end-expiratory pressure (PEEP) treatment, and wherein determining whether the subject is a candidate subject comprises determining that the subject is likely to be a responder responsive to the classification of the subject comprising subphenotype A from the two or more subphenotypes. In various embodiments, the therapy is a high positive end-expiratory pressure (PEEP) treatment, and wherein determining whether the subject is a candidate subject comprises determining that the subject is likely to be a responder responsive to the classification of the subject comprising subphenotype B from the two or more subphenotypes. In various embodiments, the therapy is a corticosteroid treatment, and wherein determining whether the subject is a candidate subject comprises determining that the subject is likely to be a responder responsive to the classification of the subject comprising subphenotype B from the two or more subphenotypes. In various embodiments, the therapy is a corticosteroid treatment, and wherein determining whether the subject is a candidate subject comprises determining that the subject is unlikely to be a responder responsive to the classification of the subject comprising subphenotype A from the two or more subphenotypes. In various embodiments, the corticosteroid treatment is methylpredinosolone or dexamethasone. In various embodiments, the therapy is a lisofylline treatment, and wherein determining whether the subject is a candidate subject comprises determining that the subject is unlikely to be a responder responsive to the classification of the subject comprising subphenotype B from the two or more subphenotypes. In various embodiments, the therapy is a lisofylline treatment, and wherein determining whether the subject is a candidate subject comprises determining that the subject is likely to be a responder responsive to the classification of the subject comprising subphenotype A from the two or more subphenotypes. In various embodiments, the therapy is a ketoconazole treatment, and wherein determining whether the subject is a candidate subject comprises determining that the subject is likely to be a responder responsive to the classification of the subject comprising subphenotype B from the two or more subphenotypes. In various embodiments, the therapy is a pulmonary artery catheter and liberal fluid treatment, and wherein determining whether the subject is a candidate subject comprises determining that the subject is likely to be a responder responsive to the classification of the subject comprising subphenotype B from the two or more subphenotypes. In various embodiments, the therapy is a pulmonary artery catheter and liberal fluid treatment, and wherein determining whether the subject is a candidate subject comprises determining that the subject is unlikely to be a responder responsive to the classification of the subject comprising subphenotype A from the two or more subphenotypes. In various embodiments, the catheter and fluid treatment comprises a central venous catheter line treatment or a pulmonary artery catheter line treatment. In various embodiments, the therapy is a recruitment maneuver, and wherein determining whether the subject is a candidate subject comprises determining that the subject is likely to be a responder responsive to the classification of the subject comprising subphenotype B from the two or more subphenotypes. In various embodiments, the therapy is a recruitment maneuver, and wherein determining whether the subject is a candidate subject comprises determining that the subject is unlikely to be a responder responsive to the classification of the subject comprising subphenotype A from the two or more subphenotypes. In various embodiments, the therapy is a statin treatment, and wherein determining whether the subject is a candidate subject comprises determining that the subject is likely to be a responder responsive to the classification of the subject comprising subphenotype B from the two or more subphenotypes. In various embodiments, the therapy is a preemptive statin treatment, and wherein determining whether the subject is a candidate subject comprises determining that the subject is likely to be a responder responsive to the classification of the subject comprising subphenotype A from the two or more subphenotypes. In various embodiments, the therapy is full enteral feeding, and wherein determining whether the subject is a candidate subject comprises determining that the subject is likely to be a responder responsive to the classification of the subject comprising subphenotype A from the two or more subphenotypes. In various embodiments, the therapy is trophic enteral feeding, and wherein determining whether the subject is a candidate subject comprises determining that the subject is likely to be a responder responsive to the classification of the subject comprising subphenotype B from the two or more subphenotypes.

Additionally disclosed herein is a non-transitory computer readable medium comprising instructions that, when executed by a processor, cause the processor to: obtain or have obtained electronic health record (EHR) data for a subject exhibiting acute respiratory distress syndrome (ARDS); and determine a classification of the subject selected from two or more subphenotypes by analyzing, using a patient subphenotype classifier, the EHR data for the subject without analyzing biomarker levels of the subject. In various embodiments, the patient subphenotype classifier receives one or more input variables comprising heart rate, mean arterial pressure, and respiratory rate. In various embodiments, the patient subphenotype classifier receives each of the input variables of heart rate, mean arterial pressure, and respiratory rate. In various embodiments, the patient subphenotype classifier further receives one or more input variables comprising arterial pH, partial pressure of oxygen, and bicarbonate. In various embodiments, the patient subphenotype classifier further receives each of the input variables comprising arterial pH, partial pressure of oxygen, and bicarbonate. In various embodiments, the patient subphenotype classifier further receives one or more input variables comprising inspirited fraction of oxygen, creatinine, and bilirubin. In various embodiments, the patient subphenotype classifier further receives each of the input variables comprising inspirited fraction of oxygen, creatinine, and bilirubin. In various embodiments, the patient subphenotype classifier further receives one or more input variables comprising partial pressure of carbon dioxide, PaO₂/FiO₂, platelet count, age, gender, positive end-expiratory pressure, and tidal volume. In various embodiments, the patient subphenotype classifier further receives each of the input variables comprising partial pressure of carbon dioxide, PaO₂/FiO₂, platelet count, age, gender, positive end-expiratory pressure, and tidal volume. In various embodiments, the patient subphenotype classifier further receives one or more input variables comprising body mass index, plateau pressure, minute ventilation, and vasopressor use in prior 24 hours. In various embodiments, the patient subphenotype classifier further receives each of the input variables comprising body mass index, plateau pressure, minute ventilation, and vasopressor use in prior 24 hours.

In various embodiments, the patient subphenotype classifier comprises a subphenotyping submodel that outputs a prediction for an ARDS subphenotype. In various embodiments, the patient subphenotype classifier comprises a mortality submodel that outputs a prediction of an ARDS mortality rate. In various embodiments, the patient subphenotype classifier comprises: (A) a subphenotyping submodel that outputs a prediction for an ARDS subphenotype; and (B) a mortality submodel that outputs a prediction of an ARDS mortality rate. In various embodiments, the prediction for the ARDS subphenotype outputted by the subphenotyping submodel serves as an input to the mortality submodel. In various embodiments, the subphenotyping submodel receives one or more input variables comprising the subject’s arterial pH, bicarbonate, creatinine, fraction of inspired oxygen (FiO₂), heart rate, arterial pressure, respiration rate, and partial pressure of oxygen (PaO₂). In various embodiments, the subphenotyping submodel receives each of the input variables of the subject’s arterial pH, bicarbonate, creatinine, fraction of inspired oxygen (FIO₂), heart rate, arterial pressure, respiration rate, and partial pressure of oxygen (PaO₂). In various embodiments, implementation of the subphenotyping submodel comprises implementing an unsupervised clustering algorithm. In various embodiments, the mortality submodel receives input variables comprising the subject’s gender and age. In various embodiments, the mortality submodel receives input variables comprising the subject’s bilirubin, partial pressure of carbon dioxide (PaCO₂), PaO₂/FiO₂, positive end expiratory pressure (PEEP), platelet count, and tidal volume. In various embodiments, the mortality submodel receives input variables comprising the subject’s arterial pH, bicarbonate, creatinine, fraction of inspired oxygen (FiO₂), heart rate, arterial pressure, respiration rate, and partial pressure of oxygen (PaO₂). In various embodiments, the mortality submodel receives 10 or more input variables comprising the prediction for the ARDS subphenotype outputted by the subphenotyping submodel, the subject’s gender, age, bilirubin, partial pressure of carbon dioxide (PaCO₂), PaO₂/FiO₂, positive end expiratory pressure (PEEP), platelet count, tidal volume, and BMI. In various embodiments, the patient subphenotype classifier has at least one of an area under receiver-operator curve (AUROC) greater than or equal to 0.689 and an area under the precision-recall curve (AUPRC) greater than or equal to 0.650.

In various embodiments, the mortality submodel receives 9 or more input variables comprising the prediction for the ARDS subphenotype outputted by the subphenotyping submodel, the subject’s gender, age, bilirubin, partial pressure of carbon dioxide (PaCO₂), PaO₂/FiO₂, positive end expiratory pressure (PEEP), platelet count, and tidal volume. In various embodiments, the patient subphenotype classifier has at least one of an area under receiver-operator curve (AUROC) greater than or equal to 0.673 and an area under the precision-recall curve (AUPRC) greater than or equal to 0.668.

In various embodiments, the mortality submodel receives 12 or more input variables comprising the prediction for the ARDS subphenotype outputted by the subphenotyping submodel, the subject’s gender, age, bilirubin, arterial pH, bicarbonate, creatinine, fraction of inspired oxygen (FIO₂), heart rate, arterial pressure, respiration rate, and partial pressure of oxygen (PaO₂). In various embodiments, the patient subphenotype classifier has at least one of an area under receiver-operator curve (AUROC) greater than or equal to 0.658 and an area under the precision-recall curve (AUPRC) greater than or equal to 0.597. In various embodiments, the mortality submodel receives 11 or more input variables comprising the prediction for the ARDS subphenotype outputted by the subphenotyping submodel, the subject’s gender, age, arterial pH, bicarbonate, creatinine, fraction of inspired oxygen (FiO₂), heart rate, arterial pressure, respiration rate, and partial pressure of oxygen (PaO₂). In various embodiments, the patient subphenotype classifier has at least one of an area under receiver-operator curve (AUROC) greater than or equal to 0.643 and an area under the precision-recall curve (AUPRC) greater than or equal to 0.532.

In various embodiments, implementation of the mortality submodel comprises implementing a supervised machine learning algorithm. In various embodiments, the instructions that cause the processor to determine the classification of the subject based on the EHR data using the patient subphenotype classifier further comprises instructions that, when executed by the processor, cause the processor to: determine that data elements of a higher rank mortality submodel are unavailable in the EHR data; and determine that data elements of the mortality submodel are available in the EHR data. In various embodiments, the instructions that cause the processor to determine the classification of the subject based on the EHR data using the patient subphenotype classifier further comprises instructions that, when executed by the processor, cause the processor to implement the mortality submodel responsive to determining that data elements of the mortality submodel are available in the EHR data. In various embodiments, the mortality submodel comprises two or more sub-models that each outputs a prediction informative for determining an ARDS mortality rate. In various embodiments, the first sub-model receives input variables comprising a first prediction for the ARDS subphenotype outputted by the subphenotyping submodel and the second sub-model receives input variables comprising a second prediction for the ARDS subphenotype outputted by the subphenotyping submodel. In various embodiments, the first sub-model receives input variables further comprising the subject’s bilirubin. In various embodiments, the second sub-model receives input variables further comprising the subject’s bilirubin, partial pressure of carbon dioxide (PaCO₂), PaO₂/FiO₂, positive end expiratory pressure (PEEP), platelet count, and tidal volume. In various embodiments, the subphenotyping submodel comprises two or more sub-models that each outputs a prediction of an ARDS subphenotype.

In various embodiments, implementation of the two or more sub-models comprises implementing unsupervised clustering algorithms. In various embodiments, the patient subphenotype classifier further comprises a pre-mortality model that outputs a prediction that serves as input to the mortality submodel. In various embodiments, implementation of the pre-mortality model comprises implementing a supervised machine learning algorithm.

In various embodiments, the mortality submodel receives, as input, 8 or more input variables. In various embodiments, the 8 or more input variables comprise at least the subject’s arterial pH, bicarbonate, creatinine, fraction of inspired oxygen (FiO₂), and heart rate. In various embodiments, the 8 or more input variables further comprise at least the subject’s airway pressure, arterial pressure, respiration rate, and partial pressure of oxygen (PaO₂). In various embodiments, the patient subphenotype classifier comprises one of a first model, a second model, a third model, and a fourth model, wherein the first model receives, as input, 13 input variables, wherein the second model receives, as input, 8 input variables, wherein the third model receives, as input, 17 input variables, and wherein the fourth model receives, as input, 13 input variables. In various embodiments, the 13 input variables of the first model comprise the subject’s arterial pH, bicarbonate, creatinine, diastolic blood pressure (BP), FiO₂, heart rate, highest mean arterial pressure, lowest mean arterial pressure, potassium, highest respiratory rate, lowest respiratory rate, SPO₂, and systolic BP. In various embodiments, the 13 input variables of the first model comprise the subject’s most recent arterial pH, lowest bicarbonate, most recent creatinine, most recent diastolic blood pressure (BP), most recent FiO₂, most recent heart rate, highest mean arterial pressure, lowest mean arterial pressure, most recent potassium, highest respiratory rate, lowest respiratory rate, most recent SPO₂, and most recent systolic BP. In various embodiments, the patient subphenotype classifier has at least one of an area under receiver-operator curve (AUROC) greater than or equal to 0.67 and an area under the precision-recall curve (AUPRC) greater than or equal to 0.40.

In various embodiments, the 8 input variables of the second model comprise the subject’s arterial pH, bicarbonate, creatinine, FiO₂, heart rate, PaO₂, mean arterial pressure, and respiratory rate. In various embodiments, the 8 input variables of the second model comprise the subject’s most recent arterial pH, lowest bicarbonate, most recent creatinine, most recent FiO₂, most recent heart rate, most recent PaO₂, most recent mean arterial pressure, and most recent respiratory rate. In various embodiments, the patient subphenotype classifier has at least one of an area under receiver-operator curve (AUROC) greater than or equal to 0.69 and an area under the precision-recall curve (AUPRC) greater than or equal to 0.42. In various embodiments, the 17 input variables of the third model comprise the subject’s age, arterial pH, bicarbonate, bilirubin, BMI, creatinine, FiO₂, gender, heart rate, PaCO₂, PaO₂/FiO₂, PaO₂, positive end-expiratory pressure (PEEP), platelet count, tidal volume, mean arterial pressure, and respiratory rate. In various embodiments, the 17 input variables of the third model comprise the subject’s age, most recent arterial pH, lowest bicarbonate, highest bilirubin, BMI, most recent creatinine, most recent FiO₂, gender, most recent heart rate, most recent PaCO₂, lowest PaO₂/FiO₂ within 24 hours following ARDS diagnosis, most recent PaO₂, most recent positive end-expiratory pressure (PEEP), lowest platelet count, lowest tidal volume, most recent mean arterial pressure, and most recent respiratory rate. In various embodiments, the patient subphenotype classifier has at least one of an area under receiver-operator curve (AUROC) greater than or equal to 0.71 and an area under the precision-recall curve (AUPRC) greater than or equal to 0.62. In various embodiments, the 13 input variables of the fourth model comprise the subject’s arterial pH, bicarbonate, BMI, creatinine, FiO₂, gender, heart rate, PaCO₂, PaO₂/FiO₂, PEEP, platelet count, mean arterial pressure, and respiratory rate. In various embodiments, the 13 input variables of the fourth model comprise the subject’s most recent arterial pH, most recent bicarbonate, BMI, most recent creatinine, most recent FiO₂, gender, most recent heart rate, most recent PaCO₂, lowest PaO₂/FiO₂ within 24 hours following ARDS diagnosis, most recent PEEP, lowest platelet count, most recent mean arterial pressure, and most recent respiratory rate. In various embodiments, the patient subphenotype classifier has at least one of an area under receiver-operator curve (AUROC) greater than or equal to 0.67 and an area under the precision-recall curve (AUPRC) greater than or equal to 0.46.

In various embodiments, the classification of the subject is selected from three or more subphenotypes. In various embodiments, the three or more subphenotypes comprise a lower risk subphenotype, a medium risk subphenotype, and a high risk subphenotype. In various embodiments, the classification of the subject is selected from three by comparing a score to two threshold values. In various embodiments, the patient subphenotype classifier has at least an area under receiver-operator curve (AUROC) greater than or equal to 0.691.

In various embodiments, the patient subphenotype classifier is trained using a training dataset comprising patient data from one or more clinical trial datasets. In various embodiments, the one or more clinical trial datasets are any of ARMA dataset, KARMA dataset, LARMA dataset, ALVEOLI dataset, EDEN dataset, FACTT dataset, SAILS dataset, eICU-CRD dataset, and the Brazillian ART dataset. In various embodiments, the patient data is derived from a sub-cohort of patients of the one or more clinical trial datasets, wherein the sub-cohort of patients are characterized by having a ratio of arterial oxygen concentration to the fraction of inspired oxygen (P/F ratio) of less than or equal to 200. In various embodiments, the patient data is derived from a sub-cohort of patients of the one or more clinical trial datasets, wherein the sub-cohort of patients are characterized by having a ratio of arterial oxygen concentration to the fraction of inspired oxygen (P/F ratio) of less than or equal to 300.

In various embodiments, the two or more subphenotypes comprise subphenotype A and subphenotype B that are characterized by differences in expression levels in one or more biomarkers. In various embodiments, the one or more biomarkers comprise one or more of PAI-1, IL-6, IL-8, IL-10, TNFR-I, TNFR-II, ICAM-1, or von Willebrand factor. In various embodiments, the one or more biomarkers comprise each of PAI-1, IL-6, IL-8, IL-10, TNFR-I, TNFR-II, ICAM-1, or von Willebrand factor.

Additionally disclosed herein is a non-transitory computer readable medium comprising instructions that, when executed by a processor, cause the processor to: obtain a classification of the subject exhibiting acute respiratory distress syndrome (ARDS), the classification of the subject selected from two or more subphenotypes and determined using a non-transitory computer readable medium disclosed herein; and identify a mortality prognosis for the subject based at least in part on the classification, wherein responsive to the classification of the subject comprising subphenotype B from the two or more subphenotypes, the mortality prognosis identified for the subject comprises high mortality risk, and wherein responsive to the classification of the subject comprising subphenotype A from the two or more subphenotypes, the mortality prognosis identified for the subject comprises low mortality risk. In various embodiments, low mortality risk comprises at least one of reduced risk of hospital mortality, reduced risk of ICU mortality, reduced risk of 28-day mortality, reduced risk of 90-day mortality, reduced risk of 180-day mortality, and reduced risk of 6-month mortality relative to high mortality risk. In various embodiments, low mortality risk further comprises positive patient outcome, wherein high mortality risk further comprises negative patient outcome, and wherein positive patient outcome comprises at least one of shorter hospital length of stay, shorter ICU length of stay and more ventilator-free days relative to negative patient outcome.

Additionally disclosed herein is a non-transitory computer readable medium comprising instructions that, when executed by a processor, cause the processor to: obtain a classification of a subject exhibiting acute respiratory distress syndrome (ARDS), the classification of the subject selected from two or more subphenotypes and determined using a non-transitory computer readable medium disclosed herein; and identify a therapy recommendation for the subject based at least in part on the classification, wherein responsive to the classification of the subject comprising subphenotype B from the two or more subphenotypes, the therapy recommendation identified for the subject comprises one or more of neuromuscular blockade (NMB) therapy or no NMB therapy, high PEEP or low PEEP, no treatment or methylprednisolone, dexamethasone, no lisofylline, ketoconazole, catheter and fluid treatment, recruitment maneuver, statins, or full or trophic enteral feeding and wherein responsive to the classification of the subject comprising subphenotype A from the two or more subphenotypes, the therapy recommendation identified for the subject comprises one or more of NMB therapy, low PEEP therapy, no methylprednisolone, no treatment or dexamethasone, no treatment or lisofylline, no treatment or ketoconazole, no combination of catheter and fluid treatment, no recruitment maneuver, statins as a preemptive therapy, or full enteral feeding.

Additionally disclosed herein is a non-transitory computer readable medium comprising instructions that, when executed by a processor, cause the processor to: for one or more subjects, obtain a classification of the subject exhibiting acute respiratory distress syndrome (ARDS), the classification of the subject selected from two or more subphenotypes and determined using a non-transitory computer readable medium disclosed herein; and determine whether the subject is a candidate subject based at least in part on the classification. In various embodiments, the therapy is a neuromuscular blockade (NMB) therapy, and wherein determining whether the subject is a candidate subject comprises determining that the subject is a likely responder responsive to the classification of the subject comprising subphenotype A from the two or more subphenotypes. In various embodiments, the therapy is a neuromuscular blockade (NMB) therapy, and wherein determining whether the subject is a candidate subject comprises determining that the subject is unlikely to be a responder responsive to the classification of the subject comprising subphenotype B from the two or more subphenotypes. In various embodiments, the therapy is a low positive end-expiratory pressure (PEEP) treatment, and wherein determining whether the subject is a candidate subject comprises determining that the subject is likely to be a responder responsive to the classification of the subject comprising subphenotype A from the two or more subphenotypes. In various embodiments, the therapy is a high positive end-expiratory pressure (PEEP) treatment, and wherein determining whether the subject is a candidate subject comprises determining that the subject is likely to be a responder responsive to the classification of the subject comprising subphenotype B from the two or more subphenotypes. In various embodiments, the therapy is a corticosteroid treatment, and wherein determining whether the subject is a candidate subject comprises determining that the subject is likely to be a responder responsive to the classification of the subject comprising subphenotype B from the two or more subphenotypes. In various embodiments, the therapy is a corticosteroid treatment, and wherein determining whether the subject is a candidate subject comprises determining that the subject is unlikely to be a responder responsive to the classification of the subject comprising subphenotype A from the two or more subphenotypes. In various embodiments, the corticosteroid treatment is methylpredinosolone or dexamethasone. In various embodiments, the therapy is a lisofylline treatment, and wherein determining whether the subject is a candidate subject comprises determining that the subject is unlikely to be a responder responsive to the classification of the subject comprising subphenotype B from the two or more subphenotypes. In various embodiments, the therapy is a lisofylline treatment, and wherein determining whether the subject is a candidate subject comprises determining that the subject is likely to be a responder responsive to the classification of the subject comprising subphenotype A from the two or more subphenotypes. In various embodiments, the therapy is a ketoconazole treatment, and wherein determining whether the subject is a candidate subject comprises determining that the subject is likely to be a responder responsive to the classification of the subject comprising subphenotype B from the two or more subphenotypes. In various embodiments, the therapy is a pulmonary artery catheter and liberal fluid treatment, and wherein determining whether the subject is a candidate subject comprises determining that the subject is likely to be a responder responsive to the classification of the subject comprising subphenotype B from the two or more subphenotypes. In various embodiments, the therapy is a pulmonary artery catheter and liberal fluid treatment, and wherein determining whether the subject is a candidate subject comprises determining that the subject is unlikely to be a responder responsive to the classification of the subject comprising subphenotype A from the two or more subphenotypes. In various embodiments, the catheter and fluid treatment comprises a central venous catheter line treatment or a pulmonary artery catheter line treatment. In various embodiments, the therapy is a recruitment maneuver, and wherein determining whether the subject is a candidate subject comprises determining that the subject is likely to be a responder responsive to the classification of the subject comprising subphenotype B from the two or more subphenotypes. In various embodiments, the therapy is a recruitment maneuver, and wherein determining whether the subject is a candidate subject comprises determining that the subject is unlikely to be a responder responsive to the classification of the subject comprising subphenotype A from the two or more subphenotypes. In various embodiments, the therapy is a statin treatment, and wherein determining whether the subject is a candidate subject comprises determining that the subject is likely to be a responder responsive to the classification of the subject comprising subphenotype B from the two or more subphenotypes. In various embodiments, the therapy is a preemptive statin treatment, and wherein determining whether the subject is a candidate subject comprises determining that the subject is likely to be a responder responsive to the classification of the subject comprising subphenotype A from the two or more subphenotypes. In various embodiments, the therapy is full enteral feeding, and wherein determining whether the subject is a candidate subject comprises determining that the subject is likely to be a responder responsive to the classification of the subject comprising subphenotype A from the two or more subphenotypes. In various embodiments, the therapy is trophic enteral feeding, and wherein determining whether the subject is a candidate subject comprising determining that the subject is likely to be a responder responsive to the classification of the subject comprising subphenotype B from the two or more subphenotypes.

Additionally, disclosed herein is a system comprising: a storage memory configured to store electronic health record (EHR) data for a subject exhibiting acute respiratory distress syndrome (ARDS); and a processor communicatively coupled to the storage memory to determine a classification of the subject selected from two or more subphenotypes by analyzing, using a patient subphenotype classifier, the EHR data for the subject without analyzing biomarker levels of the subject. In various embodiments, the patient subphenotype classifier receives one or more input variables comprising heart rate, mean arterial pressure, and respiratory rate. In various embodiments, the patient subphenotype classifier receives each of the input variables of heart rate, mean arterial pressure, and respiratory rate. In various embodiments, the patient subphenotype classifier further receives one or more input variables comprising arterial pH, partial pressure of oxygen, and bicarbonate. In various embodiments, the patient subphenotype classifier further receives each of the input variables comprising arterial pH, partial pressure of oxygen, and bicarbonate. In various embodiments, the patient subphenotype classifier further receives one or more input variables comprising inspirited fraction of oxygen, creatinine, and bilirubin. In various embodiments, the patient subphenotype classifier further receives each of the input variables comprising inspirited fraction of oxygen, creatinine, and bilirubin. In various embodiments, the patient subphenotype classifier further receives one or more input variables comprising partial pressure of carbon dioxide, PaO₂/FiO₂, platelet count, age, gender, positive end-expiratory pressure, and tidal volume. In various embodiments, the patient subphenotype classifier further receives each of the input variables comprising partial pressure of carbon dioxide, PaO₂/FiO₂, platelet count, age, gender, positive end-expiratory pressure, and tidal volume. In various embodiments, the patient subphenotype classifier further receives one or more input variables comprising body mass index, plateau pressure, minute ventilation, and vasopressor use in prior 24 hours. In various embodiments, the patient subphenotype classifier further receives each of the input variables comprising body mass index, plateau pressure, minute ventilation, and vasopressor use in prior 24 hours. In various embodiments, the patient subphenotype classifier comprises a subphenotyping submodel that outputs a prediction for an ARDS subphenotype. In various embodiments, the patient subphenotype classifier comprises a mortality submodel that outputs a prediction of an ARDS mortality rate.

In various embodiments, the patient subphenotype classifier comprises: (A) a subphenotyping submodel that outputs a prediction for an ARDS subphenotype; and (B) a mortality submodel that outputs a prediction of an ARDS mortality rate. In various embodiments, the prediction for the ARDS subphenotype outputted by the subphenotyping submodel serves as an input to the mortality submodel. In various embodiments, the subphenotyping submodel receives one or more input variables comprising the subject’s arterial pH, bicarbonate, creatinine, fraction of inspired oxygen (FiO₂), heart rate, arterial pressure, respiration rate, and partial pressure of oxygen (PaO₂). In various embodiments, the subphenotyping submodel receives each of the input variables of the subject’s arterial pH, bicarbonate, creatinine, fraction of inspired oxygen (FIO₂), heart rate, arterial pressure, respiration rate, and partial pressure of oxygen (PaO₂). In various embodiments, implementation of the subphenotyping submodel comprises implementing an unsupervised clustering algorithm. In various embodiments, the mortality submodel receives input variables comprising the subject’s gender and age. In various embodiments, the mortality submodel receives input variables comprising the subject’s bilirubin, partial pressure of carbon dioxide (PaCO₂), PaO₂/FiO₂, positive end expiratory pressure (PEEP), platelet count, and tidal volume. In various embodiments, the mortality submodel receives input variables comprising the subject’s arterial pH, bicarbonate, creatinine, fraction of inspired oxygen (FiO₂), heart rate, arterial pressure, respiration rate, and partial pressure of oxygen (PaO₂).

In various embodiments, the mortality submodel receives 10 or more input variables comprising the prediction for the ARDS subphenotype outputted by the subphenotyping submodel, the subject’s gender, age, bilirubin, partial pressure of carbon dioxide (PaCO₂), PaO₂/FiO₂, positive end expiratory pressure (PEEP), platelet count, tidal volume, and BMI. In various embodiments, the patient subphenotype classifier has at least one of an area under receiver-operator curve (AUROC) greater than or equal to 0.689 and an area under the precision-recall curve (AUPRC) greater than or equal to 0.650. In various embodiments, the mortality submodel receives 9 or more input variables comprising the prediction for the ARDS subphenotype outputted by the subphenotyping submodel, the subject’s gender, age, bilirubin, partial pressure of carbon dioxide (PaCO₂), PaO₂/FiO₂, positive end expiratory pressure (PEEP), platelet count, and tidal volume. In various embodiments, the patient subphenotype classifier has at least one of an area under receiver-operator curve (AUROC) greater than or equal to 0.673 and an area under the precision-recall curve (AUPRC) greater than or equal to 0.668.

In various embodiments, the mortality submodel receives 12 or more input variables comprising the prediction for the ARDS subphenotype outputted by the subphenotyping submodel, the subject’s gender, age, bilirubin, arterial pH, bicarbonate, creatinine, fraction of inspired oxygen (FIO₂), heart rate, arterial pressure, respiration rate, and partial pressure of oxygen (PaO₂). In various embodiments, the patient subphenotype classifier has at least one of an area under receiver-operator curve (AUROC) greater than or equal to 0.658 and an area under the precision-recall curve (AUPRC) greater than or equal to 0.597. In various embodiments, the mortality submodel receives 11 or more input variables comprising the prediction for the ARDS subphenotype outputted by the subphenotyping submodel, the subject’s gender, age, arterial pH, bicarbonate, creatinine, fraction of inspired oxygen (FiO₂), heart rate, arterial pressure, respiration rate, and partial pressure of oxygen (PaO₂). In various embodiments, the patient subphenotype classifier has at least one of an area under receiver-operator curve (AUROC) greater than or equal to 0.643 and an area under the precision-recall curve (AUPRC) greater than or equal to 0.532. In various embodiments, implementation of the mortality submodel comprises implementing a supervised machine learning algorithm. In various embodiments, the instructions that cause the processor to determine the classification of the subject based on the EHR data using the patient subphenotype classifier further comprises instructions that, when executed by the processor, cause the processor to: determine that data elements of a higher rank mortality submodel are unavailable in the EHR data; and determine that data elements of the mortality submodel are available in the EHR data. In various embodiments, the instructions that cause the processor to determine the classification of the subject based on the EHR data using the patient subphenotype classifier further comprises instructions that, when executed by the processor, cause the processor to implement the mortality submodel responsive to determining that data elements of the mortality submodel are available in the EHR data. In various embodiments, the mortality submodel comprises two or more sub-models that each outputs a prediction informative for determining an ARDS mortality rate. In various embodiments, the first sub-model receives input variables comprising a first prediction for the ARDS subphenotype outputted by the subphenotyping submodel and the second sub-model receives input variables comprising a second prediction for the ARDS subphenotype outputted by the subphenotyping submodel. In various embodiments, the first sub-model receives input variables further comprising the subject’s bilirubin. In various embodiments, the second sub-model receives input variables further comprising the subject’s bilirubin, partial pressure of carbon dioxide (PaCO₂), PaO₂/FiO₂, positive end expiratory pressure (PEEP), platelet count, and tidal volume. In various embodiments, the subphenotyping submodel comprises two or more sub-models that each outputs a prediction of an ARDS subphenotype. In various embodiments, implementation of the two or more sub-models comprises implementing unsupervised clustering algorithms. In various embodiments, the patient subphenotype classifier further comprises a pre-mortality model that outputs a prediction that serves as input to the mortality submodel. In various embodiments, implementation of the pre-mortality model comprises implementing a supervised machine learning algorithm.

In various embodiments, the mortality submodel receives, as input, 8 or more input variables. In various embodiments, the 8 or more input variables comprise at least the subject’s arterial pH, bicarbonate, creatinine, fraction of inspired oxygen (FiO₂), and heart rate. In various embodiments, the 8 or more input variables further comprise at least the subject’s airway pressure, arterial pressure, respiration rate, and partial pressure of oxygen (PaO₂). In various embodiments, the patient subphenotype classifier comprises one of a first model, a second model, a third model, and a fourth model, wherein the first model receives, as input, 13 input variables, wherein the second model receives, as input, 8 input variables, wherein the third model receives, as input, 17 input variables, and wherein the fourth model receives, as input, 13 input variables. In various embodiments, the 13 input variables of the first model comprise the subject’s arterial pH, bicarbonate, creatinine, diastolic blood pressure (BP), FiO₂, heart rate, highest mean arterial pressure, lowest mean arterial pressure, potassium, highest respiratory rate, lowest respiratory rate, SPO₂, and systolic BP. In various embodiments, the 13 input variables of the first model comprise the subject’s most recent arterial pH, lowest bicarbonate, most recent creatinine, most recent diastolic blood pressure (BP), most recent FiO₂, most recent heart rate, highest mean arterial pressure, lowest mean arterial pressure, most recent potassium, highest respiratory rate, lowest respiratory rate, most recent SPO₂, and most recent systolic BP. In various embodiments, the patient subphenotype classifier has at least one of an area under receiver-operator curve (AUROC) greater than or equal to 0.67 and an area under the precision-recall curve (AUPRC) greater than or equal to 0.40. In various embodiments, the 8 input variables of the second model comprise the subject’s arterial pH, bicarbonate, creatinine, FiO₂, heart rate, PaO₂, mean arterial pressure, and respiratory rate. In various embodiments, the 8 input variables of the second model comprise the subject’s most recent arterial pH, lowest bicarbonate, most recent creatinine, most recent FiO₂, most recent heart rate, most recent PaO₂, most recent mean arterial pressure, and most recent respiratory rate. In various embodiments, the patient subphenotype classifier has at least one of an area under receiver-operator curve (AUROC) greater than or equal to 0.69 and an area under the precision-recall curve (AUPRC) greater than or equal to 0.42. In various embodiments, the 17 input variables of the third model comprise the subject’s age, arterial pH, bicarbonate, bilirubin, BMI, creatinine, FiO₂, gender, heart rate, PaCO₂, PaO₂/FiO₂, PaO₂, positive end-expiratory pressure (PEEP), platelet count, tidal volume, mean arterial pressure, and respiratory rate. In various embodiments, the 17 input variables of the third model comprise the subject’s age, most recent arterial pH, lowest bicarbonate, highest bilirubin, BMI, most recent creatinine, most recent FiO₂, gender, most recent heart rate, most recent PaCO₂, lowest PaO₂/FiO₂ within 24 hours following ARDS diagnosis, most recent PaO₂, most recent positive end-expiratory pressure (PEEP), lowest platelet count, lowest tidal volume, most recent mean arterial pressure, and most recent respiratory rate. In various embodiments, the patient subphenotype classifier has at least one of an area under receiver-operator curve (AUROC) greater than or equal to 0.71 and an area under the precision-recall curve (AUPRC) greater than or equal to 0.62. In various embodiments, the 13 input variables of the fourth model comprise the subject’s arterial pH, bicarbonate, BMI, creatinine, FiO₂, gender, heart rate, PaCO₂, PaO₂/FiO₂, PEEP, platelet count, mean arterial pressure, and respiratory rate. In various embodiments, the 13 input variables of the fourth model comprise the subject’s most recent arterial pH, most recent bicarbonate, BMI, most recent creatinine, most recent FiO₂, gender, most recent heart rate, most recent PaCO₂, lowest PaO₂/FiO₂ within 24 hours following ARDS diagnosis, most recent PEEP, lowest platelet count, most recent mean arterial pressure, and most recent respiratory rate. In various embodiments, the patient subphenotype classifier has at least one of an area under receiver-operator curve (AUROC) greater than or equal to 0.67 and an area under the precision-recall curve (AUPRC) greater than or equal to 0.46.

In various embodiments, the classification of the subject is selected from three or more subphenotypes. In various embodiments, the three or more subphenotypes comprise a lower risk subphenotype, a medium risk subphenotype, and a high risk subphenotype. In various embodiments, the classification of the subject is selected from three by comparing a score to two threshold values. In various embodiments, the patient subphenotype classifier has at least an area under receiver-operator curve (AUROC) greater than or equal to 0.691.

In various embodiments, the patient subphenotype classifier is trained using a training dataset comprising patient data from one or more clinical trial datasets. In various embodiments, the one or more clinical trial datasets are any of ARMA dataset, KARMA dataset, LARMA dataset, ALVEOLI dataset, EDEN dataset, FACTT dataset, SAILS dataset, ROSE dataset, eICU-CRD dataset, and the Brazillian ART dataset. In various embodiments, the patient data is derived from a sub-cohort of patients of the one or more clinical trial datasets, wherein the sub-cohort of patients are characterized by having a ratio of arterial oxygen concentration to the fraction of inspired oxygen (P/F ratio) of less than or equal to 200. In various embodiments, the patient data is derived from a sub-cohort of patients of the one or more clinical trial datasets, wherein the sub-cohort of patients are characterized by having a ratio of arterial oxygen concentration to the fraction of inspired oxygen (P/F ratio) of less than or equal to 300.

In various embodiments, the two or more subphenotypes comprise subphenotype A and subphenotype B that are characterized by differences in expression levels in one or more biomarkers. In various embodiments, the one or more biomarkers comprise one or more of PAI-1, IL-6, IL-8, IL-10, TNFR-I, TNFR-II, ICAM-1, or von Willebrand factor. In various embodiments, the one or more biomarkers comprise each of PAI-1, IL-6, IL-8, IL-10, TNFR-I, TNFR-II, ICAM-1, or von Willebrand factor.

Additionally disclosed herein is a system comprising: a storage memory configured to store electronic health record (EHR) data for a subject exhibiting acute respiratory distress syndrome (ARDS); and a processor communicatively coupled to the storage memory to: obtain a classification of the subject exhibiting acute respiratory distress syndrome (ARDS), the classification of the subject selected from two or more subphenotypes and determined using the system of any one of claims 183-249; and identify a mortality prognosis for the subject based at least in part on the classification, wherein responsive to the classification of the subject comprising subphenotype B from the two or more subphenotypes, the mortality prognosis identified for the subject comprises high mortality risk, and wherein responsive to the classification of the subject comprising subphenotype A from the two or more subphenotypes, the mortality prognosis identified for the subject comprises low mortality risk.

In various embodiments, low mortality risk comprises at least one of reduced risk of hospital mortality, reduced risk of ICU mortality, reduced risk of 28-day mortality, reduced risk of 90-day mortality, reduced risk of 180-day mortality, and reduced risk of 6-month mortality relative to high mortality risk. In various embodiments, low mortality risk further comprises positive patient outcome, wherein high mortality risk further comprises negative patient outcome, and wherein positive patient outcome comprises at least one of shorter hospital length of stay, shorter ICU length of stay and more ventilator-free days relative to negative patient outcome.

Additionally disclosed herein is a system comprising: a storage memory configured to store electronic health record (EHR) data for a subject exhibiting acute respiratory distress syndrome (ARDS); and a processor communicatively coupled to the storage memory to: obtain a classification of a subject exhibiting acute respiratory distress syndrome (ARDS), the classification of the subject selected from two or more subphenotypes and determined using the system of any one of claims 183-249; and identify a therapy recommendation for the subject based at least in part on the classification, wherein responsive to the classification of the subject comprising subphenotype B from the two or more subphenotypes, the therapy recommendation identified for the subject comprises one or more of neuromuscular blockade (NMB) therapy or no NMB therapy, high PEEP or low PEEP, no treatment or methylprednisolone, dexamethasone, no lisofylline, ketoconazole, catheter and fluid treatment, recruitment maneuver, statins, or full or trophic enteral feeding and wherein responsive to the classification of the subject comprising subphenotype A from the two or more subphenotypes, the therapy recommendation identified for the subject comprises one or more of NMB therapy, low PEEP therapy, no methylprednisolone, no treatment or dexamethasone, no treatment or lisofylline, no treatment or ketoconazole, no combination of catheter and fluid treatment, no recruitment maneuver, statins as a preemptive therapy, or full enteral feeding.

Additionally disclosed herein is a system comprising: a storage memory configured to store electronic health record (EHR) data for a subject exhibiting acute respiratory distress syndrome (ARDS); and a processor communicatively coupled to the storage memory to: for one or more subjects, obtain a classification of the subject exhibiting acute respiratory distress syndrome (ARDS), the classification of the subject selected from two or more subphenotypes and determined using the system of any one of claims 183-249; and determine whether the subject is a candidate subject based at least in part on the classification.

In various embodiments, the therapy is a neuromuscular blockade (NMB) therapy, and wherein determining whether the subject is a candidate subject comprises determining that the subject is a likely responder responsive to the classification of the subject comprising subphenotype A from the two or more subphenotypes. In various embodiments, the therapy is a neuromuscular blockade (NMB) therapy, and wherein determining whether the subject is a candidate subject comprises determining that the subject is unlikely to be a responder responsive to the classification of the subject comprising subphenotype B from the two or more subphenotypes. In various embodiments, the therapy is a low positive end-expiratory pressure (PEEP) treatment, and wherein determining whether the subject is a candidate subject comprises determining that the subject is likely to be a responder responsive to the classification of the subject comprising subphenotype A from the two or more subphenotypes. In various embodiments, the therapy is a high positive end-expiratory pressure (PEEP) treatment, and wherein determining whether the subject is a candidate subject comprises determining that the subject is likely to be a responder responsive to the classification of the subject comprising subphenotype B from the two or more subphenotypes. In various embodiments, the therapy is a corticosteroid treatment, and wherein determining whether the subject is a candidate subject comprises determining that the subject is likely to be a responder responsive to the classification of the subject comprising subphenotype B from the two or more subphenotypes. In various embodiments, the therapy is a corticosteroid treatment, and wherein determining whether the subject is a candidate subject comprises determining that the subject is unlikely to be a responder responsive to the classification of the subject comprising subphenotype A from the two or more subphenotypes. In various embodiments, the corticosteroid treatment is methylpredinosolone or dexamethasone. In various embodiments, the therapy is a lisofylline treatment, and wherein determining whether the subject is a candidate subject comprises determining that the subject is unlikely to be a responder responsive to the classification of the subject comprising subphenotype B from the two or more subphenotypes. In various embodiments, the therapy is a lisofylline treatment, and wherein determining whether the subject is a candidate subject comprises determining that the subject is likely to be a responder responsive to the classification of the subject comprising subphenotype A from the two or more subphenotypes. In various embodiments, the therapy is a ketoconazole treatment, and wherein determining whether the subject is a candidate subject comprises determining that the subject is likely to be a responder responsive to the classification of the subject comprising subphenotype B from the two or more subphenotypes. In various embodiments, the therapy is a pulmonary artery catheter and liberal fluid treatment, and wherein determining whether the subject is a candidate subject comprises determining that the subject is likely to be a responder responsive to the classification of the subject comprising subphenotype B from the two or more subphenotypes. In various embodiments, the therapy is a pulmonary artery catheter and liberal fluid treatment, and wherein determining whether the subject is a candidate subject comprises determining that the subject is unlikely to be a responder responsive to the classification of the subject comprising subphenotype A from the two or more subphenotypes. In various embodiments, the catheter and fluid treatment comprises a central venous catheter line treatment or a pulmonary artery catheter line treatment. In various embodiments, the therapy is a recruitment maneuver, and wherein determining whether the subject is a candidate subject comprises determining that the subject is likely to be a responder responsive to the classification of the subject comprising subphenotype B from the two or more subphenotypes. In various embodiments, the therapy is a recruitment maneuver, and wherein determining whether the subject is a candidate subject comprises determining that the subject is unlikely to be a responder responsive to the classification of the subject comprising subphenotype A from the two or more subphenotypes. In various embodiments, the therapy is a statin treatment, and wherein determining whether the subject is a candidate subject comprises determining that the subject is likely to be a responder responsive to the classification of the subject comprising subphenotype B from the two or more subphenotypes. In various embodiments, the therapy is a preemptive statin treatment, and wherein determining whether the subject is a candidate subject comprises determining that the subject is likely to be a responder responsive to the classification of the subject comprising subphenotype A from the two or more subphenotypes. In various embodiments, the therapy is a full enteral feeding, and wherein determining whether the subject is a candidate subject comprises determining that the subject is likely to be a responder responsive to the classification of the subject comprising subphenotype A from the two or more subphenotypes. In various embodiments, the therapy is a trophic enteral feeding, and wherein determining whether the subject is a candidate subject comprises determining that the subject is likely to be a responder responsive to the classification of the subject comprising subphenotype B from the two or more subphenotypes.

### BRIEF DESCRIPTION OF THE DRAWINGS

These and other features, aspects, and advantages of the present disclosure will become better understood with regard to the following description, and accompanying drawings, where:

FIG. 1A is a flow diagram of a process for classifying subjects and determining treatment predictions for subjects, in accordance with an embodiment.

FIG. 1B shows a block diagram of an example patient classifier system, in accordance with an embodiment.

FIG. 2A shows an example flow diagram involving the implementation of a classifier, in accordance with a first embodiment.

FIG. 2B shows an example flow diagram involving the implementation of a classifier, in accordance with a second embodiment.

FIG. 2C shows an example flow diagram involving the implementation of a classifier, in accordance with a second embodiment.

FIG. 3 is a flow process of classifying patients and determining a treatment prediction for a subject, in accordance with an embodiment.

FIG. 4 illustrates an example computer for implementing the entities shown in FIGS. 1-3.

FIG. 5 depicts an example process flow for manual batch integration.

FIG. 6 depicts survival of patients in subphenotype A v. subphenotype B across the full Cleveland Clinic Dataset at 28-days (left) and 90-days (right).

FIG. 7 depicts survival of patients in subphenotype A (left) and subphenotype B (right) at 90 days for patients with (1) and without (0) neuromuscular block.

FIG. 8 depicts survival of patients at 28 days (left) and 90 days (right) across patients that are eligible (1) or not eligible (0) for Neuromuscular block according to Cleveland Clinic criteria.

FIG. 9 depicts survival of patients at 90 days with (1) and without (0) neuromuscular block for patients that are eligible (left) and ineligible (right) according to Cleveland Clinic Protocol.

FIG. 10 depicts survival of patients in subphenotype A v. subphenotype B across the Cleveland Clinic Dataset (without comorbidities) at 28-days (left) and 90-days (right).

FIG. 11 depicts survival of patients in subphenotype A (left) and subphenotype B (right) at 90 days for patients with (1) and without (0) neuromuscular block.

FIG. 12 depicts survival of patients at 28 days (left) and 90 days (right) across patients that are eligible (1) or not eligible (0) for Neuromuscular block according to Cleveland Clinic criteria.

FIG. 13 depicts survival of patients at 90 days with (1) and without (0) neuromuscular block for patients that are eligible (left) and ineligible (right) according to Cleveland Clinic Protocol.

FIG. 14 depicts survival of patients in subphenotype A v. subphenotype B across the ALVEOLI dataset at 28-days (left) and 90-days (right).

FIG. 15 depicts survival of patients in subphenotype A (left) and subphenotype B (right) at 90 days for patients with (1) and without (0) neuromuscular block.

FIG. 16 depicts survival of patients at 28 days (left) and 90 days (right) across patients that are eligible (1) or not eligible (0) for Neuromuscular block according to Cleveland Clinic criteria.

FIG. 17 depicts survival of patients at 90 days with (1) and without (0) neuromuscular block for patients that are eligible (left) and ineligible (right) according to Cleveland Clinic Protocol.

FIG. 18 depicts survival of patients in subphenotype A v. subphenotype B across the ARMA-KARMA-LARMA dataset at 28-days (left) and 90-days (right).

FIG. 19 depicts survival of patients in subphenotype A (left) and subphenotype B (right) at 90 days for patients with (1) and without (0) neuromuscular block.

FIG. 20 depicts survival of patients at 28 days (left) and 90 days (right) across patients that are eligible (1) or not eligible (0) for Neuromuscular block according to Cleveland Clinic criteria.

FIG. 21 depicts survival of patients at 90 days with (1) and without (0) neuromuscular block for patients that are eligible (left) and ineligible (right) according to Cleveland Clinic Protocol.

FIG. 22 depicts survival of patients in subphenotype A v. subphenotype B across a combined dataset at 28-days (left) and 90-days (right).

FIG. 23 depicts survival of patients in subphenotype A (left) and subphenotype B (right) at 90 days for patients with (1) and without (0) neuromuscular block.

FIG. 24 depicts survival of patients at 28 days (left) and 90 days (right) across patients that are eligible (1) or not eligible (0) for Neuromuscular block according to Cleveland Clinic criteria.

FIG. 25 depicts survival of patients at 90 days with (1) and without (0) neuromuscular block for patients that are eligible (left) and ineligible (right) according to Cleveland Clinic Protocol.

FIGS. 26A-26D show the results of training and validating the logistic regression Models 1-4.

FIGS. 27A-27C show the impact of varying the threshold on logistic regression Model 2 performance and mortality separation for the training and validation dataset.

FIG. 28 shows an example ensemble technique for performing unsupervised K-means clustering on 8 data elements and uses the subphenotype assignment (derived from the K-means cluster) as input to a supervised logistic regression algorithm with 9 additional data elements.

FIG. 29 shows an example of an ensemble model where different supervised mortality prediction algorithms are applied to the data for a given patient depending on their subphenotype from the unsupervised K-means clustering.

FIG. 30 shows an ensemble model where a combination of different supervised and unsupervised model outputs become inputs to a final ensemble algorithm that then produces a mortality score.

FIG. 31 shows a series of models ensembled in a waterfall design based on the amount of data available for a given patient.

FIG. 32 shows scatter plots of Ensemble 14 (x-axis) versus level of IL-6 (y-axis) with best-fit lines shown.

FIG. 33 shows the calibration curve for a model output as evaluated on a validation cohort.

FIG. 34 shows Kaplan-Meier survival curves for the three risk groups in APDv1.

FIGS. 35A and 35B compare the performance of the PCT mortality prognostic with the APDv1.

FIGS. 36A-C compare the Receiver Operator curves for the available severity scores against the APDv1 score for the same patients.

FIG. 37A shows ranges of variables of patients in subphenotype A and subphenotype B.

FIG. 37B shows variable values of patients in subphenotype A and subphenotype B across different datasets.

FIG. 38 shows a heat map of biomarkers available for the ARMA and ALVEOLI trials.

FIG. 39 depicts example prior distributions used for Bayesian analysis.

FIG. 40 depicts 28-Day Mortality according to groups and subphenotypes.

FIG. 41 shows heterogeneity of Treatment Effect of High PEEP in 28-Day mortality according to the subphenotypes.

FIG. 42 shows risk of 28-Day mortality and interaction between subphenotypes, PaO₂ / FiO₂ and High PEEP.

FIG. 43 shows the treatment prior’s distributions for Bayesian re-analysis of the EDEN trial.

FIG. 44 shows 60-day mortality according to subphenotype and intervention group.

FIG. 45 shows heterogeneity of treatment effect of full feeding in 60-day mortality according to subphenotype, with weakly informative priors considered. Values less than 1 indicate lower mortality.

FIG. 46 shows heterogeneity of treatment effect of full feeding in 60-day mortality according to subphenotype considering pessimistic priors.

FIG. 47 shows heterogeneity of treatment effect of full feeding in 60-day mortality according to subphenotype considering optimistic priors.

FIG. 48 depicts the percentage of patients discharged alive over time through 90 days, stratified by subphenotype and neuromuscular block intervention, and the percentage of patients reaching their final day of unassisted breathing through 28 days, stratified by subphenotype and neuromuscular block intervention.

The figures depict various embodiments of the present disclosure for purposes of illustration only. One skilled in the art will readily recognize from the following discussion that alternative embodiments of the structures and methods illustrated herein can be employed without departing from the principles of the disclosure described herein.

### DETAILED DESCRIPTION

### Definitions

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

The terms “patient” or “subject” are used interchangeably and encompass or organism, mammals including humans or non-humans (e.g., non-human primates, canines, felines, murines, bovines, equines, and porcines), whether in vivo, ex vivo, or in vitro, male or female.

The term “sample” can include a single cell or multiple cells or fragments of cells or an aliquot of body fluid, such as a blood sample, taken from a subject, by means including venipuncture, excretion, ejaculation, massage, biopsy, needle aspirate, lavage sample, scraping, surgical incision, or intervention or other means known in the art. Examples of an aliquot of body fluid include amniotic fluid, aqueous humor, bile, lymph, breast milk, interstitial fluid, blood, blood plasma, cerumen (earwax), Cowper’s fluid (pre-ejaculatory fluid), chyle, chyme, female ejaculate, menses, mucus, saliva, urine, vomit, tears, vaginal lubrication, sweat, serum, semen, sebum, pus, pleural fluid, cerebrospinal fluid, synovial fluid, intracellular fluid, and vitreous humour.

The term “obtaining or having obtained EHR data” encompasses obtaining a set of data determined from at least one sample. Obtaining a dataset encompasses obtaining a sample and processing the sample to experimentally determine the data. The phrase also encompasses receiving a set of data, e.g., from a third party that has processed the sample to experimentally determine the dataset. Additionally, the phrase encompasses mining data from at least one database or at least one publication or a combination of databases and publications. A dataset can be obtained by one of skill in the art via a variety of known ways including stored on a storage memory.

Any terms not directly defined herein shall be understood to have the meanings commonly associated with them as understood within the art of the disclosure. Certain terms are discussed herein to provide additional guidance to the practitioner in describing the compositions, devices, methods and the like of aspects of the disclosure, and how to make or use them. It will be appreciated that the same thing can be said in more than one way. Consequently, alternative language and synonyms can be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the disclosure herein.

Additionally, as used in the specification, the singular forms “a,” “an” and “the” include plural referents unless the context clearly dictates otherwise.

### Overview

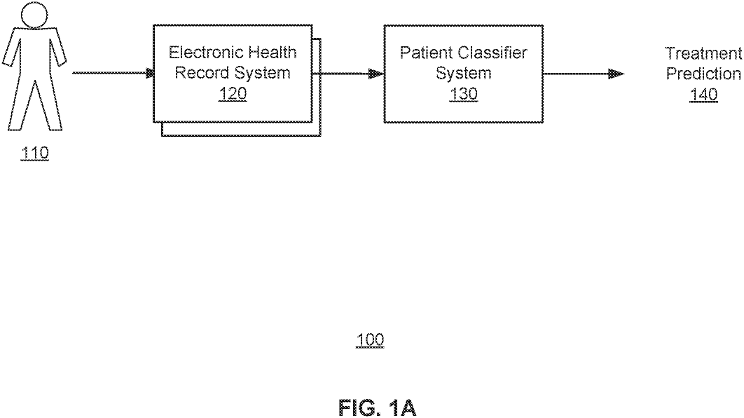

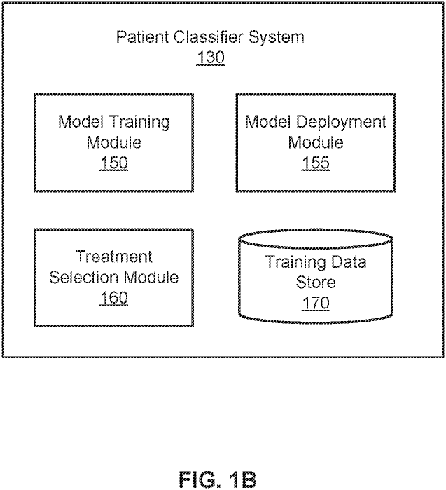

FIG. 1A is a flow diagram of a process for classifying subjects and determining treatment predictions for subjects, in accordance with an embodiment. As shown in FIG. 1A, the system environment 100 includes a subject 110, one or more electronic health record systems 120, and a patient classifier system 130.

In various embodiments, the subject 110 is an individual that was diagnosed with acute respiratory distress syndrome (ARDS). For example, the subject 110 may have been clinically diagnosed as having mild ARDS, moderate ARDS, or severe ARDS based on the Berlin definition. For example, a patient may have been clinically diagnosed with mild ARDS for exhibiting a decreased PaO₂/FiO₂ ratio of between 201-300 mmHg. As another example, a patient may have been clinically diagnosed with moderate ARDS for exhibiting a decreased PaO₂/FiO₂ ratio of between 101-200 mmHg. As another example, a patient may have been clinically diagnosed with severe ARDS for exhibiting a decreased PaO₂/FiO₂ ratio of less than 100 mmHg. In various embodiments, the individual may have been diagnosed with ARDS based on radiologic imaging (e.g., X-ray imaging) or other types of imaging (e.g., CT imaging or ultrasound imaging) that reveals pulmonary accumulation that results in symptoms of ARDS.

Generally, the electronic health record system 120 stores electronic health record (EHR) data for one or more subjects (e.g., subject 110). For example, the electronic health record system 120 may be a physician’s office, the emergency department of a hospital, the intensive care unit of a hospital, the ward of a hospital, a clinical laboratory, a research laboratory, a consumer medical device, a therapeutic device (e.g., an infusion pump), a monitoring device such as a wearable device (e.g., a heart rate monitor), or any other site. Different examples of EHR data is described further herein.

In particular embodiments, the electronic health record system 120 is operated by a party that interacts with the subject 110 (e.g., interacts with subject 110 by diagnosing the subject 110 with ARDS). For example, the electronic health record system 120 can be operated within a healthcare provider’s office and therefore, the electronic health record system 120 stores EHR data of a subject 110 that visits the healthcare provider. In various embodiments, the electronic health record system 120 is operated in a critical care setting. For example, the electronic health record system 120 can be operated within a hospital department (e.g., emergency department or intensive care unit in a hospital). Thus, the EHR data of the subject 110 can be obtained and stored by the electronic health record system 120 for subsequent analysis (e.g., by the patient classifier system 130) to identify a possible treatment for the subject 110. In various embodiments, the electronic health system 120 serves as a repository that electronically records EHR data. Here, the electronic health system 120 can serve as a third-party system that is remote from a location in which the subject 110 is observed and/or interacted with. In such embodiments, the electronic health system 120 can be transmitted the EHR data obtained from a subject 110.

In various embodiments, the electronic health record system 120 can be any of a private, public, and/or commercial source of EHR data. For example, the electronic health record system 120 can be a private medical and/or health record and/or middleware system including a patient care center record system, a clinical laboratory record system, a research laboratory record system, such as EPIC®, Cerner®, Allscripts®, MedMined™, Beaker®, and Data Innovations®, and any alternative private medical and/or health record and/or middleware system. In various embodiments, the electronic health record system 120 stores publicly- and/or commercially-available source of EHR data, including published medical record databases and scientific publications such as PhysioNet datasets including the Multiparameter Intelligent Monitoring in Intensive Care (MIMIC) datasets, Philips eICU datasets, and National Heart, Lung, and Blood Institute Biospecimen and Data Repository Information Coordinating Center (BioLINCC) datasets.

The patient classifier system 130 analyzes EHR data stored by the one or more electronic health record systems 120 and determines a treatment prediction 140 (e.g., a treatment prediction for the subject 110). In various embodiments, the patient classifier system 130 applies a patient subphenotype classifier to predict a classification for subject 110. According the classification, the patient classifier system 130 can determine a treatment prediction 140 for the subject 110 that is likely to be efficacious. In various embodiments, a patient subphenotype classifier can be a machine-learned model. In such embodiments, the patient classification system 130 may train the patient subphenotype classifier using training data and/or deploy the patient subphenotype classifier to analyze the EHR data of the subject 110.

In various embodiments, the patient classifier system 130 and the electronic health record system 120 are operated by different entities. For example, the electronic health record system 120 can be operated by a hospital or healthcare provider, and the patient classifier system 130 can be operated by a third party system that receives and analyzes EHR data stored by the electronic health record system 120. In such embodiments, the electronic health record system 120 transmits EHR data to the patient classifier system 130. The patient classifier system 130 deploys a patient subphenotype classifier and generates a prediction (e.g., treatment prediction 140). The patient classifier system 130 can provide the treatment prediction 140 to the electronic health record system 120 (e.g., to guide patient treatment using the treatment prediction 140).

In various embodiments, the electronic health record system 120 and patient classifier system 130 are implemented in a critical care setting such that a therapy prediction is to be generated for a subject 110 within a maximum amount of time. In various embodiments, the maximum amount of time is 30 minutes. In various embodiments, the maximum amount of time is 1 hour, 2 hours, 3 hours, 4 hours, 5 hours, 6 hours, 7 hours, 8 hours, 9 hours, 10 hours, 11 hours, or 12 hours. Thus, within the maximum amount of time, a therapy prediction is generated and a therapy can be selected for possible administration to the subject 110.

In various embodiments, the patient classifier system 130 and/or the electronic health record system 120 can be distributed computing systems implemented in a cloud computing environment. For example, steps performed by the patient classifier system 130 can be performed using systems in geographically different locations. In particular embodiments, the patient classifier system 130 receives EHR data from the electronic health record system 120 at a first location. The patient classifier system 130 transmits the EHR data and analyzes the EHR data to predict a classification using a patient subphenotype classifier at a second location (e.g., cloud computing). The patient classification system 130 can further transmit the classification back to the first location for subsequent use.

Cloud computing can be employed to offer on-demand access to the shared set of configurable computing resources. The shared set of configurable computing resources can be rapidly provisioned via virtualization and released with low management effort or service provider interaction, and then scaled accordingly. A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In this description and in the claims, a “cloud-computing environment” is an environment in which cloud computing is employed.

### Patient Classifier System

Turning next to FIG. 1B, it shows a block diagram of an example patient classifier system 130, in accordance with an embodiment. Here, the patient classifier system 130 may include a model training module 150, a model deployment module 155, and a treatment selection module 160. In other embodiments, the patient classifier system 130 may include additional, fewer, or different components for various applications. Similarly, the functions can be distributed among the modules in a different manner than is described here. Conventional components such as network interfaces, security functions, load balancers, failover servers, management and network operations consoles, and the like are not shown so as to not obscure the details of the system architecture.

Generally, the model training module 150 constructs a patient subphenotype classifier that is useful for deployment (e.g., by the model deployment module 155) for analyzing EHR data from a subject. In various embodiments, the model training module 150 can construct various patient subphenotype classifiers, each of which is useful for deployment (e.g., by the model deployment module 155) for analyzing EHR data from a subject. In various embodiments, different patient subphenotype classifiers can be structured to receive different input variables (e.g., different EHR data). Therefore, different patient subphenotype classifiers can analyze different EHR data to determine a classification.

In some embodiments, the training data store 170 stores the training dataset that is used to train the patient subphenotype classifier. In various embodiments, the contents of the training dataset depend on the type of the patient subphenotype classifier being trained. In general, the training dataset comprises a plurality of training samples. Each training sample i from the training dataset is associated with a retrospective subject. Each training sample i that is associated with a retrospective subject comprises EHR data for the retrospective subject. Depending on the type of the patient subphenotype classifier, each training sample i of the training dataset may further comprise additional components. For example, in embodiments in which the patient subphenotype classifier is learned via supervised learning, each training sample i from the training dataset can further include a retrospective classification for the retrospective subject associated with the training sample (e.g., a reference ground truth value).

The model deployment module 155 selects one or more patient subphenotype classifiers to be deployed for analyzing EHR data for a subject. In various embodiments, the model deployment module 155 selects and deploys one patient subphenotype classifier to predict a classification for the subject. In various embodiments, the model deployment module 155 selects and deploys multiple patient subphenotype classifiers to predict a classification for the subject. For example, the model deployment module 155 can select and deploy X different patient subphenotype classifiers, each of which determines a classification for the subject. Thus, the model deployment module 155 can compare the classifications for the subject across the different patient subphenotype classifiers and assigns a single classification for the subject. For example, the model deployment module 155 can assign a single classification for the subject that appears across a majority of the outputs of the different patient subphenotype classifiers.

In various embodiments, the model deployment module 155 selects a patient subphenotype classifier to be deployed based on the EHR data that is available. For example, assume that a patient subphenotype classifier receives Y different EHR data variables as input. If less than the Y different EHR data variables are available, the model deployment module 155 can determine whether the EHR data contains Z different EHR data variables such that a different patient subphenotype classifier that receives the Z different EHR data variables (e.g., where Z is less than Y) can be deployed. If the EHR data does not include the Z different EHR data variables, the model deployment module 155 can repeat the process and continue to search for a patient subphenotype classifier that receives fewer EHR data variables as input for which the data variables are available in the EHR data.

In various embodiments, a patient subtype classifier outputs a prediction such as a score. Here, the score can be indicative of the classification for the subject. In various embodiments, the model deployment module 155 compares the score outputted by a patient subtype classifier to one or more threshold scores to determine the classification for the subject. As an example, the patient subtype classifier may output a score between 0 and 1. The model deployment module 155 compares the score outputted by the patient subtype classifier to one or more threshold values. In various embodiments, a threshold value can be a score of 0.1, 0.2, 0.3, 0.4, 0.5, 0.6, 0.7, 0.8, or 0.9. In particular embodiments, the threshold value can be a score of 0.5. Therefore, the model deployment module 155 can compare the score outputted by the patient subtype classifier to the threshold value and classifies the subject based on whether the score is lower or higher than the threshold value.

In various embodiments, the model deployment module 155 compares the score outputted by the patient subtype classifier to two threshold values and classifies the subject based on the two comparisons. In various embodiments, the first threshold value can be a score of 0.1, 0.2, 0.3, 0.4, or 0.5. In various embodiments, the second threshold value can be a score of 0.5, 0.6, 0.7, 0.8, or 0.9. In particular embodiments, the first threshold value is a score of 0.3 and the second threshold value is a score of 0.6. In particular embodiments, the first threshold value is a score of 0.4 and the second threshold value is a score of 0.7. Therefore, the model deployment module 155 compares the score outputted by the patient subtype classifier to both the first threshold value and the second threshold value. Based on the comparisons, the model deployment module 155 classifies the subject into one of three different classifications (e.g., first classification = score is less than first threshold value, second classification = score is greater than first threshold value but less than second threshold value, and third classification = score is greater than second threshold value).

In various embodiments, the model deployment module 155 compares the score outputted by the patient subtype classifier to A different threshold values and classifies the subject based on the X comparisons. For example, the A different threshold values delineates X-1 different score ranges and therefore, based on the X comparisons, the model deployment module 155 determines that the score outputted by the patient subtype classifiers is within one of the X-1 score ranges. Therefore, the model deployment module 155 classifies the subject into a classification corresponding to the one of the X-1 score ranges.

The treatment selection module 160 selects one or more treatments for a subject according to the classification of the subject determined by the model deployment module 155. For example, the treatment selection module 160 may access a lookup table that includes previously determined correspondences between one or more treatments and the classification of the subject. Further examples of specific guided therapies according to patients subphenotypes is described herein.

In various embodiments, the treatment selection module 160 selects one treatment for the subject according to the classification of the subject. In various embodiments, the treatment selection module 160 selects two treatments for the subject according to the classification of the subject. In various embodiments, the treatment selection module 160 selects three treatments for the subject according to the classification of the subject. In various embodiments, the treatment selection module 160 selects four treatments for the subject according to the classification of the subject. In various embodiments, the treatment selection module 160 selects five treatments for the subject according to the classification of the subject.

In various embodiments, the treatment selection module 160 generates a list of the selected one or more treatments and transmits the list. For example, in some embodiments, the treatment selection module 160 transmits the list of selected one or more treatments to a third party such that the list can guide the treatment of the subject under the care of the third party. For example, the third party system can be a hospital department (e.g., intensive care unit or emergency department) at which the subject is located. Therefore, the third party system can provide one or more of the selected treatments identified and provided by the treatment selection module 160.

### Structure of a Patient Subtype Classifier

Generally, the patient subtype classifier is a predictive model that classifies a subject into one out of a plurality of possible classifications based on the EHR data of the subject. In particular embodiments, the patient subtype classifier classifies the subject in a subphenotype out of two possible subphenotypes based on the EHR data of the subject. In particular embodiments, the patient subtype classifier classifies the subject in a subphenotype out of three possible subphenotypes based on the EHR data of the subject. In particular embodiments, the patient subtype classifier classifies the subject in a subphenotype out of four, five, six, seven, eight, nine, or ten possible subphenotypes based on the EHR data of the subject. Additional examples of patient subphenotypes are described herein.

Generally, the patient subtype classifier analyzes EHR data of a subject. In particular embodiments, the patient subtype classifier does not analyze biomarker data for the subject. By analyzing EHR data and not biomarker data, such a patient subtype classifier can be rapidly implemented, which is useful in settings where time is of the essence, such as in critical care settings. Analyzing a sample to obtain biomarker data for a subject can require more resources (e.g., resources in terms of time reagent assays) than obtaining EHR data for the subject.

In various embodiments, the patient subphenotype classifier is a machine learned model. In various embodiments, the predictive model is any one of a regression model (e.g., linear regression, logistic regression, or polynomial regression), decision tree, random forest, support vector machine, Naive Bayes model, k-means cluster, or neural network (e.g., feed-forward networks, convolutional neural networks (CNN), deep neural networks (DNN), autoencoder neural networks, generative adversarial networks, or recurrent networks (e.g., long short-term memory networks (LSTM), bi-directional recurrent networks, deep bi-directional recurrent networks), or any combination thereof. In particular embodiments, the patient subphenotype classifier is a k-mean cluster model that performs unsupervised clustering of subjects according to their EHR data. In particular embodiments, the patient subphenotype classifier is a logistic regression model, such as a Bayesian logistic regression model. In various embodiments, the patient subphenotype classifier is a mixed-effect Bayesian logistic regression model. In various embodiments, the patient subphenotype classifier is a Bayesian hierarchical logistic model that is modelled as a simple regression and shrinkage model.

In various embodiments, the patient subphenotype classifier can be trained using a machine learning implemented method, such as any one of a linear regression algorithm, logistic regression algorithm, decision tree algorithm, support vector machine classification, Naive Bayes classification, K-Nearest Neighbor classification, random forest algorithm, deep learning algorithm, gradient boosting algorithm, and dimensionality reduction techniques such as manifold learning, principal component analysis, factor analysis, autoencoder regularization, and independent component analysis, or combinations thereof. In various embodiments, the predictive model is trained using supervised learning algorithms, unsupervised learning algorithms, semi-supervised learning algorithms (e.g., partial supervision), weak supervision, transfer, multi-task learning, or any combination thereof. In particular embodiments, the predictive model is trained using supervised learning algorithms.

In various embodiments, the predictive model has one or more parameters, such as hyperparameters or model parameters. Hyperparameters are generally established prior to training. Examples of hyperparameters include the learning rate, depth or leaves of a decision tree, number of hidden layers in a deep neural network, number of clusters in a k-means cluster, penalty in a regression model, and a regularization parameter associated with a cost function. Model parameters are generally adjusted during training. Examples of model parameters include weights associated with nodes in layers of neural network, support vectors in a support vector machine, and coefficients in a regression model. The model parameters of the predictive model are trained (e.g., adjusted) using the training data to improve the predictive capacity of the predictive model.

In various embodiments, the patient subphenotype classifier comprises a parametric-model. Thus, such a patient phenotype classifier can be represented as:

$$y = f \left({x}^{k} , θ\right) \qquad \text{(1A)}$$

where y denotes the prediction determined by the patient phenotype classifier, x^{k} denotes the independent variables (e.g., x¹ = EHR data), θ denotes the set of parameters, and ƒ(·) is the function.

In some embodiments, the patient phenotype classifier comprises two or more functions. In such embodiments, the model can be represented as:

$$y = {f}_{1} \left({x}_{1}^{k} , {θ}_{1}\right) ∗ {f}_{2} \left({x}_{2}^{l} , {θ}_{2}\right) \qquad \text{(1B)}$$

where the indicator “ * ” represents any mathematical operation (e.g., summation, multiplication, etc.) such that the two functions, ƒ₁ and ƒ₂, are combined to determine y, the prediction.

In some embodiments, the patient phenotype classifier comprises two or more functions where the output of a first function serves as input to a second function. In such embodiments, the model can be represented as:

$$γ = g \left(f \left({x}^{2} , θ\right)\right) \qquad \text{(1C)}$$

where ƒ is the first function and the output of ƒ serves as input to the second function g.

In some embodiments, the patient phenotype classifier comprises a plurality of functions whose outputs serve as input to one or more functions. In such embodiments, the model can be represented as:

$$y = g \left({f}_{1} \left({x}_{1}^{k} , {θ}_{1}\right)\right) ∗ {f}_{2} \left(\left({x}_{2}^{l} , {θ}_{2}\right)\right) \qquad \text{(1D)}$$

where ƒ₁ and ƒ₂ are the plurality of functions whose output serve as input to an additional function g, which outputs y, the prediction.

In certain embodiments in which x^{k} denotes multiple different independent variables (e.g., x¹ and x²), the multiple independent variables can be combined prior to being input into the function ƒ(·). For example, independent variables of different EHR data can be combined to create a new independent variable prior to being input into the function ƒ(·). For example, EHR data in the form of PaO₂ can be combined with the subject’s EHR data in the form of FiO₂ to create a new independent variable describing the ratio of the two values (e.g., PaO₂/FiO₂). In some embodiments in which x^{k} denotes multiple different independent variables (e.g., x¹ and x²), the different independent variables remain separate and distinct from one another when input into the function ƒ(·).

The function f(·) can be any function, and can comprise any combination of hyperparameters. For example, in some embodiments, the function f(·) can be an affine function given by:

$$y = f \left({x}^{k} {}_{1} θ\right) = {x}^{k} ⋅ θ \qquad \text{(2)}$$

that linearly combines independent variables x^{k} with a corresponding parameter in the set of parameters.

As another example, in some embodiments, the function ƒ(·) can be a network function given by:

$$y = f \left({x}^{k} {}_{1} θ\right) = N N \left({x}^{k} {}_{1} θ\right) \qquad \text{(3)}$$

where NN(-) is a network model. Generally, network models NN(·) can be feed-forward networks, such as artificial neural networks (ANN), convolutional neural networks (CNN), deep neural networks (DNN), and/or recurrent networks, such as long short-term memory networks (LSTM), bi-directional recurrent networks, deep bi-directional recurrent networks,and the like. A network model NN(·) can be defined by any combination of hyperparameters. For example, in a recurrent network, the network can comprise any number of hidden layers, with any number of nodes per layer, and each layer can comprise any layer type, including, but not limited to, a Masking Layer, a Long-Short Term Memory (LSTM) Layer, a Gated Recurrent Units (GRU) Layer, and a Densification Layer. Furthermore, the learning rate of the model can comprise any rate.

In even further embodiments, the function f(·) can be an ensemble of decision trees, such as a random forest or a gradient boosting classifier. In such embodiments, any number of decision trees may be incorporated into the model, and each decision tree may have any maximum depth. Furthermore, the learning rate of the model can comprise any rate.

As discussed above with regard to Equation 1, the function f(·) can be any function. For example, in some embodiments the function f(·) can be an affine function depicted in Equation 2, where x^{k} becomes x¹ or x². Alternatively, the function ƒ(·) can be a network function depicted in Equation 3, where x^{k} becomes x¹ or x². In even further embodiments, the function ƒ(·) can be an ensemble of decision trees, such as a random forest or a gradient boosting classifier.

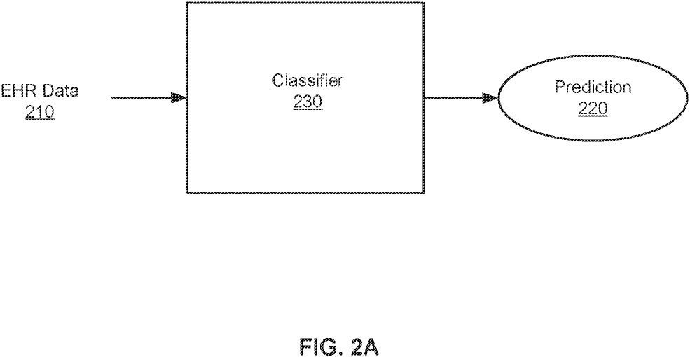

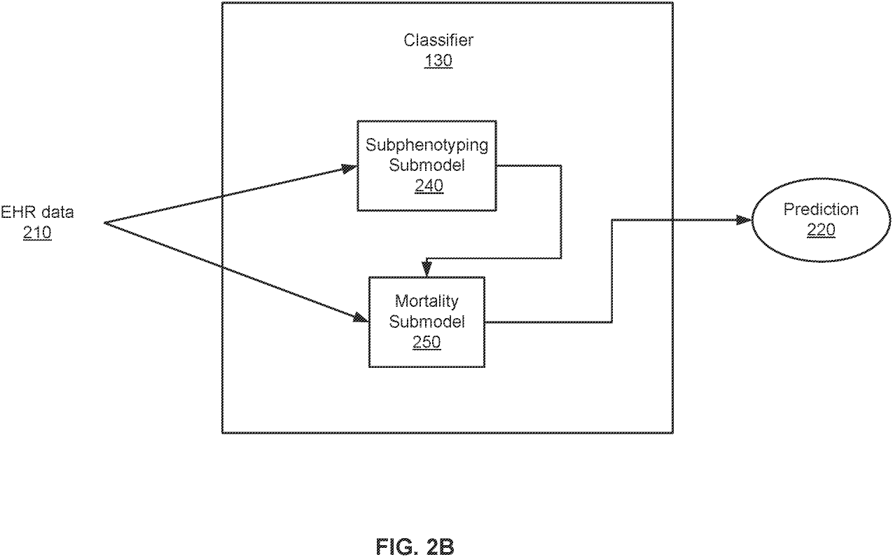

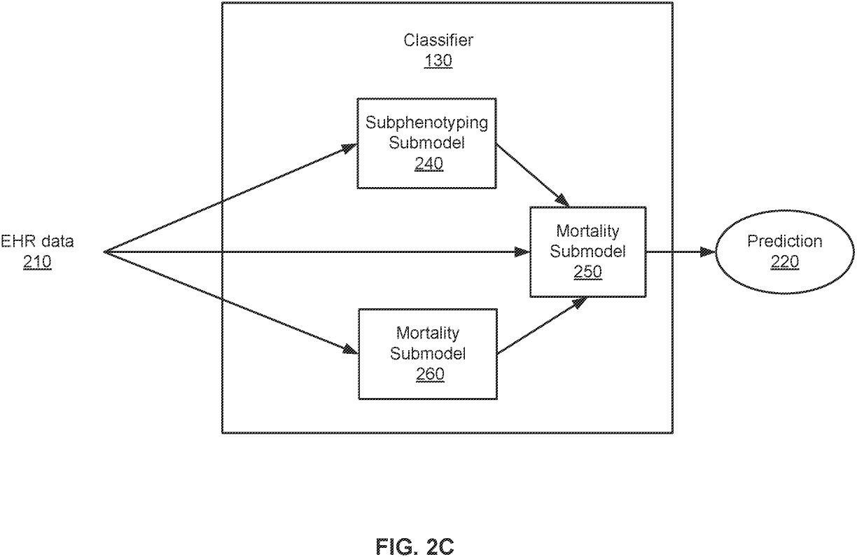

Reference is made to FIG. 2A, which shows an example flow diagram involving the implementation of a classifier 230, in accordance with a first embodiment. In various embodiments, the classifier 230 (e.g., patient subtype classifier) receives, as input, EHR data 210 for a subject. The classifier 230 analyzes the EHR data 210 and outputs a prediction 220 for the subject. In various embodiments, the prediction 220 is a classification. For example, the prediction 220 is a classification of an ARDS subphenotype (e.g., subphenotype A or subphenotype B) for the subject. In various embodiments, the prediction 220 is a score that is informative for determining a classification. As described herein, the score can be compared to one or more threshold values to determine the classification.

In various embodiments, the classifier 230 receives, as input, values of one or more different types of EHR data. Different types of EHR data for a subject include any of: arterial pH, bicarbonate levels, creatinine levels, potassium levels, fraction of inspired oxygen (FiO₂), heart rate, mean arterial pressure, respiration rate, partial pressure of oxygen (PaO₂), gender, age, bilirubin levels, partial pressure of carbon dioxide (PaCO₂), ratio of PaO₂/FiO₂, positive end expiratory pressure (PEEPR), platelet count, mean airway pressure, tidal volume, diastolic blood pressure, systolic blood pressure, plateau pressure, minute ventilation, vasopressor use, and body mass index (BMI). In various embodiments, EHR data can refer to a most recent measurement any of arterial pH, bicarbonate levels, creatinine levels, potassium levels, fraction of inspired oxygen (FiO₂), heart rate, mean arterial pressure, respiration rate, partial pressure of oxygen (PaO₂), gender, age, bilirubin levels, partial pressure of carbon dioxide (PaCO₂), ratio of PaO₂/FiO₂, positive end expiratory pressure (PEEPR), platelet count, mean airway pressure, tidal volume, diastolic blood pressure, systolic blood pressure, plateau pressure, minute ventilation, vasopressor use (e.g., use in the last 24 hours), and body mass index (BMI). As described herein, most recent measurement of EHR data is denoted using “R” that is appended after the type of EHR data. For example, a most recent measure of heart rate is denoted as “heart rate-R” or “HRATER” where the “R” notation is underlined and bolded.

In various embodiments, an alternative to a most recent measurement of EHR data can be used. In various embodiments, EHR data can be aggregated according to a standard midpoint for an EHR data input. For example, for a highest and lowest value of a EHR data input, the distance from the mean is calculated. Whichever value (highest or lowest) was furthest from the mean can be selected as a feature for input.

In various embodiments, EHR data can refer to the lowest measurement of any of arterial pH, bicarbonate levels, creatinine levels, potassium levels, fraction of inspired oxygen (FiO₂), heart rate, mean arterial pressure, respiration rate, partial pressure of oxygen (PaO₂), bilirubin levels, partial pressure of carbon dioxide (PaCO₂), ratio of PaO₂/FiO₂, positive end expiratory pressure (PEEPR), platelet count, mean airway pressure, tidal volume, diastolic blood pressure, systolic blood pressure, plateau pressure, minute ventilation, and body mass index (BMI). As described herein, lowest measurement of EHR data is denoted using “L” that is appended after the type of EHR data. For example, a lowest measure of bicarbonate is denoted as “bicarbonate-L” or “BICARL” where the “L” notation is underlined and bolded.

In various embodiments, EHR data can refer to the highest measurement of any of: arterial pH, bicarbonate levels, creatinine levels, potassium levels, fraction of inspired oxygen (FiO₂), heart rate, mean arterial pressure, respiration rate, partial pressure of oxygen (PaO₂), bilirubin levels, partial pressure of carbon dioxide (PaCO₂), ratio of PaO₂/FiO₂, positive end expiratory pressure (PEEPR), platelet count, mean airway pressure, tidal volume, diastolic blood pressure, systolic blood pressure, plateau pressure, minute ventilation, and body mass index (BMI). As described herein, highest measurement of EHR data is denoted using “H” that is appended after the type of EHR data. For example, a highest measure of bilirubin is denoted as “bilirubin-H” or “BILIH” where the “H” notation is underlined and bolded.

In various embodiments, EHR data can refer to measurements obtained at a clinically relevant time. In various embodiments, a clinically relevant time refers to a time the subject was admitted (e.g., admitted to the hospital). In various embodiments, a clinically relevant time refers to a time the subject was admitted into the emergency department or in the intensive care unit (ICU). In various embodiments, a clinically relevant time refers to a time the subject was enrolled into a clinical trial. In various embodiments, a clinically relevant time refers to a time the subject was diagnosed (e.g., diagnosed with ARDS). In various embodiments, a clinically relevant time refers to a time a clinician ordered a test for the subject. Thus, in such embodiments, the EHR can refer to the measurement at the clinically relevant time for any of arterial pH, bicarbonate levels, creatinine levels, potassium levels, fraction of inspired oxygen (FiO₂), heart rate, mean arterial pressure, respiration rate, partial pressure of oxygen (PaO₂), bilirubin levels, partial pressure of carbon dioxide (PaCO₂), ratio of PaO₂/FiO₂, positive end expiratory pressure (PEEPR), platelet count, mean airway pressure, tidal volume, diastolic blood pressure, systolic blood pressure, plateau pressure, minute ventilation, vasopressor use, and body mass index (BMI).

In various embodiments, a patient subphenotype classifier receives, as input, values of at least two different types of EHR data. In various embodiments, a patient subphenotype classifier receives, as input, values of at least three different types of EHR data. In various embodiments, a patient subphenotype classifier receives, as input, values of at least four different types of EHR data. In various embodiments, a patient subphenotype classifier receives, as input, values of at least five different types of EHR data. In various embodiments, a patient subphenotype classifier receives, as input, values of at least six different types of EHR data. In various embodiments, a patient subphenotype classifier receives, as input, values of at least seven different types of EHR data. In various embodiments, a patient subphenotype classifier receives, as input, values of at least eight different types of EHR data. In various embodiments, a patient subphenotype classifier receives, as input, values of at least nine different types of EHR data. In various embodiments, a patient subphenotype classifier receives, as input, values of at least ten different types of EHR data. In various embodiments, a patient subphenotype classifier receives, as input, values of at least eleven different types of EHR data. In various embodiments, a patient subphenotype classifier receives, as input, values of at least twelve different types of EHR data. In various embodiments, a patient subphenotype classifier receives, as input, values of at least thirteen different types of EHR data. In various embodiments, a patient subphenotype classifier receives, as input, values of at least fourteen different types of EHR data. In various embodiments, a patient subphenotype classifier receives, as input, values of at least fifteen different types of EHR data. In various embodiments, a patient subphenotype classifier receives, as input, values of at least sixteen different types of EHR data. In various embodiments, a patient subphenotype classifier receives, as input, values of at least seventeen different types of EHR data. In various embodiments, a patient subphenotype classifier receives, as input, values of at least eighteen different types of EHR data. In various embodiments, a patient subphenotype classifier receives, as input, values of at least nineteen different types of EHR data. In various embodiments, a patient subphenotype classifier receives, as input, values of at least twenty different types of EHR data.

In various embodiments, a patient subphenotype classifier receives, as input, values of two different types of EHR data. In various embodiments, a patient subphenotype classifier receives, as input, values of three different types of EHR data. In various embodiments, a patient subphenotype classifier receives, as input, values of four different types of EHR data. In various embodiments, a patient subphenotype classifier receives, as input, values of five different types of EHR data. In various embodiments, a patient subphenotype classifier receives, as input, values of six different types of EHR data. In various embodiments, a patient subphenotype classifier receives, as input, values of seven different types of EHR data. In various embodiments, a patient subphenotype classifier receives, as input, values of eight different types of EHR data. In various embodiments, a patient subphenotype classifier receives, as input, values of nine different types of EHR data. In various embodiments, a patient subphenotype classifier receives, as input, values of ten different types of EHR data. In various embodiments, a patient subphenotype classifier receives, as input, values of eleven different types of EHR data. In various embodiments, a patient subphenotype classifier receives, as input, values of twelve different types of EHR data. In various embodiments, a patient subphenotype classifier receives, as input, values of thirteen different types of EHR data. In various embodiments, a patient subphenotype classifier receives, as input, values of fourteen different types of EHR data. In various embodiments, a patient subphenotype classifier receives, as input, values of fifteen different types of EHR data. In various embodiments, a patient subphenotype classifier receives, as input, values of sixteen different types of EHR data. In various embodiments, a patient subphenotype classifier receives, as input, values of seventeen different types of EHR data. In various embodiments, a patient subphenotype classifier receives, as input, values of eighteen different types of EHR data. In various embodiments, a patient subphenotype classifier receives, as input, values of nineteen different types of EHR data. In various embodiments, a patient subphenotype classifier receives, as input, values of twenty different types of EHR data.

In various embodiments, a patient subphenotype classifier receives, as input, the following thirteen input variables: Arterial pH-R, Bicarbonate-L, creatinine -R, Diastolic BP-R, FIO2-R, Heart Rate-R, Mean arterial pressure-H, mean arterial pressure-L, potassium-R, respiratory rate-H, respiratory rate-L, most recent oxygen saturation (SPO₂—R), systolic BP-R.

In various embodiments, a patient subphenotype classifier receives, as input, the following eight input variables: Arterial pH-R, bicarbonate-L, creatinine-R, FIO₂-R, heart rate-R, PaO₂—R, mean arterial pressure-R, respiratory rate-R.

In various embodiments, a patient subphenotype classifier receives, as input, the following seventeen input variables: Age, arterial pH-R, bicarbonate-L, bilirubin-H, BMI, creatinine-R, FiO₂-R, gender, heart rate-R, PaCO₂—R, PaO₂/FiO₂-LP, PaO₂—R, PEEP-R, Platelet-L, Tidal Volume-R, mean arterial pressure-R, respiratory rate-R.

In various embodiments, a patient subphenotype classifier receives, as input, the following thirteen input variables: Arterial pH-R, bicarbonate-R, BMI, creatinine-R, FiO₂-R, gender, heart rate-R, PaCO₂—R, PaO₂/FiO₂-LP, PEEP-R, Platelets-L, mean arterial pressure-R, respiratory rate-R.

In various embodiments, a patient subphenotype classifier receives, as input, the following nine input variables: Arterial pH-R, bicarbonate-L, creatinine-R, FIO₂-R, heart rate-R, PaO₂—R, mean airway pressure-R, respiratory rate-R, bilirubin-H.

In various embodiments, a patient subphenotype classifier receives, as input, the following sixteen input variables: Age, arterial pH-R, bicarbonate-L, bilirubin-H, creatinine-R, FiO₂-R, gender, heart rate-R, PaCO₂—R, PaO₂/FiO₂-LP, PaO₂—R, PEEP-R, Platelet-L, Tidal Volume-R, mean arterial pressure-R, respiratory rate-R.

In various embodiments, a patient subphenotype classifier receives, as input, the following eight input variables: heart rate-R, mean arterial pressure-R, respiratory rate-R, arterial pH-R, PaO₂—R, FiO₂-R, bicarbonate-L, and creatinine-R. Such an example patient subphenotype classifier is described in Example 5 as Model B.1.

In various embodiments, a patient subphenotype classifier receives, as input, the following nine input variables: heart rate-R, mean arterial pressure-R, respiratory rate-R, arterial pH-R, PaO₂—R, FiO₂-R, bicarbonate-L, creatinine-R, and bilirubin-H. Such an example patient subphenotype classifier is described in Example 5 as Model B.2.

In various embodiments, a patient subphenotype classifier receives, as input, the following eleven input variables: heart rate-R, mean arterial pressure-R, respiratory rate-R, arterial pH-R, PaO₂—R, FiO₂-R, bicarbonate-L, creatinine-R, bilirubin-H, age, and gender. Such an example patient subphenotype classifier is described in Example 5 as Model B.3.

In various embodiments, a patient subphenotype classifier receives, as input, the following ten input variables: heart rate-R, mean arterial pressure-R, respiratory rate-R, arterial pH-R, PaO₂—R, FiO₂-R, bicarbonate-L, creatinine-R, age, and gender. Such an example patient subphenotype classifier is described in Example 5 as Model B.4.

In various embodiments, a patient subphenotype classifier receives, as input, the following fifteen input variables: heart rate-R, mean arterial pressure-R, respiratory rate-R, arterial pH-R, PaO₂—R, FiO₂-R, PaCO₂—R, PaO₂/FiO₂, bicarbonate-L, creatinine-R, platelet-L, age, gender, positive end-expiratory pressure-R, and tidal volume-R. Such an example patient subphenotype classifier is described in Example 5 as Model B.5.

In various embodiments, a patient subphenotype classifier receives, as input, the following sixteen input variables: heart rate-R, mean arterial pressure-R, respiratory rate-R, arterial pH-R, PaO₂—R, FiO₂-R, PaCO₂—R, PaO₂/FiO₂, bicarbonate-L, creatinine-R, bilirubin-H, platelet-L, age, gender, positive end-expiratory pressure-R, and tidal volume-R. Such an example patient subphenotype classifier is described in Example 5 as Model B.6.

In various embodiments, a patient subphenotype classifier receives, as input, the following ten input variables: heart rate-R, mean arterial pressure-R, respiratory rate-R, arterial pH-R, PaO₂—R, FiO₂-R, PaCO₂—R, bicarbonate-L, creatinine-R, and bilirubin-H. Such an example patient subphenotype classifier is described in Example 5 as Model B.7.

In various embodiments, a patient subphenotype classifier receives, as input, the following eleven input variables: heart rate-R, mean arterial pressure-R, respiratory rate-R, arterial pH-R, PaO₂—R, FiO₂-R, PaCO₂—R, bicarbonate-L, creatinine-R, bilirubin-H, and platelet-L. Such an example patient subphenotype classifier is described in Example 5 as Model B.8.

In various embodiments, a patient subphenotype classifier receives, as input, the following nine input variables: heart rate-R, mean arterial pressure-R, respiratory rate-R, arterial pH-R, PaO₂—R, FiO₂-R, PaCO₂—R, bicarbonate-L, and creatinine-R. Such an example patient subphenotype classifier is described in Example 5 as Model B.9.

In various embodiments, a patient subphenotype classifier receives, as input, the following five input variables: heart rate-R, mean arterial pressure-R, respiratory rate-R, age, and gender. Such an example patient subphenotype classifier is described in Example 5 as Model B.10.

In various embodiments, a patient subphenotype classifier receives, as input, the following twelve input variables: heart rate-R, mean arterial pressure-R, respiratory rate-R, arterial pH-R, PaO₂—R, FiO₂-R, PaCO₂—R, bicarbonate-L, creatinine-R, bilirubin-H, age, and gender. Such an example patient subphenotype classifier is described in Example 5 as Model B.11.

In various embodiments, a patient subphenotype classifier receives, as input, the following fourteen input variables: heart rate-R, mean arterial pressure-R, respiratory rate-R, arterial pH-R, PaO₂—R, FiO₂-R, PaCO₂—R, PaO₂/FiO₂, bicarbonate-L, creatinine-R, bilirubin-H, platelets-L, positive end-expiratory pressure-R, and tidal volume-R. Such an example patient subphenotype classifier is described in Example 5 as Model B.12.

In various embodiments, a patient subphenotype classifier receives, as input, the following twenty input variables: heart rate-R, mean arterial pressure-R, respiratory rate-R, arterial pH-R, PaO₂—R, FiO₂-R, PaCO₂—R, PaO₂/FiO₂, bicarbonate-L, creatinine-R, bilirubin-H, platelets-L, age, gender, body mass index, positive end-expiratory pressure-R, tidal volume-R, plateau pressure-R, minute ventilation-R, and vasopressor use in the prior 24 hours. Such an example patient subphenotype classifier is described in Example 5 as Model B.13.

In various embodiments, a patient subphenotype classifier receives, as input, the following seven input variables: heart rate-R, mean arterial pressure-R, respiratory rate-R, arterial pH-R, PaO₂—R, bicarbonate-L, and creatinine-R. Such an example patient subphenotype classifier is described in Example 5 as Model B.14.

In various embodiments, a patient subphenotype classifier receives, as input, the following six input variables: heart rate-R, mean arterial pressure-R, respiratory rate-R, arterial pH-R, PaO₂—R, and bicarbonate-L. Such an example patient subphenotype classifier is described in Example 5 as Model B.15.

In various embodiments, a patient subphenotype classifier receives, as input, the following seven input variables: heart rate-R, mean arterial pressure-R, respiratory rate-R, arterial pH-R, PaO₂—R, PaCO₂—R, and bicarbonate-L. Such an example patient subphenotype classifier is described in Example 5 as Model B.16.

In various embodiments, a patient subphenotype classifier receives, as input, the following eight input variables: heart rate-R, mean arterial pressure-R, respiratory rate-R, arterial pH-R, PaO₂—R, FiO₂-R, bicarbonate-L, and creatinine-R. Such an example patient subphenotype classifier is described in Example 7 as Model C.1.

In various embodiments, a patient subphenotype classifier receives, as input, the following eight input variables: heart rate-R, mean arterial pressure-R, respiratory rate-R, arterial pH-R, PaO₂—R, FiO₂-R, bicarbonate-L, creatinine-R, and vasopressor use in the prior 24 hours. Such an example patient subphenotype classifier is described in Example 7 as Model C.2.

In various embodiments, a patient subphenotype classifier receives, as input, the following ten input variables: heart rate-R, mean arterial pressure-R, respiratory rate-R, arterial pH-R, PaO₂—R, FiO₂-R, bicarbonate-L, creatinine-R, age, gender, and vasopressor use in the prior 24 hours. Such an example patient subphenotype classifier is described in Example 7 as Model C.3.

In various embodiments, a patient subphenotype classifier receives, as input, the following nine input variables: heart rate-R, mean arterial pressure-R, respiratory rate-R, arterial pH-R, PaO₂—R, FiO₂-R, bicarbonate-L, creatinine-R, and bilirubin-H. Such an example patient subphenotype classifier is described in Example 7 as Model C.4.

In various embodiments, a patient subphenotype classifier receives, as input, the following eleven input variables: heart rate-R, mean arterial pressure-R, respiratory rate-R, arterial pH-R, PaO₂—R, FiO₂-R, bicarbonate-L, creatinine-R, bilirubin-H, age, and gender. Such an example patient subphenotype classifier is described in Example 7 as Model C.5.

In various embodiments, a patient subphenotype classifier receives, as input, the following fourteen input variables: heart rate-R, mean arterial pressure-R, respiratory rate-R, arterial pH-R, PaO₂—R, FiO₂-R, bicarbonate-L, creatinine-R, bilirubin-H, platelets-L, positive end-expiratory pressure-R, tidal volume-R, plateau pressure-R, and vasopressor use in the prior 24 hours. Such an example patient subphenotype classifier is described in Example 7 as Model C.6.

In various embodiments, a patient subphenotype classifier receives, as input, the following thirteen input variables: heart rate-R, mean arterial pressure-R, respiratory rate-R, arterial pH-R, PaO₂—R, FiO₂-R, bicarbonate-L, creatinine-R, platelets-L, positive end-expiratory pressure-R, tidal volume-R, plateau pressure-R, and vasopressor use in the prior 24 hours. Such an example patient subphenotype classifier is described in Example 7 as Model C.7.

In various embodiments, a patient subphenotype classifier receives, as input, the following fifteen input variables: heart rate-R, mean arterial pressure-R, respiratory rate-R, arterial pH-R, PaO₂—R, FiO₂-R, bicarbonate-L, creatinine-R, platelets-L, age, gender, positive end-expiratory pressure-R, tidal volume-R, plateau pressure-R, and vasopressor use in the prior 24 hours. Such an example patient subphenotype classifier is described in Example 7 as Model C.8.

In various embodiments, a patient subphenotype classifier receives, as input, the following sixteen input variables: heart rate-R, mean arterial pressure-R, respiratory rate-R, arterial pH-R, PaO₂—R, FiO₂-R, bicarbonate-L, creatinine-R, bilirubin-H, platelets-L, age, gender, positive end-expiratory pressure-R, tidal volume-R, plateau pressure-R, and vasopressor use in the prior 24 hours. Such an example patient subphenotype classifier is described in Example 7 as Model C.9.

In various embodiments, a patient subphenotype classifier receives, as input, the following fifteen input variables: heart rate-R, mean arterial pressure-R, respiratory rate-R, arterial pH-R, PaO₂—R, FiO₂-R, bicarbonate-L, creatinine-R, bilirubin-H, platelets-L, age, gender, tidal volume-R, plateau pressure-R, and vasopressor use in the prior 24 hours. Such an example patient subphenotype classifier is described in Example 7 as Model C.10.

In various embodiments, a patient subphenotype classifier receives, as input, the following fourteen input variables: heart rate-R, mean arterial pressure-R, respiratory rate-R, arterial pH-R, PaO₂—R, FiO₂-R, bicarbonate-L, creatinine-R, bilirubin-H, platelets-L, age, tidal volume-R, plateau pressure-R, and vasopressor use in the prior 24 hours. Such an example patient subphenotype classifier is described in Example 7 as Model C.11.

In various embodiments, a patient subphenotype classifier receives, as input, the following thirteen input variables: heart rate-R, mean arterial pressure-R, respiratory rate-R, arterial pH-R, PaO₂—R, FiO₂-R, bicarbonate-L, creatinine-R, bilirubin-H, platelets-L, age, plateau pressure-R, and vasopressor use in the prior 24 hours. Such an example patient subphenotype classifier is described in Example 7 as Model C.12.

In various embodiments, a patient subphenotype classifier receives, as input, the following twelve input variables: heart rate-R, mean arterial pressure-R, respiratory rate-R, PaO₂—R, FiO₂-R, bicarbonate-L, creatinine-R, bilirubin-H, platelets-L, age, plateau pressure-R, and vasopressor use in the prior 24 hours. Such an example patient subphenotype classifier is described in Example 7 as Model C.13.

In various embodiments, a patient subphenotype classifier receives, as input, the following eleven input variables: heart rate-R, mean arterial pressure-R, respiratory rate-R, PaO₂—R, FiO₂-R, creatinine-R, bilirubin-H, platelets-L, age, plateau pressure-R, and vasopressor use in the prior 24 hours. Such an example patient subphenotype classifier is described in Example 7 as Model C.14.

In various embodiments, a patient subphenotype classifier receives, as input, the following eleven input variables: heart rate-R, mean arterial pressure-R, respiratory rate-R, PaO₂—R, FiO₂-R, bicarbonate-L, creatinine-R, bilirubin-H, platelets-L, age, and plateau pressure-R. Such an example patient subphenotype classifier is described in Example 7 as Model C.15.

In various embodiments, a patient subphenotype classifier receives, as input, the following ten input variables: heart rate-R, mean arterial pressure-R, respiratory rate-R, PaO₂—R, FiO₂-R, creatinine-R, bilirubin-H, platelets-L, age, and plateau pressure-R. Such an example patient subphenotype classifier is described in Example 7 as Model C.16.

In various embodiments, the patient subphenotype classifier is composed of two or more submodels that enable the patient subphenotype classifier to generate a prediction. Here, each of the two or more submodels of the patient subphenotype classifier can analyze EHR data of the subject. In various embodiments, the two or more submodels of the patient subphenotype classifier each analyze different EHR data of the subject. In various embodiments, the two or more submodels of the patient subphenotype classifier each analyze same EHR data of the subject. In various embodiments, the patient subphenotype classifier is composed of two submodels. In various embodiments, the patient subphenotype classifier is composed of three submodels. In various embodiments, the patient subphenotype classifier is composed of four submodels. In various embodiments, the patient subphenotype classifier is composed of five submodels. In various embodiments, the patient subphenotype classifier is composed of six submodels. In various embodiments, the patient subphenotype classifier is composed of seven submodels. In various embodiments, the patient subphenotype classifier is composed of eight submodels. In various embodiments, the patient subphenotype classifier is composed of nine submodels. In various embodiments, the patient subphenotype classifier is composed of ten submodels.

In particular embodiments, the patient subphenotype classifier is composed of at least a first model that generates a preliminary prediction as to a subphenotype of the subject and a second model that generates a prediction as to the likely mortality of the subject. As used herein, such a first model that generates a preliminary prediction of the subphenotype of the subject is referred to as a subphenotyping submodel. For example, the preliminary prediction of the subphenotype can be an indication that identifies whether the subject is preliminarily determined to be in one of a plurality of classifications. As a specific example, the subphenotyping model may perform an unsupervised clustering analysis (e.g., K-means cluster) and therefore, subphenotyping model clusters the subject according to EHR data of the subject. Therefore, the classification corresponding to the cluster of the subject can serve as the preliminary prediction of the subphenotype of the subject.

Here, a second model that generates a prediction of the likely mortality of the subject is referred to as a mortality submodel. The mortality submodel can output a prediction of a mortality score. A mortality score can be indicative of a level of mortality risk for the subject. In various embodiments, the mortality score is between 0 and 1. For example, a mortality risk closer to 1 indicates a high risk of mortality for the subject, whereas a mortality risk closer to 0 indicates a lower risk of mortality for the subject. In various embodiments, the mortality score can be the prediction outputted by the patient subphenotype classifier. Thus, the mortality score can be compared to one or more threshold values to determine a classification for the subject.

In various embodiments, the subphenotyping submodel is constructed via unsupervised learning methods. For example, the subphenotyping submodel can be constructed using unsupervised K-means clustering methods. In various methods the mortality submodel is constructed via supervised learning models.

In various embodiments, the output of one of the submodels is provided as input to another one of the submodels. For example, the output of a subphenotyping submodel can be provided as input to a mortality submodel. As another example, the output of a mortality submodel can be provided as input to a subphenotyping submodel. In various embodiments, the patient subphenotype classifier includes multiple subphenotyping submodels and one mortality submodel. For example, the patient subphenotype classifier can include two subphenotyping submodels whose outputs serve as two inputs into a single mortality submodel. For example, the patient subphenotype classifier can include three subphenotyping submodels whose outputs serve as three inputs into a single mortality submodel. In various embodiments, the patient subphenotype classifier includes one subphenotyping submodel and multiple mortality submodels. For example, the patient subphenotype classifier can include one subphenotyping submodel whose output serves as an input into each of two mortality submodels.

Reference is made to FIG. 2B, which shows an example flow diagram involving the implementation of a classifier 230, in accordance with a second embodiment. Here, the classifier 230 (e.g., patient subtype classifier) can include multiple submodels, herein denoted as a subphenotyping submodel 240 and a mortality submodel 250. The classifier 230 receives, as input, EHR data 210 for a subject. The classifier 230 analyzes the EHR data 210 and outputs a prediction 220 for the subject. In various embodiments, the prediction 220 is a classification. For example, the prediction 220 is a classification of an ARDS subphenotype (e.g., subphenotype A or subphenotype B) for the subject. In various embodiments, the prediction 220 is a score (e.g., a mortality score) that is informative for determining a classification. As described herein, the score can be compared to one or more threshold values to determine the classification.

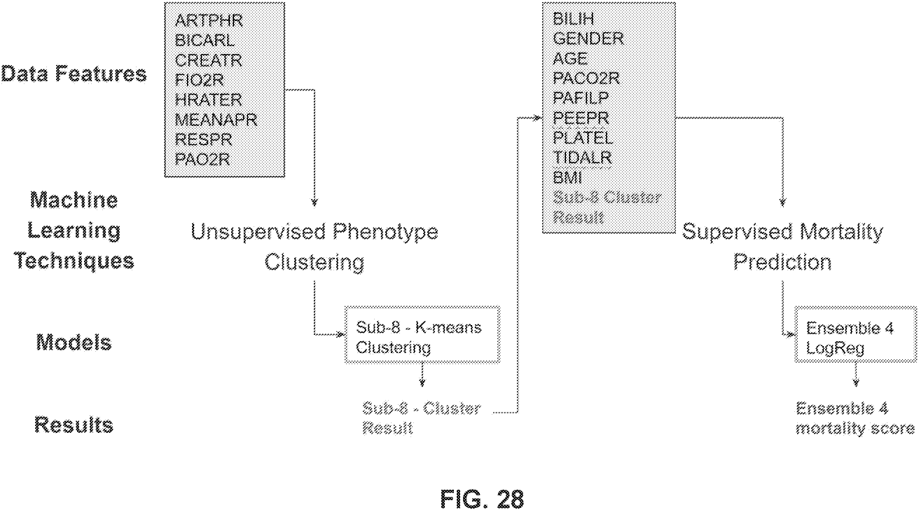

As shown in FIG. 2B, the classifier includes one subphenotyping submodel 240 whose output serves as input to one mortality submodel 250. The output of the mortality submodel 250 is the prediction 220 outputted by the classifier 230. Generally, each of the subphenotyping submodel 240 and the mortality submodel 250 receive, as input, EHR data 210. In various embodiments, the subphenotyping submodel 240 and the mortality submodel 250 receive, as input, different EHR data 210. Such an example of a classifier 230 including a subphenotyping submodel 240 and a mortality submodel 250 is described below in relation to FIG. 28.

In various embodiments, the subphenotyping submodel 240 can receive, as input, any of the combinations of EHR data described above in relation to the patient subphenotyping classifier. In particular embodiments, the subphenotyping submodel 240 receives the following eight EHR data as input: arterial pH-R, bicarbonate-L, creatinine-R, FiO₂-R, heart rate-R, mean arterial pressure-R, respiratory rate-R, and PaO₂—R. The subphenotyping submodel 240 analyzes the EHR data and outputs a preliminary prediction of the subphenotype of the subject. For example, the subphenotyping submodel 240 performs a clustering analysis (e.g., K-means clustering) and determines a preliminary prediction of the subphenotype of the subject according to the cluster in which the subject is located in.

In various embodiments, the mortality submodel 250 can receive, as input, any of the combinations of EHR data described above in relation to the patient subphenotyping classifier as well as the preliminary prediction of the subphenotype of the subject determined by the subphenotyping submodel 240. In particular embodiments, the mortality submodel 250 receives, as input, the following nine EHR data inputs: bilirubin-H, age, gender, PaCO₂—R, ratio of PaO₂—R/FiO₂-R, positive end-expiratory pressure-R, plateau pressure-R, tidal volume R, and body mass index (BMI). In addition to these nine EHR data inputs, the mortality submodel 250 receives the preliminary prediction of the subphenotype of the subject determined by the subphenotyping submodel 240.

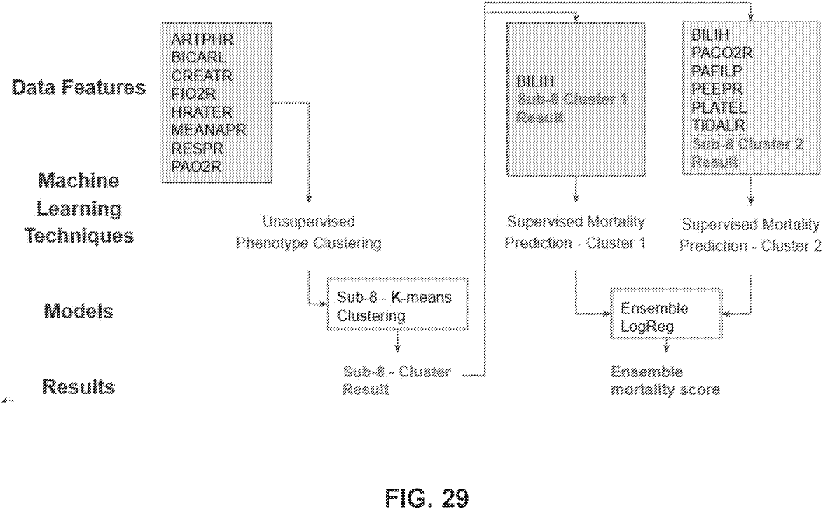

In various embodiments, the classifier 230 may include one subphenotyping submodel 240 and two mortality submodels 250. Here, the output of the subphenotyping model 240 can serve as inputs to each of the two mortality submodels 250. Such an example of a classifier 230 including a subphenotyping submodel 240 and two mortality submodels 250 is described below in relation to FIG. 29.

In various embodiments, the subphenotyping submodel can receive, as input, any of the combinations of EHR data described above in relation to the patient subphenotyping classifier. In particular embodiments, the subphenotyping submodel receives the following eight EHR data as input: arterial pH-R, bicarbonate-L, creatinine-R, FiO₂-R, heart rate-R, mean arterial pressure-R, respiratory rate-R, and PaO₂—R. The subphenotyping submodel analyzes the EHR data and outputs a preliminary prediction of the subphenotype of the subject. For example, the subphenotyping submodel performs a clustering analysis (e.g., K-means clustering) and determines a preliminary prediction of the subphenotype of the subject according to the cluster in which the subject is located in.

In various embodiments, each of the first and second mortality submodels 250 can receive, as input, any of the combinations of EHR data described above in relation to the patient subphenotyping classifier as well as the preliminary prediction of the subphenotype of the subject determined by the subphenotyping submodel. In particular embodiments, the first mortality submodel receives, as input, bilirubin-H and the preliminary prediction of the subphenotype of the subject determined by the subphenotyping submodel. In particular embodiments, the second mortality submodel receives, as input, the following six EHR data inputs: bilirubin-H, PaCO₂—R, ratio of PaO₂—R/FiO₂-R, positive end-expiratory pressure-R, tidal volume-R, and plateau pressure-R. The second mortality submodel further receives the preliminary prediction of the subphenotype of the subject determined by the subphenotyping submodel. Here, the outputs of each of the first mortality submodel and the second mortality submodels can be combined to produce a combined mortality score that is informative for classifying the subject.

Reference is made to FIG. 2C, which shows an example flow diagram involving the implementation of a classifier 230, in accordance with a third embodiment. Here, the classifier 230 (e.g., patient subtype classifier) can include multiple submodels. As shown in FIG. 2C, the classifier includes one subphenotyping submodel 240 whose output serves as input to one mortality submodel 250. Additionally, the classifier 230 includes mortality submodel 260 whose output also serves as input to mortality submodel 250. Generally, each of the subphenotyping submodel 240, mortality submodel 260, and mortality submodel 250 receive, as input, EHR data 210. In various embodiments, the subphenotyping submodel 240, mortality submodel 260, and mortality submodel 250 receive, as input, different EHR data 210. In various embodiments, subphenotyping submodel 240 and mortality submodel 260 receive the same EHR data as input but the mortality submodel 250 receives different EHR data.

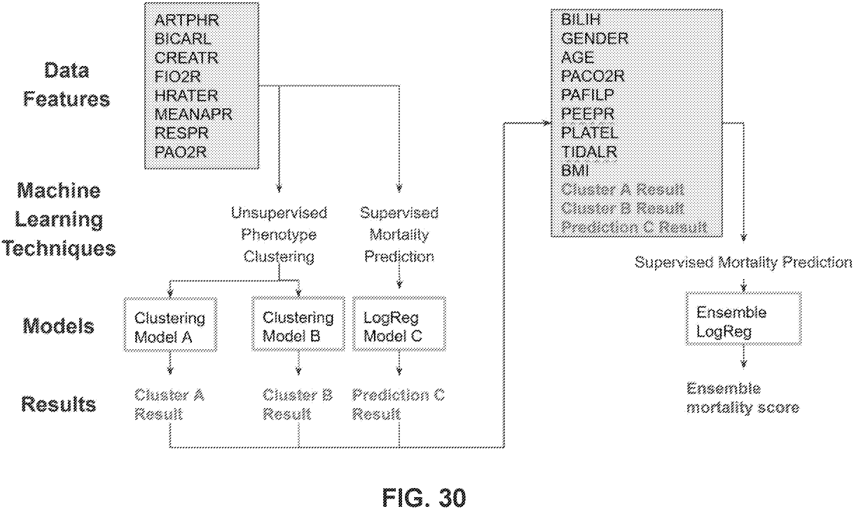

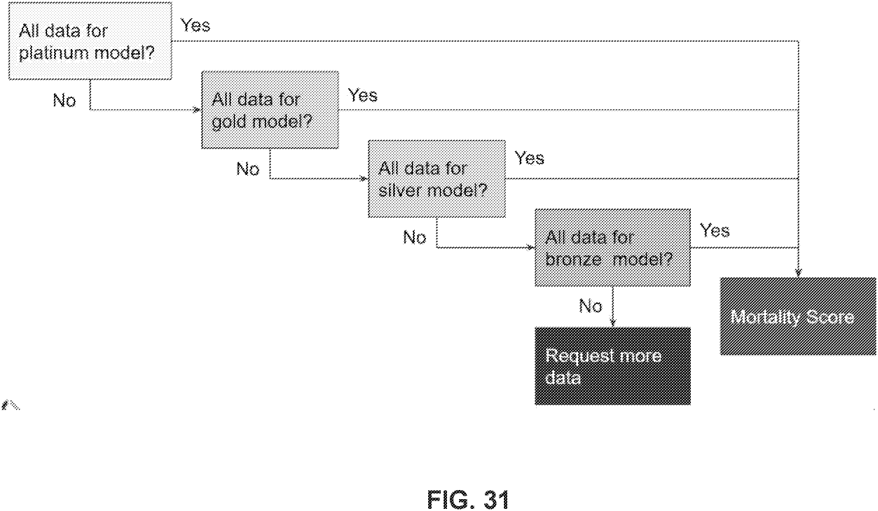

In various embodiments, a classifier 230 can include multiple subphenotyping submodels 240. For example, the classifier can include two subphenotyping submodels 240 as well as a mortality submodel 260 and mortality submodel 250. Such an example of a classifier 230 including two subphenotyping submodels 240, a mortality submodel 260, and a mortality submodel 250 is described below in relation to FIG. 30.

In various embodiments, the first subphenotyping submodel and the second subphenotyping submodel receive the same EHR data as input. For example, the first subphenotyping submodel and the second subphenotyping submodel receive, as input the following eight EHR data inputs: arterial pH-R, bicarbonate-L, creatinine-R, FiO₂-R, heart rate-R, mean arterial pressure-R, respiratory rate-R, and PaO₂—R. In various embodiments, the mortality submodel 250 receives as input the same eight EHR data inputs (e.g., arterial pH-R, bicarbonate-L, creatinine-R, FiO₂-R, heart rate-R, mean arterial pressure-R, respiratory rate-R, and PaO₂—R). Each of the outputs from the two subphenotyping models and the first mortality submodel (e.g., mortality submodel 260) are provided as input to a second mortality submodel (e.g., mortality submodel 250). In various embodiments, the mortality submodel 250 additionally receives as input the following nine EHR data inputs: bilirubin-H, age, gender, PaCO₂—R, ratio of PaO₂—R/FiO₂-R, positive end-expiratory pressure-R, plateau pressure-R, tidal volume R, and body mass index (BMI). Thus, the mortality submodel 250 receives a total of twelve inputs (e.g., 9 EHR data inputs and 3 inputs determined from other submodels). The mortality submodel 250 outputs a prediction, such as a mortality score that is informative for determining a classification of the subject.

### Training a Patient Subphenotype Classifier

As described herein, the model training module 150 as shown in FIG. 1B trains patient subphenotype classifiers. In various embodiments, a patient subphenotype classifier can be a discretely programmed model (e.g., a generalized linear model, a gradient boosting classifier, a neural network, a support vector machine, or a discriminative factor model). In some embodiments, the patient subphenotype classifier can be learned via unsupervised learning (e.g., latent class analysis, K-means clustering, principal component analysis, or unsupervised neural network). In particular embodiments, the patient subphenotype classifier is learned via K-means clustering. In some embodiments, the patient subphenotype classifier can be learned via supervised learning. For example, the patient subphenotype classifier can be a regression model or a supervised neural network. In particular embodiments, the patient subphenotype classifier is a Bayesian logistic regression model.

In various embodiments, patient subphenotype classifiers comprise a function and/or a plurality of parameters. The function captures the relationship between independent variables (e.g., EHR data) and dependent variables (e.g., a score or prediction) in the training dataset. The parameters modify the function, and are identified during training of the patient subphenotype classifier based on the training dataset. Generally, parameters of the patient subphenotype classifier are learned by a computer because it would be too difficult or too inefficient for the parameters to be identified by a human based on the training dataset due to the size and/or complexity of the training dataset. For example, if the patient subphenotype classifier is a K-means cluster, the parameters of the patient subphenotype classifier can be the positions of cluster centroids and observations assigned to each cluster.

The training dataset used to construct the patient subphenotype classifier can depend on the type of the patient subphenotype classifier. Generally, the training dataset comprises a plurality of training samples. Each training sample i from the training dataset is associated with a retrospective subject, and comprises EHR data for the retrospective subject. A retrospective subject is a subject for whom at least EHR data is known.

To train the patient subphenotype classifier, each training sample i from the training dataset is input into the patient subphenotype classifier. The patient subphenotype classifier processes these inputs as if the model were being routinely used to generate a prediction (e.g., a score). However, depending on the type of the patient subphenotype classifier, each training sample i of the training dataset may comprise additional components.

In embodiments in which the patient subphenotype classifier is learned via unsupervised learning, the patient subphenotype classifier is trained based on the basic training dataset described above. For example, in embodiments in which the patient subphenotype classifier is constructed via K-means clustering, an optimal number and configuration of clusters that both minimize differences between the training samples within each cluster, and maximize differences between the training samples between clusters, are determined. Specifically, in training the patient subphenotype classifier using K-means clustering, parameters θ that define the centroid of each cluster in the variable space of the patient subphenotype classifier are learned. Collectively, these parameters θ can mathematically modify the function to specify the dependence between independent variables (e.g., EHR data) and dependent variables (e.g., a prediction or score). The clinical significance of each cluster can be determined by examining the inputs to the patient subphenotype classifier that affect assignment of the inputs to clusters.

In embodiments in which the patient subphenotype classifier is learned via supervised learning, each training sample i from the training dataset further includes a retrospective classification (e.g., ARDS subphenotype classification) for the retrospective subject associated with the training sample. In other words, in embodiments in which the patient subphenotype classifier is learned via supervised learning, the patient subphenotype classifier is trained based in part on the known ARDS subphenotype classification of retrospective subjects associated with the training dataset.

In addition to training the patient subphenotype classifier to optimize a prediction of an ARDS subphenotype, in some embodiments, the patient subphenotype classifier can be trained to optimize other performance metrics. For example, the patient subphenotype classifier can also be trained to optimize fundamental predictive metrics, such as, for example, sensitivity and specificity of the prediction. Furthermore, the patient subphenotype classifier can be trained to optimize for any weighted combination of performance metrics.

Turning back to training of the patient subphenotype classifier using retrospective medical outcomes, after each iteration of the patient subphenotype classifier using a training sample i in the training dataset, the difference between the prediction output by the model and the retrospective classification of the retrospective subject is determined. Specifically, in embodiments in which the patient subphenotype classifier is configured to determine an ARDS classification for a subject, the patient subphenotype classifier determines the difference between the classification output by the model and the known retrospective classification for the retrospective subject.

The patient subphenotype classifier seeks to maximize improvement of the performance of the classifier by adjusting this difference between the predicted classification by the patient subphenotype classifier and the retrospective classification. For example, the patient subphenotype classifier seeks to maximize improvement by adjusting the difference between the predicted classification output by the model and the known retrospective classification. To adjust this difference, the patient subphenotype classifier can minimize or minimize a loss function for the patient subphenotype classifier. The loss function ℓ(u_{i∈S,,} θ) represents discrepancies between values of dependent variables u_{i∈S} for one or more training samples i in the training data S (e.g., known, retrospective classification). In simple terms, the loss function represents the difference between the prediction classification by the patient subphenotype classifier and the known, retrospective classification in the training dataset. There are a plurality of loss functions known to those skilled in the art, and any one of these loss functions can be utilized in generating the patient subphenotype classifier.

By minimizing or maximizing the loss function with respect to θ, values for a set of parameters θ can be determined. In some embodiments, the patient subphenotype classifier can be a parametric model in which the set of parameters θ mathematically modify the function to specify the dependence between independent variables (e.g., EHR data) and dependent variable (e.g., predicted classification). In other words, the set of parameters θ determined by minimizing or maximizing the loss function can be used to modify the function of the patient subphenotype classifier such that the outputted predicted classification is optimized. Typically, the parameters of parametric-type models that minimize or maximize the loss function are determined through gradient-based numerical optimization algorithms, such as batch gradient algorithms, stochastic gradient algorithms, and the like. Alternatively, the patient subphenotype classifier may be a non-parametric model in which the model structure is determined from the training dataset and is not strictly based on a fixed set of parameters.

In some embodiments, during training of the patient subphenotype classifier, one or more training samples i are automatically received at specified time intervals and the plurality of parameters of the patient subphenotype classifier are automatically identified using the received training samples i at specified time intervals, such that the patient subphenotype classifier is automatically updated at specified time intervals. In alternative embodiments, during training of the patient subphenotype classifier, one or more training samples i are automatically received in real-time, near real-time, delayed batch or on demand and the plurality of parameters are automatically identified in-real time using the received training samples i, such that the patient subphenotype classifier is automatically updated in-real time.

When the patient subphenotype classifier achieves a threshold level of prediction accuracy (e.g., when the predicted classifications determined by the model are sufficiently optimized), the patient subphenotype classifier is ready for use. To determine when the patient subphenotype classifier has achieved the threshold level of prediction accuracy sufficient for use, validation of the patient subphenotype classifier can be performed. Once the patient subphenotype classifier has been validated as having achieved the threshold level of prediction accuracy sufficient for use, in some embodiments, this does not preclude the model from continued training. In fact, in a preferred embodiment, despite validation, the patient subphenotype classifier continues to be automatically trained such that the set of parameters of the patient subphenotype classifier are automatically and continuously updated, such that the accuracy of the patient subphenotype classifier continues to improve.

### Electronic Health Record Data

Disclosed herein is the analysis of EHR data using patient subphenotype classifiers for predicting classifications for subjects. In various embodiments, EHR data can be collected and electronically recorded at any site prior to being provided as input into the patient subphenotype classifiers. In particular embodiments, the EHR data can be obtained from any private, public, and/or commercial source of EHR data. For example, the EHR data can be obtained from a private medical and/or health record and/or middleware system including a patient care center record system, a clinical laboratory record system, a research laboratory record system, such as EPIC®, Cerner®, Allscripts®, MedMined™, Beaker®, and Data Innovations®, and any alternative private medical and/or health record and/or middleware system. The EHR data can also be obtained from any publicly- and/or commercially-available source of EHR data, including published medical record databases and scientific publications such as PhysioNet datasets including the Multiparameter Intelligent Monitoring in Intensive Care (MIMIC) datasets, Philips eICU datasets, and National Heart, Lung, and Blood Institute Biospecimen and Data Repository Information Coordinating Center (BioLINCC) datasets. In various embodiments, the EHR data can include any of the ALVEOLI dataset, ARMA dataset, ARDSnet dataset, ARMA-KARMA-LARMA datasets, FACTT dataset, EDEN dataset, SAILS dataset, and ART dataset.

In certain embodiments, the EHR data received by the patient classifier system (e.g., patient classifier system 130 shown in FIGS. 1A and 1B) comprises an entire EHR dataset for a subject. However, in alternative embodiments, the EHR data comprises a select subset of the EHR data stored for a subject. For instance, the EHR data may solely comprise respiratory rate(s) for a subject. Similarly, in some embodiments, the EHR data can comprise EHR data received for a subject during a specified period of time. For example, in some embodiments, the EHR data may solely comprise data received for a subject over a 24-hour period of time.

In various embodiments, the EHR data can be received from multiple, distinct third-party sources and therefore, the EHR data may be represented in multiple, distinct data formats in accordance with the different third-party sources. For instance, EHR data for different subjects can be organized within different structures. As an example, in some embodiments, EHR data can be organized in delimited flat files, structured documents (e.g., JSON formatted documents), or relational databases. Furthermore, the labeling of EHR data within these different structures can differ as well. For example, in a first structure, heart rate data may be labeled as “HR,” while in a second, different structure, heart rate data may be labeled as “heart rate,” while in yet a third, different structure, heart rate data may be labeled in code. Even further, EHR data can be stored in different units. For example, a first set of EHR data describing temperature may be recorded in Fahrenheit units, while a second set of EHR data describing temperature may be recorded in Celsius units. To render all of these distinct data formats compatible with one another such that the data can be merged to form a single dataset and can be input into the patient subphenotype classifier, the distinct data formats can be transformed into a common data format. In some embodiments, the distinct data formats can be transformed into a common data format using a publicly-available data transformation model such as, for example, the OMOP Common Data Model.

In certain embodiments, prior to inputting the EHR data into the patient subphenotype classifier, the EHR data can be combined to create new EHR data. For example, the EHR data can be used to create new EHR data describing data trends over time. As another example, the EHR data can be used to create new EHR data comprising ratios or differences between different EHR data variables. In such embodiments, this new, combined EHR data can be input into the model.

In various embodiments, prior to inputting the EHR data into the patient subphenotype classifier, certain patients can be removed from analysis according to their EHR data. For example, in certain embodiments, the patient subphenotype classifier is only deployed to analyze a subset of ARDS patients. In various embodiments, a subset of ARDS patients are patients with any of mild, moderate, or severe ARDS. Patients with mild ARDS can be characterized by a P/F ratio between 200 and 300, where “P” refers to the partial pressure of oxygen (PaO₂) and “F” refers to the fraction of inspired oxygen (FiO₂). Patients with moderate ARDS can be characterized by a P/F ratio between 100 and 200. Patients with severe ARDS can be characterized by a P/F ratio less than 100. In various embodiments, patients with moderate to severe ARDS can be characterized by a P/F ratio ≤ 200. In various embodiments, patients with mild, moderate, or severe ARDS can be characterized by a P/F ratio ≤ 300. Thus, ARDS patients that are not included in the subset of ARDS patients are not analyzed.

In further embodiments, prior to inputting the EHR data into the patient subphenotype classifier, the EHR data is encoded. In some embodiments, the EHR data is encoded prior to being input into the patient subphenotype classifier. As one example, EHR data describing a heart rate of 60 beats/minute can be encoded in an array of bits as [111100]. As another example, EHR data can be encoded via K-means clustering. K-means clustering can serve to both de-identify subject EHR data, as well as to prevent effects of data-drift. For example, in a case in which EHR data describing mean and median subject body weight steadily increases, the EHR data can continuously undergo K-means clustering, and each identified cluster can be assigned a numeric index. Then, the actual subject body weight values are associated with the numeric indices, and can fluctuate over time and geography.

### Example Methods for Classifying Patients According to EHR Data

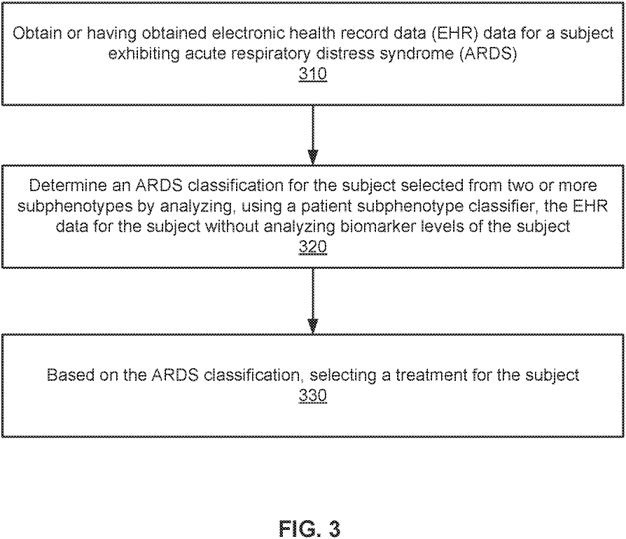

FIG. 3 is a flow process of classifying patients and determining a treatment prediction for a subject, in accordance with an embodiment. Step 310 involves obtaining or having obtained electronic health record (EHR) data for a subject exhibiting acute respiratory distress syndrome (ARDS). In various embodiments, the EHR data is obtained from a critical care setting (e.g., a hospital department such as an intensive care unit or emergency room) in which the subject is located. Step 320 involves determining an ARDS classification for the subject selected from two or more subphenotypes by analyzing, using a patient subphenotype classifier, the EHR data for the subject. For example, the patient subphenotype classifier may determine that the subject exhibits a first ARDS subphenotype out of two possible ARDS subphenotypes. As another example, the patient subphenotype classifier may determine that the subject exhibits a first ARDS subphenotype out of three possible ARDS subphenotypes. In various embodiments, the particular ARDS classification determined for the subject can be associated with underlying biology of the subject’s ARDS, such as any of hyperinflammation, hypoinflammation, hyperimmune response, or hypoimmune response.

Step 330 involves selecting a treatment for the subject based on the ARDS classification. For example, one or more treatments can be selected for administration to the subject based on the ARDS classification. As another example, one or more treatments can be selected to be withheld from the subject based on the ARDS classification. Example treatments include neuromuscular blockage (NMB) treatments, Positive End-Expiratory Pressure (PEEP), corticosteroids (e.g., methylpredinosolone or dexamethasone), lisofylline, ketoconazole, catheter and fluid treatment, recruitment maneuver, or statins. Guided therapy based on the ARDS classification is described in further detail herein.

### Patient Subphenotypes

Disclosed herein are methods, non-transitory computer readable media, and systems for classifying subjects into different ARDS patient subphenotypes by implementing a patient subphenotype classifier. In various embodiments, the patient subphenotype classifier classifies a subject into one out of two possible ARDS subphenotypes. In various embodiments, the patient subphenotype classifier classifies a subject into one out of three possible ARDS subphenotypes. In various embodiments, the patient subphenotype classifier classifies a subject into one out of four possible ARDS subphenotypes. In various embodiments, the patient subphenotype classifier classifies a subject into one out of five possible ARDS subphenotypes. In various embodiments, the patient subphenotype classifier classifies a subject into one out of more than five possible ARDS subphenotypes.

In various embodiments, ARDS subphenotypes are associated with certain biological processes of ARDS. For example, an ARDS subphenotype can be associated with a particular inflammatory response. As another example, an ARDS subphenotype can be associated with a particular immune response.

In particular embodiments, an ARDS subphenotype for a subject, herein referred to as subphenotype A, corresponds to a hypoinflammatory state. In some scenarios, a hypoinflammatory ARDS subphenotype can be correlated with better outcomes (e.g., lower mortality). In particular embodiments, an ARDS subphenotype for a subject, herein referred to as subphenotype B, corresponds to a hyperinflammatory state. In some scenarios, a hyperinflammatory ARDS subphenotype can be correlated with worse outcomes (e.g., higher mortality).

In various embodiments, ARDS subphenotypes are associated with different patient outcomes. For example, an ARDS subphenotype can be associated with better outcomes and therefore, can be referred to as a lower risk group subphenotype. As another example, an ARDS subphenotype can be associated with intermediate outcomes and therefore, can be referred to as a medium risk group. As another example, an ARDS subphenotype can be associated with worse outcomes and therefore, can be referred to as a higher risk group.

In various embodiments, different ARDS subphenotypes can be characterized by differences in expression levels of one or more biomarkers. For example, if ARDS subphenotypes as are associated with certain underlying biological processes of ARDS (e.g., inflammation or immune response), the ARDS subphenotypes can be further characterized by different expression levels in biomarkers associated with those biological processes. In various embodiments, the biomarkers can include one or more of intercellular adhesion molecule-1 (ICAM-1), interleukin-6 (IL-6), plasminogen activator inhibitor-1 (PAI-1), interleukin-8 (IL-8), interleukin-10 (IL-10); tumor necrosis factor receptor 1 (TNFR-I); tumor necrosis factor II (TNFR-II), or von Willebrand factor (VW). In particular embodiments, an ARDS subphenotype associated with a hyperinflammatory state (e.g., subphenotype B) can be characterized by increased expression levels of inflammatory markers such as one or more of ICAM-1, IL-6, PAI-1, IL-6, IL-8, IL-10, TNFR-I, TNFR-II, and VW. In particular embodiments, an ARDS subphenotype associated with a hypoinflammatory state (e.g., subphenotype A) can be characterized by decreased expression levels of inflammatory markers such as one or more of ICAM-1, IL-6, PAI-1, IL-6, IL-8, IL-10, TNFR-I, TNFR-II, and VW.

### Guided Treatments According to Patient Subphenotypes

Methods disclosed herein involve classifying a subject into one of two or more ARDS subphenotypes using a patient subphenotype classifier that analyzes EHR data of the subject. In various embodiments, the ARDS classification of the subject, is useful for guiding a treatment selection for the subject. For example, the ARDS classification can be useful for selecting a treatment for providing to the subject. As another example, the ARDS classification can be useful for determining whether a treatment is to be withheld from a subject.

In various embodiments, the ARDS classification of the subject is useful for guiding an ARDS treatment for the subject, including any one of a neuromuscular blockage (NMB) therapy, positive end-expiratory pressure (PEEP) therapy, corticosteroid therapy (e.g., methylprednisolone or dexamethasone), lisofylline, ketoconazole, catheter and fluid treatment, recruitment maneuver, statins, and feeding/nutrition.

In particular embodiments, depending on the ARDS classification, the selected treatment is to administer NMB therapy. In particular embodiments, the selected treatment is to withhold NMB therapy. In particular embodiments, the selected treatment is to administer either high PEEP or low PEEP. In particular embodiments, the selected treatment is to only administer low PEEP. In particular embodiments, the selected treatment is to administer methylprednisolone. In particular embodiments, the selected treatment is to withhold methylprednisolone. In particular embodiments, the selected treatment is to administer dexamethasone. In particular embodiments, the selected treatment is to withhold dexamethasone. In particular embodiments, the selected treatment is to withhold lisofylline. In particular embodiments, the selected treatment is to administer lisofylline. In particular embodiments, the selected treatment is to administer ketoconazole. In particular embodiments, the selected treatment is to withhold ketoconazole. In particular embodiments, the selected treatment is to provide liberal or conservative fluid management. The liberal or conservative fluid management can be provided through either a pulmonary artery catheter (PAC) or central venous catheter (CVC) line. In particular embodiments, the selected treatment is to withhold a combination of PAC line and liberal fluid. In particular embodiments, the selected treatment is to provide recruitment maneuver. In particular embodiments, the selected treatment is to withhold recruitment maneuver. In particular embodiments, the selected treatment is to administer statins. In particular embodiments, the selected treatment is to administer statins at any time. In particular embodiments, the selected treatment is to administer statins as early as possible, even prior to ARDS diagnosis (if no contraindications). In particular embodiments, the selected treatment is to administer full feeding. In particular embodiments, the selected treatment is to administer full or enteral feeding.

Table 1 below shows particular guided therapies according to the patient subphenotypes of subphenotype A and subphenotype B in accordance with an embodiment.

TABLE 1

Guided therapies according to patient subphenotypes

| | | |
|---|---|---|
| Treatment | Subphenotype B (high mortality risk) | Subphenotype A (low mortality risk) |
| Neuromuscular blockage (NMB) | No NMB therapy or administer NMB therapy | Administer NMB therapy |
| Positive End-Expiratory Pressure (PEEP) | High PEEP or low PEEP | Administer Low PEEP |
| Methylpredinosolone | No treatment or administer methylprednisolone | No methylprednisolone |
| Dexamethasone (in Covid-19 induced ARDS) | Administer dexamethasone | No treatment or administer dexamethasone |
| Lisofylline | No lisofylline | No treatment or administer lisofylline |
| Ketoconazole | Administer ketoconazole | No treatment or administer ketoconazole |
| Catheter and Fluid | Pulmonary artery catheter (PAC) or central venous catheter (CVC) line Liberal or conservative fluid management | Do not treat with combination of PAC line and liberal fluid |
| Recruitment Maneuver | Consider recruitment maneuver | No recruitment maneuver |
| Statins | Administer statins at any time | Administer statins as early as possible, even prior to ARDS diagnosis (if no contraindications) |
| Enteral Feeding | Full Feeding or Trophic Feeding | Full Feeding |

### Example Computer and System

The methods disclosed herein, are, in some embodiments, performed on one or more computers or computer systems. For example, the training and implementation of a patient subphenotype classifier can be implemented in hardware or software, or a combination of both. In one embodiment of the invention, a machine-readable storage medium is provided, the medium comprising a data storage material encoded with machine readable data which, when using a machine programmed with instructions for using said data, is capable of displaying any of the datasets and execution and results of the models described herein. The invention can be implemented in computer programs executing on programmable computers, comprising a processor, a data storage system (including volatile and non-volatile memory and/or storage elements), a graphics adapter, a pointing device, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high-level procedural or object-oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

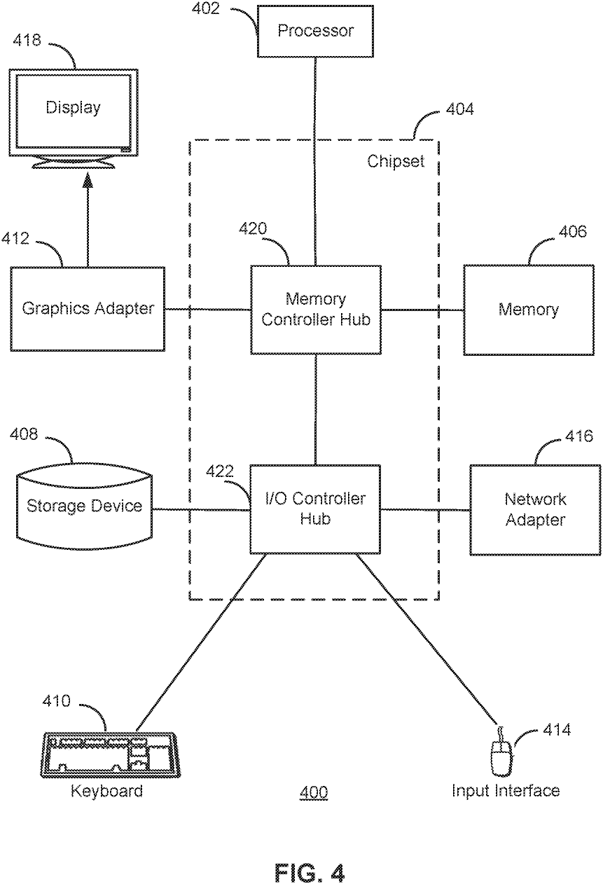

FIG. 4 illustrates an example computer for implementing the entities shown in FIGS. 1-3. The computer 400 includes at least one processor 402 coupled to a chipset 404. The chipset 404 includes a memory controller hub 420 and an input/output (I/O) controller hub 422. A memory 406 and a graphics adapter 412 are coupled to the memory controller hub 420, and a display 418 is coupled to the graphics adapter 412. A storage device 408, an input device 414, and network adapter 416 are coupled to the I/O controller hub 422. Other embodiments of the computer 400 have different architectures.

The storage device 408 is a non-transitory computer-readable storage medium such as a hard drive, compact disk read-only memory (CD-ROM), DVD, or a solid-state memory device. The memory 406 holds instructions and data used by the processor 402. The input interface 414 is a touch-screen interface, a mouse, track ball, or other type of pointing device, a keyboard, or some combination thereof, and is used to input data into the computer 400. In some embodiments, the computer 400 may be configured to receive input (e.g., commands) from the input interface 414 via gestures from the user. The graphics adapter 412 displays images and other information on the display 418. The network adapter 416 couples the computer 400 to one or more computer networks.

The computer 400 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 408, loaded into the memory 406, and executed by the processor 402.

The types of computers 400 used by the entities of FIGS. 1 or 2 can vary depending upon the embodiment and the processing power required by the entity. For example, the patient classifier system 130 can run in a single computer 400 or multiple computers 400 communicating with each other through a network such as in a server farm. The computers 400 can lack some of the components described above, such as graphics adapters 412, and displays 418.

### ADDITIONAL EMBODIMENTS

In one aspect, the disclosure provides a method for determining a subphenotype classification of a subject exhibiting acute respiratory distress syndrome (ARDS). ARDS is respiratory failure with rapid onset of widespread inflammation in the lungs. ARDS is not triggered by a single pathology-ARDS can be caused by sepsis, pneumonia, trauma, aspiration, pancreatitis, and/or other insults. A subject can be classified as subphenotype A or subphenotype B.

To classify a subject exhibiting ARDS as subphenotype A or subphenotype B, electronic health record (EHR) data is obtained for the subject. EHR data for a subject comprises an electronically-recorded set of medical and/or health information for the subject. EHR data can comprise any type of medical and/or health data for a subject, and can be collected by any means. For example, EHR data can be collected and electronically recorded at a patient care center (e.g., a physician’s office, the emergency department of a hospital, the intensive care unit of a hospital, the ward of a hospital), a clinical laboratory, a research laboratory, a remote consumer medical device, a therapeutic device (e.g., an infusion pump), a monitoring device such as a wearable device (e.g., a heart rate monitor), and any other site. EHR data can also be obtained from any private, public, and/or commercial source. In a preferred embodiment, the EHR data obtained for the subject comprises data that is routinely collected as standard-of-care for ARDS treatment. For instance, in a preferred embodiment, the EHR data obtained for the subject does not include data which must be measured outside of lab work and clinical data typically involved in standard-of-care for ARDS (e.g., with a dedicated blood test).

The EHR data for the subject is used by a patient subphenotype classifier to determine a subphenotype classification of the subject. In other words, based on the subject’s EHR data, a patient subphenotype classifier classifies the subject as subphenotype A or subphenotype B.

In alternative embodiments, rather than determining a classification of the subject exhibiting ARDS, the classification of the subject can be simply obtained. For example, in some embodiments, the classification of the subject can be pre-determined (e.g., already known).

In some embodiments, a mortality prognosis can be determined for the subject based at least in part on the classification of the subject as subphenotype A or subphenotype B. Specifically, in some embodiments, a subject classified as subphenotype B can be determined to have a mortality prognosis of high mortality risk, while a subject classified as subphenotype A can be determined to have a mortality prognosis of low mortality risk. In certain embodiments, low mortality risk can comprise at least one of reduced risk of hospital mortality, reduced risk of ICU mortality, reduced risk of 28-day mortality, reduced risk of 90-day mortality, reduced risk of 180-day mortality, and reduced risk of 6-month mortality relative to high mortality risk. In some further embodiments, low mortality risk can further comprise positive patient outcome, high mortality risk can further comprise negative patient outcome, and positive patient outcome can comprise at least one of shorter hospital length of stay, shorter ICU length of stay, and more ventilator-free days relative to negative patient outcome.

In some embodiments, a treatment recommendation can be determined for the subject based at least in part on the classification of the subject as subphenotype A or subphenotype B. Specifically, in some embodiments, the treatment recommendation for a subject classified as subphenotype B can be at least neuromuscular blockade (NMB) therapy, while the treatment recommendation for a subject classified as subphenotype A can be at least no NMB therapy. In certain embodiments, identifying the treatment recommendation for the subject can further include administering or having administered therapy to the subject based on the treatment recommendation.

In some embodiments, the patient subphenotype classifier can comprise one of a Model 1, a Model 2, a Model 3, a Model 4, a Model 5, or a Model 6. In embodiments in which the patient subphenotype classifier comprises the Model 1, the EHR data for the subject can include 13 input variables. In embodiments in which the patient subphenotype classifier comprises the Model 2, the EHR data for the subject can include 8 input variables. In embodiments in which the patient subphenotype classifier comprises the Model 3, the EHR data for the subject can include 17 input variables. In embodiments in which the patient subphenotype classifier comprises the Model 4, the EHR data for the subject can include 13 input variables. In embodiments in which the patient subphenotype classifier comprises the Model 5, the EHR data for the subject can include 9 input variables. In embodiments in which the patient subphenotype classifier comprises the Model 6, the EHR data for the subject can include 16 input variables.

In embodiments in which the patient subphenotype classifier comprises the Model 1, the EHR data for the subject can include the subject’s arterial pH, bicarbonate, creatinine, diastolic blood pressure (BP), FiO₂, heart rate, highest mean arterial pressure, lowest mean arterial pressure, potassium, highest respiratory rate, lowest respiratory rate, oxygen saturation (SPO₂), and systolic BP. More specifically, in some embodiments in which the patient subphenotype classifier comprises the Model 1, the EHR data for the subject can include the subject’s most recent arterial pH, lowest bicarbonate, most recent creatinine, most recent diastolic blood pressure (BP), most recent FiO₂, most recent heart rate, highest mean arterial pressure, lowest mean arterial pressure, most recent potassium, highest respiratory rate, lowest respiratory rate, most recent SPO₂, and most recent systolic BP.

In embodiments in which the patient subphenotype classifier comprises the Model 2, the EHR data for the subject can include the subject’s arterial pH, bicarbonate, creatinine, FiO₂, heart rate, PaO₂, mean arterial pressure, and respiratory rate. More specifically, in some embodiments in which the patient subphenotype classifier comprises the Model 2, the EHR data for the subject can include the subject’s most recent arterial pH, lowest bicarbonate, most recent creatinine, most recent FiO₂, most recent heart rate, most recent PaO₂, most recent mean arterial pressure, and most recent respiratory rate.

In embodiments in which the patient subphenotype classifier comprises the Model 3, the EHR data for the subject can include the subject’s age, arterial pH, bicarbonate, bilirubin, BMI, creatinine, FiO₂, gender, heart rate, PaCO₂, PaO₂/FiO₂, PaO₂, positive end-expiratory pressure (PEEP), platelet count, tidal volume, mean arterial pressure, and respiratory rate. More specifically, in some embodiments in which the patient subphenotype classifier comprises the Model 3, the EHR data for the subject can include the subject’s age, most recent arterial pH, lowest bicarbonate, highest bilirubin, BMI, most recent creatinine, most recent FiO₂, gender, most recent heart rate, most recent PaCO₂, lowest PaO₂/FiO₂ within 24 hours following ARDS diagnosis, most recent PaO₂, most recent positive end-expiratory pressure (PEEP), lowest platelet count, lowest tidal volume, most recent mean arterial pressure, and most recent respiratory rate.

In embodiments in which the patient subphenotype classifier comprises the Model 4, the EHR data for the subject can include the subject’s arterial pH, bicarbonate, BMI, creatinine, Fi 02, gender, heart rate, PaCO₂, PaO₂/FiO₂, PEEP, platelet count, mean arterial pressure, and respiratory rate. More specifically, in some embodiments in which the patient subphenotype classifier comprises the Model 4, the EHR data for the subject can include the subject’s most recent arterial pH, most recent bicarbonate, BMI, most recent creatinine, most recent FiO₂, gender, most recent heart rate, most recent PaCO₂, lowest PaO₂/FiO₂ within 24 hours following ARDS diagnosis, most recent PEEP, lowest platelet count, most recent mean arterial pressure, and most recent respiratory rate.

In embodiments in which the patient subphenotype classifier comprises the Model 5, the EHR data for the subject can include the subject’s arterial pH, bicarbonate, creatinine, FiO₂, heart rate, PaO₂, mean arterial pressure, bilirubin, and respiratory rate. More specifically, in some embodiments in which the patient subphenotype classifier comprises the Model 5, the EHR data for the subject can include the subject’s most recent arterial pH, lowest bicarbonate, most recent creatinine, most recent FiO₂, most recent heart rate, most recent PaO₂, most recent mean arterial pressure, highest bilirubin, and most recent respiratory rate.

In embodiments in which the patient subphenotype classifier comprises the Model 6, the EHR data for the subject can include the subject’s age, arterial pH, bicarbonate, bilirubin, creatinine, FiO₂, gender, heart rate, PaCO₂, PaO₂/FiO₂, PaO₂, positive end-expiratory pressure (PEEP), platelet count, tidal volume, mean arterial pressure, and respiratory rate. More specifically, in some embodiments in which the patient subphenotype classifier comprises the Model 6, the EHR data for the subject can include the subject’s age, most recent arterial pH, lowest bicarbonate, highest bilirubin, most recent creatinine, most recent FiO₂, gender, most recent heart rate, most recent PaCO₂, lowest PaO₂/FiO₂ within 24 hours following ARDS diagnosis, most recent PaO₂, most recent positive end-expiratory pressure (PEEP), lowest platelet count, lowest tidal volume, most recent mean arterial pressure, and most recent respiratory rate.

In embodiments in which the patient subphenotype classifier comprises the Model 1, the patient subphenotype classifier can have at least one of an area under receiver-operator curve (AUROC) of greater than or equal to 0.67 and an area under the precision-recall curve (AUPRC) of greater than or equal to 0.40.

In embodiments in which the patient subphenotype classifier comprises the Model 2, the patient subphenotype classifier can have at least one of an AUROC greater than or equal to 0.69 and an AUPRC greater than or equal to 0.42.

In embodiments in which the patient subphenotype classifier comprises the Model 3, the patient subphenotype classifier can have at least one of an AUROC greater than or equal to 0.71 and an AUPRC greater than or equal to 0.62

In embodiments in which the patient subphenotype classifier comprises the Model 4, the patient subphenotype classifier can have at least one of an AUROC greater than or equal to 0.67 and an AUPRC greater than or equal to 0.46.

In some embodiments, the patient subphenotype classifier can comprise a machine-learned model. For example, in certain embodiments, the patient subphenotype classifier can comprise at least one of a k-means clustering classifier, a logistic regression classifier, a decision tree classifier, a random forest classifier, a gradient boosting classifier, a neural network, and any other machine-learned classifier trained to determine the classification of the subject based on the EHR data.

In various embodiments, the patient subphenotype classifier is an ensemble-based model comprising two or more machine learning models. In various embodiments, an output of a first of the two or more machine learning models is used as input to a second of the two or more machine learning models. In various embodiments, a first of the two or more machine learning models of the ensemble-based model is implemented responsive to determining that data elements of the first of the two or more machine learning models are available in the EHR data. In various embodiments, a second of the two or more machine learning models of the ensemble-based model is implemented responsive to: determining that data elements of a first of the two or more machine learning models is unavailable in the EHR data; and determining that data elements of the second of the two or more machine learning models are available in the EHR data. In various embodiments, the first of the two or more machine learning models comprises more features than the second of the two or more machine learning models.

In various embodiments, subphenotype A and subphenotype B are characterized by differences in expression levels in one or more biomarkers. In various embodiments, the one or more biomarkers comprise one or more of PAI-1, IL-6, IL-8, IL-10, TNFR-I, TNFR-II, ICAM-1, or von Willebrand factor. In various embodiments, the one or more biomarkers comprise each of PAI-1, IL-6, IL-8, IL-10, TNFR-I, TNFR-II, ICAM-1, or von Willebrand factor.

Any of the steps of the method described above may be performed by any party and/or at the direction of any party. For instance, in certain embodiments, the steps of the method described above can be performed at the direction of any third-party, such as a provider of the patient subphenotype classifier. In certain further embodiments, the steps of the method described above can have been previously performed at the direction of any third-party, such as a provider of the patient subphenotype classifier.

In another aspect, the disclosure provides a computer-implemented method, including any combination of the steps mentioned above.

In another aspect, the disclosure provides a non-transitory computer-readable storage medium storing computer program instructions that when executed by a computer processor, cause the computer processor to perform any combination of the steps mentioned above.

In another aspect, the disclosure provides a system that includes a storage memory and a processor communicatively coupled to the storage memory. The storage memory is configured to store the EHR data of the subject. The processor is configured to determine the classification of the subject based on the subject’s EHR data stored in the storage memory, as discussed above. In some embodiments, the processor can be further configured to identify the treatment recommendation for the subject based at least in part on the determined classification, as discussed above. In some additional embodiments, the processor can be further configured to identify the mortality prognosis for the subject based at least in part on the determined classification, as discussed above.

Any terms not directly defined herein shall be understood to have the meanings commonly associated with them as understood within the art of the invention. Certain terms are discussed herein to provide additional guidance to the practitioner in describing the compositions, devices, methods and the like of aspects of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the invention herein.

It must be noted that, as used in the specification, the singular forms “a,” “an” and “the” include plural referents unless the context clearly dictates otherwise.

All references, issued patents and patent applications cited within the body of the specification are hereby incorporated by reference in their entirety, for all purposes.

The foregoing description of the embodiments of the disclosure has been presented for the purpose of illustration; it is not intended to be exhaustive or to limit the disclosure to the precise forms disclosed. Persons skilled in the relevant art can appreciate that many modifications and variations are possible in light of the above disclosure.

Some portions of this description describe the embodiments of the disclosure in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like.

Any of the steps, operations, or processes described herein can be performed or implemented with one or more hardware or software modules, alone or in combination with other devices. In one embodiment, a software module is implemented with a computer program product including a computer-readable non-transitory medium containing computer program code, which can be executed by a computer processor for performing any or all of the steps, operations, or processes described.

Embodiments may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments of the disclosure may also relate to a product that is produced by a computing process described herein. Such a product may include information resulting from a computing process, where the information is stored on a non-transitory, tangible computer-readable storage medium and may include any embodiment of a computer program product or other data combination described herein.

Finally, the language used in the specification has been principally selected for readability and instructional purposes, and it may not have been selected to delineate or circumscribe the inventive subject matter. It is therefore intended that the scope of the disclosure be limited not by this detailed description, but rather by any claims that issue on an application based hereon. Accordingly, the disclosure of the embodiments of the disclosure is intended to be illustrative, but not limiting, of the scope of the disclosure.

### EXAMPLES

### Example 1: Example K-Means Cluster ARDS Classifiers Differentiate Patient Populations and Guide Neuromuscular Blockade Therapy

Acute Respiratory Distress Syndrome (ARDS) is respiratory failure with rapid onset of widespread inflammation in the lungs. ARDS is not triggered by a single pathology-- it can be caused by sepsis, pneumonia, trauma, aspiration, pancreatitis, and/or other insults. Based on the hypothesis that the evaluation of ARDS subphenotypes may allow for identifying subgroups that are more homogeneous with respect to pathogenesis, and that this could potentially provide insights into patient outcomes, multiple machine learning-derived electronic health record (EHR)-based classifiers (i.e., “Models”) were developed that are capable of classifying patients into ARDS subphenotypes.

Via post-hoc analysis of the ARDSnet ALVEOLI (available at the URL: https://biolincc.nhlbi.nih.gov/studies/alveoli/), ARMA-KARMA-LARMA (available at the URL https://biolincc.nhlbi.nih.gov/studies/ardsnet/), FACTT (available at the URL https://biolincc.nhlbi.nih.gov/studies/factt/) datasets, the eICU dataset (available at the URL: eicu-crd.mit.edu/about/eicu/), the Brazilian ART dataset (available at the URL: www.ncbi.nlm.nih.gov/pubmed/28973363), and privately-provided data from the Cleveland Clinic, these Models are able to elucidate differential mortality rates in ARDS patients. Models were created using K-means clustering, with each model resulting in 2 clusters. One cluster showed a group of patients with worse sickness and worse outcomes, including higher mortality (i.e., “subphenotype B”) while the second cluster showed a distinctly separate pattern of less severe sickness and generally better outcomes, including lower mortality (i.e., “subphenotype A”). In the Model utilizing the minimal amount of EHR data (Model 2), mortality rates were significantly different, at 20.75% and 35.57% in subphenotype A and subphenotype B, respectively (binomial p-value: 1.0e-08), in a mixed training set from the three ARDSnet datasets. In the holdout dataset from the same three ARDSnet datasets, mortality rates were 23.43% and 38.57% in subphenotype A and subphenotype B, respectively (binomial p-value: 3.6e-03). Similar significant differences in morality were seen in eICU and ART datasets.

Current standard practice dictates that a patient should receive neuromuscular blockade (NMB) therapy if they have a P/F ratio < 150 and FiO₂ > 0.6. Across three datasets with NMB information available, mortality rates were 31% for patients whose treatment followed that protocol, and 29% in patients where the protocol was not followed. Patient classification is proposed herein as a new treatment guidance, wherein patients assigned to subphenotype B should receive NMB and patients assigned to subphenotype A should not. Using those guidelines, mortality was significantly reduced when the protocol was followed (28% and 36% in subphenotype B and subphenotype A, respectively (p = 0.002957)).

Overall, this work demonstrates the potential of employing an EHR-based subphenotyping classifier to identify subgroups of patients with varying mortality using readily available data. Patient subphenotype information can be combined with treatment and outcome information to identify populations of patients who have differential responses to therapy and ultimately improve treatment guidance and patient outcomes.

### Implementation

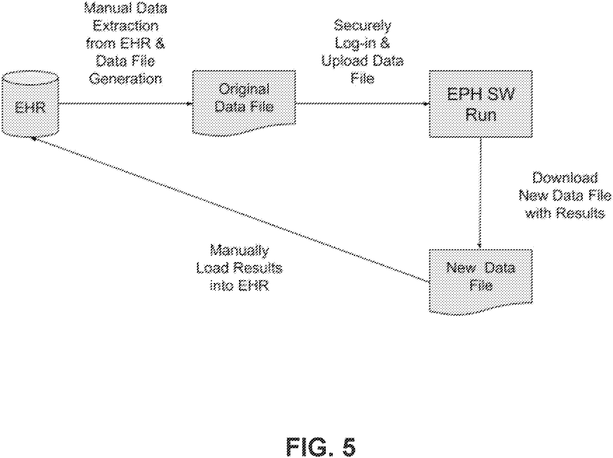

Briefly, patients are flagged for ARDS classification by one or more of Models 1-6 (e.g., patients eligible for ARDS classification by one or more of Models 1-6 are identified), and then a call of the one or more Models is made for that patient at a specific time for subphenotyping. This can be accomplished via batch integration or real-time integration. Batch integration includes collecting a batch of patients for which to run the one or more Models. Real-time integration includes continuously identifying patients for which to run the one or more Models. Batch integration can be done manually or can be automated. FIG. 5 depicts an example process flow for manual batch integration.

Furthermore, the following describes one embodiment of an example of classification of a patient via real-time integration of one or more of the Models 1-6:
1. Patient is admitted to hospital.
2. Clinical Decision Support System receives an Admission-Discharge-Transfer (ADT) message via current interoperability standards (e.g., HL7V2 or FHIR) and begins tracking the patient’s EHR.
3. The Clinical Decision Support System evaluates the patient’s EHR for inclusion criteria. Specifically, the Clinical Decision Support System determines whether the patient is on a ventilator, and whether the patient attains various clinical criteria such ARDS diagnosis, P/F ratio below a predetermined threshold, and/or any other clinical criteria. The Clinical Decision Support System identifies the patient for classification by one or more of the Models 1-6 based on the inclusion criteria.
4. The one or more Models 1-6 classify the patient.

The following describes of an example of classification of patients via batch integration of one or more of the Models 1-6:
1. Patients are admitted to hospital.
2. Hospital IT System evaluates the patients’ EHR for inclusion criteria. Specifically, the Hospital IT System determines whether the patients are on a ventilator, and whether the patients attain various clinical criteria such ARDS diagnosis, P/F ratio below a predetermined threshold, and/or any other clinical criteria. The Hospital IT System identifies patients for classification by one or more of the Models 1-6 based on the inclusion criteria.
3. The Hospital IT System creates a batch file with anonymized patient IDs and patient input variables to be processed by the one or more Models 1-6.
4. The Hospital IT System automatically uploads the batch file to Clinical Decision Support System to be processed by the one or more Models 1-6, or a user manually uploads the batch file to Clinical Decision Support System to be processed by the one or more Models 1-6. The batch file is available to the hospital automatically and/or for manual download via a secure cloud-based web application of the Clinical Decision Support System.
5. The one or more Models 1-6 classify the patients.

The following describes an example of prognostic classification of a patient by one or more of the Models 1-6
1. Patient is admitted to hospital.
2. The one or more Models 1-6 classify the patient into Subphenotype A or Subphenotype B by evaluation of the patient’s EHR.
3. The patient’s classification is provided to the hospital via Clinical Decision Support System and/or Hospital IT System.

The following describes an example of predictive (therapy guidance) classification of a patient by one or more of the Models 1-6:
1. Patient is admitted to hospital.
2. The one or more Models 1-6 classify the patient into Subphenotype A or Subphenotype B by evaluation of the patient’s EHR, and thus recommend NMB therapy (for Subphenotype B patients) or recommend no NMB therapy (for Subphenotype A patients).
3. The patient’s classification and NMB therapy recommendation is provided to the hospital via Clinical Decision Support System and/or Hospital IT System.

### Methods

This Example describes the science and techniques behind the construction of Models that are derived using machine learning and used to assign ARDS patients into subphenotypes for various purposes such as predicting mortality and guiding clinical therapy. Multiple cohort datasets with different survival rates were analyzed to evaluate the effectiveness of the methodology on different patient cohorts.

Preliminary models were developed with publicly available data from the NHLBI ARDS Network (available at the URL: www.ardsnet.org/). Specifically, the ARMA-KARMA-LARMA, ALVEOLI, and FACTT datasets were used. Potential Model inputs were collated into a single file with 2,023 subjects. A randomization algorithm was used to split the combined dataset into 64% train, 16% test, and 20% hold-out validation samples.

After models were developed on the ARDS net data, the eICU-CRD dataset (available at the URL: eicu-crd.mit.edu/about/eicu/) was queried to provide an independent dataset for validation. Patients included were those who had a diagnosis of ARDS during their ICU stay, regardless of admitting diagnoses, with non-APACHE labs and vitals sources from the 24 hours prior to the time their ARDS diagnosis was charted in the ICU (n = 2094 patients with full data).

Additional validation data was sourced from the Brazillian ART dataset (available at the URL: www.ncbi.nlm.nih.gov/pubmed/28973363). Finally, validation data was sourced from internal Cleveland Clinic data.

Commonly recorded EHR vitals, laboratory results, and ventilator information were collated into a dataset with common variable names across all datasets. Variables of interest included Arterial pH, bicarbonate, bilirubin, creatinine, systolic, diastolic, and mean arterial pressure, FiO₂, heart rate, mean airway pressure, PaCO₂, PaO₂, PaO₂/FiO₂, PEEP, platelets, potassium, respiratory rate, SpO₂, and tidal volume. If continuous data were available, the lowest and highest values prior to study enrollment (or diagnosis time in the eICU dataset) were recorded, using L as a postscript for lowest and H as a postscript for highest, as well as the most recent value (postscript of R). For PaO₂/FiO₂, the lowest value in the 24 hours following enrollment or diagnosis was also recorded (postscript of LP). Age, gender, and BMI were also recorded.

As proof of concept, an initial K-means clustering Model was developed in Alteryx (Irvine, CA). Additionally, a python version was created to enable clinical utilization across numerous operating systems without need for specialized software. ARDSnet flat files prepared as described above were read into python for Model development. Patients were excluded from the dataset if they did not have measurements for all of the input variables, which reduced the total data available based on the model implemented.

Scikit-leam’s (Pedregosa, et al., 2011) StandardScaler (available at the URL: scikit-learn.org/stable/modules/generated/sklearn.preprocessing.StandardScaler.html) was used to develop a z-score transform for each input variable based on the training data, and that scaler was then applied to both training and validation data. The scikit-leam KMeans algorithm was next used (available at the URL: scikit-learn.org/stable/modules/generated/sklearn.cluster.KMeans.html) to train 2 clusters with 20 initial seeds. After experimentation and examination of contributions to principal components of the data, six Models were developed. The six Models were optimized based on different clinical needs as described in Table 2 below. Each resultant cluster was assigned to an ARDS subphenotype (subphenotype A and subphenotype B).

TABLE 2

Phenotype subclassifiers implemented in Example 1

| Model | # Input Variables | Description | Input Variables |
|---|---|---|---|
| Model 1 | 13 | Including input variables informed by select input variables described by Calfee et al. | Arterial pH-R, Bicarbonate-L, Creatinine-R, Diastolic BP-R, FIO2-R, Heart Rate-R, Mean arterial pressure-H, mean arterial pressure-L, potassium-R, respiratory rate-H, respiratory rate-L, SPO₂-R, systolic BP-R |
| Model 2 | 8 | Developed using a minimal number of input variables that were available across all validation training sets, which are expected to be available for a majority of clinical patients, and which are included in a majority of clinical trials | Arterial pH-R, bicarbonate-L, creatinine-R, FIO₂-R, heart rate-R, PaO₂-R, mean arterial pressure-R, respiratory rate-R |
| Model 3 | 17 | Developed using a broader range of variables which provide the most information about patient status | Age, arterial pH-R, bicarbonate-L, bilirubin-H, BMI, creatinine-R, FiO₂-R, gender, heart rate-R, PaCO₂-R, PaO₂/FiO₂-LP, PaO2-R, PEEP-R, Platelet-L, Tidal Volume-L, mean arterial pressure-R, respiratory rate-R |
| Model 4 | 13 | Developed as a compromise between Models 2 and 3 | Arterial pH-R, bicarbonate-R, BMI, creatinine-R, FiO₂-R, gender, heart rate-R, PaCO₂-R, PaO₂/FiO₂-LP, PEEP-R, Platelets-L, mean arterial pressure-R, respiratory rate-R |
| Model 5 | 9 | Developed based on Model 2 with the addition of Bilirubin | Arterial pH-R, bicarbonate-L, creatinine-R, FIO₂-R, heart rate-R, PaO₂-R, mean airway pressure-R, respiratory rate-R, bilirubin-H |
| Model 6 | 16 | Developed based on Model 3 without BMI | Age, arterial pH-R, bicarbonate-L, bilirubin-H, creatinine-R, FiO₂-R, gender, heart rate-R, PaCO₂-R, PaO₂/FiO₂-LP, PaO₂-R, PEEP-R, Platelet-L, Tidal Volume-L, mean arterial pressure-R, respiratory rate-R |

While Models 1-6 were developed based on the number of input variables and the specific list of input variables provided above in Table 2, in further embodiments, additional Models are developed to include alternative numbers of input variables and alternative combinations of input variables. Specifically, additional Models are developed to include any alternative combination of the input variables listed in Table 2 above. Even further, additional Models are developed to include any alternative combination of variables, not limited to the input variables listed in Table 2 above.

Following assignment of each cluster as a subphenotype, post-hoc analysis was performed to identify differential response to therapy in various datasets. Mortality rates were compared using Chi-Square for large sample size groups, while Fisher exact test was used to compare rates in small sample-size groups. T-tests were used to compare means of numeric values.

### Results

Following Model development, the 28 day and 90 day mortality rates were calculated for each subphenotype, dataset, and Model combination. Mortality rates for subphenotype A and subphenotype B for each of Models 1-4 are shown below in Table 3. The ARDSnet datasets are split to show separate results for training versus validation. Model 1 only shows results for the ARDSnet and eICU datasets because some of the input variables were not available in the ART and Cleveland Clinic datasets. Models 2-4 were developed specifically to include input variables which were available in each validation dataset.

TABLE 3

Mortality rates of patients classified in subphenotypes A and B using Models 1-4

| Model | Use | Dataset | Mortality Metric | % ST1 | ST A mortality | % ST2 | ST B mortality | p | Chi Sq |
|---|---|---|---|---|---|---|---|---|---|
| 1 | | | | | | | | | |
| 1 | Train | ARDSnet | Dead90 | 52.9 | 19.5 | 47.1 | 36.0 | 0.000 | 40.4 |
| 1 | Val | ARDSnet | Dead90 | 55.3 | 24.4 | 44.7 | 38.1 | 0.009 | 6.8 |
| 1 | Val | eICU | Died in hospital | 61.4 | 11.4 | 38.6 | 30.7 | 0.000 | 182.8 |
| 2 | | | | | | | | | |
| 2 | Train | ARDSnet | Dead90 | 55.3 | 20.8 | 44.7 | 35.6 | 0.000 | 32.8 |
| 2 | Val | ART | Dead28 | 40.9 | 29.8 | 59.1 | 47.8 | 0.000 | 19.5 |
| 2 | Val | ARDSnet | Dead90 | 55.6 | 23.4 | 44.4 | 38.6 | 0.004 | 8.5 |
| 2 | Val | Cleveland - All | Dead90 | 19.5 | 50.0 | 80.5 | 54.3 | 0.429 | 0.6 |
| 2 | Val | Cleveland - w/o Comorbidities | Dead90 | 21.3 | 37.0 | 78.7 | 46.0 | 0.239 | 1.4 |
| 2 | Val | eICU | Died in hospital | 83.3 | 15.3 | 16.7 | 37.4 | 0.000 | 141.2 |
| 3 | | | | | | | | | |
| 3 | Train | ARDSnet | Dead90 | 54.4 | 23.2 | 45.6 | 33.8 | 0.001 | 10.4 |
| 3 | Val | ART | Dead28 | 43.2 | 29.4 | 56.8 | 46.3 | 0.063 | 3.5 |
| 3 | Val | ARDSnet | Dead90 | 57.0 | 21.1 | 43.0 | 37.2 | 0.012 | 6.3 |
| 3 | Val | Cleveland - All | Dead90 | 28.2 | 50.7 | 71.8 | 53.7 | 0.539 | 0.4 |
| 3 | Val | Cleveland - w/o Comorbidities | Dead90 | 29.3 | 39.4 | 70.7 | 45.6 | 0.378 | 0.8 |
| 3 | Val | eICU | Died in hospital | 86.9 | 19.5 | 13.1 | 53.7 | 0.000 | 37.4 |
| 4 | | | | | | | | | |
| 4 | Train | ARDSnet | Dead90 | 52.8 | 22.8 | 47.2 | 33.9 | 0.000 | 16.3 |
| 4 | Val | ART | Dead28 | 29.6 | 24.4 | 70.4 | 43.0 | 0.031 | 4.6 |
| 4 | Val | ARDSnet | Dead90 | 53.3 | 21.8 | 46.7 | 38.8 | 0.002 | 9.5 |
| 4 | Val | Cleveland - All | Dead90 | 23.0 | 55.0 | 77.0 | 53.0 | 0.698 | 0.2 |
| 4 | Val | Cleveland - w/o Comorbidities | Dead90 | 25.9 | 47.7 | 74.1 | 43.5 | 0.563 | 0.3 |
| 4 | Val | eICU | Died in hospital | 82.0 | 16.7 | 18.0 | 44.9 | 0.000 | 86.4 |

As shown in Table 3, the ARDSnet training and validation datasets and eICU dataset have a significant mortality difference across subphenotypes for each Model created. The ART dataset shows significant difference in patient prognosis for Models 2 and 4, and a p value nearing significance (p = 0.06) for Model 3.

For Models 2, 3, and 4, the Cleveland Clinic dataset did not show a significant difference in mortality (p = 0.43, 0.54, and 0.70 respectively). Upon further consultation with their clinical staff, it was determined that their data included a patient cohort which was significantly sicker than patients in the other datasets. To align Cleveland Clinic data to be more similar to the other data sources, a subset of data “Cleveland - w/o Comorbidities” was created with the following exclusion criteria:
Patients marked positive for ICU mortality with an ICU length of stay (LOS) of < 2 days
Patients with the following major comorbidities:
   ◦Active malignancy
   ◦Chronic obstructive pulmonary disease (COPD)
   ◦Idiopathic pulmonary fibrosis (IPF)
   ◦Leukemia/multiple myeloma
   ◦Lymphoma
   ◦Metastatic solid tumor
   ◦Metastatic cancer
   ◦Hepatic failure
   ◦Immunocompromised status

The resultant Cleveland Clinic subset resulted in an improved difference in mortality between subphenotypes A and B.

Based on the availability of data for future studies, Model 2 was selected for future work. Model 2 provides significant differential mortality between subphenotype A and subphenotype B, and a minimal number of input variables which are likely to be collected and stored in the EHR for nearly all patients undergoing ARDS therapy. Likewise the input variables collected are likely to be included in any clinical trials being analyzed. A detailed comparison of patient characteristics by subphenotype for each of the eight input variables of Model 2 is shown below in Table 4A and 4B and Tables 5-8. Generally, subphenotype B patients tend to be sicker than subphenotype A patients. Table 9 below summarizes additional outcomes across each dataset beyond the single mortality rate shown above using Model 2.

TABLE 4A

Subphenotype Characteristics: Training Data - Combined ARDSnet Dataset

| | Missing | Subphenotype A | Subphenotype B | P |
|---|---|---|---|---|
| n | | 666 | 536 | |
| Age | | 51.0 [40.0, 65.0] | 46.0 [36.0, 58.0] | <0.001 |
| Gender = 1 | | 382 (57.4) | 288 (53.7) | 0.23 |
| BMI | 82 | 27.3 [23.1, 31.8] | 25.8 [22.0, 30.9] | 0.001 |
| Heart Rate | | 95.9 (18.8) | 114.4 (20.4) | <0.001 |
| MAP | | 112.0 (25.1) | 103.0 (23.3) | <0.001 |
| Resp Rate | | 28.0 [23.0, 35.0] | 38.0 [32.0, 44.0] | <0.001 |
| Platelets | | 165.0 [94.0, 240.5] | 151.0 [88.0, 232.5] | 0.129 |
| Arterial pH | | 7.4 (0.1) | 7.3 (0.1) | <0.001 |
| Bicarbonate | | 23.9 (4.6) | 18.2 (4.9) | <0.001 |
| Bilirubin | 194 | 0.8 [0.5, 1.4] | 0.9 [0.5, 1.8] | 0.086 |
| Creatinine | | 0.9 [0.7, 1.3] | 1.3 [0.8, 2.1] | <0.001 |
| PaCO2 | 4 | 38.0 [34.0, 43.0] | 37.0 [32.0, 44.0] | 0.046 |
| PaO2 | | 77.0 [67.0, 94.0] | 80.0 [67.0, 103.2] | 0.028 |
| FiO₂ | | 0.5 [0.4, 0.6] | 0.7 [0.6, 1.0] | <0.001 |
| PaO2/FiO2 | 55 | 140.0 [99.0, 182.0] | 98.0 [70.2, 143.8] | <0.001 |
| PEEP | 3 | 8.0 [5.0, 10.0] | 10.0 [6.8, 13.0] | <0.001 |
| Tidal vol | 185 | 500 [420, 600] | 500 [400, 600] | 0.162 |

TABLE 4B

Subphenotype Characteristics: Validation Data - Combined ARDSnet Dataset

| | Missing | Subphenotype A | Subphenotype B | P |
|---|---|---|---|---|
| n | | 175 | 140 | |
| Age | | 52.0 [40.5, 67.0] | 47.0 [37.0, 59.0] | 0.009 |
| Gender = 1 | | 108 (61.7) | 71 (50.7) | 0.065 |
| BMI | 22 | 27.8 [23.2, 32.2] | 26.6 [22.8, 31.6] | 0.412 |
| Heart Rate | | 96.2 (18.7) | 111.2 (21.1) | <0.001 |
| MAP | | 112.7 (26.9) | 102.0 (24.3) | <0.001 |
| Resp Rate | | 29.0 [23.0, 37.0] | 36.0 [30.0, 40.2] | <0.001 |
| Platelets | 3 | 178.5 [116.0, 276.0] | 157.0 [75.8, 238.2] | 0.011 |
| Arterial pH | | 7.4 (0.1) | 7.3 (0.1) | <0.001 |
| Bicarbonate | | 24.2 (4.4) | 18.0 (5.0) | <0.001 |
| Bilirubin | 54 | 0.8 [0.5, 1.3] | 1.0 [0.7, 2.1] | 0.002 |
| Creatinine | | 0.9 [0.7, 1.2] | 1.4 [0.9, 2.3] | <0.001 |
| PaCO2 | 3 | 37.0 [34.0, 44.0] | 37.0 [31.0, 46.0] | 0.673 |
| PaO2 | | 76.0 [68.0, 87.5] | 75.0 [65.8, 99.2] | 0.922 |
| FiO2 | | 0.5 [0.4, 0.6] | 0.8 [0.6, 1.0] | <0.001 |
| PaO2/FiO2 | 12 | 130.0 [92.0, 170.0] | 99.0 [68.0, 137.5] | <0.001 |
| PEEP | 1 | 8.0 [5.0, 10.0] | 10.0 [8.0, 14.0] | <0.001 |
| Tidal vol | 47 | 500 [445, 655] | 500 [410, 600] | 0.12 |

TABLE 5

Subphenotype Characteristics: Validation Data - eICU Dataset

| | Missing | Subphenotype A | Subphenotype B | P |
|---|---|---|---|---|
| n | | 2696 | 563 | |
| Age | | 68.0 [57.0, 78.0] | 67.0 [55.5, 77.5] | 0.18 |
| Gender = 1 | | 1444 (53.6) | 300 (53.3) | 0.942 |
| BMI | 123 | 27.9 [23.5, 33.9] | 27.3 [22.0, 30.9] | 0.026 |
| Heart Rate | | 77.6 (17.6) | 91.1 (21.6) | <0.001 |
| MAP | | 63.7 (17.9) | 59.9 (22.9) | <0.001 |
| Resp Rate | | 14.0 [11.0, 18.0] | 18.0 [14.0, 23.0] | <0.001 |
| Platelets | 97 | 198.0 [143.0, 266.0] | 196.0 [124.0, 279.0] | 0.179 |
| Arterial pH | | 7.4 (0.1) | 7.3 (0.1) | <0.001 |
| Bicarbonate | | 26.0 (5.9) | 18.8 (5.6) | <0.001 |
| Bilirubin | 1580 | 0.6 [0.4, 1.0] | 0.7 [0.5, 1.4] | <0.001 |
| Creatinine | | 1.0 [0.7, 1.5] | 1.9 [1.1, 3.3] | <0.001 |
| PaCO2 | 59 | 41.0 [35.0, 50.3] | 40.0 [32.0, 50.0] | 0.002 |
| PaO2 | | 89.4 [69.0, 124.0] | 118.0 [76.0, 219.0] | <0.001 |
| FiO2 | | 0.4 [0.4, 0.6] | 1.0 [0.6, 1.0] | <0.001 |
| PaO2/FiO2 | | 157.8 [98.3, 240.4] | 118.5 [68.9, 230.5] | <0.001 |
| PEEP | 1856 | 5.0 [5.0, 5.6] | 5.0 [5.0, 8.0] | 0.004 |
| Tidal vol | 2044 | 450 [400, 500] | 450 [400, 500] | 0.618 |

Note: Subphenotypes were assigned to 3,259 patient stays in eICU. Of the 3,259 patients, 2,623 (80.48%) had a ‘Full therapy’ care directive during their stay, 305 (9.36%) had a ‘Do not resuscitate’ directive, 87 had no recorded care directive, and the remaining 244 had a care directive less than full therapy, or a combination of directives over their stay. Of the patients with ‘Full therapy’ as the only directive during their stay, mortality was 29.5% in Subphenotype B (116/393) and 10.3% in Subphenotype A (223/2165) (p < 0.0000).

TABLE 6

Subphenotype Characteristics: Validation Data - ART Dataset

| | Missing | Subphenotype A | Subphenotype B | P |
|---|---|---|---|---|
| n | | 271 | 479 | |
| Age | | 54.0 [37.0, 65.0] | 51.0 [36.0, 63.0] | 0.076 |
| Gender = 1 | | 179 (66.1) | 287 (59.9) | 0.113 |
| BMI | 560 | 28.9 [24.6, 35.1] | 28.4 [25.0, 32.8] | 0.299 |
| Heart Rate | | 87.6 (18.5) | 109.6 (22.6) | <0.001 |
| MAP | | 81.7 (12.7) | 78.5 (14.1) | 0.001 |
| Resp Rate | | 24.0 [20.0, 28.0] | 26.0 [22.0, 32.0] | <0.001 |
| Platelets | 37 | 185.0 [126.5, 285.2] | 171.0 [93.0, 258.0] | 0.012 |
| Arterial pH | | 7.4 (0.1) | 7.2 (0.1) | <0.001 |
| Bicarbonate | | 27.3 (6.8) | 21.1 (4.4) | <0.001 |
| Bilirubin | 241 | 0.6 [0.4, 1.2] | 0.8 [0.4, 1.7] | 0.005 |
| Creatinine | | 0.9 [0.7, 1.4] | 1.6 [1.0, 2.6] | <0.001 |
| PaCO2 | | 47.0 [41.0, 56.0] | 53.0 [43.0, 65.0] | <0.001 |
| PaO2 | | 116.0 [79.5, 156.5] | 110.0 [81.0, 155.5] | 0.674 |
| FiO2 | | 0.7 [0.5, 0.8] | 0.8 [0.7, 1.0] | <0.001 |
| PaO2/FiO2 | | 116.0 [79.5, 156.5] | 110.0 [81.0, 155.5] | 0.664 |
| PEEP | | 10.0 [10.0, 14.0] | 14.0 [10.0, 14.0] | <0.001 |
| Tidal vol | | 360 [320, 410] | 350 [300, 399] | <0.001 |

TABLE 7

Subphenotype Characteristics: Validation Data - Cleveland Clinic Dataset (Full Dataset)

| | Missing | Subphenotype A | Subphenotype B | P |
|---|---|---|---|---|
| n | | 102 | 431 | |
| Age | | 59.5 [47.2, 70.8] | 56.0 [44.0, 66.0] | 0.099 |
| Gender = 1 | | 67 (65.7) | 224 (52.0) | 0.017 |
| BMI | | 30.6 [23.5, 39.4] | 30.4 [25.2, 36.3] | 0.932 |
| Heart Rate | | 98.7 (24.8) | 122.1 (24.8) | <0.001 |
| MAP | | 63.4 (13.0) | 56.7 (12.7) | <0.001 |
| Resp Rate | | 29.0 [25.0, 35.0] | 39.0 [32.0, 46.0] | <0.001 |
| Platelets | | 180.0 [109.5, 255.0] | 148.0 [77.0, 220.5] | 0.006 |
| Arterial pH | | 7.4 (0.1) | 7.3 (0.1) | <0.001 |
| Bicarbonate | | 27.4 (6.7) | 20.0 (5.5) | <0.001 |
| Bilirubin | 6 | 0.6 [0.4, 1.3] | 0.8 [0.4, 2.1] | 0.045 |
| Creatinine | | 1.1 [0.7, 1.7] | 1.7 [1.1, 2.8] | <0.001 |
| PaCO2 | | 41.0 [36.0, 51.0] | 42.0 [35.0, 50.1] | 0.868 |
| PaO2 | | 82.5 [67.7, 97.5] | 87.0 [69.2, 117.5] | 0.093 |
| FiO2 | | 0.6 [0.5, 0.8] | 1.0 [0.7, 1.0] | <0.001 |
| PaO2/FiO2 | 1 | 134.0 [100.0, 186.0] | 113.0 [79.0, 170.6] | 0.002 |
| PEEP | 10 | 8.0 [7.5, 10.0] | 10.0 [8.0, 14.0] | <0.001 |
| Tidal vol | 19 | 486 [436, 545] | 480 [413, 546] | 0.373 |

TABLE 8

Subphenotype Characteristics: Validation Data - Cleveland Clinic Dataset (Without Comorbidities)

| | Missing | Subphenotype A | Subphenotype B | P |
|---|---|---|---|---|
| n | | 53 | 201 | |
| Age | | 54.0 [43.0, 66.0] | 54.0 [41.0, 64.0] | 0.524 |
| Gender = 1 | | 32 (60.4) | 104 (51.7) | 0.334 |
| BMI | | 32.4 [26.4, 44.2] | 30.7 [25.4, 37.9] | 0.189 |
| Heart Rate | | 97.6 (24.7) | 121.5 (23.9) | <0.001 |
| MAP | | 63.2 (14.7) | 57.4 (12.6) | 0.011 |
| Resp Rate | | 29.0 [24.0, 33.0] | 37.0 [31.0, 45.0] | <0.001 |
| Platelets | | 182.0 [92.0, 272.0] | 152.0 [85.0, 211.0] | 0.072 |
| Arterial pH | | 7.4 (0.1) | 7.3 (0.1) | <0.001 |
| Bicarbonate | | 26.5 (6.1) | 19.7 (5.6) | <0.001 |
| Bilirubin | 3 | 0.7 [0.4, 1.7] | 0.7 [0.4, 1.7] | 0.315 |
| Creatinine | | 1.1 [0.7, 1.8] | 1.1 [0.7, 1.8] | 0.001 |
| PaCO2 | | 41.0 [36.0, 48.0] | 41.0 [36.0, 48.0] | 0.989 |
| PaO2 | | 80.0 [67.0, 94.0] | 80.0 [67.0, 94.0] | 0.084 |
| FiO2 | | 0.6 [0.5, 0.8] | 0.6 [0.5, 0.8] | <0.001 |
| PaO2/FiO2 | 1 | 129.7 [100.1, 171.8] | 129.7 [100.1, 1701.8] | 0.161 |
| PEEP | 2 | 8.0 [7.0, 10.0] | 8.0 [7.0, 10.0] | 0.002 |
| Tidal vol | 8 | 485 [436, 514] | 485 [436, 514] | 0.854 |

TABLE 9

Additional outcomes of patients classified using Model 2 in subphenotype A or subphenotype across different EHR databases

| | | ALVEOLI | | | ARMA | | | FACTT | | |
|---|---|---|---|---|---|---|---|---|---|---|
| Metric | value | Subphenotype A | Subphenotype B | p | Subphenotype A | Subphenotype B | p | Subphenotype A | Subphenotype B | p |
| n | | 313 | 208 | | 224 | 211 | | 504 | 437 | |
| VentFreeDays | | 21.0 [11.0,24.0] | 7.5 [0.0,20.0] | <0.001 | 19.0 [0.0,25.0] | 9.0 [0.0,21.0] | <0.001 | 19.0 [5.0,23.0] | 13.0 [0.0,21.0] | <0.001 |
| Days under MV | | | | | | | | | | |
| ICU LOS | | | | | | | | | | |
| Hospital LOS | | | | | | | | | | |
| ICU expired | 1 | | | | | | | | | |
| Hospital expired | 1 | | | | | | | | | |
| Dead28 | 1 | 44 (14.1) | 73 (35.1) | | | | | 89(17.7) | 126 (28.8) | |
| Dead90 | 1 | 53 (16.9) | 87 (41.8) | | 54 (24.1) | 77 (36.5) | | 113 (22.4) | 150 (34.3) | |
| Dead6mo | 1 | | | | | | | | | |

TABLE 9 (continued)

| | | eICU | ART | | | | |
|---|---|---|---|---|---|---|---|
| Metric | Value | Subphenotype A | Subphenotype B | p | Subphenotype A | Subphenotype B | p |
| n | | | | | 215 | 365 | |
| VentFreeDays | | | | | | | |
| Days under MV | | 4.0 (4.4) | 4.0 (4.7) | 0.946 | 13.0 [8.0,20.5] | 14.0 [8.0,20.0] | 0.769 |
| ICU LOS | | 2.8 [1.5,5.4] | 2.6 [1.1,5.7] | 0.049 | | | |
| Hospital LOS | | 8.6 [5.1,14.7] | 7.3 [3.1,15.6] | <0.001 | | | |
| ICU expired | 1 | 231 (8.5) | 138 (25.6) | | 94 (43.7) | 234 (64.1) | |
| Hospital expired | 1 | 404 (15.3) | 199 (37.5) | | 103 (48.1) | 242 (66.3) | |
| Dead28 | 1 | | | | 60 (31.4) | 135 (47.9) | |
| Dead90 | 1 | | | | | | |
| Dead6mo | 1 | | | | 51 (32.5) | 105 (46.9) | |
| 28 d survival | | | | | | | |
| 90 d survival | | | | | | | |
| hospitaldischargelocation | Death | 422 (15.6) | 204 (38.1) | <0.001 | | | |
| hospitaldischargelocation | Home | 1351 (50.0) | 180 (33.6) | | | | |
| hospitaldischargelocation | Nursing Home | 30 (1.1) | 4 (0.7) | | | | |
| hospitaldischargelocation | Other | 100 (3.7) | 31 (5.8) | | | | |
| hospitaldischargelocation | Other External | 125 (4.6) | 20 (3.7) | | | | |
| hospitaldischargelocation | Other Hospital | 128 (4.7) | 26 (4.9) | | | | |
| hospitaldischargelocation | Rehabilitation | 145 (5.4) | 15 (2.8) | | | | |
| hospitaldischargelocation | SNF | 399 (14.8) | 55 (10.3) | | | | |
| predictedicumortality | | 0.1 [0.0,0.2] | 0.2 [0.1,0.4] | <0.001 | | | |
| predictedhospitalmortality | | 0.1 [0.1,0.3] | 0.3 [0.1,0.5] | <0.001 | | | |
| predictediculos | | 5.5 (2.1) | 6.4 (2.1) | <0.001 | | | |
| predictedhospitallos | | 13.1 (5.3) | 14.1 (5.7) | 0.002 | | | |

TABLE 9 (continued)

| | | Cleveland - all | | | Cleveland - no MCC | | |
|---|---|---|---|---|---|---|---|
| Metric | value | Subphenotype A | Subphenotype B | p | Subphenotype A | Subphenotype B | p |
| n | | 104 | 429 | | 54 | 200 | |
| VentFreeDays | | 9.4 (9.8) | 7.0 (9.3) | 0.028 | 11.7 (10.0) | 7.6 (9.3) | 0.008 |
| Days under MV | | 12.9 (9.0) | 14.0 (11.8) | 0.286 | 12.1 (9.1) | 14.4 (12.1) | 0.132 |
| ICU LOS | | 13.0 [7.8,20.0] | 13.0 [7.0,21.0] | 0.932 | 12.5 [7.0,20.0] | 12.0 [7.0,20.0] | 0.835 |
| Hospital LOS | | 16.0 [12.0,25.0] | 19.0 [11.0,28.0] | 0.38 | 16.0 [11.0,25.8] | 17.5 [10.0,26.0] | 0.934 |
| ICU expired | 1 | 40 (38.5) | 213 (49.7) | | 16 (29.6) | 85 (42.5) | |
| Hospital expired | 1 | 42 (40.4) | 221 (51.5) | | 16 (29.6) | 87 (43.5) | |
| Dead28 | 1 | 43 (41.3) | 202 (47.1) | | 17 (31.5) | 80 (40.0) | |
| Dead90 | 1 | 52 (50.0) | 233 (54.3) | | 20 (37.0) | 92 (46.0) | |
| Dead6mo | 1 | | | | | | |
| 28 d survival | | 28.0 [13.0,28.0] | 25.0 [9.0,28.0] | 0.111 | 28.0 [15.0,28.0] | 28.0 [10.0,28.0] | 0.1 |
| 90 d survival | | 30.5 [13.0,90.0] | 25.0 [9.0,90.0] | 0.18 | 59.5 [15.0,90.0] | 32.5 [10.0,90.0] | 0.128 |
| hospitaldischargelocation | Death | | | | | | |
| hospitaldischargelocation | Home | | | | | | |
| hospitaldischargelocation | Nursing Home | | | | | | |
| hospitaldischargelocation | Other | | | | | | |
| hospitaldischargelocation | Other External | | | | | | |
| hospitaldischargelocation | Other Hospital | | | | | | |
| hospitaldischargelocation | Rehabilitation | | | | | | |
| hospitaldischargelocation | SNF | | | | | | |
| predictedicumortality | | | | | | | |
| predictedhospitalmortality | | | | | | | |
| predictediculos | | | | | | | |
| predictedhospitallos | | | | | | | |

In almost every mortality metric (ICU, hospital, 28 day, 90 day, and 6 month mortality), subphenotype B had a significantly higher mortality rate. Similarly, in the eICU dataset, subphenotype B patients also had a significantly higher predicted mortality risk. In addition to a lower mortality rate, patients in subphenotype A have significantly more ventilator free days in all datasets except in the eICU dataset, which had a lower acuity patient demographic and ART. ART’s analysis does not take the recruitment maneuvers of the study intervention into account. Patients in the Cleveland Clinic dataset did not have a significant difference in ICU or hospital LOS. However, eICU subphenotype A patients had significantly longer LOS for both metrics, even though patients in subphenotype B had significantly higher predicted ICU and hospital LOS.

Table 10 below compares subphenotype A and subphenotype B mortalities from Model 2 with the mortality of the APACHE III and SOFA cutoffs using the metrics of true positives (TP), false positives (FP), false negatives (FN), true negatives (TN), sensitivity, specificity, positive predictive value (PPV), negative predictive value (NPV), and F1, which provides a balanced metric of sensitivity and PPV. The F1 values of Model 2 did not achieve the F1 of APACHE and SOFA. However, the number of input variables of Model 2 is lower and, in the case of APACHE, does not rely upon prior knowledge of a patient’s existing comorbidities.

TABLE 10

Mortality rates of patients classified in subphenotype A and subphenotype B as well as metrics of true positives (TP), false positives (FP), false negatives (FN), true negatives (TN), sensitivity, specificity, positive predictive value (PPV), negative predictive value (NPV), and F1, which provides a balanced metric of sensitivity and PPV

| Dataset | Method | TP | FP | FN | TN | Sensitivity | Specificity | PPV | NPV | F1 | Subphenotype A (Low Risk) Mortality | Subphenotype B (High Risk) Mortality |
|---|---|---|---|---|---|---|---|---|---|---|---|---|
| FACTT | APACHE | 226 | 403 | 33 | 252 | 87% | 38% | 36% | 88% | 51% | 12% | 36% |
| FACTT | Model 2 | 147 | 277 | 112 | 397 | 57% | 59% | 35% | 78% | 43% | 22% | 35% |
| eICU | APACHE | 331 | 646 | 206 | 1688 | 62% | 72% | 34% | 89% | 44% | 11% | 34% |
| eICU | Model 2 | 170 | 298 | 367 | 2036 | 32% | 87% | 36% | 85% | 34% | 15% | 36% |
| CC - All | APACHE | 260 | 202 | 21 | 40 | 93% | 17% | 56% | 66% | 70% | 34% | 56% |
| CC - All | Model 2 | 231 | 191 | 50 | 51 | 82% | 21% | 55% | 50% | 66% | 50% | 55% |
| CC - All | SOFA | 213 | 129 | 68 | 116 | 76% | 47% | 62% | 63% | 68% | 37% | 62% |
| CC - All | Model 2 | 231 | 193 | 50 | 52 | 82% | 21% | 54% | 51% | 66% | 49% | 54% |
| CC - w/o comorbid | APACHE | 102 | 113 | 7 | 27 | 94% | 19% | 47% | 79% | 63% | 21% | 47% |
| CC - w/o comorbid | Model 2 | 91 | 107 | 18 | 33 | 83% | 24% | 46% | 65% | 59% | 35% | 46% |
| CC - w/o comorbid | SOFA | 87 | 66 | 22 | 75 | 80% | 53% | 57% | 77% | 66% | 23% | 57% |
| CC - w/o comorbid | Model 2 | 91 | 107 | 18 | 34 | 83% | 24% | 46% | 65% | 59% | 35% | 46% |

Furthermore, Model 2 appears to provide information which supplements the APACHE and SOFA scores. A new variable was created which concatenates each of the Model 2 subphenotype A and subphenotype B scores with each of the APACHE scores and SOFA scores. Table 11 below shows differential mortality when each of the subphenotype A and subphenotype B scores from Model 2 were combined with the APACHE cutoff scores. This technique adds an additional level of separation in identifying patient risk. Of note, the lowest mortality is typically seen when subphenotype B scores are mixed with the low-risk mortality APACHE scores (i.e., “ST A AP0”).

Similar results in differential mortality when each of the subphenotype A and subphenotype B scores from Model 2 were combined with the SOFA cutoff scores are shown in Table 12 below for the Cleveland Clinic full dataset (i.e., “CC-All”) and for the Cleveland Clinic with comorbidities removed dataset (i.e., “CC- w/o comorbid”). In this case, subphenotype A cases above the SOFA cutoff score have the highest mortality rate.

TABLE 11

Different mortality rates when scores are combined with APACHE cutoff scores

| | Subphenotype B AP 1 | | Subphenotype A AP 1 | | Subphenotype B AP 0 | | Subphenotype A AP 0 | | Mortality | | | | |
|---|---|---|---|---|---|---|---|---|---|---|---|---|---|
| | Alive | Dead | Alive | Dead | Alive | Dead | Alive | Dead | ST B AP1 | ST A AP1 | ST B AP0 | ST A AP0 | p |
| FACTT | 227 | 142 | 176 | 84 | 50 | 5 | 202 | 28 | 38% | 32% | 9% | 12% | <0.0000 |
| eICU | 125 | 140 | 521 | 191 | 173 | 30 | 1515 | 176 | 53% | 27% | 15% | 10% | <0.0000 |
| CC - All | 171 | 222 | 31 | 38 | 20 | 9 | 20 | 12 | 56% | 55% | 31% | 38% | 0.0138 |
| CC - w/o comorbid | 93 | 88 | 20 | 14 | 14 | 3 | 13 | 4 | 49% | 41% | 18% | 24% | 0.0248 |

TABLE 12

Different mortality rates when scores are combined with SOFA cutoff scores

| | Subphenotype B SOFA 1 | | Subphenotype A SOFA 1 | | Subphenotype B SOFA 0 | | Subphenotype A SOFA 0 | | Mortality | | | | |
|---|---|---|---|---|---|---|---|---|---|---|---|---|---|
| | Alive | Dead | Alive | Dead | Alive | Dead | Alive | Dead | ST B S 1 | ST A S 1 | ST B S 0 | ST A S 0 | p |
| CC - All | 116 | 186 | 13 | 27 | 77 | 45 | 39 | 23 | 62% | 68% | 37% | 37% | <0.0000 |
| CC - w/o comorbid | 59 | 74 | 7 | 13 | 48 | 17 | 27 | 5 | 56% | 65% | 26% | 16% | <0.0000 |

### Treatment Guidance: NMB Therapy

Data provided by the Cleveland Clinic identified six potential adjuvant interventions for ARDS patients. Current guidance from the Cleveland Clinic dictates that an ARDS patient is eligible for the first two adjunctive ARDS therapies of proning and NMB within 48 hours of diagnosis if their P/F ratio < 150 and FiO₂ > 0.6. Based on the availability of data (228 patients receiving NMB and 76 patients receiving proning), NMB was identified as a first target for differential analysis within subphenotypes A and B of Model 2.

Previous studies have shown conflicting results about the benefits of NMB early in ARDS therapy (ROSE study, PETAL clinical trials network, 2019; ACURASYS study, Papazian, L., available at URL: www.nejm.org/doi/full/10.1056/NEJMoa1005372, 2010). The ROSE study was a US-based study of NMB with sedation. Raw 90-day in-hospital mortality in the NMB intervention group was 42.5% compared with 42.8% in the control group. There were no differences in the additional endpoints measured, and the study was concluded early due to futility. The ACURASYS study showed that patients who received NMB early in their ARDS treatment had significantly lower mortality after adjusting for baseline PaO₂/FiO₂ and Simplified Acute Physiology II score. Raw mortality rates were 31.6% in the group receivi NMB and 40.7% in the placebo group. Because of the conflicting results and varying methodologies of the studies, there is not an international consensus on use of NMB in ARDS.

Confusion matrices were created to understand the impact of giving NMB versus not giving an NMB when a patient either qualified or did not qualify for NMB using the Cleveland Clinic Protocol. Sample sizes in Cleveland Clinic dataset alone were small, so the additional datasets were queried. ARMA-KARMA-LARMA and ALVEOLI provided relatively large sample sizes with a good mix of treatment and non-treatment. FACTT did not include data on NMB utilization. eICU had a large sample size, but the total number of patients receiving NMB was small. The ART dataset was excluded from this analysis for several reasons. First, in the ART arm of the ART dataset, almost every patient received NMB as part of their recruitment maneuver. Within the ARDSnet control arm, there was still a very high mortality rate, with outcomes not aligned with the other studies.

The data in Tables 13 and 14 suggests that patients in subphenotype B may benefit (or at least not be harmed) from NMB regardless of whether they meet eligibility criteria defined by the PaO₂/FiO₂ and FiO₂ criteria. Conversely, it appears that patients in subphenotype A are harmed by NMB, regardless of their PaO₂/FiO₂ and FiO₂.

TABLE 13

Morality Rates for Cleveland Clinic Protocol (i.e., “Protocol 2”)
Cleveland - all data

| | Subphenotype A survived | Subphenotype A deceased | Mortality | Subphenotype B survived | Subphenotype B deceased | Mortality |
|---|---|---|---|---|---|---|
| Overall Mortality | 52 | 50 | 49% | 196 | 233 | 54% |
| Regardless of Eligibility | | | | | | |
| Received NMB | 5 | 14 | 74% | 74 | 93 | 56% |
| Did not receive NMB | 47 | 38 | 45% | 122 | 140 | 53% |
| Eligible for prone/NMB | | | | | | |
| Received NMB | 5 | 10 | 67% | 59 | 82 | 58% |
| Did not receive NMB | 15 | 11 | 42% | 65 | 83 | 56% |
| Not Eligible for Prone/NMB | | | | | | |
| Received NMB | 0 | 4 | 100% | 15 | 11 | 42% |
| Did not receive NMB | 32 | 27 | 46% | 57 | 57 | 50% |
| | Cleveland - comorbidities removed | | | | | |
| | Subphenotype A survived | Subphenotype 2 deceased | Mortality | Subphenotype A survived | Subphenotype B deceased | Mortality |
| Overall Mortality | 34 | 20 | 37% | 108 | 92 | 46% |
| Regardless of Eligibility | | | | | | |
| Received NMB | 4 | 7 | 64% | 37 | 30 | 45% |
| Did not receive NMB | 30 | 13 | 30% | 71 | 62 | 47% |
| Eligible for prone/NMB | | | | | | |
| Received NMB | 4 | 5 | 56% | 27 | 28 | 51% |
| Did not receive NMB | 10 | 1 | 9% | 39 | 34 | 47% |
| Received NMB | 0 | 2 | 100% | 10 | 2 | 17% |
| Did not receive NMB | 20 | 12 | 38% | 32 | 28 | 47% |

TABLE 14

Morality Rates for Cleveland Clinic Protocol (i.e., “Protocol 2”)
eICU

| | Subphenotype A survived | Subphenotype A deceased | Mortality | Subphenotype B survived | Subphenotype B deceased | Mortality |
|---|---|---|---|---|---|---|
| Overall Mortality | 2243 | 404 | 15% | 332 | 199 | 37% |
| Regardless of Eligibility | | | | | | |
| Received NMB | 9 | 7 | 44% | 2 | 7 | 78% |
| Did not receive NMB | 1046 | 213 | 17% | 157 | 97 | 38% |
| Eligible for prone/NMB | | | | | | |
| Received NMB | 8 | 6 | 43% | 2 | 7 | 78% |
| Did not receive NMB | 378 | 132 | 26% | 76 | 69 | 48% |
| Not Eligible for Prone/NMB | | | | | | |
| Received NMB | 1 | 1 | 50% | | | |
| Did not receive NMB | 669 | 79 | 11% | 81 | 28 | 26% |
| | ARMA-KARMA-LARMA | | | | | |
| | Subphenotype A survived | Subphenotype A deceased | Mortality | Subphenotype B survived | Subphenotype B deceased | Mortality |
| Overall Mortality | 170 | 54 | 24% | 134 | 77 | 36% |
| Regardless of Eligibility | | | | | | |
| Received NMB | 46 | 26 | 36% | 64 | 44 | 41% |
| Did not receive NMB | 124 | 31 | 20% | 70 | 33 | 32% |
| Received NMB | 35 | 17 | 33% | 52 | 40 | 43% |
| Did not receive NMB | 54 | 18 | 25% | 51 | 24 | 32% |
| Received NMB | 11 | 6 | 35% | 12 | 4 | 25% |
| Did not receive NMB | 81 | 19 | 19% | 31 | 13 | 30% |
| Overall Mortality | 259 | 52 | 17% | 121 | 77 | 39% |
| Regardless of Eligibility | | | | | | |
| Received NMB | 46 | 15 | 25% | 34 | 37 | 52% |
| Did not receive NMB | 213 | 37 | 15% | 87 | 40 | 31% |
| Eligible for prone/NMB | | | | | | |
| Received NMB | 23 | 6 | 21% | 28 | 35 | 56% |
| Did not receive NMB | 72 | 14 | 16% | 67 | 29 | 30% |
| Not Eligible for Prone/NMB | | | | | | |
| Received NMB | 23 | 9 | 28% | 6 | 2 | 25% |
| Did not receive NMB | 163 | 32 | 16% | 26 | 13 | 33% |

Based on those observations, the hypothesis is that a protocol for NMB administration where NMB is administered if a patient is in subphenotype B and NMB is not administered if a patient is in subphenotype A (i.e., “Protocol 1”), will outperform a NMB protocol where a patient receives NMB if their PaO₂/FiO₂ > 150 and FiO₂ > 0.6 (i.e., “Protocol 2”).

Table 15 below depicts the hypothetical NMB Protocol 2, in which an ARDS patient receives NMB therapy if the patient’s PaO₂/FiO₂ < 150 and FiO₂ < 0.6, according to the Cleveland Clinic protocol. A patient was classified as ‘Protocol Followed’ if they met the Cleveland Clinic protocol and received NMB, or if they did not meet the Cleveland Clinic protocol and did not receive NMB. Patients classified as “Protocol Not Followed” were those who met Cleveland Clinic protocol and did not receive NMB, or did not meet Cleveland Clinic protocol but received NMB anyway.

TABLE 15

Results from a hypothetical NMB Protocol 2

| | Protocol Followed | | | Protocol Not Followed | | | | |
|---|---|---|---|---|---|---|---|---|
| | Alive | Dead | Mortality | Alive | Dead | Mortality | Chi sq | P |
| Cleveland | 83 | 73 | 47% | 59 | 39 | 40% | 1.196 | 0.274115 |
| ARMA | 176 | 79 | 31 % | 128 | 52 | 29% | 0.219 | 0.6396 |
| ALVEOLI | 241 | 75 | 24% | 168 | 52 | 24% | 0.001 | 1 |
| Total | 500 | 227 | 31% | 355 | 143 | 29% | 0.883 | 0.3474 |

Table 16 below depicts the hypothetical NMB Protocol 1, in which an ARDS patient classified as subphenotype B by Model 2 receives NMB therapy and in which an ARDS patient classified as subphenotype A by Model 2 does not receive NMB therapy. A patient was classified as ‘Protocol Followed’ if they were classified as subphenotype B by Model 2 and received NMB, or if they were classified as subphenotype A by Model 2 and did not receive NMB. Patients classified as “Protocol Not Followed” were those who were classified as subphenotype B by Model 2 and did not receive NMB, or were classified as subphenotype A by Model 2 but received NMB anyway.

TABLE 16

Results from a hypothetical NMB Protocol 1

| | Protocol Followed | | | Protocol Not Followed | | | | |
|---|---|---|---|---|---|---|---|---|
| | Alive | Dead | Mortality | Alive | Dead | Mortality | Chi sq | p |
| Cleveland | 67 | 43 | 39% | 75 | 69 | 48% | 1.971 | 0.1604 |
| ARMA | 188 | 75 | 29% | 116 | 56 | 33% | 0.807 | 0.369 |
| ALVEOLI | 247 | 74 | 23% | 133 | 55 | 29% | 2.411 | 0.1205 |
| Total | 502 | 192 | 28% | 324 | 180 | 36% | 8.834 | 0.002957 |

Table 15 shows that the overall mortality rate across the Cleveland, ARMA, and ALVEOLI datasets was higher among patients whose care followed Protocol 2 (i.e., the Cleveland Clinic protocol) than it was for patients who were not treated according to Protocol 2 (i.e., the Cleveland Clinic protocol). Following Protocol 2 did not result in a significant difference in mortality (p = 0.3474). In contrast, Table 16 shows that using Protocol 1 (i.e., subphenotyping using Model 2), each dataset showed reduced mortality. While a significant mortality reduction was not identified for any individual dataset, the combination of data from each of the three datasets did show a significant reduction in mortality using Protocol 1 (p = 0.002957).

Additional outcomes are shown in Tables 17 and 18 below for both Protocols 1 and 2. subphenotype A patients who did not receive NMB had more ventilator free days across all datasets. While subphenotype B patients who received NMB benefited from lower mortality rates, they did not see a reduction in ventilator free days. In the 90 day survival rates, patients in subphenotype A who received NMB had significantly lower survival than the other treatment groups, followed by patients in subphenotype B who did not receive NMB. Similar relationships are seen for Protocol 2. However, the relationships for Protocol 2 are not as strong.

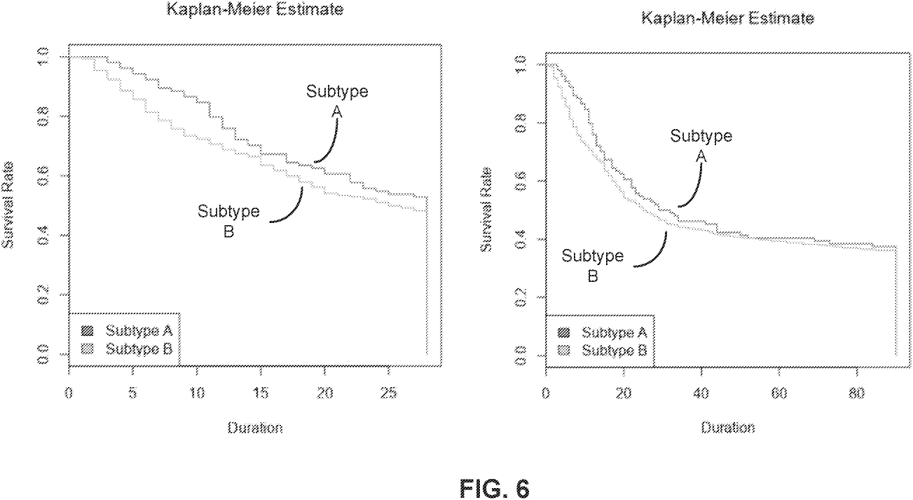

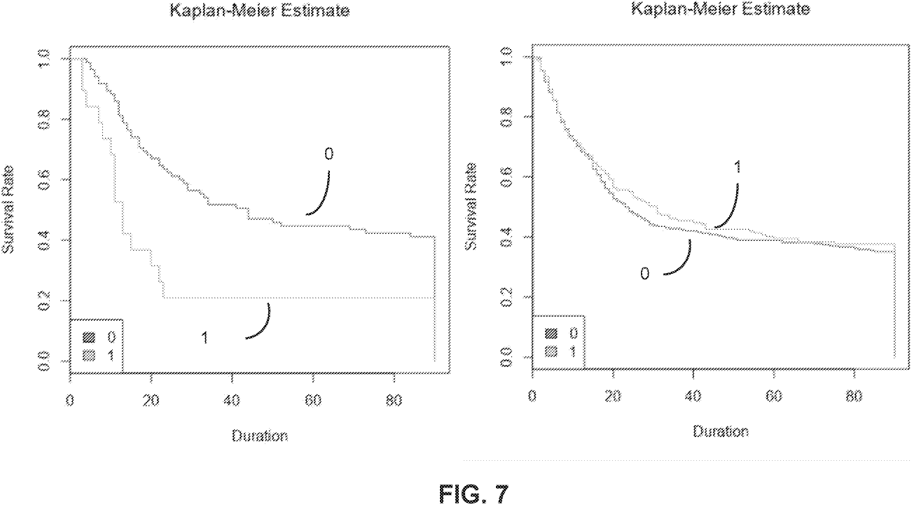

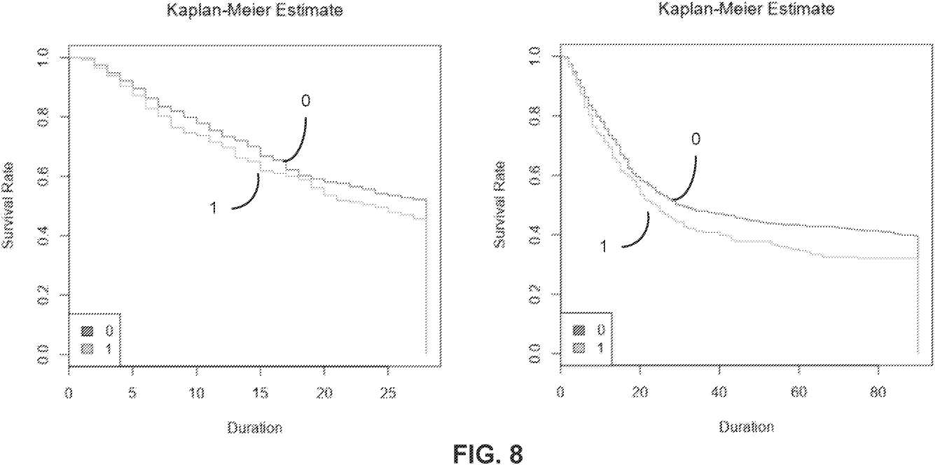

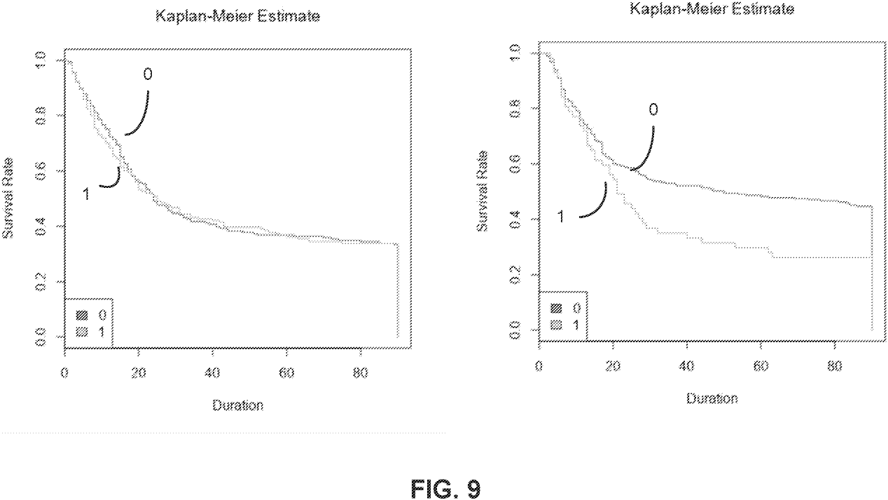

FIGS. 6-25 provide Kaplan Meier survival curves for both Protocols 1 and 2 studied. Specifically, FIG. 6 depicts survival of patients in subphenotype A v. subphenotype B across the full Cleveland Clinic Dataset at 28-days (left) and 90-days (right). FIG. 7 depicts survival of patients in subphenotype A (left) and subphenotype B (right) at 90 days for patients with (1) and without (0) neuromuscular block. FIG. 8 depicts survival of patients at 28 days (left) and 90 days (right) across patients that are eligible (1) or not eligible (0) for Neuromuscular block according to Cleveland Clinic criteria. FIG. 9 depicts survival of patients at 90 days with (1) and without (0) neuromuscular block for patients that are eligible (left) and ineligible (right) according to Cleveland Clinic Protocol.

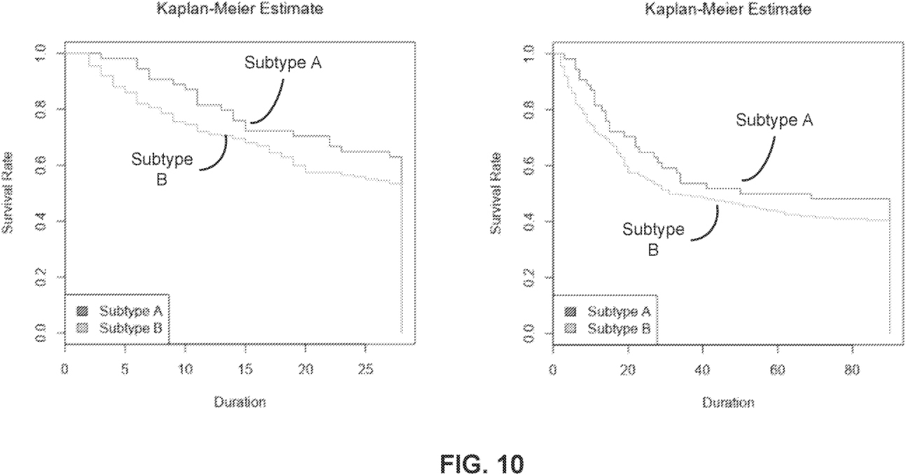

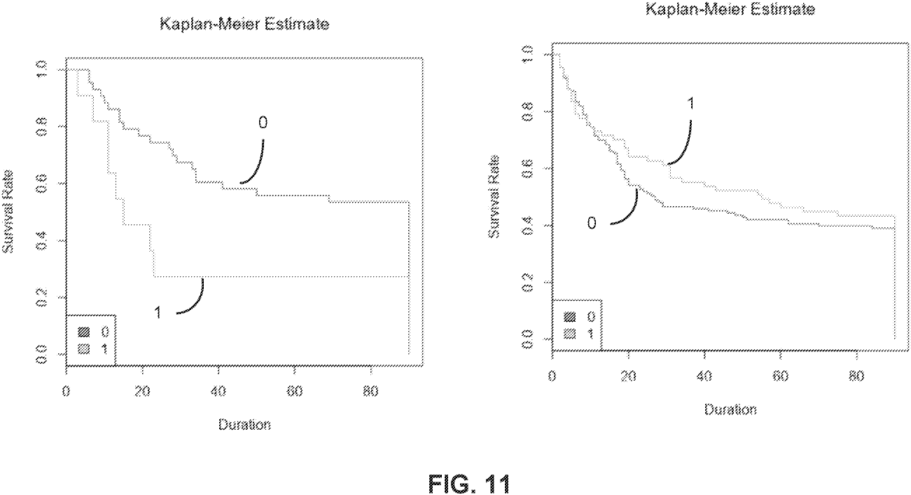

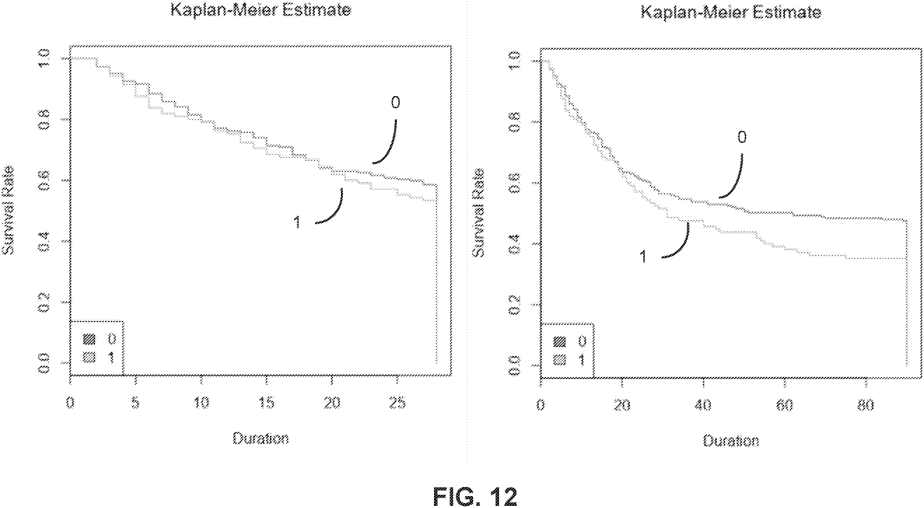

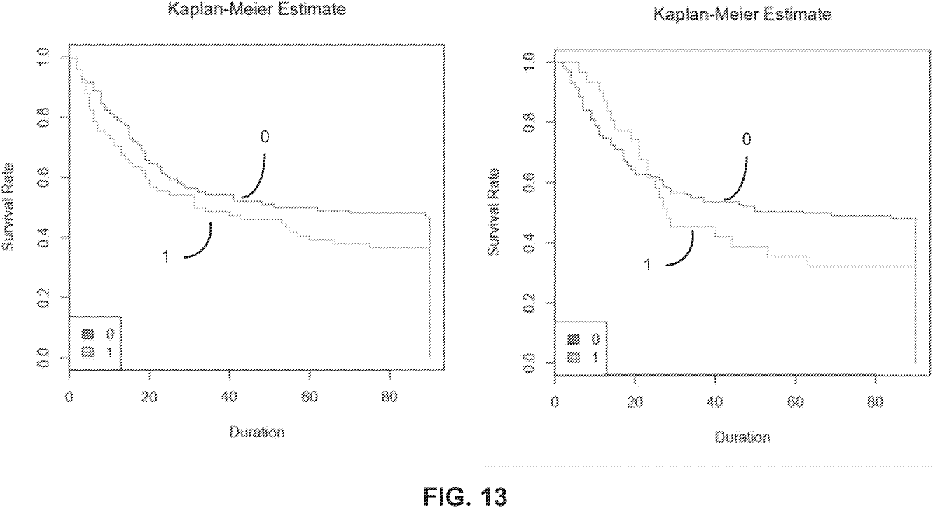

FIGS. 10-13 relate to analysis on the Cleveland Clinic Dataset (without comorbidities). FIG. 10 depicts survival of patients in subphenotype A v. subphenotype B across the Cleveland Clinic Dataset (without comorbidities) at 28-days (left) and 90-days (right). FIG. 11 depicts survival of patients in subphenotype A (left) and subphenotype B (right) at 90 days for patients with (1) and without (0) neuromuscular block. FIG. 12 depicts survival of patients at 28 days (left) and 90 days (right) across patients that are eligible (1) or not eligible (0) for Neuromuscular block according to Cleveland Clinic criteria. FIG. 13 depicts survival of patients at 90 days with (1) and without (0) neuromuscular block for patients that are eligible (left) and ineligible (right) according to Cleveland Clinic Protocol.

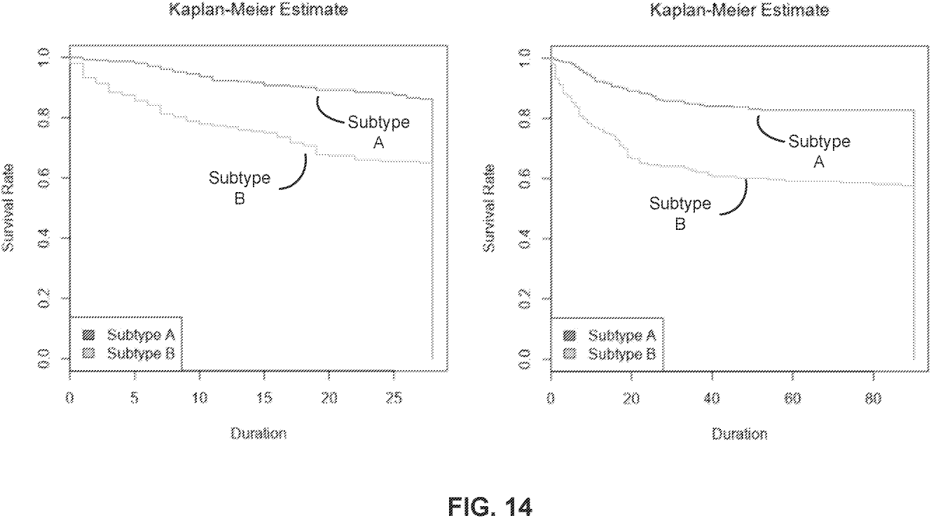

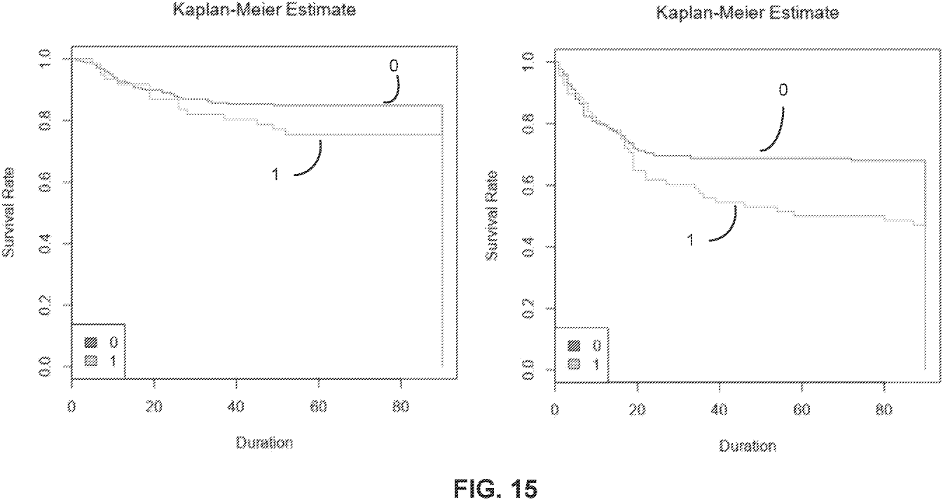

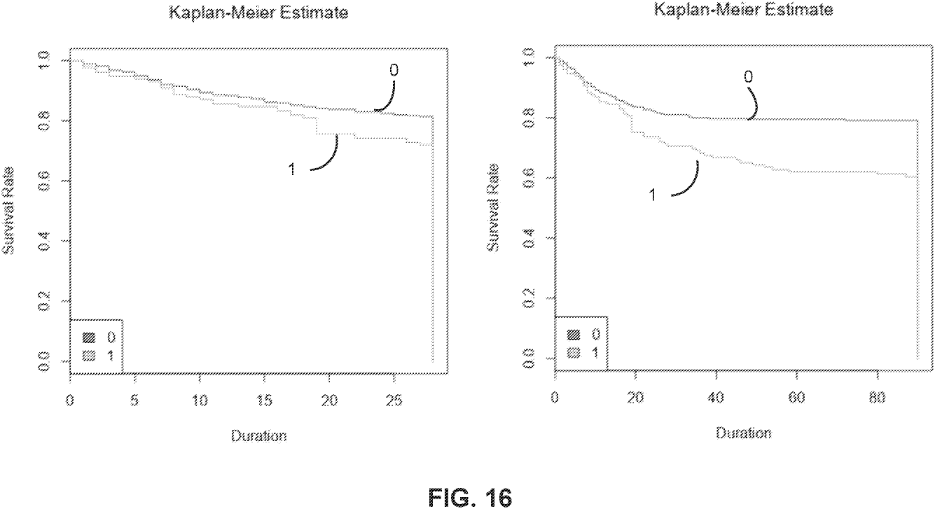

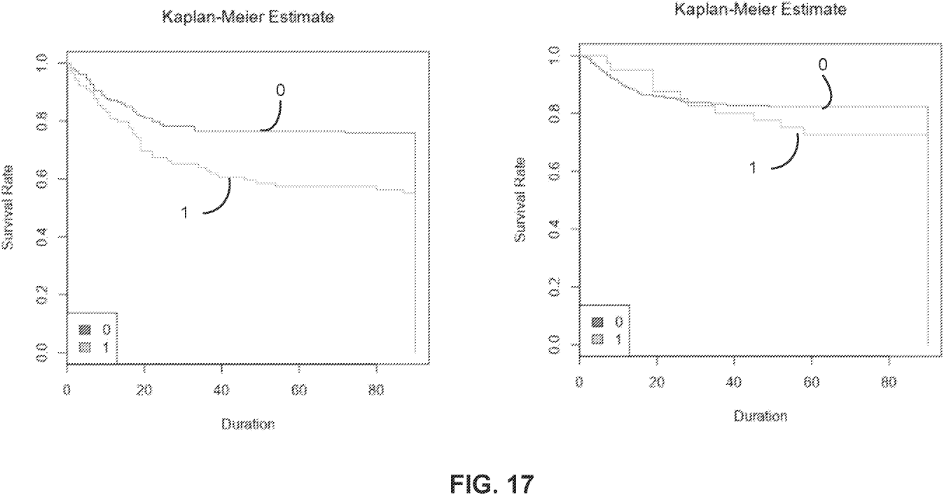

FIGS. 14-17 relate to analysis on the ALVEOLI dataset. FIG. 14 depicts survival of patients in subphenotype A v. subphenotype B across the ALVEOLI dataset at 28-days (left) and 90-days (right). FIG. 15 depicts survival of patients in subphenotype A (left) and subphenotype B (right) at 90 days for patients with (1) and without (0) neuromuscular block. FIG. 16 depicts survival of patients at 28 days (left) and 90 days (right) across patients that are eligible (1) or not eligible (0) for Neuromuscular block according to Cleveland Clinic criteria. FIG. 17 depicts survival of patients at 90 days with (1) and without (0) neuromuscular block for patients that are eligible (left) and ineligible (right) according to Cleveland Clinic Protocol.

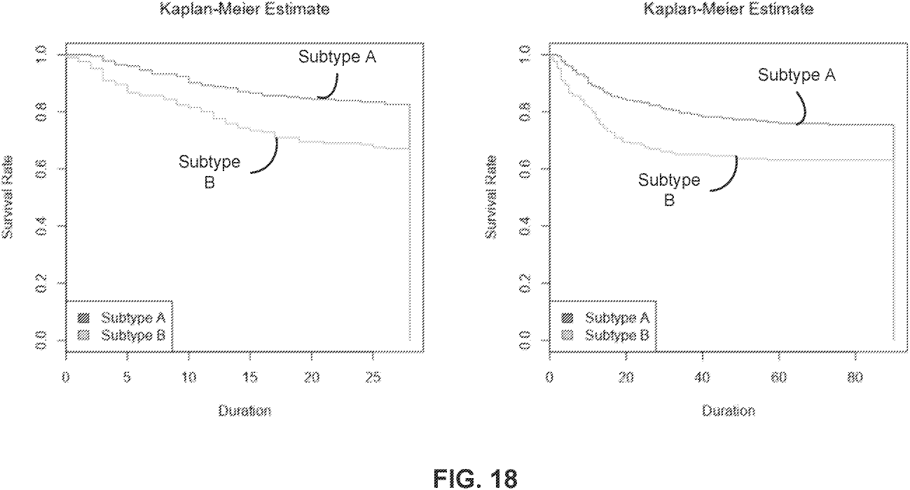

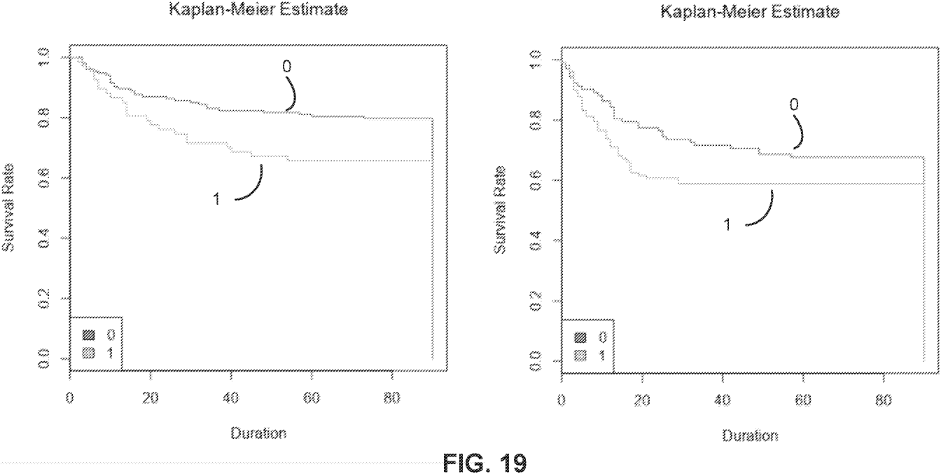

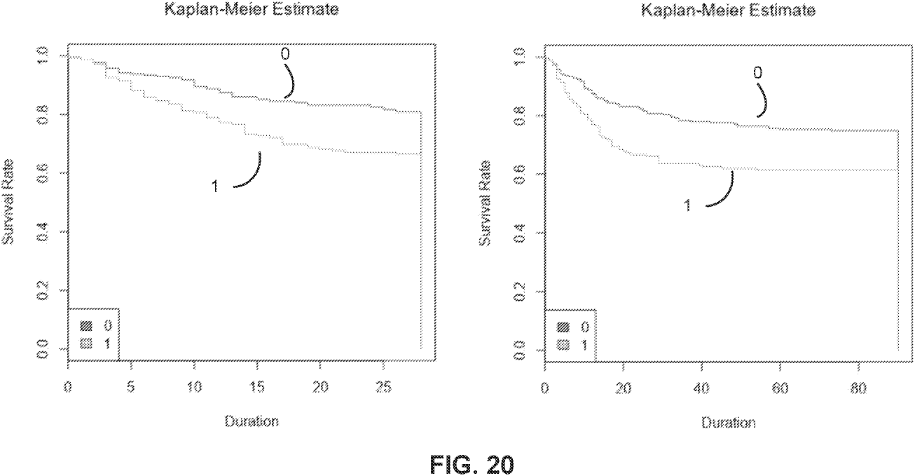

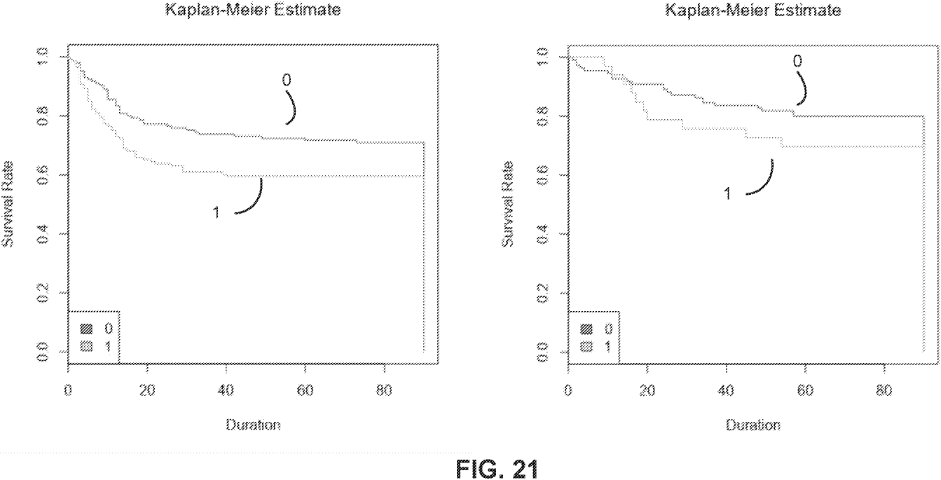

FIGS. 18-21 relate to analysis on the ARMA-KARMA-LARMA dataset. FIG. 18 depicts survival of patients in subphenotype A v. subphenotype B across the ARMA-KARMA-LARMA dataset at 28-days (left) and 90-days (right). FIG. 19 depicts survival of patients in subphenotype A (left) and subphenotype B (right) at 90 days for patients with (1) and without (0) neuromuscular block. FIG. 20 depicts survival of patients at 28 days (left) and 90 days (right) across patients that are eligible (1) or not eligible (0) for Neuromuscular block according to Cleveland Clinic criteria. FIG. 21 depicts survival of patients at 90 days with (1) and without (0) neuromuscular block for patients that are eligible (left) and ineligible (right) according to Cleveland Clinic Protocol.

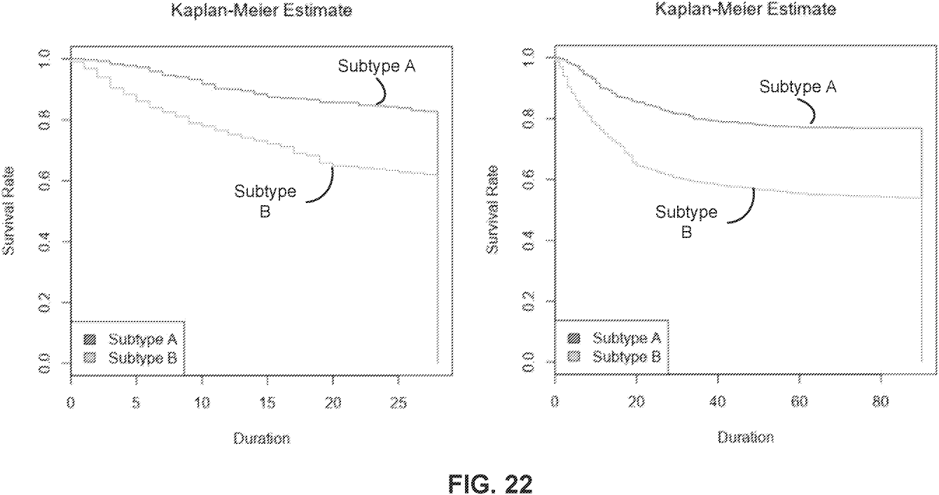

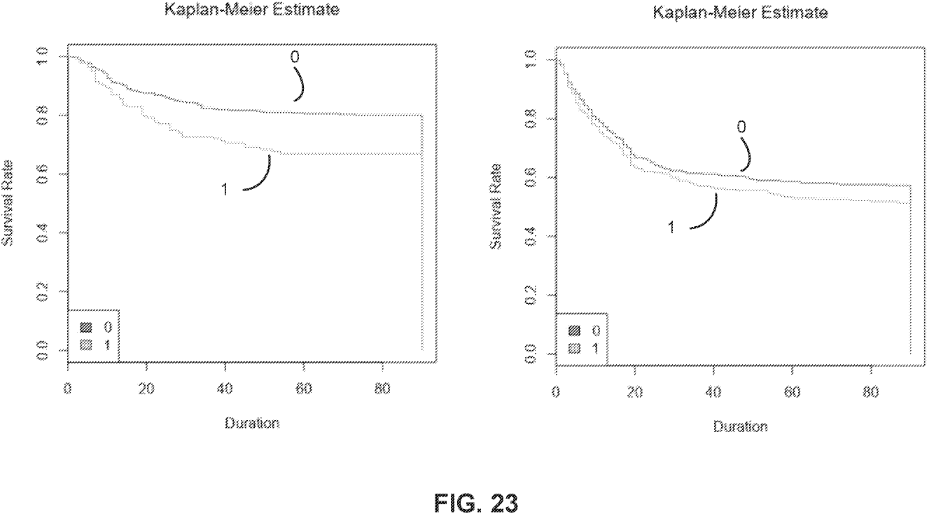

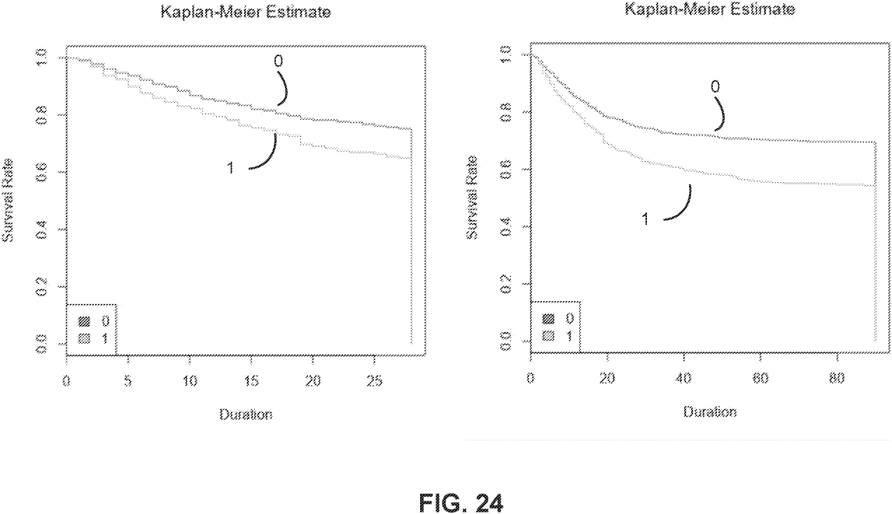

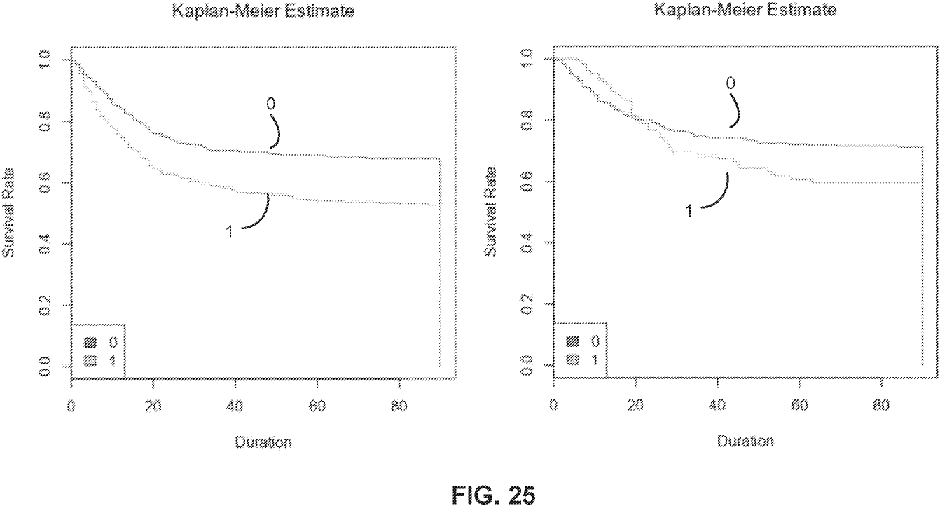

FIGS. 22-25 relate to analysis on the combined dataset (Cleveland Clinic Dataset (Without Comorbidities, plus ALVEOLI and ARMA-KARMA-LARMA Datasets). FIG. 22 depicts survival of patients in subphenotype A v. subphenotype B across the combined dataset at 28-days (left) and 90-days (right). FIG. 23 depicts survival of patients in subphenotype A (left) and subphenotype B (right) at 90 days for patients with (1) and without (0) neuromuscular block. FIG. 24 depicts survival of patients at 28 days (left) and 90 days (right) across patients that are eligible (1) or not eligible (0) for Neuromuscular block according to Cleveland Clinic criteria. FIG. 25 depicts survival of patients at 90 days with (1) and without (0) neuromuscular block for patients that are eligible (left) and ineligible (right) according to Cleveland Clinic Protocol.

TABLE 17

Subphenotype vs Neuromuscular Blockade

| | | ALVEOLI | | | | | ARMA | | | | |
|---|---|---|---|---|---|---|---|---|---|---|---|
| Metric | value | A + NMB | A - NMB | B + NMB | B - NMB | P-Value | A + NMB | A - NMB | B + NMB | B - NMB | P-Value |
| n | | 61 | 250 | 71 | 127 | | 69 | 155 | 108 | 103 | |
| Days under MV | | | | | | | | | | | |
| VentFreeDays | | 14.0 [0.0, 20.0] | 22.0 [14.0, 24.0] | 0.0 [0.0, 12.5] | 17.0 [0.0, 23.0] | <0.00 1 | 9.0 [0.0, 20.0] | 21.0 [8.0, 25.0] | 0.0 [0.0, 18.0] | 16.0 [0.0, 23.8] | <0.00 1 |
| ICU LOS | | | | | | | | | | | |
| Hospital LOS | | | | | | | | | | | |
| ICU expired | 0 | | | | | | | | | | |
| ICU expired | 1 | | | | | | | | | | |
| Hospital expired | 0 | 48 (78.7) | 220 (88.0) | 46 (64.8) | 95 (74.8) | <0.00 1 | 45 (65.2) | 123 (79.4) | 62 (57.4) | 70 (68.0) | 0.002 |
| Hospital expired | 1 | 13 (21.3) | 30 (12.0) | 25 (35.2) | 32 (25.2) | | 24 (34.8) | 32 (20.6) | 46 (42.6) | 33 (32.0) | |
| Dead28 | 0 | 50 (82.0) | 218 (87.2) | 44 (62.0) | 89 (70.1) | <0.00 1 | | | | | |
| Dead28 | 1 | 11 (18.0) | 32 (12.8) | 27 (38.0) | 38 (29.9) | | | | | | |
| Dead90 | 0 | 46 (75.4) | 213 (85.2) | 34 (47.9) | 87 (68.5) | <0.00 1 | 46 (66.7) | 124 (80.0) | 64 (59.3) | 70 (68.0) | 0.003 |
| Dead90 | 1 | 15 (24.6) | 37 (14.8) | 37 (52.1) | 40 (31.5) | | 23 (33.3) | 31 (20.0) | 44 (40.7) | 33 (32.0) | |
| 28 d survival | | | | | | | | | | | |
| 90 d survival | | | | | | | | | | | |

TABLE 17 (cont)

| | | Cleveland - all | | | | | Cleveland - no MCC | | | | |
|---|---|---|---|---|---|---|---|---|---|---|---|
| Metric | value | A + NMB | A - NMB | B + NMB | B - NMB | P-Value | A + NMB | A - NMB | B + NMB | Subphenot ype B -NMB | P-Value |
| n | | 19 | 85 | 167 | 262 | | 11 | 43 | 67 | 133 | |
| Days under MV | | 16.9 (13.3) | 12.0 (7.6) | 16.7 (13.3) | 12.3 (10.3) | <0.001 | 18.3 (11.8) | 10.5 (7.6) | 19.1 (16.0) | 12.0 (8.8) | <0.00 1 |
| VentFreeDay s | | 0.8 (3.4) | 11.3 (9.8) | 5.0 (7.9) | 8.2 (9.9) | <0.001 | 1.5 (4.5) | 14.3 (9.3) | 5.4 (8.2) | 8.8 (9.6) | <0.00 1 |
| ICU LOS | | 13.0 [6.5, 25.0] | 13.0 [8.0, 18.0] | 14.0 [8.0, 25.5] | 11.0 [7.0, 19.8] | 0.039 | 15.0 [8.5, 30.0] | 12.0 [7.0, 17.0] | 14.0 [7.0, 26.5] | 11.0 [7.0, 17.0] | 0.065 |
| Hospital LOS | | 15.0 [8.0, 33.0] | 17.0 [13.0, 24.0] | 21.0 [13.0, 31.5] | 18.0 [10.0, 27.0] | 0.232 | 15.0 [9.5, 34.5] | 16.0 [12.0, 24.5] | 21.0 [11.5, 32.0] | 16.0 [10.0, 25.0] | 0.277 |
| ICU expired | 0 | 6(31.6) | 58 (68.2) | 77 (46.1) | 139 (53.1) | 0.002 | 5 (45.5) | 33 (76.7) | 39 (58.2) | 76 (57.1) | 0.088 |
| ICU expired | 1 | 13 (68.4) | 27 (31.8) | 90 (53.9) | 123 (46.9) | | 6 (54.5) | 10 (23.3) | 28 (41.8) | 57 (42.9) | |
| Hospital expired | 0 | 6(31.6) | 56 (65.9) | 76 (45.5) | 132 (50.4) | 0.006 | 5 (45.5) | 33 (76.7) | 39 (58.2) | 74 (55.6) | 0.07 |
| Hospital expired | 1 | 13 (68.4) | 29 (34.1) | 91 (54.5) | 130 (49.6) | | 6 (54.5) | 10 (23.3) | 28 (41.8) | 59 (44.4) | |
| Dead28 | 0 | 5 (26.3) | 56 (65.9) | 89 (53.3) | 138 (52.7) | 0.012 | 4 (36.4) | 33 (76.7) | 44 (65.7) | 76 (57.1) | 0.033 |
| Dead28 | 1 | 14 (73.7) | 29 (34.1) | 78 (46.7) | 124 (47.3) | | 7 (63.6) | 10 (23.3) | 23 (34.3) | 57 (42.9) | |
| Dead90 | 0 | 5 (26.3) | 47 (55.3) | 74 (44.3) | 122(46.6) | 0.108 | 4 (36.4) | 30 (69.8) | 37 (55.2) | 71 (53.4) | 0.144 |
| Dead90 | 1 | 14 (73.7) | 38 (44.7) | 93 (55.7) | 140 (53.4) | | 7 (63.6) | 13 (30.2) | 30 (44.8) | 62 (46.6) | |
| 28 d survival | | 13.0 [9.0, 22.5] | 28.0 [15.0, 28.0] | 28.0 [9.0, 28.0] | 23.5 [9.0, 28.0] | 0.006 | 15.0 [11.0, 25.5] | 28.0 [24.5, 28.0] | 28.0 [10.5, 28.0] | 27.0 [10.0, 28.0] | 0.02 |

TABLE 18

Cleveland Clinic Neuromuscular Blockade Eligibility vs Neuromuscular Blockade Received

| | | ALVEOLI | | | | | ARMA | | | | |
|---|---|---|---|---|---|---|---|---|---|---|---|
| Metric | value | CC Eligible + NMB | CC Eligible -NMB | Not CC Eligible + NMB | Not CC Eligible -NMB | P- Value | CC Eligible + NMB | CC Eligible -NMB | Not CC Eligible + NMB | Not CC Eligible -NMB | P-Value |
| n | | 92 | 182 | 40 | 195 | | 144 | 147 | 33 | 111 | |
| Days under MV | | | | | | | | | | | |
| VentFreeDa ys | | 0.0 [0.0, 15.0] | 19.0 [2.0, 23.0] | 13.5 [0.8, 19.0] | 22.0 [14.0, 24.0] | <0.001 | 0.0 [0.0, 18.0] | 16.5 [0.0, 24.0] | 13.0 [0.0, 23.0] | 22.0 [10.0, 25.0] | <0.001 |
| ICU LOS | | | | | | | | | | | |
| Hospital LOS | | | | | | | | | | | |
| ICU expired | 0 | | | | | | | | | | |
| ICU expired | 1 | | | | | | | | | | |
| Hospital expired | 0 | 63 (68.5) | 146 (80.2) | 31 (77.5) | 169 (86.7) | 0.004 | 84 (58.3) | 105 (71.4) | 23 (69.7) | 88 (79.3) | 0.004 |
| Hospital expired | 1 | 29 (31.5) | 36 (19.8) | 9 (22.5) | 26 (13.3) | | 60 (41.7) | 42 (28.6) | 10 (30.3) | 23 (20.7) | |
| Dead28 | 0 | 61 (66.3) | 143 (78.6) | 33 (82.5) | 164 (84.1) | 0.007 | | | | | |
| Dead28 | 1 | 31 (33.7) | 39 (21.4) | 7 (17.5) | 31 (15.9) | | | | | | |
| Dead90 | 0 | 51 (55.4) | 139 (76.4) | 29 (72.5) | 161 (82.6) | <0.001 | 87 (60.4) | 105 (71.4) | 23 (69.7) | 89 (80.2) | 0.008 |
| Dead90 | 1 | 41 (44.6) | 43 (23.6) | 11 (27.5) | 34 (17.4) | | 57 (39.6) | 42 (28.6) | 10 (30.3) | 22 (19.8) | |
| 28 d survival | | | | | | | | | | | |
| 90 d survival | | | | | | | | | | | |

TABLE 18 (cont.)

| | | Cleveland - all | | | | | Cleveland - no MCC | | | | |
|---|---|---|---|---|---|---|---|---|---|---|---|
| Metric | value | CC Eligible + NMB | CC Eligible - NMB | Not CC Eligible + NMB | Not CC Eligible -NMB | P- Value | CC Eligible + NMB | CC Eligible -NMB | Not CC Eligible + NMB | Not CC Eligible -NMB | P- Value |
| n | | 156 | 174 | 30 | 173 | | 64 | 84 | 14 | 92 | |
| Days under MV | | 17.5 (13.3) | 13.3 (10.7) | 13.0 (12.4) | 11.1 (8.5) | <0.001 | 19.2 (15.4) | 12.5 (8.6) | 18.4 (15.9) | 10.8 (8.4) | <0.001 |
| VentFreeDay s | | 4.1 (7.0) | 7.9 (9.6) | 7.5 (10.5) | 10.1 (10.2) | <0.001 | 4.0 (6.8) | 9.4 (9.4) | 8.6 (10.9) | 10.8 (10.2) | <0.001 |
| ICU LOS | | 14.0 [8.8, 26.2] | 13.0 [8.0, 21.0] | 12.0 [7.0, 20.0] | 11.0 [6.0, 17.0] | 0.004 | 14.5 [7.0, 27.0] | 13.0 [8.0, 19.2] | 16.5 [11.2, 27.5] | 10.0 [6.0, 16.0] | 0.016 |
| Hospital LOS | | 21.0 [12.8, 32.2] | 20.0 [11.0, 27.0] | 16.5 [11.0, 26.2] | 16.0 [11.0, 25.0] | 0.089 | 20.0 [9.8, 33.0] | 19.0 [11.0, 25.2] | 22.0 [14.5, 35.0] | 14.0 [10.0, 22.2] | 0.096 |
| ICU expired | 0 | 67 (42.9) | 94 (54.0) | 16 (53.3) | 103 (59.5) | 0.025 | 33 (51.6) | 53 (63.1) | 11 (78.6) | 56 (60.9) | 0.233 |
| ICU expired | 1 | 89 (57.1) | 80 (46.0) | 14 (46.7) | 70 (40.5) | | 31 (48.4) | 31 (36.9) | 3 (21.4) | 36(39.1) | |
| Hospital expired | 0 | 66 (42.3) | 87 (50.0) | 16 (53.3) | 101 (58.4) | 0.035 | 33 (51.6) | 51 (60.7) | 11 (78.6) | 56 (60.9) | 0.272 |
| Hospital expired | 1 | 90 (57.7) | 87 (50.0) | 14 (46.7) | 72 (41.6) | | 31 (48.4) | 33 (39.3) | 3 (21.4) | 36(39.1) | |
| Dead28 | 0 | 78 (50.0) | 93 (53.4) | 16 (53.3) | 101 (58.4) | 0.5 | 37 (57.8) | 52 (61.9) | 11 (78.6) | 57 (62.0) | 0.552 |
| Dead28 | 1 | 78 (50.0) | 81 (46.6) | 14 (46.7) | 72 (41.6) | | 27 (42.2) | 32 (38.1) | 3 (21.4) | 35 (38.0) | |
| Dead90 | 0 | 64 (41.0) | 80 (46.0) | 15 (50.0) | 89 (51.4) | 0.29 | 31 (48.4) | 49 (58.3) | 10 (71.4) | 52 (56.5) | 0.387 |
| Dead90 | 1 | 92 (59.0) | 94 (54.0) | 15 (50.0) | 84 (48.6) | | 33 (51.6) | 35 (41.7) | 4 (28.6) | 40 (43.5) | |
| 28 d survival | | 25.5 [9.0, 28.0] | 24.0 [11.2, 28.0] | 25.0 [8.0, 28.0] | 28.0 [11.0, 28.0] | 0.672 | 28.0 [8.5, 28.0] | 28.0 [14.8, 28.0] | 28.0 [24.2, 28.0] | 28.0 [11.0, 28.0] | 0.557 |
| 90 d survival | | 25.5 [9.0, 90.0] | 24.0 [11.2, 90.0] | 26.0 [8.0, 90.0] | 33.0 [11.0, 90.0] | 0.574 | 32.5 [8.5, 90.0] | 41.0 [14.8, 90.0] | 90.0 [24.5, 90.0] | 33.5 [11.0, 90.0] | 0.324 |

Unlike supervised learning which requires data to be labeled with patient outcomes, unsupervised learning draws inferences from the data without awareness of associated patient outcomes. By using K-means clustering analysis as an unsupervised learning approach, this methodology elucidated hidden patterns in ARDS patients. Two ARDS subphenotypes, subphenotype B (high-mortality) and subphenotype A (low-mortality,) were consistently observed by applying K-means clustering to clinical trial and clinical practice data. Comparison of the physiological characteristics of the two subphenotypes shows distinct characteristics between subphenotypes, indicating potential for guided treatment.

The identified subphenotypes were analyzed to identify differential responses to treatment. A potential explanation for the differences in patient outcomes between subphenotypes is that patients in one group are more likely to experience micro-asynchrony. Another potential explanation for the differences in patient outcomes between subphenotypes is that subphenotype B patients are inflamed whereas subphenotype A patients are not inflamed. NMBs have an anti-inflammatory effect. Reducing inflammation in subphenotype B patients may block an immune over-response, whereas patients in subphenotype A may experience normal immune response and the anti-inflammatory effect of the NMBs stops their functioning immune system from doing its job. Another potential explanation for the differences in patient outcomes between subphenotypes is that patients in subphenotype B have additional underlying comorbidities that make it harder to wean them from NMB and ventilator use.

The methods disclosed herein are intended to be used by healthcare professionals to determine a prognostic mortality risk associated with ARDS. It is intended for use on patients having or suspected of having ARDS. The result of the ARDS prognostic tool is intended to be used in conjunction with other clinical assessments by healthcare professionals to assist with triage and/or prioritization of critically ill patients. The ARDS therapy guidance tool is machine learning software that analyzes data from the EHR and is intended to be used by healthcare professionals as aid in assessing patients for whom treatment with NMB agents is being considered.

### Example 2: Example Logistic Regression ARDS Classifiers Differentiate Patient Populations

Using the same datasets and Model input variables outlined above in Example 1, rather than using a K-means clustering Model, binary classifiers were trained to predict patient mortality by assigning each patient to a high mortality risk group or to a low mortality risk group. While in some embodiments, the binary classifiers may be trained using a variety of machine learning methods (e.g., logistic regression classifier, decision tree classifier, random forest classifier, gradient boosting classifier, neural net, and others), in this particular embodiment the Scikit-leam (Pedregosa, et al., 2011) tool kit was used to train a standard scalar (https://scikit-learn.org/stable/modules/generated/sklearn.preprocessing.StandardScaler.html) for each input variable and then fit a logistic regression (https://scikit-learn.org/stable/modules/generated/sklearn.linear_model.LogisticRegression.html) to the resulting scaled input variables.

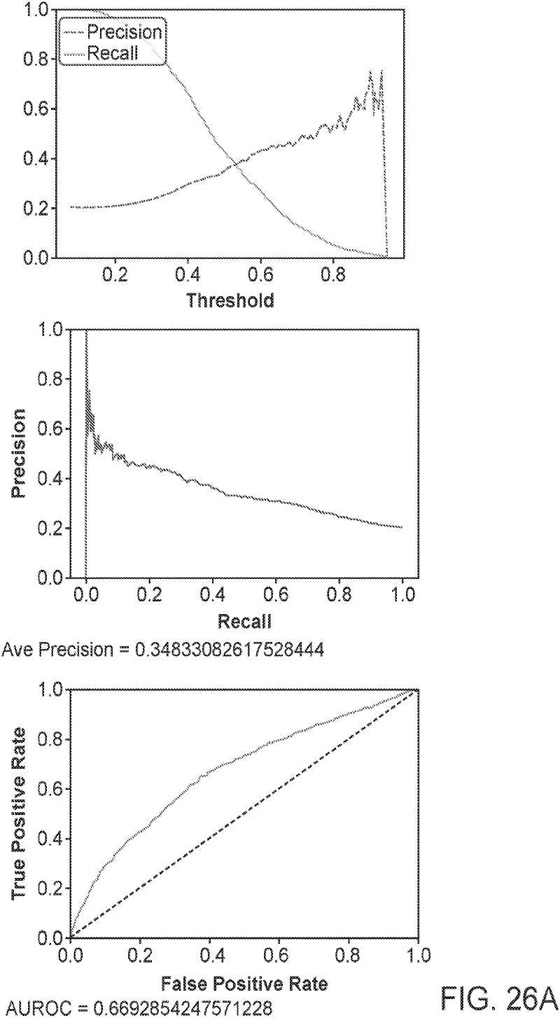

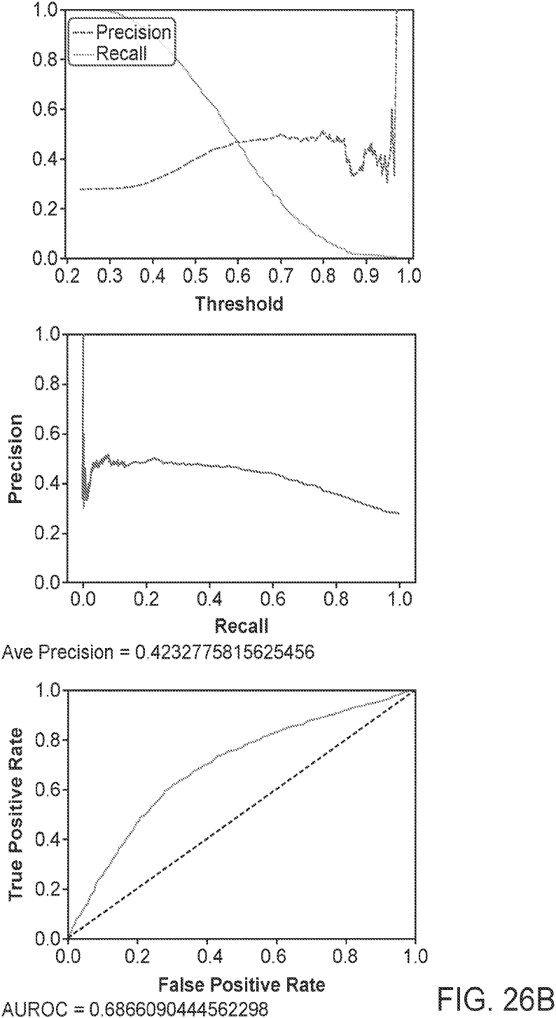

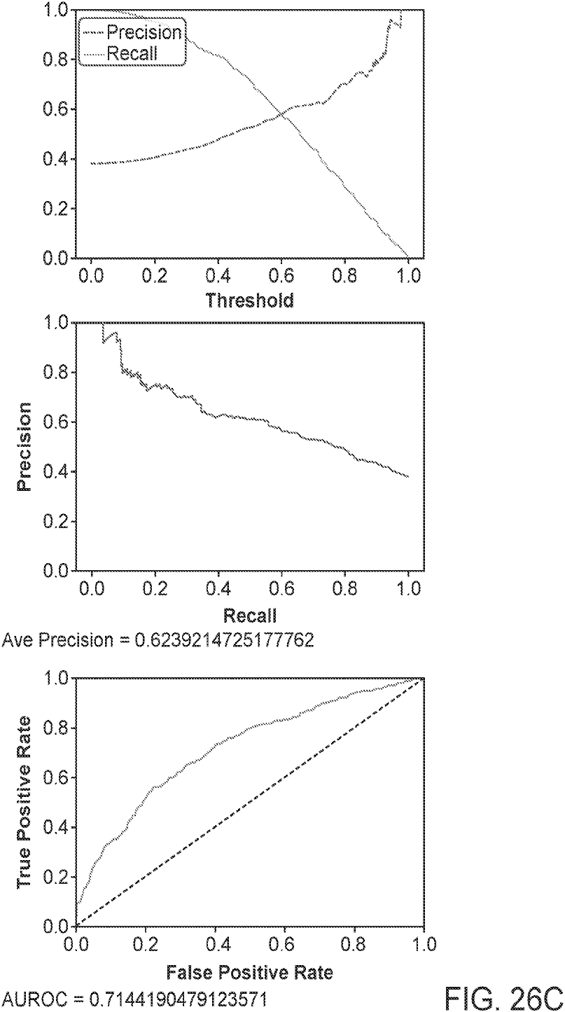

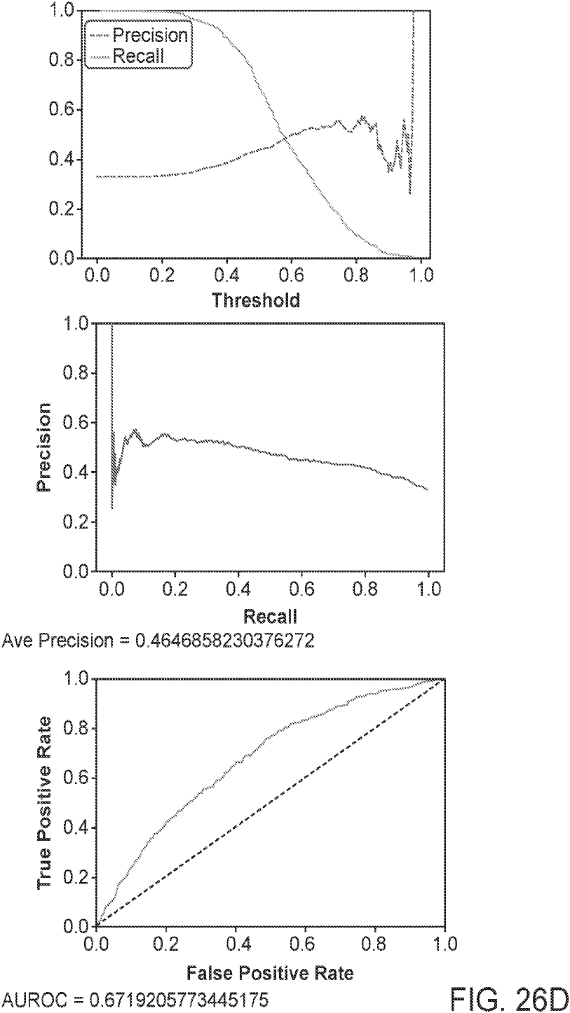

Table 19 below presents the input variables of the logistic regression Models 1-4. FIGS. 26A-26D show the results of training and validating the logistic regression Models 1-4.

TABLE 19

Input variables for logistic regression models 1-4

| | | | | |
|---|---|---|---|---|
| | Model 1 | Model 2 | Model 3 | Model 4 |
| Input Variables | Arterial pH-R, Bicarbonate-L, Creatinine-R, Diastolic BP-R, FIO₂- R, Heart Rate-R, Mean arterial pressure-H, mean arterial pressure-L, potassium-R, respiratory rate-H, respiratory rate-L, SPO₂—R, systolic BP-R | Arterial pH-R, bicarbonate-L, creatinine-R, FIO₂- R, heart rate-R, PaO₂—R, mean arterial pressure-R, respiratory rate-R | Age, arterial pH-R, bicarbonate-L, bilirubin-H, BMI, creatinine-R, FiO₂- R, gender, heart rate-R, PaCO₂—R, PaO₂/FiO₂-LP, PaO₂—R, PEEP-R, Platelet-L, Tidal Volume-R, mean arterial pressure-R, respiratory rate-R | Arterial pH-R, bicarbonate-R, BMI, creatinine-R, FiO₂-R, gender, heart rate-R, PaCO₂—R, PaO₂/FiO₂-LP, PEEP-R, Platelets-L, mean arterial pressure-R, respiratory rate-R |

Table 20 below depicts key logistic regression Model performance metrics including the training and validation area under the receiver-operator curve (AUROC) and the training and validation area under the precision-recall curve (AUPRC).

TABLE 20

Performance metrics of logistic regression models 1-4

| Model | AUROC - Train | AUROC - Validate | AUPRC - Train | AUPRC - Validate |
|---|---|---|---|---|
| Model 1 | 0.67 | 0.67 | 0.42 | 0.40 |
| Model 2 | 0.65 | 0.69 | 0.40 | 0.42 |
| Model 3 | 0.75 | 0.71 | 0.54 | 0.62 |
| Model 4 | 0.67 | 0.67 | 0.43 | 0.46 |

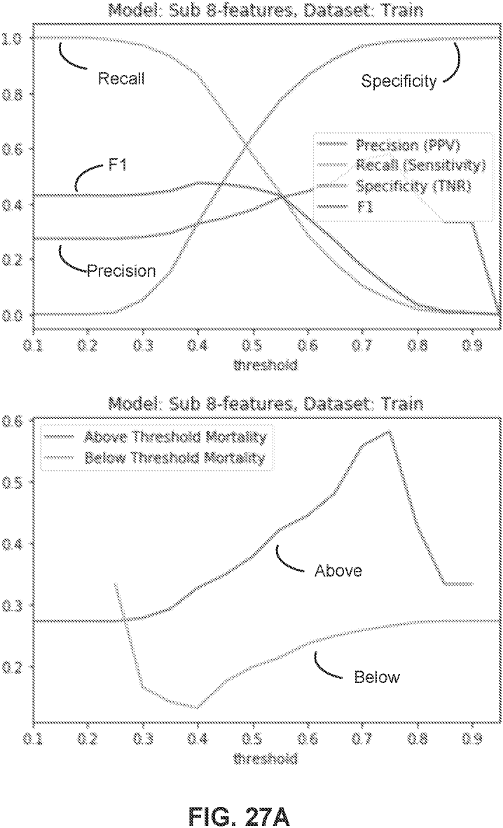

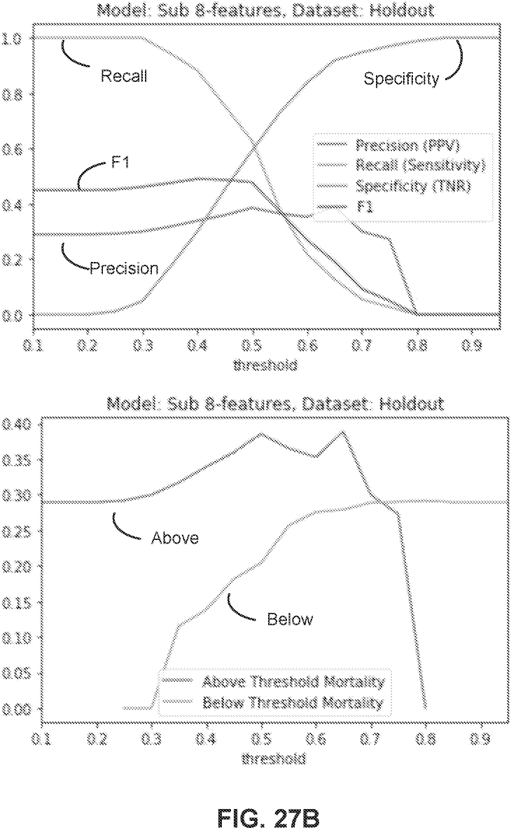

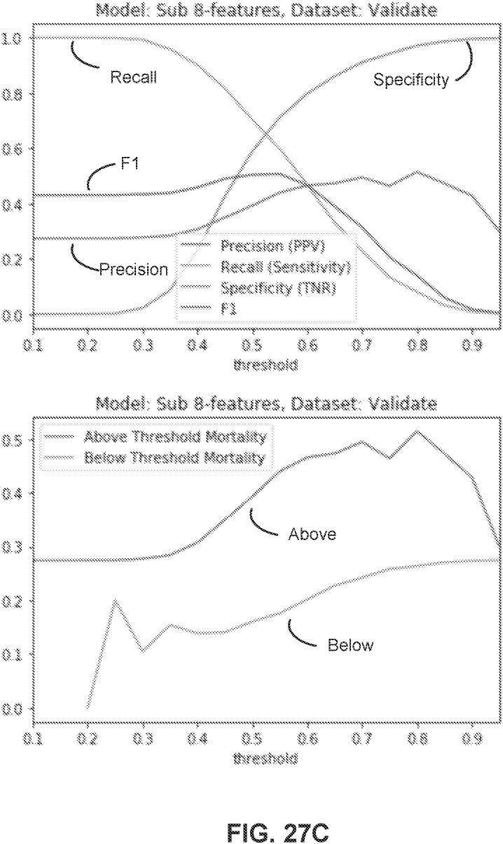

To further evaluate the clinical utility of logistic regression Models 1-4, the impact of tuning the threshold used to turn a decimal score between 0 and 1 output by the logistic regression Model into a 1 (dead) or 0 (alive) prediction, was examined. FIGS. 27A-27C below shows the impact of varying the threshold on logistic regression Model 2 performance and mortality separation for the training and validation datasets. Specifically, FIG. 27A shows the impact using the training dataset (e.g., 64% of ARDSNet blended dataset). FIG. 27B shows the impact using a holdout dataset (e.g., 20% of ARDSNet dataset). FIG. 27C shows the impact using a validation dataset (e.g., combination of eICU, ART, Cleveland Clinic, and remaining ARDSnet Datasets). Similar analysis may also be performed for logistic regression Models 1, 3, and 4 as well.

Table 21 below depicts logistic regression Model 2 performance metrics with scores tuned to various prediction thresholds. Specifically, Table 21 below depicts that there are one or more prediction thresholds for which logistic regression Model 2′s performance metrics meet or exceed those of procalcitonin (PCT) as a mortality predictor (Schuetz et al., 2017). Underlined values in Table 21 indicates where logistic regression Model 2 matches or exceeds PCT performance on the subset of their patients who were in the ICU on Day 4. In contrast to PCT, which requires multiple blood tests on Day 0 or 1 of ARDS diagnosis and then again on Day 4 to provide a prognosis, the Models presented herein provide a prognostic immediately following ARDS diagnosis if the Model input variables have been measured in the previous 24 hours.

TABLE 21

Performance metrics according to thresholds

| Dataset | Optimal threshold | Precision (PPV) | NPV | Recall (Sensitivity) | Specificity (TNR) | F1 |
|---|---|---|---|---|---|---|
| Train (ARDSNet) | 0.04 | 32.7% | 86.7% | 86.6% | 32.9% | 47.5% |
| | 0.425 | 33.7% | 84.4% | 79.9% | 40.8% | 47.4% |
| Validate (Across sources) | 0.45 | 35.0% | 86.0% | 81.6% | 42.6% | 48.9% |
| Holdout (ARDSNet) | 0.425 | 34.0% | 81.8% | 80.0% | 36.7% | 47.7% |

Table 22 below confirms that logistic regression Model 2 produces similar mortality risk stratification to the k-means clustering Models discussed above, as well as to PCT.

TABLE 22

Mortality stratification according to thresholds

| Dataset | Optimal threshold | N Above threshold | Above Threshold Mortality | N Below Threshold | Below Threshold Mortality |
|---|---|---|---|---|---|
| Train | 0.04 | 871 | 32.7% | 331 | 13.3% |
| (ARDSNet) | 0.425 | 780 | 33.7% | 422 | 15.6% |
| Validate (Across sources) | 0.45 | 3127 | 35.0% | 1759 | 14.0% |
| Holdout (ARDSNet) | 0.425 | 259 | 34.0% | 121 | 18.2% |

### Example 3: Ensemble Based Models for Mortality Prediction and Treatment Guidance

### Methods

There are a number of ensemble techniques which can be used to improve algorithm performance. The general concept of ensembling models involves taking the output from one or more models and using that output as input feature(s) for another model, potentially along with additional new data features.

Using the same data sources and model features as outlined in the EHR-based ARDS Subphenotyper for Mortality Prediction and Treatment Guidance Technical Note, an additional set of ARDS mortality classifiers was developed by ensembling output from the K-means clustering-derived ARDS subphenotype with additional features. FIG. 28 shows an example ensemble technique for performing unsupervised K-means clustering on 8 data elements, and uses the subphenotype assignment (derived from the K-means cluster) as input to a supervised logistic regression algorithm with 9 additional data elements. Generally, the output of one model can be used as an input variable to a second model. The second model may or may not have overlapping input variables with the original model.

In this specific case, the Sub-8 K-means clustering model was used as input to the various classifier models. Classifier models were evaluated both with and without the 8 features of Sub8. Table 23 below shows an example of the variables input to the ensemble models.

TABLE 23

Data elements used in example Ensemble models

| | | | | | | | |
|---|---|---|---|---|---|---|---|
| Column Name | Description | Timing / Calculation * within 24 hours prior to ARDS diagnosis / study enrollment | Sub-8 phenotype | Platinum (E4) | Gold (E2) | Silver (E5) | Bronze (E17) |
| Sub-8 phenotype | Subphenotype Output from Sub-8 K-means cluster | | | y | y | y | y |
| ARTPHR | Arterial pH | Most recent* | y | | | y | y |
| BICARL | Bicarbonate | Lowest* | y | | | y | y |
| CREATR | Creatinine | Most recent* | y | | | y | y |
| FIO2R | FiO₂ (Fraction of inspired oxygen) | Most recent* | y | | | y | y |
| HRATER | Heart rate | Most recent* | y | | | y | y |
| MEANAP R | Mean arterial pressure | Most recent* | y | | | y | y |
| RESPR | Respiration rate | Most recent* | y | | | y | y |
| PAO2R | PaO₂ (Partial Pressure of Oxygen) | Most recent* | y | | | y | y |
| GENDER | Gender | 1 = Male, 2 = Female | | y | y | y | y |
| AGE | Age | At admission | | y | y | y | y |
| BILIH | Bilirubin | Highest* | | y | y | y | |
| PACO2R | PaCO₂ (Partial Pressure of Carbon Dioxide) | Most recent* | | y | y | | |
| PAFILP | PaO₂ / FiO₂ | Lowest on day of diagnosis or enrollment | | y | y | | |
| PEEPR | Positive End Expiratory Pressure | Most recent* | | y | y | | |
| PLATEL | Platelet count | Lowest* | | y | y | | |
| TIDALR | Tidal volume | Most recent* | | y | y | | |
| BMI | Body Mass Index | At admission | | y | | | |

Alternatively, an ensemble model may be built which creates a different model (in this case a logistic regression model) for each subphenotype from the input K-means cluster (FIG. 29). Specifically, FIG. 29 shows an example of an ensemble model where different supervised mortality prediction algorithms are applied to the data for a given patient depending on their subphenotype from the unsupervised K-means clustering. In this case, separate mortality prediction models would be created for each subphenotype from the original K-means clustering subphenotyping classifier. The secondary algorithms could have different input variables with different weights, and could even use different underlying machine learning algorithms.

Alternatively, a combination of model outputs (K-means clustering, logistic or linear regression, GMM clustering, etc with the same or different input variables), could be used in combination as inputs to an ensembling algorithm, whose output could then be used to predict an ARDS prognosis or other outcome (FIG. 30). Specifically, FIG. 30 shows an ensemble model where a combination of different supervised and unsupervised model outputs become inputs to a final ensemble algorithm that then produces a mortality score.

An ensemble of models could also include a series of models which would be applied based on the amount of data available. For the example below, if all data elements are available, the top performing model could be used. If some data elements are unavailable for a given patient or EHR system, a second line model (the gold model shown here) using fewer data elements could be used. If not all of those elements are available, a third line model could be used, and so on. Specifically, FIG. 31 shows a series of models ensembled in a waterfall design based on the amount of data available for a given patient.

### Results

A number of ensembled models were created. FIG. 28 is an example workflow for Ensemble 4, the “Platinum” model in Table 24. Eight features were input to K-means clustering. The output subphenotype from clustering was input to a logistic regression model, with 9 additional variables. Performance of the ensembled model is shown in the “Platinum” column of Table 24. The 17-features maximized AUROC, NPV, and sensitivity.

In critical care settings where patients are often treated according their height-based ideal weight rather than their actual admission weight, patient weight is not always recorded in the EHR, and thus the patient BMI may not be available. In that case, a second line model (marked gold below) using 16 inputs can be ensembled in the algorithm suite. In this example, the model follows the flow of FIG. 28, but excludes the BMI element. Similarly, third (Ensemble 5) and fourth (Ensemble 17) line models were derived to maximize the population of patients who can be scored on the algorithm while optimizing performance for patients who have the most available data.

TABLE 24

Performance of various ensemble models

| Model (# features) Validation Performance | Platinum (17) (E4, th = 0.45 ) | Gold (16) (E2, th = 0.425) | Silver (11) (E5, th = 0.55) | Bronze (10) (E17, th=0.55) |
|---|---|---|---|---|
| Mortality Difference (high rate / low rate, high/low factor) | 52.5% / 27.4% (1.91 x) | 55.1% / 34.2% (1.61 x) | 50.0% / 28.7% (1.74 x) | 46.8% / 26.3% (1.78 x) |
| Sensitivity | 78.5% (74.0 - 82.5%) | 77.4% (73.8 -80.7%) | 77.7% (74.4 -80.6%) | 79.0% (76.3 -81.4%) |
| Specificity | 44.5% (40.0 - 49.0%) | 40.8% (37.0 -44.8%) | 41.6% (38.4 -44.8%) | 39.6% (37.1 -42.2%) |
| PPV | 52.5% (48.4 - 56.7%) | 55.1% (51.7 -58.5%) | 50.0% (47.0 -52.9%) | 46.8% (44.3 -49.2%) |
| NPV | 72.6% (67.1 - 77.5%) | 65.8% (60.9 -70.4%) | 71.3% (67.3 -75.0%) | 73.7% (70.4 -76.7%) |
| AUROC | 0.689 | 0.673 | 0.658 | 0.643 |
| AUPRC | 0.650 | 0.668 | 0.597 | 0.532 |

### Predicting Biomarker Levels

Using the same data sources and model features outlined in the EHR-based ARDS subphenotyper for Mortality Prediction and Treatment Guidance Technical note (K-means Cluster model 2, trained on ARMA-ALVEOLI-FACTT), the patient’s subphenotype was used to evaluate levels of circulating plasma biomarkers measured on the day of study randomization in the ARMA and ALVEOLI studies. Two sample t-tests or Kruskal-Wallis tests were used to identify differences in biomarker levels, depending on whether the biomarker level had a normal distribution. Based on the difference of biomarker levels between subphenotypes, an EHR-only based algorithm could be used to predict specific levels of biomarkers, or ratios of biomarkers.

As shown in Table 25, in both datasets, Subphenotype B (higher mortality subphenotype) exhibited increased levels of ICAM-1 and IL-6. In the ARMA dataset, subphenotype B was further indicative of increased circulating levels of IL-8, sTNFR1, PAI-1, VWF, IL-10 and sTNFR2.

TABLE 25

Subphenotypes A and B display significant difference in biomarker levels for a broad range of biomarkers. Biomarker data shown as median (interquartile range); ICAM-1 = intercellular adhesion molecule-1; IL-6 = interleukin-6; PAI-1 = plasminogen activator inhibitor-1; IL-8 = interleukin-8, IL-10 = interleukin-10; TNFR-I = tumor necrosis factor receptor 1; TNFR-II = tumor necrosis factor II, VW = von Willebrand factor

| ALVEOLI Trial | Subphenotype B N=172 | Subphenotype A N=318 | p-value |
|---|---|---|---|
| In-hospital mortality, n (%) | 50 (29.1) | 52 (16.4) | 0.001 |
| ICAM-1 (ng/mL) | 1038.6 [744.9, 1586.7] | 831.9 [582.3, 1221.3] | <0.001 |
| IL-6 (pg/mL) | 637.5 [158.0, 2823.0] | 175.0 [78.8,422.0] | <0.001 |
| ARMA trial | Subphenotype B N=197 | Subphenotype B N=201 | p-value |
| In-hospital mortality, n (%) | 71 (36.0) | 48 (23.9) | 0.011 |
| PAI-1 (ng/mL) | 264.9 (577.6) | 115.4 (172.9) | 0.007 |
| IL-6 (pg/mL) | 682.0 [255.5, 2018.5] | 176.0 [72.8, 399.8] | <0.001 |
| IL-8 (pg/mL) | 86.0 [43.5, 239.5] | 34.0 [0.0, 72.0] | <0.001 |
| IL-10 (pg/mL) | 39.3 [12.5, 89.1] | 0.0 [0.0, 29.5] | <0.001 |
| TNFR-I (pg/mL) | 5760.5 [3198.2, 11253.2] | 2315.0 [1704.0, 3476.0] | <0.001 |
| TNFR-II (pg/mL) | 14630.5 [9236.5, 27460.2] | 6019.0 [4646.5, 8571.0] | <0.001 |
| ICAM-1 (ng/mL) | 855.1 [552.4, 1357.7] | 604.4 [350.6, 839.0] | <0.001 |
| VW (% control) | 386.0 [212.2, 560.2] | 306.5 [167.8, 417.2] | 0.019 |

Four biomarkers were correlated with Ensembles 14 (17 features) and Ensemble 4 (8 features, K-means Cluster 8 plus bilirubin subphenotype) to see if there was a correlation between biomarker level and predictor score. Pearson correlation identifies linear correlation, whereas Spearman correlation nonparametrically quantifies rank correlation (the largest values in X correlate with largest values in Y and smallest values in X correlate with smallest values in Y, but not necessarily in a linear manner). Table 26 shows that correlation with biomarkers varies by algorithm. IL6 exhibited a moderate Spearman correlation with Ensemble 14 score.

TABLE 26

Example data shows varying levels of correlation depending on biomarker, type of correlation and algorithm

| | Pearson Correlation to Ensemble 14 | Spearman Correlation to Ensemble 14 | Pearson Correlation to Ensemble 4 | Spearman Correlation to Ensemble 44 |
|---|---|---|---|---|
| IL6 | 0.221639 | 0.475682 | 0.271703 | 0.307956 |
| PAI1 0 | 0.252682 | 0.148296 | 0.21822 | 0.264941 |
| IL8 | 0.076357 | 0.367898 | 0.152016 | 0.326185 |
| IL10 | 0.172776 | 0.285312 | 0.227885 | 0.227885 |

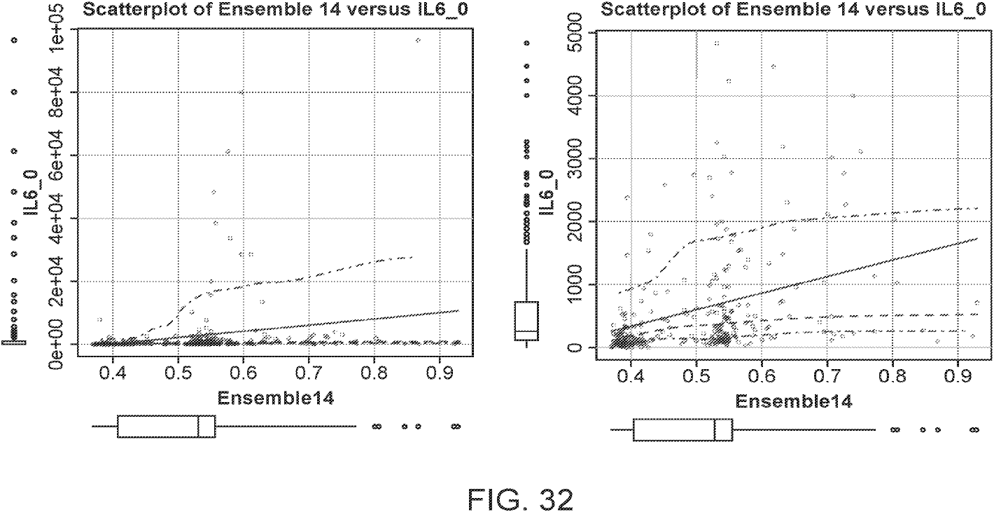

Scatter plots of Ensemble 14 score versus level of IL-6 (FIG. 32) visually show the correlation described in Table 26. Specifically, FIG. 32 shows scatter plots of Ensemble 14 (x-axis) versus level of IL-6 (y-axis) with best-fit lines shown. The left plot of FIG. 32 includes all data, whereas the right plot of FIG. 32 excludes values of IL-6 more than 5,000. In each plot, the solid line shows linear regression fit, the dashed line shows the non-parametric local regression (locally estimated scatterplot smoothing - LOESS) smoothed over 50 data points, and the dash-dot lines show the root-mean-square positive and negative residuals from the LOESS line. This suggests that an EHR-based algorithm could be tuned to predict a biomarker level, a ratio of biomarker levels, or another continuous clinical variable.

### Example 4: Ensemble Based Models for Classifying Patients Into More Than Two Mortality Risk Groups

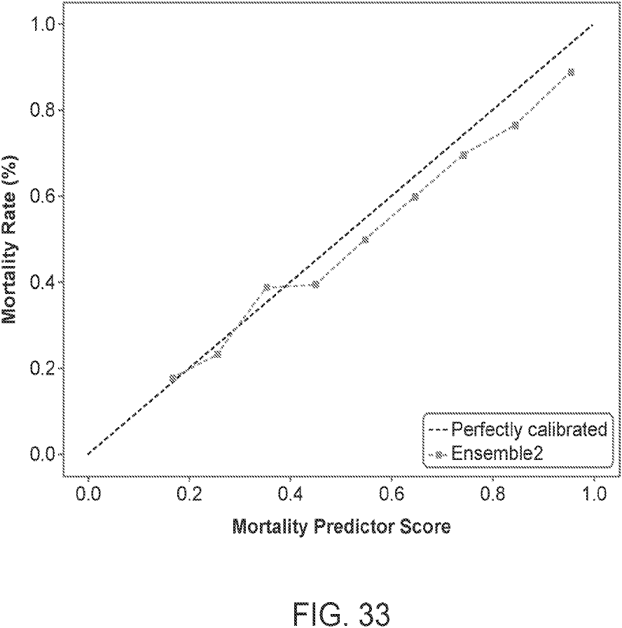

In addition to the binary high risk / low risk mortality predictions discussed in the above examples, the results from the ARDS mortality prediction algorithms can be used with more than one score threshold to produce more than two risk groups. In one embodiment, the ARDS Prognostic Digital version 1 (APDvl), the Gold ensemble model described in Table 23 is used with two prediction score thresholds to produce three categories of mortality risk: lower, medium, and higher. FIG. 33 shows the calibration curve for a model output as evaluated on a validation cohort. FIG. 33 specifically shows the calibration curve for APDvl mortality prediction logistic regression. Scores were binned into 10 intervals from 0 - 1, and for each bin the average mortality prediction score was compared to the observed mortality rate (line and markers). The closer the observed performance is to the 1:1 dashed line, the greater the ability of the model to predict mortality. There is good agreement between the average mortality prediction from APDv1 and the observed mortality across all the whole range of logistic regression scores.

Mortality prediction score thresholds of 0.3 and 0.6 are used to categorize patients into lower risk, medium risk, and higher risk categories. The mortality separation for the three APDvl risk groups is shown in Table 27 for the validation cohort. The 95% confidence intervals for the three groups do not overlap, and the chi-squared p-value for mortality rate separation between the three groups is 8.40e-22. The lower risk and higher risk groups are likely to be most useful in informing clinical decisions; they cover 11.0% and 31.4% of the validation population, respectively, with 42.4% of the population falling into one of those two groups.

TABLE 27

Count of patients in each APDvl risk group for the validation data, and in-hospital mortality rates with 95% confidence intervals. Mortality rates for each risk group have nonoverlapping confidence intervals, and chi-squared p-value for mortality separation = 8.40e-22

| | Lower Risk Group | Medium Risk Group | Higher Risk Group | Total |
|---|---|---|---|---|
| N (%) | 136 (11.0%) | 711 (57.6%) | 388 (31.4%) | 1235 |
| In-hospital Mortality Rate (95% confidence Interval) | 22.1% (15.6 - 30.1%) | 43.5% (39.8 - 47.2%) | 66.8% (61.8 - 71.4%) | 48.4% |

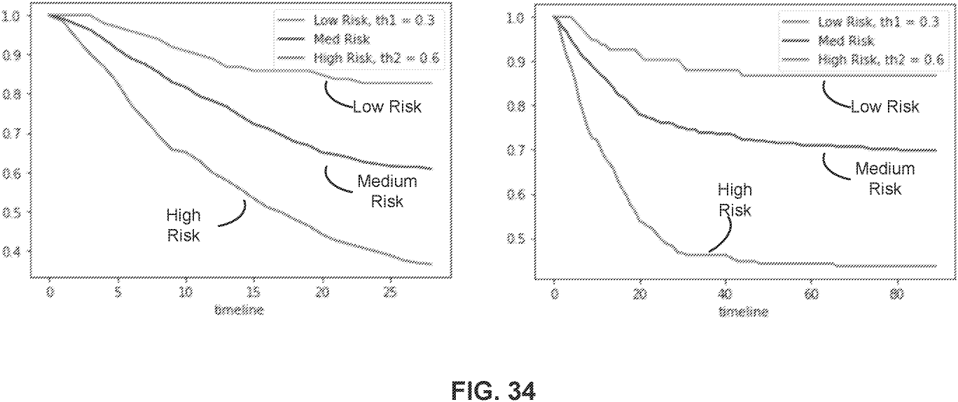

To visualize the separation of the APDvl risk groups, Kaplan-Meier survival curves were implemented. Specifically, FIG. 34 shows Kaplan-Meier survival curves for the three risk groups in APDvl. Logrank p-value for significance of separation = 1.3e-19. These 28-day (left panel of FIG. 34) and 90-day (right panel of FIG. 34) survival curves include all patients in the validation cohort for whom the 28-day and 90-day survival times are known. This includes most of the patients in the ART and Cleveland Clinic data sets. The eICU data set is limited to in-hospital mortality information, from which 28-day and 90-day survival times have been inferred only for cases where the patient died in hospital or their hospital stay extended beyond the relevant survival times.

There are two useful baselines in comparing APDvl performance to other commonly accepted approaches for predicting the mortality of critically ill patients such as those with COVID-19 pneumonia: procalcitonin (PCT) and the APACHE and SAPS severity scores. While neither Procalcitonin nor APACHE and SAPS are directly used for the in-hospital mortality prognosis of ARDS patients, they are simply used as surrogate market indicators for performance to guide product development.

In comparing the results of APDvl to procalcitonin, the FDA-approved procalcitonin assay is intended to be used as a mortality prognostic for sepsis patients. This is a relevant benchmark as most COVID-19 patients with ARDS would also meet Sepsis-3 criteria (infection with dysregulated immune response causing life-threatening organ dysfunction). However, the PCT mortality prognostic requires measuring procalcitonin levels in the patients’ blood on Day 0 or Day 1 and again on Day 4 in order to find whether the level has dropped by 80% or more over that time. This means the PCT prognostic result is not available to the clinical team until four days into treating the patient; in contrast APDvl uses clinical variables measured in the 24 hours prior to the patients’ ARDS diagnosis and is available without waiting to collect further data.

The MOSES study that validated the usefulness of PCT as a mortality prognostic found that their low risk group had an average 28-day mortality of 10.7% (6.6 - 14.9%) compared with 20.4% (16.3 - 24.4%) for their high risk group. Given that the overall mortality rate for their intent to diagnose (ITD) population was 16.9% compared to 48.4% for the validation cohort, these rates cannot be directly compared to the APDv1 lower and higher risk group mortality rates. However the relative risk ratio of their high to low mortality groups is 1.9 while the relative risk ratio of the APDvl high to low mortality groups is 3.0.

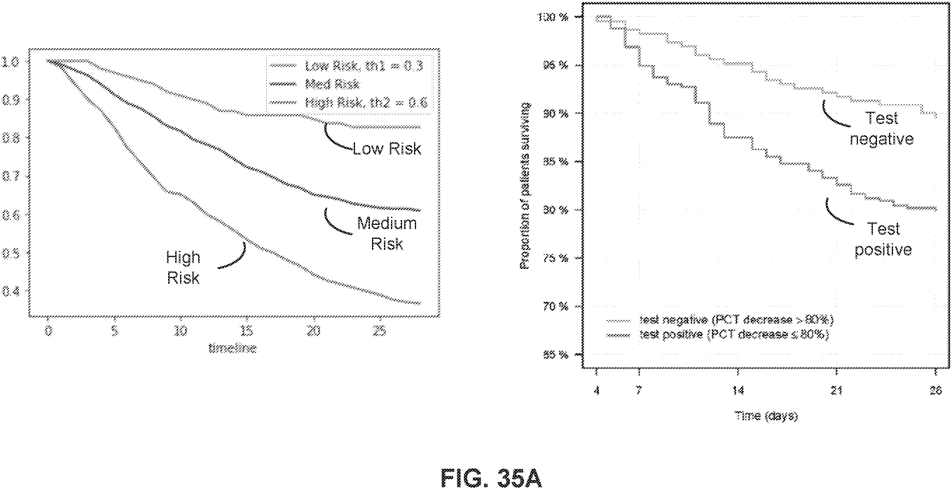

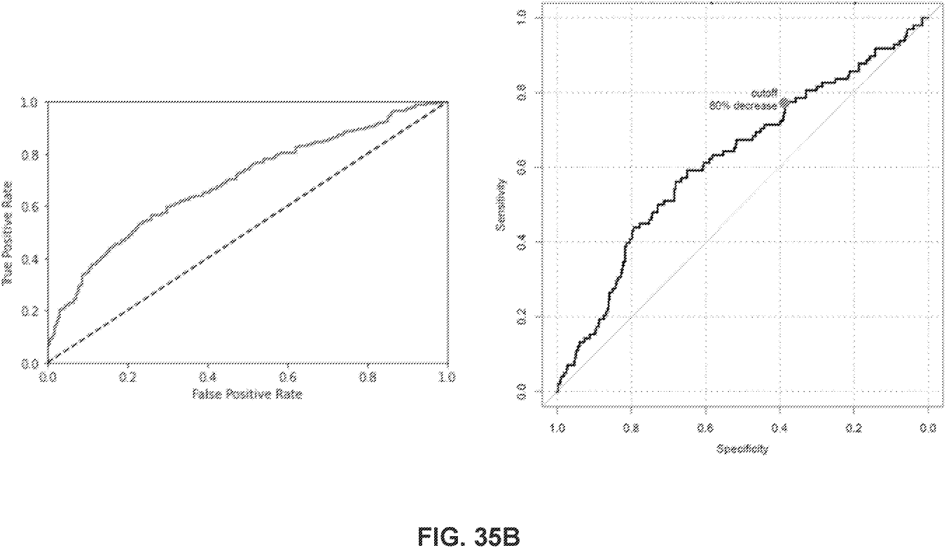

FIGS. 35A and 35B compare the performance of the PCT mortality prognostic with the APDv1. Specifically, FIGS. 35A-35B shows the comparison of prognostic performance for Procalcitonin (from the MOSES study intent to diagnosis population, right panel of FIG. 35A) and EPH APDv1 (validation cohort, left panel FIG. 35). AUROC = Area under the Receiver Operator Curve. Both studies showed significant survival curve separation, however due to the increased mortality of the ARDS population in the validation cohort, the high risk group has a much steeper survival drop than the PCT ITD cohort. The area under the receiver operator (AUROC) curve for PCT in the MOSES ITD group was 0.621 and the AUROC for the APDvl is 0.691.

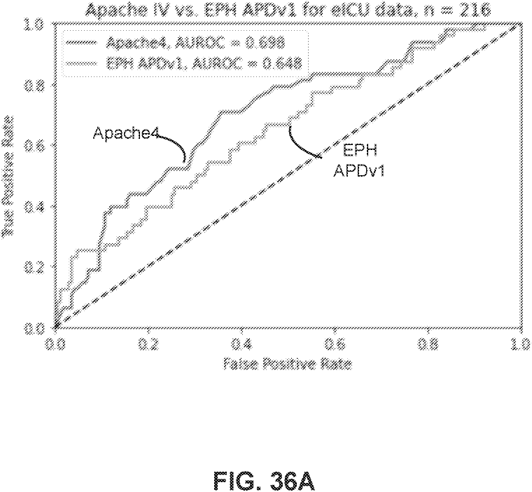

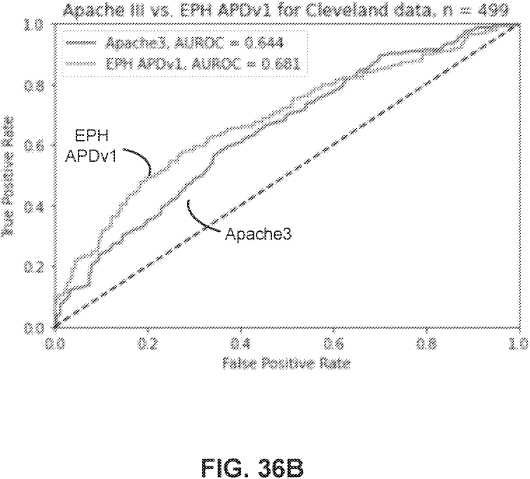

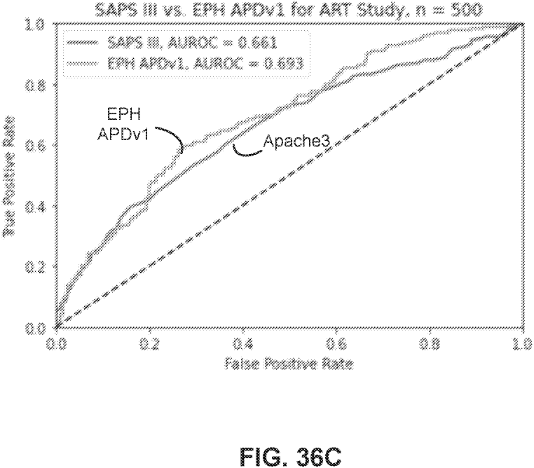

Severity scores (e.g., APACHE and SAPS scores) have been developed to compare the severity of illness for critically ill patients. In the validation data sets, the Cleveland Clinic ARDS data set and the eICU observational data sets provided Apache III scores for each patient and the ART data set provided SAPS III scores for each patient. FIGS. 36A-C compare the Receiver Operator curves for the available severity scores against the APDvl score for the same patients. The AUROC for APDvl is comparable to or better than the severity scores, despite using fewer variables and requiring less knowledge of patient history and comorbidities.

The Berlin criteria, which is a diagnostic criteria of timing, chest imaging, origin of edema, and hypoxemia for the assessment of ARDS severity can be used to determine the patient mortality risk. However, it has several weaknesses:
1. It is dependent on radiographic diagnostic methods which may not be immediately available and require specialized skill sets to determine clinical severity.
2. AUROC of 0.577 (95% CI, 0.561-0.593) for predictive validity for mortality.
3. COVID-19 induced ARDS may not fit the Berlin criteria for onset and radiographic severity.

The ARDS Prognostic Digital described herein Example 4 provides a strong separation between lower and higher risk groups of ARDS patients with performance comparable to or better than currently available prognostic tools for ARDS patients, with faster and easier data collection than those comparable tools. System and methods described herein evaluate patient mortality risk in three categories for a validation population with an overall mortality rate of 48.4% - the lower risk group has an average mortality rate of 22.1% (95% confidence interval of 15.6 - 30.1%), the medium risk group has an average mortality rate of 43.5% (39.8 - 47.2%), and the higher risk group has an average mortality rate of 66.8% (61.8 -71.4%). For the validation population of 1235 patients, 11% fall in the lower risk group and 31% fall in the higher risk group, with a combined 42% of patients with an actionable recommendation.

This performance is comparable to or better than currently-available FDA-approved mortality risk assessment tools such as procalcitonin and often used severity indicators such as SAPS and APACHE scores. Additionally, it is faster than PCT (the mortality risk is estimated on Day 1, not Day 4 of the ICU stay) and requires less information and fewer lab tests than the APACHE score.

### Example 5: Subtyped ARDS Patients Respond Differently to Varying Levels of PEEP

The objective of the present study is: 1) to describe how clinical and biological meaningful ARDS subphenotypes can be created using a minimum set of collectable clinical variables from ARDS patients with PaO₂/FiO₂ < 300, without the use of biomarkers; 2) to assess the heterogeneity of treatment effect (HTE) of different levels of PEEP (higher or lower) on mortality at the latest follow-up according to subphenotypes determined by K-means clustering clusters derived from clinical characteristics of patients with ARDS; and lastly 3) to assess the heterogeneity in the treatment effect of different levels of PEEP if only ARDS patients with PaO₂/FiO₂ < 200 are used to develop the subphenotypes.

The Berlin definition of acute respiratory distress syndrome (ARDS) encompasses acute hypoxemic respiratory failure due to a wide variety of etiologies. ARDS consensus definitions to date, including the Berlin definition, have solely relied on clinical variables, which help with early identification of patients and ensure implementation of standardized management and appropriate inclusion of patients in clinical trials. Clinical risk stratification currently depends on the PaO₂/FiO₂ ratio only. However, due to the inclusion of heterogeneous conditions exhibited within the syndrome, there are significant clinical and biological differences making ARDS challenging to treat.

These differences amongst ARDS patients are associated with variation in risk of disease development and progression, potentially generating differential responses to treatments and interventions. Therefore, identifying groups of patients who have similar clinical, physiologic, or biomarker traits becomes relevant as it can help with stratification of patients based on disease severity or risk of death, enrichment in clinical trials, and better targeting of therapies and interventions. These different groups can be defined as ARDS subphenotypes.

Two ARDS subphenotypes (hypoinflammatory and hyperinflammatory) have been consistently identified based on previous studies using Latent Class Analysis (LCA) and machine learning classifier models, showing that mortality and other clinical outcomes are worse in the hyperinflammatory subphenotype. However, these models are complex, and significant barriers exist in their implementation and use in clinical practice. Existing models use up to 40 predictor variables, including biomarkers and other variables that are not easily and readily available at the bedside which makes generalizability of some models very limited.

Recent publications have provided models with a parsimonious set of variables, but these models were mostly developed using biomarker profiles, which again limits its clinical utility. Furthermore, most previously reported studies have used data from randomized controlled trials conducted by a single network, raising questions about the generalizability of these results to different ARDS populations. Therefore, the aim of this study was to develop and validate a model using a small number of easily available clinical variables and evaluate whether it can identify ARDS subphenotypes in different populations.

A retrospective study was performed in a de-identified dataset pooling data from six randomized clinical trials in patients with ARDS, namely: ARMA, ALVEOLI, FACTT, EDEN, SAILS, and ART. The patients in the ARMA, ALVEOLI, FACTT, EDEN and SAILS trials were eligible if they met the American-European consensus for ARDS, including patients with a PaO₂ / FiO₂ ratio < 300 up to 48 hours before enrollment. From 1996 to 2013, these trials respectively enrolled 902, 549, 1000, 1000 and 745 patients and tested a variety of interventions. The multinational ART trial enrolled 1010 patients diagnosed with moderate to severe ARDS according to the Berlin criteria (PaO₂ / FiO₂ ratio < 200) for less than 72 hours of duration and assessed two different ventilatory strategies, between 2011 and 2017.

To avoid biases due to high mortality in the patients in the high tidal volume group of the ARMA study, which is not standard of care since the beginning of 2000, only patients receiving low tidal volume in that study were included (n= 473). All patients from each of the remaining trials were eligible for inclusion in this analysis, with an expected final sample size of 4,777 adult ARDS patients.

Data from the ARDSnet studies is publicly available from the NHLBI ARDS Network and data from the ART trial can be requested from study authors.

Baseline characteristics of the patients in the training and validation sets are presented in Table 28. Pneumonia was the prevailing etiology followed by sepsis and aspiration in all trials. Between 29.3% to 72.7% of the patients were receiving vasopressors at the time of randomization. At randomization, PaO₂ / FiO₂ ratio ranged from 112 (75 - 158) to 134 (96 -185) mmHg, and PEEP from 8 (5 - 10) to 12 (10 - 14) cmH₂O across trials. Mortality at 60 days for the ARDSnet trials ranged from 22.7% to 30.1%, while in the ART trial mortality at 28 days was 58.8%.

Datasets from the six trials were evaluated to identify a set of clinical variables which were most available across all datasets closest to time of randomization. The list of potential elements was then further refined to include only the ones that are frequently observed in the routine care of ARDS patients at the time of its diagnosis. To make a K-means clustering algorithm of potential rapid clinical use, elements which would not be commonly found in the electronic health records (EHR) at the time of ARDS diagnosis, such as biomarker levels, ARDS risk factors, therapeutics for organ support apart from mechanical ventilation settings, treatment assignment, severity scores, and clinical outcomes were excluded from model development.

After all assessment, 16 variables that are routinely collected as part of the usual care and which were uniformly present in all the trials were considered, including: age, gender, arterial pH, PaO₂, PaCO₂, bicarbonate, creatinine, bilirubin, platelets, heart rate, respiratory rate, mean arterial pressure, positive end-expiratory pressure (PEEP), plateau pressure, FiO₂, and tidal volume adjusted for predicted body weight (mL/kg PBW). The PBW was calculated as equal to 50 + 0.91 (centimeters of height - 152.4) in males, and 45.5 + 0.91 (centimeters of height - 152.4) in females. These variables were grouped into five domains named demographics, arterial blood gases, laboratory values, vital signs, and ventilatory variables. Plateau pressure was excluded due to a high rate of missingness across the trials included in the training set.

Data preprocessing was performed before modeling, and the pooled dataset was assessed for completeness and consistency. Patients with values out of the plausible physiological range for a specific variable were excluded from the final analysis. The training dataset was constructed using data from the two largest ARDSnet trials, EDEN and FACTT. The validation dataset was sourced from the four remaining trials: ALVEOLI, ARMA, SAILS, and ART. Means and standard deviations for z-scoring variables were calculated from the training dataset and subsequently applied to the validation data.

Baseline and outcome data were presented according to the assigned subphenotype. Continuous variables were presented as medians with their interquartile ranges and categorical variables as total number and percentage. Proportions were compared using Fisher exact tests and continuous variables were compared using the Wilcoxon rank-sum test. Study outcomes were further compared using the median and mean absolute differences for continuous and categorical values, respectively.

For the model development, the K-means clustering algorithm was used. K-means is one of the simplest and most commonly used classes of clustering algorithms. In critical care research, unsupervised machine learning techniques have already been used in several studies, attempting to find homogeneous subgroups within a broad heterogeneous population. This specific algorithm identifies a K number of clusters in a dataset by finding K centroids within the n-dimensional space of clinical features.

For feature selection, different sets of candidate variables were tested to assess their ability to produce significantly different mortality probabilities in each cluster using the minimum amount of readily available clinical data. For each set of candidate variables, the optimal number of clusters was determined by comparing models with between 2 and 5 clusters, using the Elbow method and the Calinski-Harabasz index. Information about the methods for selecting number of clusters are provided in the supplemental material.

Subsequently, the biological meaningfulness of each cluster was evaluated using their clinical, laboratory, and (when available) biomarker data. Then, each cluster was assigned a subphenotype label (Subphenotype A or Subphenotype B) All iterations in model development were conducted on the training set and the generalizability of the final model was assessed using the validation dataset.

K-means clustering analysis is structured to ignore cases with missing data. No assumption was made for missingness and therefore a complete case analysis was conducted. Model development and evaluation was performed using Python version 3.8 and scikit-leam 0.23.1.

The primary outcome was 60-day mortality for ARDSnet trials and 28-day mortality for the ART trial. Secondary outcomes were 90-day mortality, number of ventilator free days at day 28, and the duration of mechanical ventilation in survivors within the first 28 days post enrollment.

In total, 16 models were tested on ALVEOLI and ART for the differential effect of treatment on PEEP strategy according to subphenotype assignment. Variables in each of the 16 models (denoted as Model B.1, Model B.2...) are shown in Table 29. The testing involved employing a logistic regression model incorporating an interaction term for the product of subphenotype and mortality (28, 60, 90 and 180 day). For the ART trial, also included into the logistic regression model was the hospital of inclusion as a random effect.

Quantile models were used to assess ventilator-free days. Quantile models considered a T = 0.50 and an asymmetric Laplace distribution. P values were extracted after 1,000 bootstrap samplings and the effect estimate is the median difference. p-values <0.05 were considered statistically significant.

Among all trials and clinical measurements available closest to randomization, there were 20 variables that were considered not only routinely collected during care but also uniformly present in all trials. Sixteen different combinations of features were investigated in model development (Table 29). These combinations were defined based on the perceived clinical importance of each variable and their combinations, aiming for a minimum set of variables. According to the Elbow method and the Calinski-Harabasz index, two was the optimal number of K-means clusters among all sixteen models. The cluster of patients assigned to subphenotype B clearly had clinical and laboratory signs compatible with higher inflammation and worst outcomes (e.g., higher mortality). On the other hand, the cluster of patients assigned to subphenotype A exhibited signs of less inflammation and better outcomes (e.g., lower mortality).

The correlation between the 15 variables selected for K-means clustering is shown in Table 30. The strongest correlation was between PEEP and FiO₂ (r = 0.49). The optimal number of clusters based on both the Elbow method and the Calinski-Harabasz index determined that two clusters were a better fit than a higher number of clusters.

Further analysis was conducted across a subset of the 16 models. Specifically, across ten of the models (e.g., Models B.2, B.3, B.4, B.6, B.7, B.8, B.10, B.11, B.12, and B.16), absolute mortality difference between subphenotype A and subphenotype B ranged from 3.9% to 13.1% for the FACTT study and between 0.1% to 8.1% for EDEN. The models with the highest 60-day absolute mortality separation between subphenotypes for each of the two trials in the training set were then further evaluated. Models B.2, B.4, and B.8 were consistently amongst the models with highest separation. Of the 3 models with the highest mortality separation, Model B.2 was selected for further investigation, as it required the fewest variables (Table 29).

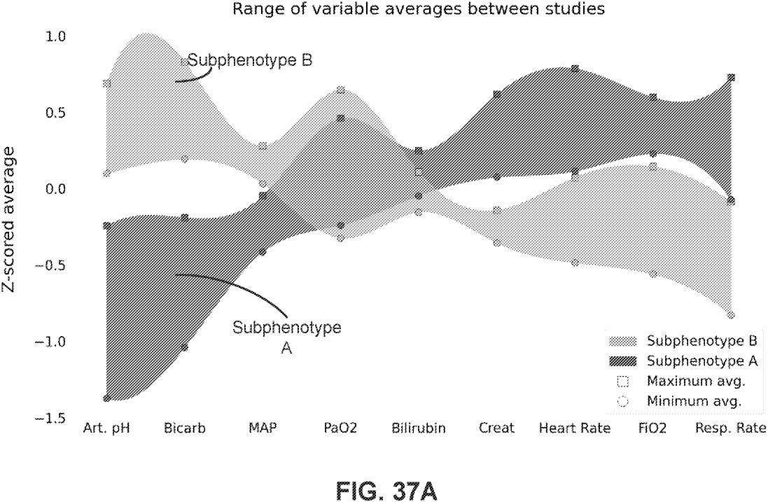

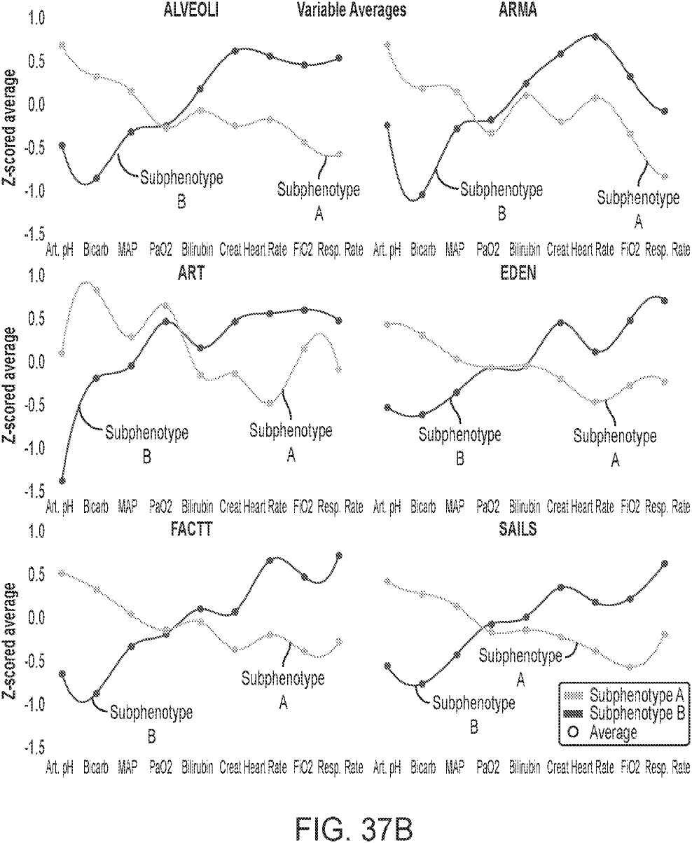

Based on model B.2, only nine clinical and laboratory variables were included to identify the two distinct subphenotypes in ARDS patients, namely: heart rate, mean arterial pressure, respiratory rate, bilirubin, bicarbonate, creatinine, PaO₂, arterial pH, and FiO₂. For each variable in the model, opposing measurements could be observed for each subphenotype. Specifically, FIG. 37A shows ranges of variables of patients in Subphenotype A and Subphenotype B. FIG. 37B shows variable values of patients in Subphenotype A and Subphenotype B across different datasets. For the ARDSnet trials, the incidence of subphenotype A patients varied from 57.8% (EDEN) to 73.6% (ARMA), and 41.5% of ART patients were part of subphenotype A. Across all trials, patients in subphenotype B had higher severity of illness, rate of vasopressor, heart rate, respiratory rate, creatinine, and bilirubin, as well as lower platelets, pH, BUN, and bicarbonate compared to patients in subphenotype A (Table 31, 32, and 33). In addition, 28-, 60-, and 90-day mortality rate was higher in patients in subphenotype B in all trials (Table 34). Likewise, for each trial, ventilator-free days at day 28 was lower in patients in subphenotype B compared to subphenotype A, and duration of ventilation in survivors was longer in subphenotype B.

Reference is now made to FIG. 37A which depicts differences of the variables included in the K-means cluster algorithm among subphenotypes: Square symbols represent the study with the highest mean z score for each subphenotype; Circles represent the study with the lowest mean z score for each subphenotype. The bands are exclusively to help visualize the opposite trends of the variables on the different clusters; Art.pH: arterial pH; Bicarb: bicarbonate; MAP: mean arterial pressure; Creat: creatinine; Resp.Rate: respiratory rate. Patients assigned to subphenotype A were drawn from K-means cluster 1, and patients assigned to subphenotype B were drawn from K-means cluster 2. Additionally, FIG. 37B shows variable averages for each of the studies (ALVEOLI and ARMA). The circles shown in FIG. 37B represent the averages for each variable. The lines are exclusively to help visualize the opposite trends of the variables on the different subphenotypes. Abbreviations: Art. pH is arterial pH, Bicarb is bicarbonate, MAP is mean arterial pressure, Creat is creatinine and Resp. Rate is respiratory rate

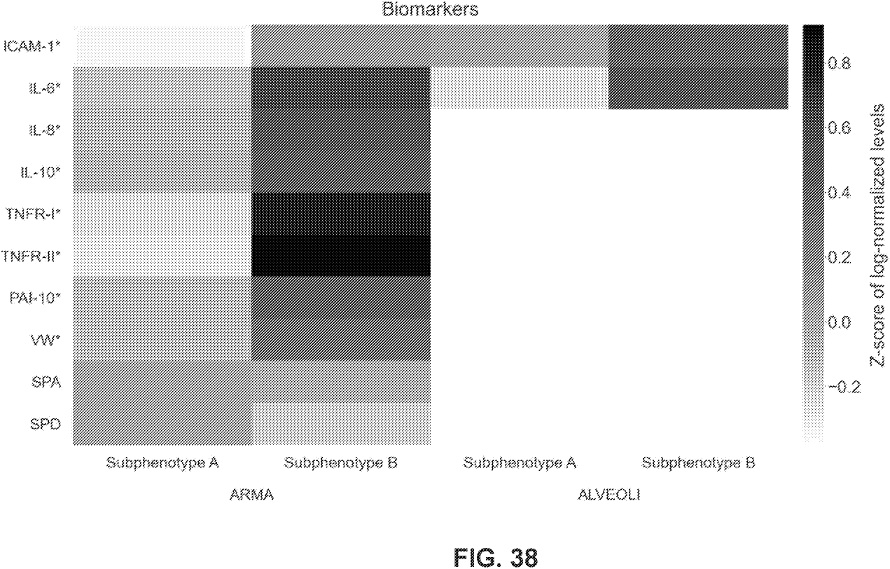

After comparing the clinical characteristics of the K-means clusters based on model B.2, each K-means cluster was assigned to represent a distinct subphenotype of ARDS, with patients in K-means cluster 1 assigned to subphenotype A, and patients in K-means cluster 2 assigned to subphenotype B. Using blood biomarker information available for a subset of patients from both ARMA and ALVEOLI, subphenotype B showed increased levels of pro-inflammatory markers when compared to subphenotype A (FIG. 38 and Table 35A). FIG. 38 shows a heat map of biomarkers available for the ARMA and ALVEOLI trials. For better visualization and due to difference in scales, the values were log-normalized and z-scored. Subphenotypes A and B are shown separately to highlight their differences.

Furthermore, the other 15 models (e.g., models other than model B.2) were also used to generate two clusters of patients that represent two distinct subphenotypes of ARDS, with patients in K-means cluster 1 assigned to subphenotype A, and patients in K-means cluster 2 assigned to subphenotype B. Table 35B shows the levels of IL-6 in patients of each subphenotype generated by any of the 16 different K-means clustering models. Generally, IL-6 is elevated in subphenotype B patients in comparison to subphenotype A patients.

Additionally, Tables 36-51 show the implementation of the 16 different models for guiding PEEP differential treatment response according to subphenotype assignments based on ARDS severity (e.g., P/F < 200 or P/F < 300 patients) from the ALVEOLI study. Additionally, Tables 52-67 show the implementation of the 16 different models for guiding PEEP differential treatment response according to subphenotype assignments based on ARDS severity (e.g., P/F < 200 or P/F < 300 patients) from the ART study. Generally, the subphenotype assignments of patients across both the ALVEOLI study and the ART study show that within Subphenotype A, patients receiving low PEEP had lower mortality with more ventilator free days, while results were less consistent in Subphenotype B. This suggests that patients in Subphenotype A benefit from lower PEEP, but contrary to current treatment guidelines for ARDS, patients within Subphenotype B may or may not benefit from lower PEEP.

This study has several strengths. First, it is the largest cohort of patients that has been studied to develop distinct phenotypes of ARDS patients. Moreover, the validation cohort included patients from the ART trial, enabling the validation of the model in the contemporaneous population of a large international randomized clinical trial in addition to the ARDSnet studies used in other subphenotyping studies. Second, the subphenotyping classifier was developed exclusively on the training set and then validated across multiple separate datasets and nevertheless similar separation in mortality was seen between the two subphenotypes across all trials. Third, the K-means algorithm was used to identify the subphenotypes, and the results obtained with this technique can be easily interpreted by clinicians and implemented in clinical practice. Lastly, this is the first phenotyping study that has used easily available clinical variables to identify ARDS phenotypes, which allows for early identification of these patients in the clinical care at the bedside. Using this algorithm with a small number of routinely collected variables could enable the model to be applied in trials that either retrospectively or prospectively assess interventions targeted to each subphenotype.

TABLE 28

Baseline Characteristics and Clinical Outcomes in the Included Trials

| | Training s et (n = 1998) | | | Validation set (n = 2775) | | |
|---|---|---|---|---|---|---|
| | EDEN | FACTT | ALVEOLI | ARMA | ART | SAILS |
| | (n = 1000) | (n = 998) | (n = 549) | (n = 472) | (n = 1010) | (n = 744) |
| Age, year* | 52.0 (42.0 - 63.0) | 49.0 (38.0 - 60.8) | 50.0 (39.0 - 65.0) | 50.0 (37.8 - 65.0) | 52.0 (36.0 - 64.0) | 55.0 (42.0 - 66.0) |
| Male gender - no. (%)* | 510 (51.0) | 533 (53.4) | 302 (55.0) | 285 (60.4) | 631 (62.5) | 365 (49.0) |
| Body mass index, kg/m² | 28.8 (24.0 - 34.8) | 27.3 (23.2 - 32.5) | 26.7 (22.5 - 30.7) | 25.8 (22.6 - 30.6) | 28.8 (25.0 - 33.8) | 28.6 (23.8 - 34.6) |
| Caucasian - no. (%) | 762 (79.7) | 641 (64.2) | 412 (75.0) | 355 (75.2) | --- | 589 (79.2) |

Etiology - no. (%)

| Pneumonia | 650 (65.0) | 471 (47.2) | 221 (40.3) | 145 (30.7) | 555 (55.0) | 526 (70.7) |
|---|---|---|---|---|---|---|
| Sepsis | 147 (14.7) | 231 (23.1) | 120 (21.9) | 125 (26.5) | 196 (19.4) | 147 (19.8) |
| Aspiration | 96 (9.6) | 149 (14.9) | 84 (15.3) | 72 (15.3) | 58 (5.7) | 49 (6.6) |
| Trauma | 36 (3.6) | 74 (7.4) | 45 (8.2) | 59 (12.5) | 31 (3.1) | 6 (0.8) |
| Other | 71 (7.1) | 73 (7.3) | 79 (14.4) | 71 (15.0) | 170 (16.8) | 16 (2.2) |
| Prognostic scores | | | | | | |
| APACHE III | 73.0 (59.0 - 89.0) | 78.0 (62.0 - 94.0) | 78.0 (64.0 - 93.0) | 83.0 (70.0 - 97.0) | --- | 76.0 (61.0 - 92.0) |
| SAPS III | --- | --- | --- | --- | 63.0 (50.2 - 75.0) | |
| Use of vasopressor -no. (%) | 489 (48.9) | 397 (40.5) | 156 (29.3) | 147 (31.3) | 734 (72.7) | 395 (54.2) |
| Vital signs | | | | | | |
| Temperature, °C | 37.3 (36.8 - 37.9) | 37.5 (36.9 - 38.2) | 37.6 (37.0 - 38.2) | 37.7 (37.0 - 38.2) | --- | 37.3 (36.7 - 37.9) |
| Heart rate, bpm* | 94 (81 - 108) | 102.0 (87.0 - 117.0) | 101.0 (86.0 - 114.0) | 104.0 (91.0 - 118.0) | 101.0 (85.0 - 118.0) | 95.0 (83.0 - 108.0) |
| Mean arterial Pressure, mmHg* | 74.0 (67.0 - 82.0) | 75.0 (67.0 - 86.0] | 76.5 (69.0 - 85.3) | 76.8 (69.0 - 87.3) | 77.0 (70.0 - 87.0) | 75.0 (67.0 - 84.5) |
| SpO₂, % | 95 (93 - 98) | 96 (93 - 98) | 96 (93 - 97) | 95 (93 - 97) | --- | 96 (94 - 99) |
| Urine output in 24 hours, mL | 1325 (799 - 2132) | 1668 (1080 - 2685) | 1845 (1127 - 2925) | 2020 (1256 - 2973) | 1300 (600 - 2123) | 1328 (735 - 2177) |
| Laboratory tests | | | | | | |
| Hematocrit, % | 30 (26 - 34) | 30.0 (26.0 - 34.0) | 31.0 (28.0 - 34.0) | 30.0 (28.0 - 34.0) | --- | 31.0 (27.0 - 36.0) |
| White blood cell count, 10⁹/L | 12.0 (7.8 - 16.7) | 11.8 (7.2 - 17.1) | 11.6 (7.7-15.7) | 11.5 (7.5 - 16.2) | --- | 13.9 (8.7 - 20.0) |
| Platelets, 10⁹/L* | 169 (108 - 241) | 183 (106 - 258) | 157 (83 - 247) | 135 (80 - 211) | 175 (106 - 263) | 167 (96 - 247) |
| Creatinine, mg/dL* | 1.2 (0.8 - 2.0) | 1.0 (0.7 - 1.5) | 1.0 (0.7 - 1.7) | 1.1 (0.8 - 1.7) | 1.3 (0.8 - 2.2) | 1.0 (0.7 - 1.7) |
| Bilirubin, mg/dL* | 0.8 (0.5 - 1.4) | 0.8 (0.5 - 1.6) | 0.8 (0.5 - 1.5) | 1.0 (0.6 - 2.1) | 0.8 (0.4 - 1.5) | 0.8 (0.5 - 1.4) |
| Arterial blood gas | | | | | | |
| pH* | 7.36 (7.30 - 7.42) | 7.37 (7.30 - 7.43) | 7.40 (7.34 - 7.44) | 7.41 (7.35 - 7.45) | 7.28 (7.19 - 7.36) | 7.37 (7.31 - 7.42) |
| PaO₂, mmHg* | 83 (68 - 108) | 79 (67 - 100) | 77 (67- 93) | 76.5 (67 - 93) | 112 (81 - 155) | 83 (69 - 103) |
| PaO₂ / FiO₂ | 125 (86 - 178) | 118 (80 - 163) | 134 (96 - 185) | 112 (75 - 158) | 112 (81 - 155) | 133 (89 - 178) |
| PaCO₂, mmHg* | 38 (34 - 45) | 39 (34 - 45) | 38 (33 - 43) | 36 (31 - 41) | 50 (42 - 62) | 39 (34 - 45) |
| Bicarbonate, mmol/L* | 21.0 (18.0 - 25.0) | 21.0 (17.4 - 25.0) | 22.0 (18.0 - 26.0) | 22.0 (18.0 - 25.0) | 22.9 (19.4 - 26.3) | 22.0 (18.0 - 25.0) |
| Ventilatory variables | | | | | | |
| Tidal volume, mL* | 410 (360 - 470) | 450 (400 - 510) | 500 (420 - 600) | 700 (600 - 750) | 350 (308 - 400) | 400 (350 - 460) |
| Per PBW, mL/kg PBW | 6.3 (6.0 - 7.3) | 7.1 (6.1 - 8.1) | 7.9 (6.6 - 9.4) | 10.2 (9.0 - 11.3) | 5.9 (5.1 - 6.1) | 6.2 (6.0 - 7.1) |
| Plateau pressure, cmH₂O | 24.0 (20.0 - 27.0) | 26.0 (22.0 - 30.0) | 26.0 (22.0 - 31.0) | 29.0 (24.8 - 34.0) | 26.0 (22.0 - 29.0) | 24.0 (19.0 - 28.0) |
| PEEP, cmH₂O* | 10 (5 -12) | 10 (5 - 12) | 10 (5 - 12) | 8 (5 - 10) | 12 (10 - 14) | 10 (5 - 11) |
| Respiratory rate, breaths/min* | 25 (20 - 30) | 25 (20 - 31) | 22 (16 - 29) | 19 (15 - 24) | 25 (20 - 30) | 25 (20 - 30) |
| FiO₂* | 0.60 (0.50 - 0.80) | 0.60 (0.50 - 0.80) | 0.60 (0.50 - 0.80) | 0.60 (0.50 - 0.74) | 0.70 (0.60 - 1.00) | 0.60 (0.40 - 0.70) |
| Clinical outcomes | | | | | | |
| 28-day mortality - no. (%) ^{#} | 194 (19.4) | 231 (23.1) | 125 (22.8) | 119 (25.2) | 528 (52.3) | 172 (23.1) |
| 60-day mortality - no. (%)^{##} | 227 (22.7) | 268 (26.9) | 144 (26.2) | 141 (30.1) | 594 (58.8) | 199(26.7) |
| 90-day mortality - no. (%) | 233 (23.3) | 283 (28.6) | 148 (27.5) | 143 (30.8) | 611(60.5) | 204 (27.4) |
| Ventilator-free days at day 28 | 20.0 (0.0 - 24.0) | 17.0 (0.0 - 23.0) | 18.0 (0.0 - 24.0) | 13.0 (0.0 - 23.0) | 0.0 (0.0 - 13.0) | 20.0 (0.0 - 25.0) |
| Duration of ventilation in survivors, days | 7.0 (4.0 - 13.0) | 8.0 (5.0 - 16.0) | 8.0 (4.0 - 14.0) | 8.0 (4.0 - 15.0) | 13.0 (8.0 - 20.0) | 6.0 (4.0 - 11.0) |

Data are median (quartile 25^{th} - quartile 75^{th}) or N (%)
Abbreviations: APACHE denotes Acute Physiology and Chronic Health Evaluation, and SAPS denotes Simplified Acute Physiology Score.
* Variables selected for K-means cluster detection; # Primary outcome for ART trial; ^{##} Primary outcome for ARDSnet trials

TABLE 29

List of variables in each model

| | Vitals | | | Arterial blood gas | | | | | Labs | | | | Demographics | | | Mechanical Ventilation Parameters | | | | Organ support |
|---|---|---|---|---|---|---|---|---|---|---|---|---|---|---|---|---|---|---|---|---|
| Model | HRATER | MEANAPR | RESPR | ARTPHR | PAO2R | FIO2R | PACO2 | PAFILP | BICARL | CREATR | BILIH | PLATEL | AGE | GENDER | BMI | PEEPR | TIDALR | PPLATR | TMVNTR | VASOL24 |
| B.1 | X | X | X | X | X | X | | | X | X | | | | | | | | | | |
| B.2 | X | X | X | X | X | X | | | X | X | X | | | | | | | | | |
| B.3 | X | X | X | X | X | X | | | X | X | X | | X | X | | | | | | |
| B.4 | X | X | X | X | X | X | | | X | X | | | X | X | | | | | | |
| B.5 | X | X | X | X | X | X | X | X | X | X | | X | X | X | | X | X | | | |
| B.6 | X | X | X | X | X | X | X | X | X | X | X | X | X | X | | X | X | | | |
| B.7 | X | X | X | X | X | X | X | | X | X | X | | | | | | | | | |
| B.8 | X | X | X | X | X | X | X | | X | X | X | X | | | | | | | | |
| B.9 | X | X | X | X | X | X | X | | X | X | | | | | | | | | | |
| B.10 | X | X | X | | | | | | | | | | X | X | | | | | | |
| B.11 | X | X | X | X | X | X | X | | X | X | X | | X | X | | | | | | |
| B.12 | X | X | X | X | X | X | X | X | X | X | X | X | | | | X | X | | | |
| B.13 | X | X | X | X | X | X | X | X | X | X | X | X | X | X | X | X | X | X | X | X |
| B.14 | X | X | X | X | X | | | | X | X | | | | | | | | | | |
| B.15 | X | X | X | X | X | | | | X | | | | | | | | | | | |
| B.16 | X | X | X | X | X | | X | | X | | | | | | | | | | | |

HRATER: Heart Rate; MEANAPR: Mean Arterial Pressure; RESPR: Respiratory Rate; ARTPHR: Arterial pH; PAO2R: Partial Pressure of Oxygen; FiO2R: Inspirited fraction of oxygen; PACO2: Partial Pressure of Carbon Dioxide; PAFILP: PaO2/FiO2; BICARBL: Bicarbonate; CRETAR: Creatinine; BILIH: bilirubin; PLATEL: platelets; BMI: Body Mass Index, PEEPR: Positive End-Expiratory Pressure; TIDALR: Tidal Volume; PPLATR: Plateau Pressure; TVVNTR: Minute ventilation; VASOL24: vasopressor use prior 24 h

TABLE 30

Correlation among fifteen routinely collected variables, close to the time of randomization

| | Age | pH | HCO₃ | Bili | Creat | FiO₂ | Gender | HR | MAP | PaCO₂ | PaO₂ | PEEP | Plat | RR | V_{T}/PBW |
|---|---|---|---|---|---|---|---|---|---|---|---|---|---|---|---|
| Age | 1.00 | 0.06 | -0.04 | -0.02 | 0.11 | -0.13 | 0.00 | -0.27 | -0.12 | -0.11 | -0.06 | -0.22 | 0.00 | -0.11 | 0.03 |
| pH | 0.06 | 1.00 | 0.40 | -0.04 | -0.16 | -0.26 | -0.01 | -0.18 | 0.15 | -0.39 | 0.00 | -0.20 | 0.05 | -0.21 | 0.07 |
| HCO₃ | -0.04 | 0.40 | 1.00 | -0.08 | -0.28 | -0.05 | -0.02 | -0.18 | 0.08 | 0.44 | 0.02 | -0.05 | 0.15 | -0.24 | -0.07 |
| Bili | -0.02 | -0.04 | -0.08 | 1.00 | 0.06 | -0.03 | -0.04 | 0.01 | -0.04 | -0.01 | 0.03 | 0.01 | -0.20 | 0.04 | -0.01 |
| Creat | 0.11 | -0.16 | -0.28 | 0.06 | 1.00 | -0.04 | -0.08 | -0.04 | -0.01 | -0.14 | 0.00 | -0.06 | -0.12 | 0.02 | 0.00 |
| FiO₂ | -0.13 | -0.26 | -0.05 | -0.03 | -0.04 | 1.00 | 0.03 | 0.13 | -0.06 | 0.18 | 0.11 | 0.49 | 0.06 | 0.21 | -0.02 |
| Gender | 0.00 | -0.01 | -0.02 | -0.04 | -0.08 | 0.03 | 1.00 | -0.03 | -0.05 | -0.04 | -0.06 | 0.02 | 0.09 | 0.09 | 0.19 |
| HR | -0.27 | -0.18 | -0.18 | 0.01 | -0.04 | 0.13 | -0.03 | 1.00 | -0.02 | 0.03 | -0.04 | 0.12 | -0.05 | 0.22 | 0.08 |
| MAP | -0.12 | 0.15 | 0.08 | -0.04 | -0.01 | -0.06 | -0.05 | -0.02 | 1.00 | -0.03 | 0.01 | -0.01 | 0.06 | -0.04 | 0.00 |
| PaCO₂ | -0.11 | -0.39 | 0.44 | -0.01 | -0.14 | 0.18 | -0.04 | 0.03 | -0.03 | 1.00 | -0.04 | 0.17 | 0.11 | -0.05 | -0.17 |
| PaO₂ | -0.06 | 0.00 | 0.02 | 0.03 | 0.00 | 0.11 | -0.06 | -0.04 | 0.01 | -0.04 | 1.00 | -0.09 | -0.04 | -0.09 | 0.03 |
| PEEP | -0.22 | -0.20 | -0.05 | 0.01 | -0.06 | 0.49 | 0.02 | 0.12 | -0.01 | 0.17 | -0.09 | 1.00 | 0.00 | 0.33 | -0.15 |
| Plat | 0.00 | 0.05 | 0.15 | -0.20 | -0.12 | 0.06 | 0.09 | -0.05 | 0.06 | 0.11 | -0.04 | 0.00 | 1.00 | -0.05 | 0.03 |
| RR | -0.11 | -0.21 | -0.24 | 0.04 | 0.02 | 0.21 | 0.09 | 0.22 | -0.04 | -0.05 | -0.09 | 0.33 | -0.05 | 1.00 | -0.31 |
| V_{T}/PBW | 0.03 | 0.07 | -0.07 | -0.01 | 0.00 | -0.02 | 0.19 | 0.08 | 0.00 | -0.17 | 0.03 | -0.15 | 0.03 | -0.31 | 1.00 |

Data are Pearson correlation coefficients.
Abbreviations: Bili denotes bilirubin, Creat is creatinine, HR is heart rate, MAP is mean arterial pressure, PEEP is positive end-expiratory pressure, Plat is platelets, RR is respiratory rate and V_{T}/PBW is tidal volume per predicted body weight.

TABLE 31

Baseline Characteristics and Clinical Outcomes According to Subphenotype and Trial in the Training Set

| | FACTT | | | EDEN | | |
|---|---|---|---|---|---|---|
| | Subphenotype A | Subphenotype B | p value | Subphenotype A | Subphenotype B | p value |
| | (n = 407) | (n = 294) | | (n = 449) | (n = 328) | |
| Age, year* | 50.0 (40.0 - 63.0) | 47.0 (36.0 - 58.0) | 0.002 | 53.0 (44.0 - 63.0) | 51.0 (41.0 - 62.2) | 0.183 |
| Male gender - no. (%) | 223 (54.8) | 151 (51.4) | 0.411 | 233 (51.9) | 168 (51.2) | 0.910 |
| Body mass index, kg/m² | 27.5 (23.3 - 32.1) | 27.4 (23.0 - 32.7) | 0.938 | 29.1 (24.6 - 34.5) | 28.5 (23.4 - 35.1) | 0.476 |
| Caucasian - no. (%) | 269 (66.1) | 177 (60.2) | 0.129 | 349 (81.5) | 237 (75.7) | 0.067 |

| | | | | |
|---|---|---|---|---|
| Etiology - no. (%) < 0.001 0.003 | | | | |
| Pneumonia | 201 (49.4) | 139 (47.3) | 296 (65.9) | 217 (66.2) |
| Sepsis | 78 (19.2) | 101 (34.4) | 50 (11.1) | 60 (18.3) |
| Aspiration | 67 (16.5) | 30 (10.2) | 45 (10.0) | 27 (8.2) |
| Trauma | 24 (5.9) | 8 (2.7) | 24 (5.3) | 5 (1.5) |
| Other | 37 (9.1) | 16 (5.4) | 34 (7.6) | 19 (5.8) |

Prognostic scores

| APACHE III | 69.0 (56.0 - 84.0) | 91 (76.0 - 105.0) | < 0.001 | 66.0 (54.0 - 79.0) | 84.0 (71.0 - 100.2) | < 0.001 |
|---|---|---|---|---|---|---|
| Use of vasopressor - no. (%) | 118 (29.5) | 189 (64.9) | < 0.001 | 187 (41.6) | 209 (63.7) | < 0.001 |
| Vital signs | | | | | | |
| Temperature, °C | 37.5 (36.8 - 38.2) | 37.6 (37.0 - 38.4) | 0.371 | 37.3 (36.8 - 37.8) | 37.3 (36.7 - 38.1) | 0.212 |
| Heart rate, bpm | 95.0 (81.0 - 110.0) | 114 (102 - 126) | < 0.001 | 89 (77 - 102) | 101 (89 - 116) | < 0.001 |
| Mean arterial Pressure, mmHg | 76.0 (68.0 - 88.0) | 71.0 (65.0 - 80.8) | < 0.001 | 77.0 (68.0 - 84.0) | 71.0 (66.0 - 80.0) | < 0.001 |
| SpO₂, % | 96 (93 - 98) | 95 (92 - 97) | < 0.001 | 96 (94 - 98) | 95 (92 - 98) | 0.032 |
| Urine output in 24 hours, mL | 1785 (1192 - 2853) | 1370 (842 - 2446) | < 0.001 | 1505 (977 - 2250) | 1165 (566 - 1816) | < 0.001 |
| Laboratory tests | | | | | | |
| Hematocrit, % | 30.0 (26.0 - 33.0) | 30.0 (24.2 - 35.0) | 0.272 | 30.0 (26.0 - 34.0) | 30.0 (26.0 - 35.0) | 0.919 |
| White blood cell count, 10⁹/L | 11.6 (7.3 - 16.3) | 11.7 (5.6 - 17.9) | 0.972 | 11.4 (7.7 - 15.5) | 12.7 (7.7 - 19.0) | 0.019 |
| Platelets, 10⁹/L | 195 (118.5 - 268) | 158 (87 - 237) | < 0.001 | 163 (108 - 241) | 164 (103 - 227) | 0.552 |
| Creatinine, mg/dL | 0.9 (0.7 - 1.3) | 1.4 (1.0 - 2.0) | < 0.001 | 1.0 (0.7 - 1.5) | 1.6 (1.0 - 2.8) | < 0.00 |
| Bilirubin, mg/dL | 0.7 (0.5 - 1.3) | 0.9 (0.5 - 2.0) | 0.003 | 0.8 (0.5 - 1.3) | 0.8 (0.5 - 1.7) | 0.128 |
| Arterial blood gas | | | | | | |
| pH* | 7.41 (7.36 - 7.45) | 7.29 (7.23 - 7.35) | < 0.001 | 7.40 (7.35 - 7.44) | 7.30 (7.24 - 7.35) | < 0.001 |
| PaO₂, mmHg | 78 (68 - 100) | 78 (65 - 99) | 0.240 | 83 (70 - 107) | 81 (67 - 107) | 0.416 |
| PaO₂ / FiO₂ | 132 (92 - 173) | 89 (65 - 126) | < 0.001 | 133 (98 - 193) | 101 (73 - 162) | < 0.001 |
| PaCO₂, mmHg | 39 (34 - 44) | 38.5 (33 - 47.9) | 0.877 | 38 (34 - 44) | 38 (33 - 46) | 0.55 |
| Bicarbonate, mmol/L | 24.0 (21.0 - 27.0) | 17.0 (14.0 - 20.0) | < 0.001 | 23.0 (21.0 - 26.0) | 18.5 (15.0 - 21.0) | < 0.001 |
| Ventilatory variables | | | | | | |
| Tidal volume, mL | 450 (400 - 530) | 450 (382 - 500) | 0.009 | 420 (356 - 487) | 400 (350 - 450) | 0.032 |
| Per PBW, mL/kg PBW | 7.1 (6.3 - 8.4) | 7.0 (6.0, 8.0) | 0.058 | 6.3 (6.0 - 7.5) | 6.1 (6.0 - 7.3) | 0.079 |
| Plateau pressure, cmH₂O | 25.0 (20.0 - 29.0) | 28.0 (24.0 - 32.0) | < 0.001 | 23.0 (19.0 - 27.0) | 24.0 (21.0 - 28.0) | 0.004 |
| PEEP, cmH₂O | 8 (5 - 10) | 10 (8 - 14) | < 0.001 | 10 (5 - 10) | 10 (8 - 14) | < 0.001 |
| Respiratory rate, breaths/min | 22 (18 - 27) | 30 (24 - 35) | < 0.001 | 22 (19 - 26) | 30 (25 - 35) | < 0.001 |
| FiO₂ | 0.50 (0.40 - 0.70) | 0.80 (0.60 - 1.00) | < 0.001 | 0.60 (0.45 - 0.70) | 0.80 (0.60 - 1.00) | < 0.001 |

Data are mean ± standard deviation, median (quartile 25^{th} - quartile 75^{th}) or N (%)
Abbreviations: APACHE denotes Acute Physiology and Chronic Health Evaluation, V_{T}/PBW denotes tidal volume per predicted body weight.

TABLE 32

Baseline Characteristics and Clinical Outcomes According to the Subphenotype and Two Trials in the Validation Set

| | ALVEOLI | | | ARMA | | |
|---|---|---|---|---|---|---|
| | Subphenotype A | Subphenotype B | p value | Subphenotype A | Subphenotype B | p value |
| | (n = 336) | (n = 157) | | (n = 279) | (n = 100) | |
| Age, year* | 53.0 (39.0 - 66.2) | 46.0 (37.0 - 60.0) | 0.007 | 49.0 (37.0 - 64.0) | 47.5 (36.0 - 61.0) | 0.180 |
| Male gender - no. (%) | 188 (56.0) | 86 (54.8) | 0.883 | 169 (60.6) | 61 (61.0) | 0.965 |
| Body mass index, kg/m² | 27.0 (22.9 - 31.1) | 25.2 (21.7 - 30.2) | 0.050 | 25.8 (23.0 - 30.2) | 24.4 (21.5 - 29.7) | 0.057 |
| Caucasian - no. (%) | 263 (78.3) | 102 (65.0) | 0.002 | 220 (78.9) | 65 (65.0) | 0.009 |
| Etiology - no. (%) 0.001 < 0.001 | | | | | | |
| Pneumonia | 130 (38.7) | 66 (42.0) | | 83 (29.7) | 30 (30.0) | |
| Sepsis | 63 (18.8) | 50 (31.8) | | 64 (22.9) | 43 (43.0) | |
| Aspiration | 55 (16.4) | 19 (12.1) | | 44 (15.8) | 14 (14.0) | |
| Trauma | 33 (9.8) | 5 (3.2) | | 43 (15.4) | 4 (4.0) | |
| Other | 55 (16.4) | 17 (10.8) | | 45 (16.1) | 9 (9.0) | |
| Prognostic scores | | | | | | |
| APACHE III | 71. (59.0 - 83.0) | 93.0 (80.0 - 110.0) | < 0.001 | 77.0 (66.0 - 90.5) | 97.0 (81.8 (110.0) | < 0.001 |
| Use of vasopressor - no. (%) | 65 (20.1) | 80 (51.3) | < 0.001 | 77 (27.6) | 52 (52.5) | < 0.001 |
| Vital signs | | | | | | |
| Temperature, °C | 37.6 (37.1 - 38.2) | 37.7 (36.9 - 38.3) | 0.778 | 37.6 (37.1 - 38.1) | 37.6 (36.8 - 38.4) | 0.803 |
| Heart rate, bpm | 97.5 (83.0 - 109.00) | 111.0 (97.0 - 126) | < 0.001 | 101.0 (89.0 - 112.5) | 118 (105.0 - 128.0) | < 0.001 |
| Mean arterial Pressure, mmHg | 77.3 (77.0 - 87.3) | 73.3 (65.0 - 80.3) | < 0.001 | 78.0 (70.7 - 88.0) | 70.5 (64.9 - 80.4) | < 0.001 |
| SpO₂, % | 96 (94 - 97) | 95 (92 - 97) | 0.005 | 95 (93 - 98) | 95.5 (93 - 97) | 0.799 |
| Urine output in 24 hours, mL | 2065 (1355 - 3255) | 1433 (569 - 2189) | < 0.001 | 2100 (1375 - 3096) | 1525 (816 - 2650) | 0.001 |
| Laboratory tests | | | | | | |
| Hematocrit, % | 31.0 (28.0 - 34.0) | 31.0 (27.0 - 35.0) | 0.617 | 30.0 (28.0 - 33.0) | 31.0 (28.0 - 34.0) | 0.299 |
| White blood cell count, 10⁹/L | 11.7 (8.1 - 15.3) | 10.7 (6.4 - 15.8) | 0.166 | 11.9 (7.7 - 16.7) | 9.8 (5.4 - 16.7) | 0.057 |
| Platelets, 10⁹/L | 173 (94 - 266) | 141 (57 - 214) | 0.001 | 139 (80 - 212) | 125 (72 - 196) | 0.260 |
| Creatinine, mg/dL | 0.9 (0.7 - 1.3) | 1.5 (0.9 - 3.0) | < 0.001 | 1.0 (0.7 - 1.4) | 1.8 (1.2 - 3.2) | < 0.00 |
| Bilirubin, mg/dL | 0.8 (0.5 - 1.4) | 0.9 (0.4 - 1.8) | 0.289 | 1.0 (0.6 - 2.1) | 1.1 (0.7 - 27) | 0.106 |
| Arterial blood gas | | | | | | |
| pH* | 7.42 (7.38 - 7.45) | 7.31 (7.24 - 7.36) | < 0.001 | 7.42 (7.38 - 7.47) | 7.33 (7.28 - 7.37) | < 0.00 |
| PaO₂, mmHg | 78 (68 - 93) | 74 (65 - 92) | 0.082 | 75 (66 - 91) | 81 (68 - 96) | 0.106 |
| PaO₂/FiO₂ | 149 (109 - 192) | 103 (74 - 136) | < 0.001 | 118 (83 - 160) | 99 (68 - 137) | 0.006 |
| PaCO₂, mmHg | 38 (34 - 43) | 36 (31 - 42) | 0.046 | 37 (31 - 41) | 34 (28.8 - 39.2) | 0.003 |
| Bicarbonate, mmol/L | 24 (21 - 27) | 17 (13 - 20) | < 0.001 | 23 (20 - 26) | 16 (13 - 19) | < 0.001 |
| Ventilatory variables | | | | | | |
| Tidal volume, mL | 500 (437 - 600) | 480 (400 - 572) | 0.002 | 700 (600 - 750) | 700 (550 - 700) | 0.198 |
| Per PBW, mL/kg PBW | 8.0 (6.9 - 9.5) | 7.4 (6.2 - 9.2) | 0.006 | 10.1 (9.2 - 11.1) | 10.6 (9.0 - 11.4) | 0.383 |
| Plateau pressure, cmH₂O | 25.0 (21.0 - 30.0) | 29.0 (24.0 - 33.0) | < 0.001 | 29.0 (24.0 - 34.0) | 31.0 (27.0 - 36.0) | 0.018 |
| PEEP, cmH₂O | 10 (5 - 10) | 10 (8 - 14) | < 0.001 | 8 (5 - 10) | 10 (5 - 12) | 0.150 |
| Respiratory rate, breaths/min | 20 (15 - 25) | 30 (24 - 35) | < 0.001 | 18 (14 - 21) | 24 (18.8 - 28) | < 0.001 |
| FiO₂ | 0.50 (0.44 - 0.65) | 0.75 (0.60 - 1.00) | < 0.001 | 0.60 (0.50 - 0.70) | 0.70 (0.59 - 0.96) | < 0.001 |

Data are mean ± standard deviation, median (quartile 25^{th} - quartile 75^{th}) or N (%)
Abbreviations: APACHE denotes Acute Physiology and Chronic Health Evaluation, V_{T}/PBW denotes tidal volume per predicted body weight.

TABLE 33

Baseline Characteristics and Clinical Outcomes According to the Subphenotype and Two Trials in the Validation Set

| | SAILS | | | ART | | |
|---|---|---|---|---|---|---|
| | Subphenotype A (n = 319) | Subphenotype B (n = 188) | p value | Subphenotype A (n = 211) | Subphenotype B (n = 298) | p value |
| Age, year* | 57.0 (46.0 - 67.0) | 53.5 (39.0 - 65.0) | 0.035 | 54.0 (37.0 - 65.0) | 50.0 (35.2 - 61.0) | 0.075 |
| Male gender - no. (%) | 150 (47.0) | 100 (53.2) | 0.211 | 136 (64.5) | 181 (60.7) | 0.448 |
| Body mass index, kg/m² | 28.5 (23.9 - 34.6) | 29.8 (23.2 - 35.1) | 0.903 | 28.8 (24.6 - 35.6) | 28.4 (25.0 - 31.7) | 0.367 |
| Caucasian - no. (%) | 250 (78.4) | 140 (74.5) | 0.369 | --- | --- | |

| Etiology - no. (%) | | | 0.709 | | | 0.052 |
|---|---|---|---|---|---|---|
| Pneumonia | 228 (71.5) | 127 (67.6) | | 113 (53.6) | 171 (57.4) | |
| Sepsis | 63 (19.7) | 39 (20.7) | | 38 (18.0) | 59 (19.8) | |
| Aspiration | 19 (6.0) | 15 (8.0) | | 13 (6.2) | 16 (5.4) | |
| Trauma | 3 (0.9) | 1 (0.5) | | 10 (4.7) | 2 (0.7) | |
| Other | 6 (1.9) | 6 (3.2) | | 37 (17.5) | 50 (16.8) | |
| Prognostic scores | | | | --- | --- | |
| APACHE III | 70.0 (56.0 - 84.0) | 92.0 (75.0 - 105.8) | < 0.001 | | | |
| SAPS III | --- | --- | --- | 62 (50 - 71) | 66 (53 - 75) | 0.010 |
| Use of vasopressor - no. (%) | 150 (47.8) | 142 (78.5) | < 0.001 | 130 (61.6) | 242 (81.2) | < 0.001 |
| Vital signs | | | | | | |
| Temperature, °C | 37.2 (36.7 - 37.8) | 37.3 (36.7 - 38.0) | 0.346 | --- | --- | |
| Heart rate, bpm | 91.0 (80.5 - 103.0) | 102.0 (88.8 - 117.0) | < 0.001 | 90.0 (73.0 - 103.0) | 112.0 (97.2 - 126.0) | < 0.001 |
| Mean arterial Pressure, mmHg | 78.0 (69.5 - 88.0) | 70.0 (63.0 - 78.) | < 0.001 | 80.0 (73.5 - 89.0) | 75.0 (70.0 - 83.0) | < 0.001 |
| SpO₂, % | 96 (95 - 99) | 96 (93 - 99) | 0.270 | --- | --- | |
| Urine output in 24 hours, mL | 1570 (852 - 2383) | 920 (350 - 1665) | < 0.001 | --- | --- | |
| Laboratory tests | | | | | | |
| Hematocrit, % | 31 (27 - 35) | 31 (28 - 37) | 0.142 | --- | --- | |
| White blood cell count, 10⁹/L | 13.6 (8.5 - 18.1) | 15.4 (9.8 - 23.3) | 0.009 | --- | --- | |
| Platelets, 10⁹/L | 164 (96 - 238) | 131 (80 - 223) | 0.032 | 177 (120 - 292) | 169 (90 - 256) | 0.048 |
| Creatinine, mg/dL | 1.0 (0.7 - 1.5) | 1.4 (0.9 - 2.6) | < 0.001 | 1.0 (0.7 - 1.5) | 1.7 (1.0 - 2.8) | < 0.001 |
| Bilirubin, mg/dL | 0.8 (0.5 - 1.4) | 0.8 (0.5 - 1.6) | 0.630 | 0.6 (0.4 - 1.2) | 0.9 (0.4 - 1.7) | 0.002 |
| Arterial blood gas | | | | | | |
| pH* | 7.39 (7.35 - 7.44) | 7.31 (7.24 - 7.35) | < 0.001 | 7.4 (7.3 - 7.4) | 7.2 (7.2 - 7.3) | < 0.001 |
| PaO₂, mmHg | 82 (68 - 101) | 86 (72 - 111.2) | 0.112 | 118 (82 - 158) | 104 (78 - 152) | 0.065 |
| PaO₂ / FiO₂ | 139 (98 - 195) | 107 (74 - 159) | < 0.001 | 118 (82 - 158) | 104 (78 - 152) | 0.065 |
| PaCO₂, mmHg | 38 (34 - 45) | 38 (32 - 44) | 0.423 | 46 (41 - 56) | 53 (42 - 65) | < 0.001 |
| Bicarbonate, mmol/L | 23 (20 - 26) | 17 (14 - 21) | < 0.001 | 25.2 (22.5 - 28.8) | 20.6 (17.8 - 23.4) | < 0.001 |
| Ventilatory variables | | | | | | |
| Tidal volume, mL | 420 (360 - 480) | 400 (340 - 450) | 0.016 | 360 (320 - 400) | 350 (300 - 397.8) | 0.008 |
| Per PBW, mL/kg PBW | 6.4 (6.0 - 7.3) | 6.1 (5.9 - 7.0) | 0.030 | 6.0 (5.3 - 6.1) | 5.9 (5.1 - 6.1) | 0.034 |
| Plateau pressure, cmH₂O | 22.0 (18.0 - 27.0) | 25.0 (20.0 - 29.0) | 0.003 | 24.0 (21.0 - 28.0) | 27.0 (23.0 - 30.0) | < 0.001 |
| PEEP, cmH₂O | 8 (5 - 10) | 10 (8 - 13) | 0.001 | 10 (10 - 14) | 12 (10 - 14) | < 0.001 |
| Respiratory rate, breaths/min | 23 (19 - 27) | 30 (24 - 35) | < 0.001 | 24 (20 - 28) | 30 (24 - 34) | < 0.001 |
| FiO₂ | 0.50 (0.40 - 0.60) | 0.70 (0.50 - 0.90) | < 0.001 | 0.70 (0.60 - 0.80) | 0.80 (0.70 - 1.00) | < 0.001 |

Data are mean ± standard deviation, median (quartile 25^{th} - quartile 75^{th}) or N (%)
Abbreviations: APACHE denotes Acute Physiology and Chronic Health Evaluation, V_{T}/PBW denotes tidal volume per predicted body weight...

TABLE 34

Clinical Outcomes According to Subphenotype in Each Trial

| | Subphenotype A | Subphenotype B | Difference (95% Cl) | p value |
|---|---|---|---|---|
| Training set | | | | |
| FACTT | n = 407 | n = 294 | | |
| 60-day mortality - no. (%) | 94 (23.1) | 102 (34.7) | 11.6% (4.9% to 18.3%) | 0.001 |
| 90-day mortality - no. (%) | 103 (25.4) | 106 (36.3) | 10.9% (4.1% to 17.8%) | 0.002 |
| Ventilator-free days at day 28 | 19.0 (0.0 - 24.0) | 10.0(0.0 - 21.0) | -9.0 (-11.9 to -6.1) | < 0.001 |
| Duration of ventilation in survivors, days | 8.0 (4.0 - 13.0) | 10.0(7.0 - 19.0) | 2.0 (0.5 to 3.5) | < 0.001 |
| EDEN | n = 449 | n = 328 | | |
| 60-day mortality - no. (%) | 87 (19.4) | 90 (27.4) | 8.1% (2.1% to 14.0%) | 0.010 |
| 90-day mortality - no. (%) | 90 (20.0) | 93 (28.4) | 8.3% (2.3% to 14.3%) | 0.009 |
| Ventilator-free days at day 28 | 21.0 (0.0 - 25.0) | 15.0 (0.0 - | 22.2) -6.0 (-8.1 to -3.9) | < 0.001 |
| Duration of ventilation in survivors, days | 6.0 (4.0 - 11.0) | 8.0 (6.0 - 18.0) | 2.0 (0.9 to 3.1) | < 0.001 |
| Validation set | | | | |
| ALVEOLI | n = 336 | n = 157 | | |
| 60-day mortality - no. (%) | 59 (17.6) | 68 (43.3) | 25.8% (17.7% to 33.8%) | < 0.001 |
| 90-day mortality - no. (%) | 60 (18.1) | 70 (45.5) | 27.3% (19.2% to 35.5%) | < 0.001 |
| Ventilator-free days at day 28 | 21.0 (4.8 - 25.0) | 2.0 (0.0 - 19.0) | -19.0 (-20.8 to -17.2) | < 0.001 |
| Duration of ventilation in survivors, days | 7.0 [4.0,13.0] | 11.0 (6.0 - | 22.2) 4.0 (2.1 to 5.9) | < 0.001 |
| ARMA | n = 279 | n = 100 | | |
| 60-day mortality - no. (%) | 69 (24.8) | 42 (42.0) | 17.2% (6.9% to 27.5%) | 0.002 |
| 90-day mortality - no. (%) | 70 (25.5) | 42 (42.0) | 16.5% (6.0% to 26.9%) | 0.003 |
| Ventilator-free days at day 28 | 17.0 (0.0 - 24.0) | 2.0 (0.0 - 19.0) | -15.0 (-18.6 to -11.4) | < 0.001 |
| Duration of ventilation in survivors, days | 7.0 (4.0 - 13.8) | 11.0 (5.0 -18.0) | 4.0 (1.5 to 6.5) | 0.018 |
| SAILS | n = 319 | n = 188 | | |
| 60-day mortality - no. (%) | 80 (25.1) | 60 (31.9) | 6.8% (-1.2% to 14.9%) | 0.119 |
| 90-day mortality - no. (%) | 81 (25.4) | 63 (33.5) | 8.1% (0.0% to 16.3%) | 0.063 |
| Ventilator-free days at day 28 | 21.0 (0.0 - 25.0) | 16.0 (0.0 - 23.0) | -5.0 (-7.3 to -2.7) | < 0.001 |
| Duration of ventilation in survivors, days | 6.0 (3.0 - 10.0) | 8.0 (5.0 - 14.0) | 2.0 (0.7 to 3.3) | < 0.001 |
| ART | n = 211 | n = 298 | | |
| 28-day mortality - no. (%) | 81 (38.4) | 180 (60.4) | 22.0% (13.4% to 30.7%) | < 0.001 |
| Ventilator-free days at day 28 | 0.0 (0.0 - 17.0) | 0.0 (0.0 - 7.8) | -0.0 (-1.0 to 1.0) | < 0.001 |
| Duration of ventilation in survivors, days | 12.0 (8.0 - 20.0) | 13.5 (8.0 - 20.0) | 2.0 (-0.3 to 4.2) | 0.570 |

Data are median (quartile 25^{th} - quartile 75^{th}) or N (%). Difference is mean difference with (95% CI) for binomial variables and median difference with (95% CI) for continuous variables
Abbreviations: CI is confidence interval.

TABLE 35A

Biomarker levels by study and subphenotype generated by Model B.2

| | ARMA | | | | ALVEOLI | | | |
|---|---|---|---|---|---|---|---|---|
| | Subphenotype A (n = 279) | Subphenotype B (n = 100) | Median Difference (95% CI) | p value | Subphenotype A (n = 336) | Subphenotype B (n = 157) | Median Difference CI) (95% | p value |
| ICAM-1 | 654.0 (399.0 - 959.4) | 888.0 (550.0 - 1365.3) | 234 (60.3 to 407.8) | 0.002 | 847.9 (585.7 - 1227.1) | 1070.4 (748.2 - 1588.8) | 219.4 (90.4 to 348.4) | < 0.001 |
| IL-6 | 214.0 (91.8 - 553.5) | 966.0 (291.0 - 2200.0) | 749.1 (589.9 to 908.2) | < 0.001 | 182.5 (85.5 - 435.2) | 775.0 (148.0 - 2846.5) | 592 (515.5 to 668.6) | < 0.001 |
| PAI-1 | 65.3 (37.8 - 109.5) | 101.7 (50.8 - 291.6) | 41 (18.3 to 63.7) | 0.001 | Not assessed | Not assessed | --- | --- |
| IL-8 | 46.0 (2.0 - 91.0) | 106.9 (43.8 - 281.4) | 60.9 (35.6 to 86.2) | < 0.001 | Not assessed | Not assessed | --- | --- |
| IL-10 | 16.0 (0.0 - 40.3) | 47.9 (0.0 - 120.7) | 31.9 (20.2 to 43.6) | < 0.001 | Not assessed | Not assessed | --- | --- |
| TNFR-I | 2604.0 (1950.0 - 3777.0) | 6897.0 (3622.5 - 12281.5) | 4293 (3323.6 to 5262.4) | < 0.001 | Not assessed | Not assessed | --- | --- |
| TNFR-II | 6581.0 (4958.0 - 9658.0) | 18611.0 (12262.5 - 35652.0) | 12030 (9577.5 to 14482.5) | < 0.001 | Not assessed | Not assessed | --- | --- |
| SPA | 29.0 (11.8 - 68.0) | 25.0 (10.5 - 40.0) | -4 (-19.9 to 11.9) | 0.398 | Not assessed | Not assessed | --- | --- |
| SPD | 76.0 (36.2 - 145.2) | 59.0 (30.0 - 125.0) | -18 (-52.6 to 16.6) | 0.254 | Not assessed | Not assessed | --- | --- |
| VW | 308.0 (165.5 - 431.0) | 384.0 (246.0 - 549.0) | 76 (-26.5 to 178.5) | 0.045 | Not assessed | Not assessed | --- | --- |

Data are median (quartile 25^{th} - quartile 75^{th}).
Abbreviations: 95%CI denotes 95% confidence interval, ICAM-1 is intercellular adhesion molecule-1, IL-6 is interleukin-6, PAI-1 is plasminogen activator inhibitor-1, IL-8 is interleukin-8, IL-10 is interleukin-10, TNFR-I is tumor necrosis factor receptor 1, TNFR-II is tumor necrosis factor II, SPA is surfact protein A, SPD is surfact Protein D and VW is Von Willebrand factor.

TABLE 35B

IL-6 biomarker levels by study and subphenotype generated using the 16 different models

| | ARMA | | | ALVEOLI | | |
|---|---|---|---|---|---|---|
| | Subphenotype A (Median) | Subphenotype B (Median) | Median Fold Change | Subphenotype A (Median) | Subphenotype B (Median) | Median Fold Change |
| Model B.1 | 207.5 | 742 | 3.58 | 182 | 727 | 3.99 |
| Model B.2 | 214 | 966 | 4.51 | 182.5 | 775 | 4.25 |
| Model B.3 | 217 | 731 | 3.37 | 179 | 778 | 4.35 |
| Model B.4 | 217 | 719 | 3.31 | 178 | 757.5 | 4.26 |
| Model B.5 | 229 | 562.5 | 2.46 | 193 | 537.5 | 2.78 |
| Model B.6 | 228 | 548 | 2.40 | 194 | 499.5 | 2.57 |
| Model. B.7 | 210 | 1037 | 4.94 | 183 | 776.5 | 4.24 |
| Model B.8 | 206 | 1001.5 | 4.86 | 182 | 950 | 5.22 |
| Model B.9 | 217 | 854 | 3.94 | 183 | 637.5 | 3.48 |
| Model B.10 | 413.5 | 229 | 0.55 | 225 | 250 | 1.11 |
| Model B.11 | 219 | 742 | 3.39 | 182 | 757 | 4.16 |
| Model B.12 | 249 | 472 | 1.90 | 192.5 | 499.5 | 2.59 |
| Model B.13 | 222 | 542 | 2.44 | 165.5 | 537.5 | 3.25 |
| Model B.14 | 217 | 700 | 3.23 | 183 | 740 | 4.04 |
| Model B.15 | 221 | 718 | 3.25 | 176 | 776.5 | 4.41 |
| Model B.16 | 221 | 720 | 3.26 | 175 | 794 | 4.54 |

TABLE 36

PEEP differential treatment response, according to subphenotype assignment when training the B.1 model on ARDS patients from ALVEOLI study

| | PF<200 | | | | | PF<300 | | | | |
|---|---|---|---|---|---|---|---|---|---|---|
| | Subphenotype B | | Subphenotype A | | p-value | Subphenotype B | | Subphenotype A | | p-value |
| B.1 Model | High PEEP | Low PEEP | High PEEP | Low PEEP | | High PEEP | Low PEEP | High PEEP | Low PEEP | |
| DEAD60, n (%) | 34 (45.9) | 29 (44.6) | 31 (22.0) | 22 (16.7) | 0.53 | 38 (43.7) | 35 (43.2) | 37 (21.0) | 26 (14.8) | 0.329 |
| DEAD90, n (%) | 36 (48.6) | 29 (44.6) | 31 (22.0) | 23 (17.4) | 0.763 | 40 (46.0) | 36 (44.4) | 37 (21.0) | 26 (14.8) | 0.362 |
| VFD, median (IQR) | 0.0 (0.0 18.0) | 0.0 (0.0 - 19.0) | 21.0 (0.0 - 24.0) | 19.0 (5.8-24.0) | 0.631 | 0.0 (0.0 - 18.0) | 0.0 (0.0 - 21.0) | 21.0 (0.0 - 24.0) | 20.0 (8.8 -25.0) | 0.222 |

TABLE 37

PEEP differential treatment response, according to subphenotype assignment when training the B.2 model on ARDS patients from ALVEOLI study

| | PF<200 | | | | | PF<300 | | | | |
|---|---|---|---|---|---|---|---|---|---|---|
| | Subphenotype B | | Subphenotype A | | p-value | Subphenotype B | | Subphenotype | | p-value |
| B.2 Model | High PEEP | Low PEEP | High PEEP | Low PEEP | | High PEEP | Low PEEP | High PEEP | Low PEEP | |
| DEAD60, n (%) | 31 (47.0) | 28 (47.5) | 31 (22.1) | 19 (14.8) | 0.291 | 34 (42.5) | 34 (44.2) | 37 (21.6) | 22 (13.3) | 0.135 |
| DEAD90, n (%) | 33 (50.0) | 28 (47.5) | 31 (22.1) | 20 (15.6) | 0.402 | 36 (45.0) | 34 (44.2) | 37 (21.6) | 23 (13.9) | 0.222 |
| VFD, median (IQR) | 0.0 (0.0 - 18.0) | 0.0 (0.0 - 19.0) | 21.0 (0.0 24.0) | 18.5 (7.5 -24.0) | 0.644 | 1.0 (0.0 18.0) | 5.0 (0.0 - 21.0) | 21.0 (0.0 24.5) | 20.0 (9.0 -25.0) | 0.087 |

TABLE 38

PEEP differential treatment response, according to subphenotype assignment when training the B.3 model on ARDS patients from ALVEOLI study

| | PF<200 | | | | | PF<300 | | | | |
|---|---|---|---|---|---|---|---|---|---|---|
| | Subphenotype | | Subphenotype A | | p-value | Subphenotype B | | Subphenotype A | | p-value |
| B.3 Model | High PEEP | Low PEEP | High PEEP | Low PEEP | | High PEEP | Low PEEP | High PEEP | Low PEEP | |
| DEAD60, n (%) | 28 (41.8) | 26 (44.8) | 34 (24.5) | 21 (16.3) | 0.183 | 32 (40.0) | 33 (41.8) | 39 (22.8) | 23 (14.1) | 0.128 |
| DEAD90, n (%) | 30 (44.8) | 26 (44.8) | 34 (24.5) | 22 (17.1) | 0.3 | 34 (42.5) | 33 (41.8) | 39 (22.8) | 24 (14.7) | 0.213 |
| VFD, median (IQR) | 2.0 (0.0 19.0) | 0.0 (0.0 19.0) | 21.0 (0.0 24.0) | 19.0 (6.0 -24.0) | 0.636 | 2.0 (0.0 18.0) | 5.0 (0.0 21.0) | 21.0 (0.0 24.5) | 20.0 (9.0 -25.0) | 0.077 |

TABLE 39

PEEP differential treatment response, according to subphenotype assignment when training the B.4 model on ARDS patients from ALVEOLI study

| | PF<200 | | | | | PF<300 | | | | |
|---|---|---|---|---|---|---|---|---|---|---|
| | Subphenotype B | | Subphenotype A | | p-value | Subphenotype B | | Subphenotype A | | p-value |
| B.4 Model | High PEEP | Low PEEP | High PEEP | Low PEEP | | High PEEP | Low PEEP | High PEEP | Low PEEP | |
| DEAD60, n (%) | 31 (41.9) | 29 (44.6) | 34 (24.1) | 22 (16.7) | 0.212 | 36 (42.4) | 34 (41.5) | 39 (21.9) | 27 (15.4) | 0.346 |
| DEAD90, n (%) | 33 (44.6) | 29 (44.6) | 34 (24.1) | 23 (17.4) | 0.353 | 38 (44.7) | 34 (41.5) | 39 (21.9) | 28 (16.0) | 0.52 |
| VFD, median (IQR) | 0.0 (0.0 - 19.5) | 0.0 (0.0 - 19.0) | 21.0 (0.0 24.0) | 19.0 (5.8 -24.0) | 0.629 | 0.0 (0.0 - 18.0) | 2.5 (0.0 - 21.0) | 21.0 (0.0 - 24.0) | 20.0 (8.5 -25.0) | 0.226 |

TABLE 40

PEEP differential treatment response, according to subphenotype assignment when training the B.5 model on ARDS patients from ALVEOLI study

| | PF<200 | | | | | PF<300 | | | | |
|---|---|---|---|---|---|---|---|---|---|---|
| | Subphenotype B | | Subphenotype A | | p-value | Subphenotype B | | Subphenotype A | | p-value |
| B.5 Model | High PEEP | Low PEEP | High PEEP | Low PEEP | | High PEEP | Low PEEP | High PEEP | Low PEEP | |
| DEAD60, n (%) | 23 (37.1) | 19 (40.4) | 27 (25.2) | 15 (15.5) | 0.159 | 28 (38.9) | 23 (40.4) | 28 (21.5) | 18 (13.5) | 0.205 |
| DEAD90, n (%) | 24 (38.7) | 19 (40.4) | 27 (25.2) | 16 (16.5) | 0.258 | 29 (40.3) | 23 (40.4) | 28 (21.5) | 19 (14.3) | 0.313 |
| VFD, median (IQR) | 1.0 (0.0 20.8) | 0.0 (0.0 19.5) | 21.0 (0.0 24.0) | 19.0 (10.0 -24.0) | 0.659 | 1.0 (0.0 - 19.2) | 0.0 (0.0 - 19.0) | 21.0 (0.2 - 25.0) | 22.0 (11.0 -25.0) | 0.999 |

TABLE 41

PEEP differential treatment response, according to subphenotype assignment when training the B.6 model on ARDS patients from ALVEOLI study

| | PF<200 | | | | | PF<300 | | | | |
|---|---|---|---|---|---|---|---|---|---|---|
| | Subphenotype B | | Subphenotype A | | p-value | Subphenotype B | | Subphenotype A | | p-value |
| B.6 Model | High PEEP | Low PEEP | High PEEP | Low PEEP | | High PEEP | Low PEEP | High PEEP | Low PEEP | |
| DEAD60, n (%) | 22 (38.6) | 19 (42.2) | 26 (24.5) | 13 (13.8) | 0.121 | 25 (37.3) | 22 (40.0) | 28 (22.0) | 16 (12.8) | 0.129 |
| DEAD90, n (%) | 23 (40.4) | 19 (42.2) | 26 (24.5) | 14 (14.9) | 0.165 | 26 (38.8) | 22 (40.0) | 28 (22.0) | 17 (13.6) | 0.196 |
| VFD, median (IQR) | 5.0 (0.0 21.0) | 0.0 (0.0 19.0) | 21.0 (0.0 - 24.0) | 19.5 (11.0 -24.0) | 0.389 | 2.0 (0.0 19.5) | 0.0 (0.0 19.5) | 21.0 (0.0 - 25.0) | 21.0 (11.0 -25.0) | 1 |

TABLE 42

PEEP differential treatment response, according to subphenotype assignment when training the B.7 model on ARDS patients from ALVEOLI study

| | PF<200 | | | | | PF<300 | | | | |
|---|---|---|---|---|---|---|---|---|---|---|
| | Subphenotype B | | Subphenotype A | | p-value | Subphenotype B | | Subphenotype A | | p-value |
| B.7 Model | High PEEP | Low PEEP | High PEEP | Low PEEP | | High PEEP | Low PEEP | High PEEP | Low PEEP | |
| DEAD60, n (%) | 32 (47.8) | 28 (47.5) | 30 (21.6) | 19 (14.8) | 0.356 | 34 (42.5) | 33 (44.6) | 37 (21.6) | 23 (13.7) | 0.143 |
| DEAD90, n (%) | 34 (50.7) | 28 (47.5) | 30 (21.6) | 20 (15.6) | 0.533 | 36 (45.0) | 33 (44.6) | 37 (21.6) | 24 (14.3) | 0.23 |
| VFD, median (IQR) | 0.0 (0.0 - 17.5) | 0.0 (0.0 - 19.0) | 21.0 (0.0 - 24.0) | 18.5 (7.5 -24.0) | 0.634 | 1.0 (0.0 - 18.0) | 2.5 (0.0 - 20.5) | 21.0 (0.0 24.5) | 20.0 (8.8 -25.0) | 0.086 |

TABLE 43

PEEP differential treatment response, according to subphenotype assignment when training the B.8 model on ARDS patients from ALVEOLI study

| | PF<200 | | | | | PF<300 | | | | |
|---|---|---|---|---|---|---|---|---|---|---|
| | Subphenotype B | | Subphenotype A | | p-value | Subphenotype B | | Subphenotype A | | p-value |
| B.8 Model | High PEEP | Low PEEP | High PEEP | Low PEEP | | High PEEP | Low PEEP | High PEEP | Low PEEP | |
| DEAD60, n (%) | 28 (46.7) | 24 (51.1) | 22 (19.8) | 15 (14.3) | 0.287 | 29 (43.9) | 29 (48.3) | 26 (18.8) | 16 (11.8) | 0.141 |
| DEAD90, n (%) | 29 (48.3) | 24 (51.1) | 22 (19.8) | 16 (15.2) | 0.354 | 30 (45.5) | 29 (48.3) | 26 (18.8) | 17 (12.5) | 0.187 |
| VFD, median (IQR) | 1.0 (0.0 - 18.5) | 0.0 (0.0 - 19.0) | 21.0 (0.0 24.0) | 19.0 (9.0 -24.0) | 0.671 | 1.0 (0.0 18.0) | 0.0 (0.0 19.0) | 21.5 (1.5 24.8) | 20.5 (10.8 -25.0) | 0.603 |

TABLE 44

PEEP differential treatment response, according to subphenotype assignment when training the B.9 model on ARDS patients from ALVEOLI study

| | PF<200 | | | | | PF<300 | | | | |
|---|---|---|---|---|---|---|---|---|---|---|
| | Subphenotype B | | Subphenotype A | | p-value | Subphenotype B | | Subphenotype A | | p-value |
| B.9 Model | High PEEP | Low PEEP | High PEEP | Low PEEP | | High PEEP | Low PEEP | High PEEP | Low PEEP | |
| DEAD60, n (%) | 35 (45.5) | 28 (44.4) | 30 (21.7) | 23 (17.2) | 0.584 | 38 (44.2) | 34 (43.0) | 37 (20.9) | 27 (15.2) | 0.413 |
| DEAD90, n (%) | 37 (48.1) | 28 (44.4) | 30 (21.7) | 24 (17.9) | 0.806 | 40 (46.5) | 35 (44.3) | 37 (20.9) | 27 (15.2) | 0.449 |
| VFD, median (IQR) | 0.0 (0.0 - 18.0) | 0.0 (0.0 - 19.0) | 21.0(0.0- 24.0) | 18.5(5.2-24.0) | 0.621 | 0.0 (0.0 - 18.0) | 0.0 (0.0 - 21.0) | 21.0 (0.0 - 24.0) | 20.0 (6.5 -25.0) | 0.223 |

TABLE 45

PEEP differential treatment response, according to subphenotype assignment when training the B.10 model on ARDS patients from ALVEOLI study

| | PF<200 | | | | | PF<300 | | | | |
|---|---|---|---|---|---|---|---|---|---|---|
| | Subphenotype B | | Subphenotype A | | p-value | Subphenotype B | | Subphenotype A | | p-value |
| B.10 Model | High PEEP | Low PEEP | High PEEP | Low PEEP | | High PEEP | Low PEEP | High PEEP | Low PEEP | |
| DEAD60, n (%) | 37 (29.4) | 34 (31.8) | 28 (31.1) | 17 (18.9) | 0.087 | 43 (27.6) | 41 (28.3) | 33 (27.7) | 26 (20.5) | 0.272 |
| DEAD90, n (%) | 38 (30.2) | 35 (32.7) | 29 (32.2) | 17 (18.9) | 0.067 | 45 (28.8) | 42 (29.0) | 34 (28.6) | 26 (20.5) | 0.272 |
| VFD, median (IQR) | 17.5(0.0- 23.0) | 11.0(0.0- 22.0) | 15.5(0.0- 23.8) | 18.5(7.2-24.0) | 0.592 | 17.5 (0.0 - 23.2) | 16.0 (0.0 - 24.0) | 18.0 (0.0 - 24.0) | 19.0(0.0-24.0) | 0.499 |

TABLE 46

PEEP differential treatment response, according to subphenotype assignment when training the B.11 model on ARDS patients from ALVEOLI study

| | PF<200 | | | | | PF<300 | | | | |
|---|---|---|---|---|---|---|---|---|---|---|
| | Subphenotype B | | Subphenotype A | | p-value | Subphenotype B | | Subphenotype A | | p-value |
| B.11 Model | High PEEP | Low PEEP | High PEEP | Low PEEP | | High PEEP | Low PEEP | High PEEP | Low PEEP | |
| DEAD60, n (%) | 27 (40.3) | 26 (44.1) | 35 (25.2) | 21 (16.4) | 0.144 | 32 (38.6) | 32 (42.1) | 39 (23.2) | 24 (14.5) | 0.092 |
| DEAD90, n (%) | 29 (43.3) | 26 (44.1) | 35 (25.2) | 22 (17.2) | 0.246 | 34 (41.0) | 32 (42.1) | 39 (23.2) | 25 (15.1) | 0.153 |
| VFD, median (IQR) | 2.0 (0.0 - 19.0) | 0.0 (0.0 - 19.0) | 21.0(0.0- 24.0) | 18.5(5.8-24.0) | 0.638 | 5.0 (0.0 - 20.0) | 2.5 (0.0 - 21.0) | 21.0 (0.0 - 24.2) | 20.0 (9.0 -25.0) | 0.996 |

TABLE 47

PEEP differential treatment response, according to subphenotype assignment when training the B.12 model on ARDS patients from ALVEOLI study

| | PF<200 | | | | | PF<300 | | | | |
|---|---|---|---|---|---|---|---|---|---|---|
| | Subphenotype B | | Subphenotype A | | p-value | Subphenotype B | | Subphenotype A | | p-value |
| B.12 Model | High PEEP | Low PEEP | High PEEP | Low PEEP | | High PEEP | Low PEEP | High PEEP | Low PEEP | |
| DEAD60, n (%) | 24 (42.9) | 17 (40.5) | 24 (22.4) | 15 (15.5) | 0.514 | 28 (40.0) | 22 (40.7) | 25 (20.2) | 16 (12.7) | 0.251 |
| DEAD90, n (%) | 25 (44.6) | 17 (40.5) | 24 (22.4) | 16 (16.5) | 0.685 | 29 (41.4) | 22 (40.7) | 25 (20.2) | 17 (13.5) | 0.352 |
| VFD, median (IQR) | 0.0 (0.0 - 20.2) | 2.5 (0.0 - 20.8) | 21.0(0.0- 24.0) | 19.0(9.0-24.0) | 0.673 | 1.0(0.0- 18.8) | 2.5(0.0- 19.8) | 21.5(5.5- 25.0) | 21.0(11.0-25.0) | 0.606 |

TABLE 48

PEEP differential treatment response, according to subphenotype assignment when training the B.13 model on ARDS patients from ALVEOLI study

| | PF<200 | | | | | PF<300 | | | | |
|---|---|---|---|---|---|---|---|---|---|---|
| | Subphenotype B | | Subphenotype A | | p-value | Subphenotype B | | Subphenotype A | | p-value |
| B.13 Model | High PEEP | Low PEEP | High PEEP | Low PEEP | | High PEEP | Low PEEP | High PEEP | Low PEEP | |
| DEAD60, n (%) | 23 (46.0) | 14 (35.0) | 19 (24.1) | 10 (13.3) | 0.667 | 26 (41.3) | 17 (32.1) | 21 (22.8) | 13 (14.3) | 0.749 |
| DEAD90, n (%) | 24 (48.0) | 14 (35.0) | 19 (24.1) | 11 (14.7) | 0.884 | 27 (42.9) | 17 (32.1) | 21 (22.8) | 14 (15.4) | 0.944 |
| VFD, median (IQR) | 0.0 (0.0 - 17.8) | 7.5 (0.0 - 19.2) | 20.0 (0.0 - 24.0) | 20.0 (11.0 -25.0) | 0.999 | 2.0 (0.0 - 18.0) | 14.0 (0.0 - 20.0) | 22.0 (0.0 - 25.0) | 22.0 (11.0 -25.5) | 0.66 |

TABLE 49

PEEP differential treatment response, according to subphenotype assignment when training the B.14 model on ARDS patients from ALVEOLI study

| | PF<200 | | | | | PF<300 | | | | |
|---|---|---|---|---|---|---|---|---|---|---|
| | Subphenotype B | | Subphenotype A | | p-value | Subphenotype B | | Subphenotype A | | p-value |
| B.14 Model | High PEEP | Low PEEP | High PEEP | Low PEEP | | High PEEP | Low PEEP | High PEEP | Low PEEP | |
| DEAD60, n (%) | 32 (46.4) | 26 (40.6) | 33(22.6) | 25 (18.8) | 0.997 | 35 (41.2) | 34 (42.0) | 40 (22.5) | 27 (15.3) | 0.23 |
| DEAD90, n (%) | 33 (47.8) | 26 (40.6) | 34 (23.3) | 26 (19.5) | 0.889 | 36 (42.4) | 34 (42.0) | 41 (23.0) | 28 (15.9) | 0.272 |
| VFD, median (IQR) | 0.0(0.0- 18.0) | 5.5(0.0- 20.2) | 21.0(0.0- 24.0) | 18.0(4.0-24.0) | 0.318 | 2.0 (0.0 - 18.0) | 5.0 (0.0 - 21.0) | 21.0 (0.0 - 24.0) | 20.0 (7.5 -25.0) | 0.232 |

TABLE 50

PEEP differential treatment response, according to subphenotype assignment when training the B.15 model on ARDS patients from ALVEOLI study

| | PF<200 | | | | | PF<300 | | | | |
|---|---|---|---|---|---|---|---|---|---|---|
| | Subphenotype B | | Subphenotype A | | p-value | Subphenotype B | | Subphenotype A | | p-value |
| B.15 Model | High PEEP | Low PEEP | High PEEP | Low PEEP | | High PEEP | Low PEEP | High PEEP | Low PEEP | |
| DEAD60, n (%) | 33 (47.1) | 27 (42.9) | 32 (21.9) | 24 (17.9) | 0.865 | 37 (44.0) | 33 (40.2) | 38 (21.0) | 28 (16.0) | 0.672 |
| DEAD90, n (%) | 34 (48.6) | 27 (42.9) | 33(22.6) | 25 (18.7) | 0.966 | 38 (45.2) | 33 (40.2) | 40 (22.1) | 29 (16.6) | 0.694 |
| VFD, median (IQR) | 0.0(0.0- 15.8) | 5.0(0.0- 21.0) | 21.0(0.0- 24.0) | 18.0(4.0-24.0) | 0.011 | 0.0 (0.0 - 18.0) | 8.5 (0.0 - 21.0) | 21.0 (0.0 - 24.0) | 20.0 (5.5 -25.0) | 0.222 |

TABLE 51

PEEP differential treatment response, according to subphenotype assignment when training the B.16 model on ARDS patients from ALVEOLI study

| | PF<200 | | | | | PF<300 | | | | |
|---|---|---|---|---|---|---|---|---|---|---|
| | Subphenotype B | | Subphenotype A | | p-value | Subphenotype B | | Subphenotype A | | p-value |
| B.16 Model | High PEEP | Low PEEP | High PEEP | Low PEEP | | High PEEP | Low PEEP | High PEEP | Low PEEP | |
| DEAD60, n (%) | 31 (45.6) | 24 (42.1) | 34(23.0) | 27 (19.3) | 0.863 | 37 (44.0) | 33 (41.8) | 38 (21.0) | 28 (15.7) | 0.535 |
| DEAD90, n (%) | 32 (47.1) | 24 (42.1) | 35 (23.6) | 28 (20.0) | 0.952 | 38 (45.2) | 33 (41.8) | 40 (22.1) | 29 (16.3) | 0.549 |
| VFD, median (IQR) | 0.0(0.0- 16.2) | 5.0(0.0- 21.0) | 21.0(0.0- 24.0) | 17.5(1.5-24.0) | 0.011 | 0.0 (0.0 - 18.0) | 7.0 (0.0 - 21.0) | 21.0 (0.0 - 24.0) | 20.0 (5.2 -25.0) | 0.222 |

TABLE 52

PEEP differential treatment response, according to subphenotype assignment when training the B.1 model on ARDS patients from ART study

| | PF<200 | | | | | PF<300 | | | | |
|---|---|---|---|---|---|---|---|---|---|---|
| | Subphenotype B | | Subphenotype A | | p-value | Subphenotype B | | Subphenotype A | | p-value |
| B.1 Model | High PEEP | Low PEEP | High PEEP | Low PEEP | | High PEEP | Low PEEP | High PEEP | Low PEEP | |
| DEAD28, n (%) | 138 (57.0) | 148 (61.4) | 44 (33.6) | 58 (43.0) | 0.491 | 135 (59.2) | 141 (63.5) | 47 (32.4) | 65 (42.2) | 0.44 |
| DEAD90, n (%) | 156 (64.5) | 164 (68.0) | 60 (45.8) | 71 (52.6) | 0.721 | 152 (66.7) | 155 (69.8) | 64 (44.1) | 80 (51.9) | 0.586 |
| VFD, median (IQR) | 0.0 (0.0- 11.8) | 0.0 (0.0- 5.0) | 2.0 (0.0- 17.0) | 0.0 (0.0-14.5) | 0.233 | 0.0 (0.0- 9.2) | 0.0 (0.0- 2.0) | 5.0 (0.0- 18.0) | 0.0 (0.0-14.0) | 0.031 |

TABLE 53

PEEP differential treatment response, according to subphenotype assignment when training the B.2 model on ARDS patients from ART study

| | PF<200 | | | | | PF<300 | | | | |
|---|---|---|---|---|---|---|---|---|---|---|
| | Subphenotype B | | Subphenotype A | | p-value | Subphenotype B | | Subphenotype A | | p-value |
| B.2 Model | High PEEP | Low PEEP | High PEEP | Low PEEP | | High PEEP | Low PEEP | High PEEP | Low PEEP | |
| DEAD28, n (%) | 94 (59.1) | 85 (58.6) | 33 (33.0) | 49 (46.7) | 0.109 | 95 (60.9) | 85 (59.9) | 32 (31.1) | 49 (45.4) | 0.079 |
| DEAD90, n (%) | 103 (64.8) | 99 (68.3) | 44 (44.0) | 58 (55.2) | 0.429 | 103 (66.0) | 99 (69.7) | 44 (42.7) | 58 (53.7) | 0.465 |
| VFD, median (IQR) | 0.0 (0.0 - 10.0) | 0.0 (0.0 - 7.0) | 10.0(0.0- 18.0) | 0.0 (0.0 -15.0) | 1 | 0.0 (0.0 - 8.0) | 0.0 (0.0 - 6.2) | 10.0 (0.0 - 18.0) | 0.0 (0.0 -16.2) | 0.837 |

TABLE 54

PEEP differential treatment response, according to subphenotype assignment when training the B.3 model on ARDS patients from ART study

| | PF<200 | | | | | PF<300 | | | | |
|---|---|---|---|---|---|---|---|---|---|---|
| | Subphenotype B | | Subphenotype A | | p-value | Subphenotype B | | Subphenotype A | | p-value |
| B.3 Model | High PEEP | Low PEEP | High PEEP | Low PEEP | | High PEEP | Low PEEP | High PEEP | Low PEEP | |
| DEAD28, n (%) | 93 (57.4) | 86 (57.0) | 34 (35.1) | 48 (48.5) | 0.122 | 91 (59.1) | 83 (58.0) | 36 (34.3) | 51 (47.7) | 0.103 |
| DEAD90, n (%) | 102 (63.0) | 100 (66.2) | 45 (46.4) | 57 (57.6) | 0.41 | 100 (64.9) | 97 (67.8) | 47 (44.8) | 60 (56.1) | 0.381 |
| VFD, median (IQR) | 0.0 (0.0 - 11.0) | 0.0 (0.0 - 8.0) | 5.0 (0.0 - 17.0) | 0.0 (0.0 - 14.5) | 0.836 | 0.0 (0.0 - 11.0) | 0.0 (0.0 - 8.5) | 5.0 (0.0 - 17.0) | 0.0 (0.0 - 13.5) | 0.828 |

TABLE 55

PEEP differential treatment response, according to subphenotype assignment when training the B.4 model on ARDS patients from ART study

| | PF<200 | | | | | PF<300 | | | | |
|---|---|---|---|---|---|---|---|---|---|---|
| | Subphenotype B | | Subphenotype A | | p-value | Subphenotype B | | Subphenotype A | | p-value |
| B.4 Model | High PEEP | Low PEEP | High PEEP | Low PEEP | | High PEEP | Low PEEP | High PEEP | Low PEEP | |
| DEAD28, n (%) | 132 (56.4) | 148 (60.4) | 50 (36.0) | 58 (44.3) | 0.558 | 129 (58.9) | 137 (62.6) | 53 (34.4) | 69 (43.9) | 0.415 |
| DEAD90, n (%) | 149 (63.7) | 163 (66.5) | 67 (48.2) | 72 (55.0) | 0.64 | 145 (66.2) | 151 (68.9) | 71 (46.1) | 84 (53.5) | 0.575 |
| VFD, median (IQR) | 0.0(0.0- 11.8) | 0.0(0.0- 7.0) | 1.0(0.0- 17.0) | 0.0(0.0-14.0) | 0.629 | 0.0 (0.0- 10.5) | 0.0 (0.0- 3.5) | 2.5 (0.0- 17.0) | 0.0 (0.0-14.0) | 0.309 |

TABLE 56

PEEP differential treatment response, according to subphenotype assignment when training the B.5 model on ARDS patients from ART study

| | PF<200 | | | | | PF<300 | | | | |
|---|---|---|---|---|---|---|---|---|---|---|
| | Subphenotype B | | Subphenotype A | | p-value | Subphenotype B | | Subphenotype A | | p-value |
| B.5 model | High PEEP | Low PEEP | High PEEP | Low PEEP | | High PEEP | Low PEEP | High PEEP | Low PEEP | |
| DEAD28, n (%) | 131 (55.7) | 149 (60.6) | 42 (33.6) | 41 (38.7) | 0.947 | 136 (54.4) | 152 (59.8) | 37 (33.6) | 38 (38.8) | 0.999 |
| DEAD90, n (%) | 151 (64.3) | 162 (65.9) | 56 (44.8) | 57 (53.8) | 0.376 | 160 (64.0) | 167 (65.7) | 47 (42.7) | 52 (53.1) | 0.313 |
| VFD, median (IQR) | 0.0 (0.0 - 11.0) | 0.0 (0.0 - 7.8) | 5.0 (0.0 - 18.0) | 0.0 (0.0 - 15.0) | 1 | 0.0 (0.0 - 11.0) | 0.0 (0.0 - 8.8) | 4.0 (0.0 - 18.0) | 0.0 (0.0 - 13.8) | 0.634 |

TABLE 57

PEEP differential treatment response, according to subphenotype assignment when training the B.6 model on ARDS patients from ART study

| | PF<200 | | | | | PF<300 | | | | |
|---|---|---|---|---|---|---|---|---|---|---|
| | Subphenotype B | | Subphenotype A | | p-value | Subphenotype B | | Subphenotype A | | p-value |
| B.6 Model | High PEEP | Low PEEP | High PEEP | Low PEEP | | High PEEP | Low PEEP | High PEEP | Low PEEP | |
| DEAD28, n (%) | 92 (57.1) | 92 (58.2) | 31 (33.0) | 39 (44.3) | 0.253 | 95 (54.3) | 100 (55.9) | 28 (35.0) | 31 (46.3) | 0.312 |
| DEAD90, n (%) | 102 (63.4) | 103 (65.2) | 41 (43.6) | 51 (58.0) | 0.191 | 107 (61.1) | 116 (64.8) | 36 (45.0) | 38 (56.7) | 0.432 |
| VFD, median (IQR) | 0.0 (0.0 - 11.0) | 0.0 (0.0 - 10.8) | 8.0 (0.0 - 17.8) | 0.0 (0.0 - 11.8) | 0.129 | 0.0 (0.0 - 11.5) | 0.0 (0.0 - 10.5) | 7.5 (0.0 - 18.0) | 0.0 (0.0 - 14.5) | 0.66 |

TABLE 58

PEEP differential treatment response, according to subphenotype assignment when training the B.7 model on ARDS patients from ART study

| | PF<200 | | | | | PF<300 | | | | |
|---|---|---|---|---|---|---|---|---|---|---|
| | Subphenotype B | | Subphenotype A | | p-value | Subphenotype B | | Subphenotype A | | p-value |
| B.7 Model | High PEEP | Low PEEP | High PEEP | Low PEEP | | High PEEP | Low PEEP | High PEEP | Low PEEP | |
| DEAD28, n (%) | 95 (58.6) | 94 (59.5) | 32 (33.0) | 40 (43.5) | 0.276 | 94 (60.3) | 87 (59.2) | 33 (32.0) | 47 (45.6) | 0.095 |
| DEAD90, n (%) | 105 (64.8) | 108 (68.4) | 42 (43.3) | 49 (53.3) | 0.522 | 102 (65.4) | 101 (68.7) | 45 (43.7) | 56 (54.4) | 0.454 |
| VFD, median (IQR) | 0.0 (0.0 - 10.8) | 0.0 (0.0 - 7.0) | 10.0(0.0 - 18.0) | 0.0 (0.0 - 17.0) | 0.516 | 0.0 (0.0 - 8.5) | 0.0 (0.0 - 7.5) | 10.0 (0.0 - 18.0) | 0.0 (0.0 - 15.5) | 0.68 |

TABLE 59

PEEP differential treatment response, according to subphenotype assignment when training the B.8 model on ARDS patients from ART study

| | PF<200 | | | | | PF<300 | | | | |
|---|---|---|---|---|---|---|---|---|---|---|
| | Subphenotype B | | Subphenotype A | | p-value | Subphenotype B | | Subphenotype A | | p-value |
| B.8 Model | High PEEP | Low PEEP | High PEEP | Low PEEP | | High PEEP | Low PEEP | High PEEP | Low PEEP | |
| DEAD28, n (%) | 91 (58.7) | 85 (58.2) | 32 (32.0) | 46 (46.0) | 0.102 | 92 (59.4) | 84 (59.2) | 31 (31.0) | 47 (45.2) | 0.102 |
| DEAD90, n (%) | 101 (65.2) | 99 (67.8) | 42 (42.0) | 55 (55.0) | 0.282 | 101 (65.2) | 98 (69.0) | 42 (42.0) | 56 (53.8) | 0.421 |
| VFD, median (IQR) | 0.0 (0.0 - 10.5) | 0.0 (0.0 - 7.0) | 10.0(0.0- 18.0) | 0.0 (0.0 - 15.0) | 0.837 | 0.0 (0.0 - 10.0) | 0.0 (0.0 - 3.5) | 10.0 (0.0 - 18.0) | 0.0 (0.0 - 16.2) | 0.404 |

TABLE 60

PEEP differential treatment response, according to subphenotype assignment when training the B.9 model on ARDS patients from ART study

| | PF<200 | | | | | PF<300 | | | | |
|---|---|---|---|---|---|---|---|---|---|---|
| | Subphenotype B | | Subphenotype A | | p-value | Subphenotype B | | Subphenotype A | | p-value |
| B.9 Model | High PEEP | Low PEEP | High PEEP | Low PEEP | | High PEEP | Low PEEP | High PEEP | Low PEEP | |
| DEAD28, n (%) | 142 (57.0) | 151 (60.6) | 40 (32.3) | 55 (43.3) | 0.312 | 137 (58.8) | 145 (61.7) | 45 (32.1) | 61 (43.3) | 0.256 |
| DEAD90, n (%) | 162 (65.1) | 166 (66.7) | 54 (43.5) | 69 (54.3) | 0.253 | 156 (67.0) | 160 (68.1) | 60 (42.9) | 75 (53.2) | 0.242 |
| VFD, median (IQR) | 0.0 (0.0 - 11.0) | 0.0 (0.0 - 7.0) | 4.5 (0.0 - 18.0) | 0.0 (0.0 - 14.0) | 1 | 0.0 (0.0 - 9.0) | 0.0 (0.0 - 6.0) | 6.5 (0.0 - 18.0) | 0.0 (0.0 - 14.0) | 0.61 |

TABLE 61

PEEP differential treatment response, according to subphenotype assignment when training the B.10 model on ARDS patients from ART study

| | PF<200 | | | | | PF<300 | | | | |
|---|---|---|---|---|---|---|---|---|---|---|
| | Subphenotype B | | Subphenotype A | | p-value | Subphenotype B | | Subphenotype A | | p-value |
| B.10 Model | High PEEP | Low PEEP | High PEEP | Low PEEP | | High PEEP | Low PEEP | High PEEP | Low PEEP | |
| DEAD28, n (%) | 148 (46.7) | 170 (54.5) | 101 (53.2) | 106 (56.4) | 0.485 | 148 (46.7) | 170 (54.5) | 101 (53.2) | 106 (56.4) | 0.485 |
| DEAD90, n (%) | 176 (55.5) | 197(63.1) | 118(62.1) | 117 (62.2) | 0.245 | 176 (55.5) | 197 (63.1) | 118 (62.1) | 117 (62.2) | 0.245 |
| VFD, median (IQR) | 0.0 (0.0 - 15.0) | 0.0 (0.0 - 10.0) | 0.0 (0.0 - 13.0) | 0.0 (0.0 - 13.0) | 0.183 | 0.0 (0.0- 15.0) | 0.0 (0.0- 10.0) | 0.0 (0.0- 13.0) | 0.0 (0.0- 13.0) | 0.183 |

TABLE 62

PEEP differential treatment response, according to subphenotype assignment when training the B.11 model on ARDS patients from ART study

| | PF<200 | | | | | PF<300 | | | | |
|---|---|---|---|---|---|---|---|---|---|---|
| | Subphenotype B | | Subphenotype A | | p-value | Subphenotype B | | Subphenotype A | | p-value |
| B.11 Model | High PEEP | Low PEEP | High PEEP | Low PEEP | | High PEEP | Low PEEP | High PEEP | Low PEEP | |
| DEAD28, n (%) | 93 (57.4) | 91 (58.3) | 34 (35.1) | 43 (45.7) | 0.275 | 91 (58.3) | 89 (58.9) | 36 (35.0) | 45 (45.5) | 0.264 |
| DEAD90, n (%) | 102 (63.0) | 105 (67.3) | 45 (46.4) | 52 (55.3) | 0.656 | 101 (64.7) | 103 (68.2) | 46 (44.7) | 54 (54.5) | 0.517 |
| VFD, median (IQR) | 0.0 (0.0 - 11.0) | 0.0 (0.0 - 7.2) | 5.0 (0.0 - 17.0) | 0.0 (0.0 - 15.0) | 0.685 | 0.0 (0.0 - 11.0) | 0.0 (0.0 - 8.0) | 4.0 (0.0 - 17.0) | 0.0 (0.0 - 15.0) | 0.832 |

TABLE 63

PEEP differential treatment response, according to subphenotype assignment when training the B.12 model on ARDS patients from ART study

| | PF<200 | | | | | PF<300 | | | | |
|---|---|---|---|---|---|---|---|---|---|---|
| | Subphenotype B | | Subphenotype A | | p-value | Subphenotype B | | Subphenotype A | | p-value |
| B.12 Model | High PEEP | Low PEEP | High PEEP | Low PEEP | | High PEEP | Low PEEP | High PEEP | Low PEEP | |
| DEAD28, n (%) | 92 (58.2) | 95 (59.0) | 31 (32.0) | 36 (42.4) | 0.279 | 97 (53.3) | 106 (57.3) | 26 (35.6) | 25 (41.0) | 0.874 |
| DEAD90, n (%) | 103 (65.2) | 107 (66.5) | 40 (41.2) | 47 (55.3) | 0.182 | 114 (62.6) | 122 (65.9) | 29 (39.7) | 32 (52.5) | 0.369 |
| VFD, median (IQR) | 0.0 (0.0 - 11.0) | 0.0 (0.0 - 8.0) | 8.0 (0.0 - 18.0) | 0.0 (0.0 - 15.0) | 1 | 0.0 (0.0 - 12.8) | 0.0 (0.0 - 11.0) | 7.0 (0.0 - 18.0) | 0.0 (0.0 - 14.0) | 0.679 |

TABLE 64

PEEP differential treatment response, according to subphenotype assignment when training the B.13 model on ARDS patients from ART study

| | PF<200 | | | | | PF<300 | | | | |
|---|---|---|---|---|---|---|---|---|---|---|
| | Subphenotype B | | Subphenotype A | | p-value | Subphenotype B | | Subphenotype A | | p-value |
| B.13 Model | High PEEP | Low PEEP | High PEEP | Low PEEP | | High PEEP | Low PEEP | High PEEP | Low PEEP | |
| DEAD28, n (%) | 19 (65.5) | 17 (47.2) | 14 (40.0) | 19 (47.5) | 0.128 | 20 (60.6) | 24 (54.5) | 13 (41.9) | 12 (37.5) | 0.928 |
| DEAD90, n (%) | 21 (72.4) | 20 (55.6) | 17 (48.6) | 24 (60.0) | 0.09 | 22 (66.7) | 27 (61.4) | 16 (51.6) | 17 (53.1) | 0.676 |
| VFD, median (IQR) | 0.0 (0.0 - 0.0) | 0.0 (0.0 - 14.0) | 0.0 (0.0 - 18.0) | 0.0(0.0 - 15.5) | 0.001 | 0.0 (0.0- 11.0) | 0.0 (0.0- 13.0) | 0.0 (0.0- 18.5) | 0.0 (0.0 - 17.0) | 0.592 |

TABLE 65

PEEP differential treatment response, according to subphenotype assignment when training the B.14 model on ARDS patients from ART study

| | PF<200 | | | | | PF<300 | | | | |
|---|---|---|---|---|---|---|---|---|---|---|
| | Subphenotype B | | Subphenotype A | | p-value | Subphenotype B | | Subphenotype A | | p-value |
| B.14 model | High PEEP | Low PEEP | High PEEP | Low PEEP | | High PEEP | Low PEEP | High PEEP | Low PEEP | |
| DEAD28, n (%) | 124 (59.6) | 126 (62.1) | 58 (35.2) | 80 (46.2) | 0.234 | 126 (61.2) | 129 (63.2) | 56 (33.5) | 77 (44.8) | 0.203 |
| DEAD90, n (%) | 139 (66.8) | 140 (69.0) | 77 (46.7) | 95 (54.9) | 0.444 | 139 (67.5) | 143 (70.1) | 77 (46.1) | 92 (53.5) | 0.569 |
| VFD, median (IQR) | 0.0 (0.0 - 8.2) | 0.0 (0.0 - 2.5) | 3.0 (0.0 - 17.0) | 0.0 (0.0 - 14.0) | 0.317 | 0.0 (0.0 - 7.8) | 0.0 (0.0 - 2.0) | 5.0 (0.0 - 18.0) | 0.0 (0.0 - 14.0) | 0.167 |

TABLE 66

PEEP differential treatment response, according to subphenotype assignment when training the B.15 model on ARDS patients from ART study

| | PF<200 | | | | | PF<300 | | | | |
|---|---|---|---|---|---|---|---|---|---|---|
| | Subphenotype B | | Subphenotype A | | p-value | Subphenotype B | | Subphenotype A | | p-value |
| B.15 model | High PEEP | Low PEEP | High PEEP | Low PEEP | | High PEEP | Low PEEP | High PEEP | Low PEEP | |
| DEAD28, n (%) | 119 (59.5) | 129 (62.0) | 63 (36.4) | 77 (45.8) | 0.343 | 124 (62.3) | 127 (62.6) | 58 (33.3) | 79 (45.7) | 0.093 |
| DEAD90, n (%) | 131 (65.5) | 143 (68.8) | 85 (49.1) | 92 (54.8) | 0.796 | 134 (67.3) | 141 (69.5) | 82 (47.1) | 94 (54.3) | 0.53 |
| VFD, median (IQR) | 0.0 (0.0 - 11.0) | 0.0 (0.0 - 2.2) | 0.0 (0.0 - 17.0) | 0.0 (0.0 - 15.0) | 0.001 | 0.0 (0.0 - 8.5) | 0.0 (0.0 - 2.0) | 2.0 (0.0 - 18.0) | 0.0 (0.0 - 15.0) | 0.005 |

TABLE 67

PEEP differential treatment response, according to subphenotype assignment when training the B.16 model on ARDS patients from ART study

| | PF<200 | | | | | PF<300 | | | | |
|---|---|---|---|---|---|---|---|---|---|---|
| | Subphenotype B | | Subphenotype A | | p-value | Subphenotype B | | Subphenotype A | | p- |
| B.16 model | High PEEP | Low PEEP | High PEEP | Low PEEP | | High PEEP | Low PEEP | High PEEP | Low PEEP | value |
| DEAD28, n (%) | 124 (60.8) | 131 (62.1) | 58 (34.3) | 75 (45.5) | 0.173 | 124 (62.0) | 128 (62.7) | 58 (33.5) | 78 (45.3) | 0.123 |
| DEAD90, n (%) | 137 (67.2) | 144 (68.2) | 79 (46.7) | 91 (55.2) | 0.344 | 135 (67.5) | 141 (69.1) | 81 (46.8) | 94 (54.7) | 0.431 |
| VFD, median (IQR) | 0.0 (0.0 - 9.2) | 0.0 (0.0 - 2.5) | 2.0 (0.0 - 18.0) | 0.0(0.0 - 15.0) | 0.12 | 0.0 (0.0 - 9.2) | 0.0 (0.0 - 1.2) | 2.0 (0.0 - 18.0) | 0.0 (0.0 - 15.0) | 0.005 |

### Example 6: Further Example That Subtyped ARDS Patients Respond Differently to Varying Levels of PEEP

This is a retrospective study in a de-identified dataset pooling data from two randomized clinical trials in patients with ARDS, namely: the ALVEOLI and the ART trial. Patients in the ALVEOLI trial were eligible if they met the American-European Consensus Criteria for ARDS, including patients with a PaO₂ / FiO₂ ratio < 300 up to 48 hours before enrollment, and assessed a strategy using the high vs. low PEEP table. The ART trial enrolled patients with moderate to severe ARDS according to Berlin criteria (PaO₂ / FiO₂ ratio < 200) for less than 72 hours’ duration, and assessed two different ventilatory strategies, titrated PEEP with recruitment maneuvers vs. low PEEP according to ARDSNet PEEP FiO₂ table. Although the datasets come from rigorous well controlled trials, the pooled dataset was assessed for completeness and consistency.

Subphenotypes were determined by clusters derived from clinical characteristics of patients with ARDS. Briefly, a K-means clustering algorithm was used to develop a model including only variables that are routinely collected and inputted in electronic health records during the care of ARDS patients and were highly available closest to time of randomization. Data used to develop the model were acquired from the clinical trials ARMA, ALVEOLI, EDEN, FACTT, SAILS and ART. EDEN and FACCT were used for the training set. The trials ARMA, ALVEOLI, SAILS and ART were used for validation. The final model segregated patients into two subphenotypes (A and B) using nine of their clinical characteristics: pH, PaO2, mean arterial pressure, bicarbonate, bilirubin, creatinine, FiO₂, heart rate, and respiratory rate. Subphenotype B exhibits clinical and laboratory signals compatible with higher inflammation while subphenotype A shows the opposite. Lastly, subphenotype B has higher mortality than subphenotype A.

Heterogeneity of treatment effect of different levels of PEEP was assessed following a Bayesian hierarchical logistic model for the primary outcome. All hierarchical models were modelled as a simple regression and shrinkage model. The hierarchical models partially pool the data and shrink the estimates in each subphenotype towards the overall estimate, with shrinkage proportional to the size of the subphenotype. While traditional subgroup analyses are at higher risk of increased type 1 error due to exaggeration of the subgroup effects, the proposed hierarchical model limits this risk through shrinkage. For all analyses, weakly informative priors will be used, aiming to encompass all plausible effect sizes. Since the sample size of the pooled dataset is expected to be large, probably the likelihood will dominate the posteriors.

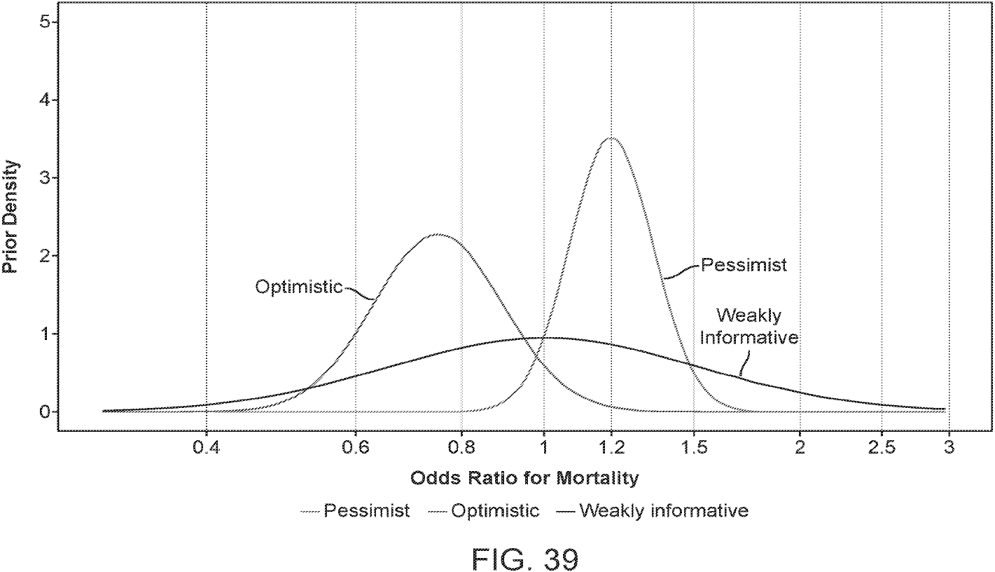

The priors were used to reflect varying degrees of beliefs for benefit or harm of higher levels of PEEP. The treatment prior’s distributions are shown in FIG. 39.

The prior was a normally distributed prior with mean 0 and variance 2.25 (prior risk with a 95% probability between 5% and 95%). This prior was used for all analysis including the sensitivity analysis with optimistic and pessimistic priors. For a shrinking parameter, the prior was a normally distributed prior with mean of 0 and variance of Ω, where Ω is the shrinkage factor having a half-normally distributed prior with variance of 1. This prior was used for all analysis including the sensitivity analysis with optimistic and pessimistic priors.

For treatment effect, a weakly informative prior was used to produce results essentially dependent on data from the analysis. This was a normally distributed prior with mean of 0 and standard deviation of 0.421 (variance of 0.177). In this prior, there is 90% probability of an 0.50 < OR < 2.00. Additionally, an optimistic prior was defined to represent archetypes of prior belief that higher PEEP effectively lowers mortality. This was a normally distributed prior with mean of -0.287 and standard deviation of 0.174 (variance of 0.030). This prior distribution was centered at an OR of 0.75 based on the assumed relative risk of death used to power the ART trial (OR ≤ 0.75) with a probability of an OR > 1.00 of 5%. Furthermore, a pessimistic prior was defined to represent archetypes of prior belief that higher PEEP increases mortality. This was a normally distributed prior with mean of 0.183 and standard deviation of 0.113 (variance of 0.012). This prior distribution was centered at a OR of 1.20 based on the relative risk of death found in the ART trial with a probability of OR < 1.00 of 5%.

For the interaction term between treatment group and PaO₂ / FiO₂ (sub-analysis 1), the prior was a normally distributed prior with mean 0 and standard deviation of 0.100 (variance of 0.010) for both terms. This prior distribution corresponds to an OR with mean of 1.00 with 95% prior probability of an OR among 0.82 to 1.22 for a 1-point increase in PaO₂ / FiO₂. For subphenotype and PaO₂ / FiO₂ (sub-analysis 2), the prior was a normally distributed prior with mean 0 and standard deviation of 0.100 (variance of 0.010) for both terms. This prior distribution corresponds to an OR with mean of 1.00 with 95% prior probability of an OR among 0.82 to 1.22.

All described Bayesian models were done using a Markov Chain Monte Carlo simulation with four chains. All models will consider a burn-in of 1,000 iterations, with sampling from a further 10,000 iterations for each chain. All chains were required to be free of divergent transitions and additional sampler settings (adapt_delta) were tuned accordingly until this is achieved. To monitor convergence, trace plots, and the Gelman-Rubin convergence diagnostic (Rhat < 1.01) were used for all parameters.

Subphenotype A is characterized by less inflammation, lower severity of illness, improved ventilator-free days and mortality compared with subphenotype B. The subphenotypes were validated as described in Example 5. All analyses are presented in the pooled population combining the ALVEOLI and ART populations and stratified by the study. The primary outcome was 28-day mortality. No secondary outcome was assessed. Continuous data were presented as median (interquartile range) and compared with the Wilcoxon rank-sum test, and categorical data were presented as number and percentage and compared with Fisher exact tests.

For the primary outcome, in addition to the odds ratio (OR) with 95% credible interval (CrI), the probability of the following OR was considered as possible thresholds for the minimum clinically important treatment effect: 1) OR < 1.00; 2) OR < 0.97; and 3) OR < 0.90. To assess the possibility of harm, the probability of harm, defined as a OR > 1.00 (null), is also reported.

To further understand the interaction according to subphenotypes and baseline hypoxemia on HTE for PEEP strategy, the within-phenotype association between higher levels of PEEP and mortality in a mixed-effect Bayesian logistic regression model according to PaO₂:FiO₂ was used. In this model, interactions between PaO₂:FiO₂ groups (stratified into six groups) and allocation groups, subphenotypes and allocation groups, and subphenotypes and PaO₂:FiO₂ groups were included. Also, to assess the interaction according to subphenotypes and baseline driving pressure on HTE for PEEP strategy, the within-phenotype association between higher levels of PEEP and mortality in a mixed-effect Bayesian logistic regression model according to baseline driving pressure was used. In this model, interactions between baseline driving pressure groups (stratified into six groups) and allocation groups, subphenotypes and allocation groups, and subphenotypes and baseline driving pressure groups were included. The model considered a Bernoulli distribution, with studies as random effect and with starting values randomly generated. All priors will be drawn from normal distributions and were weakly informative.

All effect estimates were drawn from the median of the posterior distribution and the 95% CrI from the 95th percentile of the distribution. Additional analyses considering pessimistic and optimistic priors were conducted as sensitivity analyses for the primary HTE analysis. All analyses were performed using the R software (R, version 4.0.2, Core Team, Vienna, Austria, 2016) with the beanz package and Stan through brms.

A total of 1559 ARDS patients from both ALVEOLI and ART trials were considered for this analysis. The majority of the patients were male, and pneumonia was the prevailing etiology followed by sepsis and aspiration in all trials (Table 68). There was no difference in any outcome according to randomization group in the ALVEOLI trial, and in the ART trial ventilator-free days at day 28 were lower in the ART group.

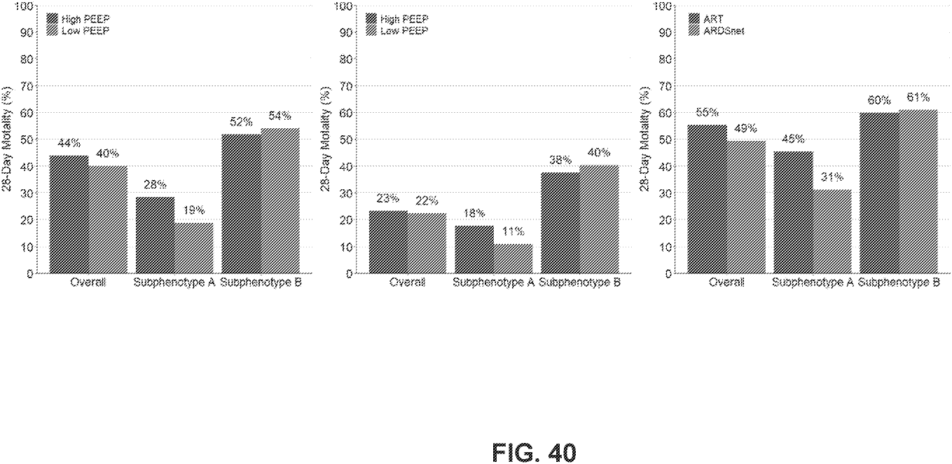

Baseline characteristics of the patients according to the subphenotype in the pooled cohort are described in Table 68. Overall, patients in subphenotype B had statistically detectably higher severity of illness, rate of vasopressor use, heart rate, creatinine, and bilirubin, as well as lower platelets, pH, BUN and bicarbonate compared to patients in subphenotype A (Table 68). 28-day mortality was higher and ventilator-free days at day 28 was lower in patients in subphenotype B. 28-day mortality was lower in patients in the low PEEP group in subphenotype A, and it was higher in the high PEEP group in subphenotype B. This can be seen in Table 68 as well as FIG. 40, which depicts 28-Day Mortality according to groups and subphenotypes.

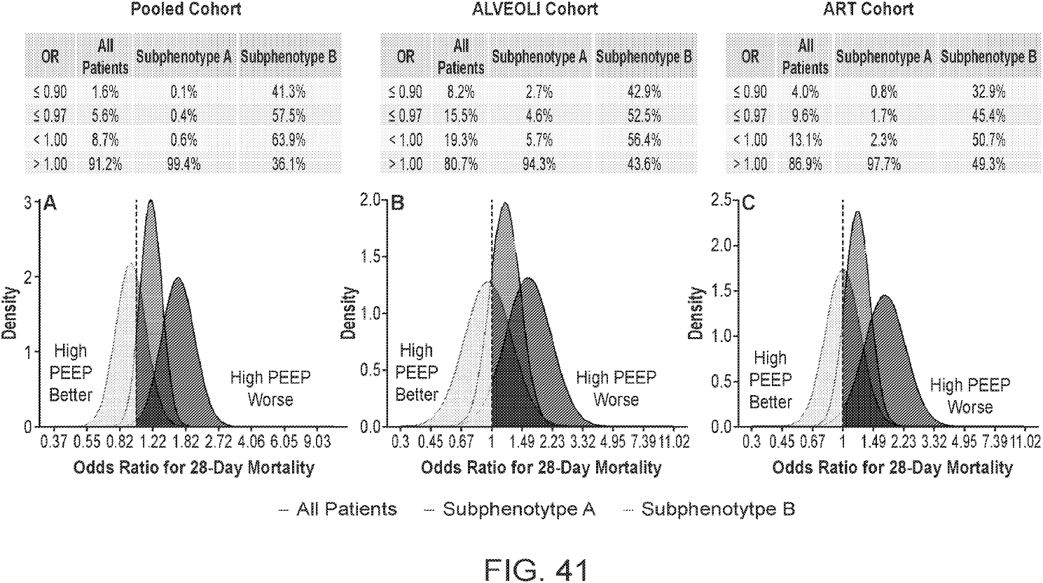

High PEEP resulted in higher risk for 28-day mortality compared to low PEEP in patients in subphenotype A (OR, 1.66 [95% CrI, 1.13 to 2.47]), with a probability of benefit in this subphenotype of only 0.6% (Table 70 and FIG. 41). Specifically, FIG. 41 shows heterogeneity of Treatment Effect of High PEEP in 28-Day mortality according to the subphenotypes. FIG. 41 Left panel: Pooled cohort; FIG. 41 Middle Panel: ALVEOLI cohort; FIG. 41 Right Panel: ART cohort. Weakly informative priors considered. Values less than 1 indicate lower mortality. Abbreviations: OR is odds ratio, and PEEP is positive end-expiratory pressure.

On the other hand, high PEEP did not affect the mortality of patients in subphenotype B (OR, 0.94 [95% CrI, 0.65 to 1.34]; probability of benefit of 63.9%). The probability that assignment to the high PEEP group results in lower OR for 28-day mortality in patients in subphenotype B (more beneficial), compared to subphenotype A, was 98.3%. The signal of the findings was similar in the individual cohorts and the use of different priors did not materially change these findings (Table 69).

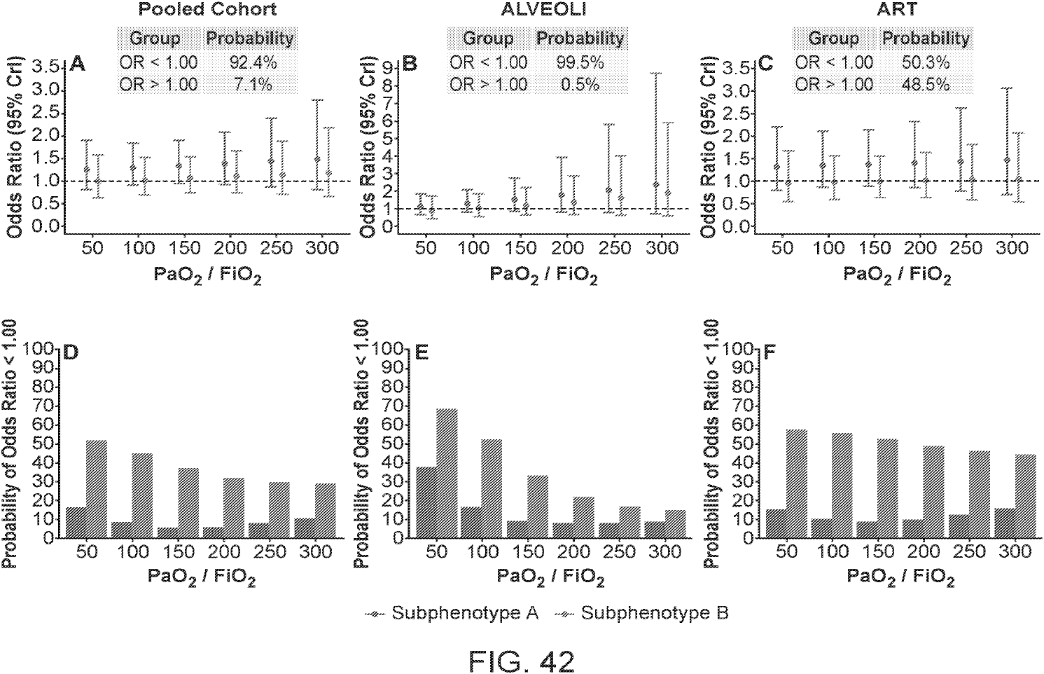

The results of the model assessing interactions between subphenotypes, PaO₂ / FiO₂ and use of high PEEP is shown in FIG. 42. Specifically, FIG. 42 shows risk of 28-Day mortality and interaction between subphenotypes, PaO₂ / FiO₂ and High PEEP. Upper panels, OR for the interaction between high PEEP, subphenotype and six different cut-offs of PaO₂ / FiO₂ categories are presented. OR < 1.0 represent a favorable outcome and > 1.0 represent unfavorable outcome with the use of high PEEP. Lower panels, probability of benefit (OR < 1.00) with high PEEP according to different thresholds of PaO₂ / FiO₂ ratios. In both upper and lower panels, the left group in each comparison is subphenotype A and the right group in each comparison is subphenotype B. Abbreviations: OR is odds ratio, and CrI is credible interval.

The probability of benefit of high PEEP was always higher in patients in subphenotype B compared to subphenotype A, especially with more severe hypoxemia. The probability of benefit of high PEEP was always higher in patients in subphenotype B compared to subphenotype A, but this probability decreased with increase in baseline driving pressure.

Using subphenotypes previously derived from routine clinical variables, this study demonstrates heterogeneity of treatment effect with regards to PEEP strategies. Subphenotype A, characterized by lower severity of illness and inflammation, had a 99.4% probability of harm when assigned to a high PEEP strategy. The overall sicker subphenotype B was more likely to benefit from a high PEEP strategy compared to A, but overall the mortality in subphenotype B between strategies did not meaningfully differ. These mortality differences between subphenotypes were maintained even when stratified by PaO2:FiO2 ratio or driving pressure. They were also stable across all priors in the Bayesian analyses.

TABLE 68

Baseline Characteristics and Clinical Outcomes According to Allocation Group and Subphenotypes for Pooled data

| | Subphenotype A | | Subphenotype B | | |
|---|---|---|---|---|---|
| | High PEEP (n = 279) | Low PEEP (n = 268) | High PEEP (n = 222) | Low PEEP (n = 233) | p value |
| Age, year | 55.0 (40.0 - 67.0) | 50.0 (36.0 - 65.0) | 49.0 (37.2 - 61.0) | 48.0 (35.0 - 59.0) | < 0.001 |
| Male gender - no. (%) | 163 (58.4) | 161 (60.1) | 131 (59.0) | 136 (58.4) | 0.977 |
| Body mass index, kg/m² | 27.7 (23.7 - 31.8) | 26.9 (22.8 - 31.3) | 26.9 (22.8 - 29.8) | 26.4 (22.1 - 31.5) | 0.233 |
| Caucasian - no. (%) | 140 (81.9) | 123 (74.5) | 51 (63.7) | 51 (66.2) | 0.006 |
| Etiology - no. (%) | | | | | < 0.001 |
| Pneumonia | 39 (14.0) | 29 (10.8) | 16 (7.2) | 19 (8.2) | |
| Sepsis | 54 (19.4) | 38 (14.2) | 28 (12.6) | 39 (16.7) | |
| Aspiration | 124 (44.4) | 119 (44.4) | 118 (53.2) | 119(51.1) | |
| Trauma | 44 (15.8) | 57 (21.3) | 59 (26.6) | 50 (21.5) | |
| Other | 18 (6.5) | 25 (9.3) | 1 (0.5) | 6 (2.6) | |
| Prognostic Scores | | | | | |
| APACHE III | 73.0 (58.5 - 85.0) | 70.0 (59.0 - 82.0) | 97.0 (79.5 - 111.0) | 92.0(80.0 - 105.0) | < 0.001 |
| SAPS III | 62.0 (53.0 - 71.0) | 61.0 (48.0 - 71.0) | 69.0 (57.5 - 77.0) | 64.0 (50.0 - 73.0) | 0.008 |
| Use of vasopressor - no. (%) | 104 (38.2) | 91 (34.7) | 156 (70.3) | 166 (71.6) | < 0.001 |
| Vital signs | | | | | |
| Temperature, °C | 37.6 (37.1 - 38.2) | 37.6 (37.0 - 38.1) | 37.5 (36.8 - 38.2) | 37.8 (37.0 - 38.3) | 0.639 |
| Heart rate, bpm | 94.0 (78.0 - 108.0) | 94.0(82.8 - 107.0) | 113.0 (99.0 - 127.0) | 110.0 (95.0 - 125.0) | < 0.001 |
| Mean arterial Pressure, mmHg | 78.0 (71.3 - 86.5) | 78.0(71.3 - 88.4) | 75.0 (68.0 - 82.3) | 73.0 (68.0 - 82.0) | < 0.001 |
| SpO₂, % | 96.0 (93.0 - 97.0) | 96.0 (94.0 - 97.0) | 94.0 (91.8 - 96.2) | 96.0 (92.0 - 98.0) | 0.006 |
| Urine output in 24 hours, mL | 1840 (1100 -2) | 1978 (1348 -2) | 1170 (500 - 1) | 1100 (414 - 2) | < 0.001 |

Laboratory tests

| Hematocrit, % | 31.0 (28.5 - 34.0) | 30.0 (27.0 - 34.0) | 31.5 (27.8 - 35.0) | 30.0 (26.0 - 34.0) | 0.273 |
|---|---|---|---|---|---|
| White blood cell count, 10⁹/L | 12.4 (7.9 - 16.6) | 11.1 (8.3 - 14.3) | 9.1 (6.0 - 14.4) | 12.6 (7.0 - 16.1) | 0.072 |
| Platelets, 10⁹/L | 171.0 (95.5 - 262.0) | 180.0 (111.2 - 285.5) | 167.0 (77.0 - 255.5) | 155.0 (81.0 - 243.0) | 0.014 |
| Creatinine, mg/dL | 1.0 (0.7 - 1.4) | 0.9 (0.7 - 1.4) | 1.8 (1.0 - 2.7) | 1.5 (0.9 - 3.0) | < 0.001 |
| Bilirubin, mg/dL | 0.7 (0.4 - 1.2) | 0.8 (0.5 - 1.4) | 1.0 (0.5 - 2.0) | 0.8 (0.4 - 1.6) | 0.002 |
| Arterial blood gas | | | | | |
| pH* | 7.39 (7.34 - 7.44) | 7.41 (7.36 - 7.45) | 7.25 (7.20 - 7.32) | 7.23 (7.17-7.31) | < 0.001 |
| PaO₂, mmHg | 84.0 (69.0 - 115.5) | 87.5 (72.0 - 121.0) | 86.5 (69.2 - 135.5) | 93.0 (71.0 - 132.0) | 0.200 |
| PaO₂ / FiO₂ | 140.0 (100.0 - 178.0) | 136.0 (98.0 - 173.0) | 107.0 (77.0 - 154.5) | 103.0 (78.0 - 143.0) | < 0.001 |
| PaCO₂, mmHg | 42.0 (36.0 - 47.0) | 40.0(35.8 - 46.0) | 45.0 (37.0 - 57.8) | 46.0 (36.0 - 62.0) | < 0.001 |
| Bicarbonate, mmol/L | 24.0 (21.0 - 27.1) | 24.0(21.3 - 27.8) | 19.9 (16.6 - 22.3) | 19.7 (16.0 - 22.7) | < 0.001 |
| Ventilatory variables | | | | | |
| Tidal volume, mL | 420.0 (350.0 - 535.0) | 450.0 (370.0 - 550.0) | 380.0 (320.0 - 450.0) | 377.5 (310.0 - 440.0) | < 0.001 |
| Per PBW, mL/kg PBW | 6.7 (6.0 - 8.2) | 6.9 (6.0 - 8.6) | 6.0 (5.4 - 6.8) | 6.0 (5.3 - 6.9) | < 0.001 |
| Plateau pressure, cmH₂O | 24.0 (21.0 -29.0) | 25.0 (22.0 - 30.0) | 27.0 (23.0 - 30.0) | 28.0 (24.0 - 31.0) | • < 0.001 |
| PEEP, cmH₂O | 10.0 (8.0 - 12.8) | 10.0 (8.0 - 12.0) | 12.0 (10.0 - 14.0) | 12.0 (10.0 - 15.0) | < 0.001 |
| Driving Pressure, cmH₂O | 14.0 (11.0 - 18.0) | 15.0 (12.0 - 19.0) | 14.0 (11.0 - 18.0) | 15.0 (12.0 - 18.0) | 0.065 |
| Respiratory rate, breaths/min | 21.0 (17.0 - 26.0) | 20.0 (16.0 - 26.0) | 30.0 (24.0 - 35.0) | 29.0 (24.0 - 34.0) | < 0.001 |
| FiO₂ | 0.60 (0.50 - 0.78) | 0.60 (0.50 - 0.70) | 0.70 (0.60 - 1.00) | 0.80 (0.60 - 1.00) | < 0.001 |
| Clinical outcomes | | | | | |
| 28-day mortality - no. (%) | 79 (28.3) | 50 (18.7) | 115 (51.8) | 126 (54.1) | < 0.001 |
| Ventilator-free days at day 28 | 15.0 (0.0 - 22.0) | 16.0 (0.0 - 23.0) | 0.0 (0.0 - 13.0) | 0.0 (0.0 - 14.0) | < 0.001 |
| Duration of ventilation, days | 8.0 (5.0 - 16.0) | 9.0 (5.0 - 16.0) | 12.0 (8.0 - 21.0) | 12.0 (8.0 - 20.0) | < 0.001 |
| Among survivors | 8.0 (5.0 - 15.2) | 9.0 (5.0 - 16.8) | 15.0 (8.0 - 28.0) | 12.0 (8.0 - 21.5) | < 0.001 |

Data are median (quartile 25^{th} - quartile 75^{th}) or N (%) Abbreviations: APACHE denotes Acute Physiology and Chronic Health Evaluation, and SAPS denotes Simplified Acute Physiology Score.

TABLE 69

Heterogeneity of Treatment Effect With 28-Day Mortality as Outcome

| | Pooled Cohort (n = 1002) | | ALVEOLI (n = 493) | | ART Study (n = 509) | |
|---|---|---|---|---|---|---|
| | Odds Ratio (95%CrI) | Probability of OR < 1.00 | Odds Ratio (95%CrI) | Probability of OR < 1.00 | Odds Ratio (95%CrI) | Probability of OR < 1.00 |
| Weakly informative prior* | | | | | | |
| All patients | 1.20 (0.93 to 1.55) | 8.7% | 1.19 (0.80 to 1.76) | 19.3% | 1.21 (0.87 to 1.68) | 13.1% |
| Subphenotype A | 1.66 (1.13 to 2.47) | 0.6% | 1.61 (0.90 to 2.94) | 5.7% | 1.73 (1.01 to 2.98) | 2.3% |
| Subphenotype B | 0.94 (0.65 to 1.34) | 63.9% | 0.95 (0.51 to 1.73) | 56.4% | 1.00 (0.63 to 1.55) | 50.7% |
| Probability of lower OR in Subphenotype B | 98.3% | | 89.0% | | 94.0% | |

Optimistic prior*

| All patients | 1.01 (0.82 to 1.24) | 47.0% | 0.90 (0.69 to 1.19) | 76.5% | 0.96 (0.75 to 1.22) | 64.2% |
|---|---|---|---|---|---|---|
| Subphenotype A | 1.61 (1.09 to 2.42) | 0.9% | 1.54 (0.87 to 2.82) | 7.5% | 1.65 (0.98 to 2.85) | 3.3% |
| Subphenotype B | 0.96 (0.66 to 1.38) | 59.2% | 0.99 (0.53 to 1.77) | 51.5% | 1.02 (0.65 to 1.58) | 46.7% |
| Probability of lower OR in Subphenotype B | 97.1% | | 85.1% | | 91.2% | |
| Pessimistic prior* | | | | | | |
| All patients | 1.21 (1.02 to 1.43) | 1.4% | 1.21 (1.00 to 1.47) | 2.7% | 1.21 (1.01 to 1.46) | 2.0% |
| Subphenotype A | 1.61 (1.09 to 2.43) | 1.1% | 1.54 (0.87 to 2.83) | 7.7% | 1.64 (0.97 to 2.88) | 3.8% |
| Subphenotype B | 0.96 (0.66 to 1.39) | 57.6% | 1.01 (0.54 to 1.81) | 48.8% | 1.03 (0.65 to 1.61) | 44.7% |
| Probability of lower OR in Subphenotype B | 96.8% | | 83.7% | | 90.3% | |

CrI: credible interval; OR: odds ratio

### Example 7: EHR-Based ARDS Subphenotyper for Guidance of Differential Treatments

Different training data sets than those used in Examples 1-4 are described here for generating additional models. For example, models were trained on the ARDSnet EDEN and FACTT datasets, and then the results were assessed for differential treatment response. In another alternate training, a specific subset of patients were selected for training from a greater patient population. For example, among the FACTT and EDEN datasets, a population of only patients with moderate to severe ARDS (as characterized by a P/F ratio <= 200 or as characterized by a P/F ratio <= 300) were selected from the entire dataset.

A number of potential features sets were originally examined for their use in the ARDS subphenotyper and mortality predictor. After detailed data audit, a number of additional potential models were examined as shown below (Table 70). The goal of examining the alternate feature sets was to identify the combination of features which provided the maximum biologic meaningfulness (by mortality, biomarker levels, and clinical values) with the smallest possible combination of variables, while covering at least 75% patients in the training data.

After a candidate feature set was identified, the optimal number of K-means clusters was determined by comparing a number of factors, including the elbow criterion method, the Calinski-Harabasz method, and the Silhouette score(“2.3. Clustering — Scikit-Learn 0.23.2 Documentation″ n.d.)(2.3. Clustering — scikit-learn 0.23.2...), across K-means models of 2, 3, 4, and 5 clusters. Feature selection and the number of clusters were selected based on the evaluation on the test set. The validation set was then used to assess the generalizability of the model.

TABLE 70

Models and respective input features

| | | Vitals | | | Arterial Blood Gas | | |
|---|---|---|---|---|---|---|---|
| Model | Name | HRATER | MEANAPR | RESPR | ARTPHR | PAO2R | FIO2R |
| C.1 | Sub8 | X | X | X | X | X | X |
| C.2 | Sub8 + VASOL24 | X | X | X | X | X | X |
| C.3 | SUB8 + age, gender, VASOL24 | X | X | X | X | X | X |
| C.4 | Sub9 | X | X | X | X | X | X |
| C.5 | Sub9 + age, gender | X | X | X | X | X | X |
| C.6 | Sub9 + ventInfo | X | X | X | X | X | X |
| C.7 | Sub9 + ventInfo -BILIH | X | X | X | X | X | X |
| C.8 | Sub9 + everything - BILIH | X | X | X | X | X | X |
| C.9 | Sub 9 + everything | X | X | X | X | X | X |
| C.10 | Sub9 + Everything Except PEEP | X | X | X | X | X | X |
| C.11 | Sub9 + Everything Except PEEP,Gender | X | X | X | X | X | X |
| C.12 | Sub9 + Everything Except PEEP, Gender, TIDAL | X | X | X | X | X | X |
| C.13 | Sub9 + Everything Except PEEP, Gender, TIDAL, ARTPHR | X | X | X | | X | X |
| C.14 | Sub9 + Everything Except PEEP, Gender, TIDAL, ARTPHR, BICARL | X | X | X | | X | X |
| C.15 | Sub9 + Everything Except PEEP, Gender, TIDAL, ARTPHR, VASOL24 | X | X | X | | X | X |
| C.16 | Sub9 + Everything Except PEEP, Gender, TIDAL, ARTPHR, BICARL, VASOL24 | X | X | X | | X | X |

TABLE 70 continued

Models and respective input features

| | Labs | | | Demographics | | | Mechanical Ventilation Parameters | | | Organ Support |
|---|---|---|---|---|---|---|---|---|---|---|
| Model | BICARL | CREATR | BILIH | PLATEL | AGE | GENDER | PEEPR | TIDALR | PPLATR | VASOL24 |
| C.1 | X | X | | | | | | | | |
| C.2 | X | X | | | | | | | | X |
| C.3 | X | X | | | X | X | | | | X |
| C.4 | X | X | X | | | | | | | |
| C.5 | X | X | X | | X | X | | | | |
| C.6 | X | X | X | X | | | X | X | X | X |
| C.7 | X | X | | X | | | X | X | X | X |
| C.8 | X | X | | X | X | X | X | X | X | X |
| C.9 | X | X | X | X | X | X | X | X | X | X |
| C.10 | X | X | X | X | X | X | | X | X | X |
| C.11 | X | X | X | X | X | | | X | X | X |
| C.12 | X | X | X | X | X | | | | X | X |
| C.13 | X | X | X | X | X | | | | X | X |
| C.14 | | X | X | X | X | | | | X | X |
| C.15 | X | X | X | X | X | | | | X | |
| C.16 | | X | X | X | X | | | | X | |

### Guiding Differential Treatment Response

A combination of data sources or subsets of data sources were combined as training data to create an ARDS subphenotyper or mortality predictor using a machine learning algorithm (such as K-means, logistic regression, XG boost, Neural networks, or another machine learning algorithm). The algorithm was applied to another retrospective or prospective data set of ARDS patients. Below, embodiments of differential treatment analysis are described with respect to various clinical interventions based on group assignment made by any machine learning algorithm. Example clinical interventions include NMB Therapy (as described above in Example 1), low or high positive end expiratory pressure (PEEP) which represents a ventilator setting, corticosteroids (e.g., methylprednisolone or dexamethasone, lisofylline (anti-inflammatory), ketoconazole (anti-fungal), catheter and fluid management, recruitment maneuver (ventilator strategy), and statins.

The different clinical interventions were considered for differential treatment response using various combinations of training data, model feature sets, validation data, and recorded interventions. Differential response was examined using numerous outcomes, including mortality, ventilator free days, or ventilator days.

### PEEP and Recruitment Maneuver

Positive End-Expiratory Pressure (PEEP) is the amount of pressure above atmospheric pressure remaining in the airway at the end of the respiratory cycle (exhalation) in mechanically ventilated patients. Current guidelines recommend high PEEP in patients with moderate or severe ARDS (Papazian et al. 2019; Fan et al. 2017). However, the ideal level of PEEP may also be correlated with a patient’s phenotype.

High PEEP and low PEEP treatments are provided to patients based on the patient’s fraction of inspired oxygen (FiO₂) level. Further details of high and low PEEP in relation to patient FiO₂ levels are described in Brower RG et al. “Higher versus lower positive end-expiratory pressures in patients with the acute respiratory distress syndrome.” N Engl J Med. 2004 Jul 22;351(4):327-36, which is incorporated by reference in its entirety. In particular, the allowable combinations of PEEP and FiO₂ are shown below in Tables 71A-71C. Therefore, a low PEEP treatment for a patient would refer to a particular PEEP (cm H₂O) based on the corresponding FiO₂ level of the patient shown in Table 71A. Similarly, a high PEEP treatment for a patient would refer to a particular PEEP (cm H₂O) based on the corresponding FiO₂ level of the patient shown in Table 71B or 71C.

TABLE 71A

Allowable combination of PEEP and FiO₂ in lower-PEEP group

| FiO₂ | PEEP (cm H₂O) |
|---|---|
| 0.3 | 5 |
| 0.4 | 5 or 8 |
| 0.5 | 8 or 10 |
| 0.6 | 10 |
| 0.7 | 10, 12, or 14 |
| 0.8 | 14 |
| 0.9 | 14, 16, or 18 |
| 1.0 | 18-24 |

TABLE 71B

Allowable combination of PEEP and FiO₂ in Higher-PEEP group (before protocol changed to use higher levels of PEEP)

| FiO₂ | PEEP (cm H₂O) |
|---|---|
| 0.3 | 5. 8, 10, 12, or 14 |
| 0.4 | 14 or 16 |
| 0.5 | 16 or 18 |
| 0.5-0.8 | 20 |
| 0.8 | 22 |
| 0.9 | 22 |
| 1.0 | 22-24 |

TABLE 71C

Allowable combination of PEEP and FiO₂ in Higher-PEEP group (after protocol changed to use higher levels of PEEP)

| FiO₂ | PEEP (cm H₂O) |
|---|---|
| 0.3 | 12 or 14 |
| 0.4 | 14 or 16 |
| 0.5 | 16 or 18 |
| 0.5-0.8 | 20 |
| 0.8 | 22 |
| 0.9 | 22 |
| 1.0 | 22-24 |

Recruitment maneuvers in ARDS are periods of sustained increased transpulmonary pressure (through increased PEEP) designed to help re-open (recruit) collapsed alveoli. Recommendations about recruitment maneuvers in ARDS are mixed, with some saying “recruitment maneuvers should probably not be used routinely in ARDS patients” (Papazian et al. 2019) and others recommending for recruitment maneuvers with moderate or severe ARDS (Fan et al. 2017). Again, some patients may benefit from increased PEEP via recruitment maneuvers whereas others may benefit from lower levels of PEEP.

To evaluate these hypotheses, K-means clustering was applied using Model C.4 described above in Table 70. In particular, Model C.4 includes the following features: recent arterial pH (Arterial pH-R), lowest bicarbonate (bicarbonate-L), recent creatinine (creatinine-R), recent FiO₂ (FiO₂-R), recent heart rate (heart rate-R), recent PaO₂ (PaO₂—R), recent mean arterial pressure (mean arterial pressure-R), recent respiratory rate (respiratory rate-R), and highest bilirubin (bilirubin-H).

In the first iteration, the training data consisted of all patients enrolled in the FACTT and EDEN ARDSnet studies. Patients who did not have measurements for each of the 9 data elements used were excluded from the training dataset. The resulting K-means algorithm was then applied to the ALVEOLI and ART studies (described previously). Key outcomes, including 60 and 90-day mortality (ALVEOLI), 28 and 180-day mortality (ART), ventilator free days, and number of days on ventilator were calculated for each treatment arm of each phenotype, as shown in Tables 72A and 72B below. Mortality was assessed by a logistic regression model incorporating the subphenotype (based on K-means cluster assignment) and an interaction term. Due to overdispersion and excessive zeros, the ventilator and ventilator-free days were compared among the subphenotypes considering a mixed-effect generalized linear model with zero-inflated negative binomial distribution. Models were unadjusted and included the hospital of inclusion as a random effect if hospital information was available. A two-sided p-value < 0.05 was considered evidence of statistical significance. Statistical analysis was performed in R, version 4.0.2.

TABLE 72A

Key clinical endpoints for each subphenotype and study arm for ALVEOLI study

| ALVEOLI Model C.4 | Subphenotype B | | Subphenotype A | | p-value |
|---|---|---|---|---|---|
| | High PEEP N=81 | Low PEEP N=75 | High PEEP N=170 | Low PEEP N=167 | m/n |
| Dead60, (%) | 42 | 45.3 | 21.8 | 13.2 | 0.09 |
| Dead90,(%) | 45 | 46.6 | 22.2 | 14 | 0.15 |
| VFD, mean (SD) | 8.4 (9.9) | 9.6 (10.6) | 15.7 (10.6) | 16.2 (9.9) | 0.019/0.29 |
| VM days, mean (SD) | 15.2 (9.2) | 12.2 (8.6) | 9.4 (8) | 10.1 (7.9) | |

TABLE 72B

Key clinical endpoints for each subphenotype and study arm for ART study

| ART Model C.4 | Subphenotype B | | Subphenotype A | | p-value |
|---|---|---|---|---|---|
| | High PEEP N=142 | Low PEEP N=154 | High PEEP N=108 | Low PEEP N=105 | m/n |
| Dead60, (%) | 59.1 | 61 | 46.3 | 31.4 | 0.09 |
| Dead90, (%) | 70.4 | 66.2 | 55.6 | 43.8 | 0.55 |
| VFD, mean (SD) | 4.5 (7.9) | 4.5 (7.4) | 6.7 (8.7) | 10 (9.4) | 0.019/0.015 |
| VM days, mean (SD) | 14.4 (8.2) | 14.7 (7.4) | 14.9 (8.4) | 13.2 (7.7) | |

In both ALVEOLI and ART there was a trend toward significance in mortality, and a significant difference in ventilator free days between subphenotype and study arms. Within subphenotype B (the high mortality subphenotype), patients receiving high PEEP had slightly lower mortality in both studies; however, within subphenotype A, the group receiving low PEEP had lower mortality with more ventilator free days. This suggests that contrary to current treatment guidelines for ARDS, patients within subphenotype A may benefit from lower PEEP.

Findings for the ALVEOLI study aligned with the findings of Calfee et al (Calfee et al. 2014). Within Calfee’s Phenotype 2 (similar to Endpoint Health subphenotype B), mortality was reduced and ventilator-free and organ failure-free days were increased among patients receiving high PEEP. Conversely, Phenotype 1 patients (similar to Endpoint Health subphenotype A) experienced lower mortality when they received low PEEP, though there was little change in ventilator-free and organ failure-free days.

While the findings here show similar results to Calfee et al, they are distinguishable because they are based on a generalizable K-means clustering model which can be applied across numerous data sets, whereas Calfee’s work was trained and evaluated on the same data set. This suggests that the results here could be applied prospectively to data outside of the ALVEOLI data set. The similar findings in ART support this claim.

Characteristics of Subphenotype A show that these patients tend to not be as sick as Subphenotype B patients. They have lower mortality and more ventilator free days. At the time of enrollment, the mean PaO₂/FiO₂ (P/F ratio) for ALVEOLI was 117.4 (SD = 58.2) for Subphenotype B and 156.2 (SD = 63.3) for Subphenotype A. It was hypothesized that the differential mortality seen due to high and low PEEP may have been due to the proportion of patients with moderate or severe ARDS in each subphenotype compared to patients with mild ARDS. To test this hypothesis, a secondary set of models was created which was only trained and tested on patients with moderate to severe ARDS, removing the possibility of patients with mild ARDS contributing to a false differential response.

In this iteration, the training set still consisted of patients from FACTT and EDEN, however, only patients with moderate or severe ARDS (P/F ratio <= 200) were included in the training data set. A new K-means model was created using the same readily-available data features defined previously. The model was then applied to the ALVEOLI and ART data sets, but again excluding patients with a P/F ratio > 200. Table 73 shows the results. (NOTE: the ART trial originally only excluded patients with a P/F ratio <= 200, so no additional patients were excluded from that study). The same post-hoc analysis was performed to identify statistically significant differences in outcomes.

TABLE 73

Differential treatment response for subphenotypes when only patients with moderate to severe ARDS were included in the K-means clustering training and testing data sets. Model was trained and tested on patients with P/F ratio <200

| ALVEOLI Model C.4 | Subphenotype B | | Subphenotype A | | p-value |
|---|---|---|---|---|---|
| | High PEEP N=67 | Low PEEP N=64 | High PEEP N=122 | Low PEEP N=127 | m/n |
| Dead60, n (%) | 47.7 | 45.3 | 22.1 | 14.1 | 0.35 |
| Dead90, n (%) | - | - | - | - | - |
| VFD, mean (SD) | 8.1 (9.8) | 9.3 (10.3) | 15 (10.4) | 16.1 (9.6) | 0.13/0.27 |
| VM days, mean (SD) | 14.5 (8.9) | 11.6 (7.5) | 10.4 (8.4) | 10.6 (7.4) | |
| ART Model C.4 | Subphenotype B | | Subphenotype A | | p-value |
| | High PEEP N=142 | Low PEEP N=154 | High PEEP N=108 | Low PEEP N=105 | m/n |
| Dead60, n (%) | 59.1 | 61 | 46.3 | 31.4 | 0.09 |
| Dead90, n (%) | 70.4 | 66.2 | 55.6 | 43.8 | 0.55 |
| VFD, mean (SD) | 4.5 (7.9) | 4.5 (7.4) | 6.7 (8.7) | 10 (9.4) | 0.019/0.015 |
| VM days, mean (SD) | 14.4 (8.8) | 14.7 (7.4) | 14.9 (8.4) | 13.2 (7.7) | |

While mortality was not statistically significant in the ALVEOLI data, there was a decrease in 60-day mortality among subphenotype A patients who received low PEEP therapy. In ART, the difference in mortality across all subphenotypes and treatment arms neared significance, with subphenotype A patients with low PEEP showing reduced mortality, and subphenotype B patients who received high PEEP showing reduced mortality. subphenotype A patients with low PEEP also had significantly more ventilator free days.

### Corticosteroids (LASRS Study)

The dataset from the LASRS study was used for analysis. The LASRS study involved administration of corticosteroids, specifically methylprednisolone. K-means clustering was applied using Model C.4 described above in Table 70 and patients were separated into two subphenotypes based on the K-means cluster. Tables 74A-74C show the characteristics of the different subphenotypes. Overall mortality was 40% in Subphenotype B and 28.57% in Subphenotype A (p = 0.3287). Within Subphenotype B, mortality rates were 40% regardless of whether the patient received methylprednisolone or a placebo; however, in Subphenotype A, mortality was 50% in the cohort receiving methylprednisolone, compared with 9.09% in the placebo cohort (p = 0.0382).

TABLE 74A

All patients. Chi-squared = 0.9541, df= 1, p-value = 0.3287

| | Type | Dead 90: 0 0 | Dead 90: 1 0 | Total |
|---|---|---|---|---|
| Subphenotvpe B | Frequency | 57 | 38 | 95 |
| | Percent | 60 | 40 | - |
| Subphenotype A | Frequency | 15 | 6 | 21 |
| | Percent | 71.43 | 28.57 | - |
| Total | Frequency | 72 | 44 | 118 |

TABLE 74B

Subphenotype B patients. Chi-squared = 0.0000, df= 1, p-value = 1.000

| Intervention | Type | Dead 90: 0 0 | Dead 90: 1 0 | Total |
|---|---|---|---|---|
| Methylprednisolone | Frequency | 27 | 18 | 45 |
| | Percent | 60 | 40 | - |
| Placebo | Frequency | 30 | 20 | 50 |
| | Percent | 60 | 40 | - |
| Total | Frequency | 57 | 38 | 95 |

TABLE 74C

Subphenotype A patients. Chi-squared = 4.2955, df= 1, p-value = 0.0382

| Intervention | Type | Dead 90: 0 0 | Dead 90: 1 0 | Total |
|---|---|---|---|---|
| Methylprednisolone | Frequency | 5 | 5 | 10 |
| | Row Percent | 50 | 50 | - |
| Placebo | Frequency | 10 | 1 | 11 |
| | Row Percent | 90.91 | 9.09 | - |
| Total | Frequency | 15 | 6 | 21 |

Observation: Patients that meet the LASRS inclusion criteria that are identified by the test to be in Subphenotype A exhibit higher mortality (50%) when treated with methylprednisolone vs. placebo (9.1%). Hypothesis: Hydrocortisone harms ARDS patients in Subphenotype A. Therefore, when considering methylprednisolone treatment for ARDS patients, the subphenotyping test should be run and methylprednisolone should be avoided for patients identified by the test to be in Subphenotype A.

### Corticosteroids (CoDEX Study)

The dataset from the CoDEX study was used for analysis. The CoDEX study involved treating COVID-19 patients with dexamethasone. K-means clustering was applied using Model C.4 described above in Table 70 and patients were separated into two clusters assigned to Subphenotype A and Subphenotype B. Tables 75A and 75B show the corresponding results. The number of ventilator free days increased by 101% in Subphenotype B patients who received dexamethasone versus placebo; however, the number of vent free days increased by only 45% in patients in Subphenotype A (p = 0.03309).

TABLE 75A

28-day Mortality

| Overall | Subphenotype B | | | Subphenotype A | | | p | |
|---|---|---|---|---|---|---|---|---|
| 58.86% | 68.18% | | | 55.56% | | | 0.1039 | |
| | Dexamethasone | Control | p | Dexamethasone | Control | p | | p interaction |
| | 62.50% | 73.53% | 0.3363 | 54.72% | 56.52% | 0.8570 | | 0.51364 |

TABLE 75B

Vent Free Days

| Overall | Subphenotype B | | | Subphenotype A | | | p | |
|---|---|---|---|---|---|---|---|---|
| 5.51 | 3.77 | | | 7.54 | | | 0.0070 | |
| | Dexamethasone | Control | p | Dexamethasone | Control | p | | p interaction |
| | 5.09 | 2.53 | 0.16773 | 8.81 | 6.07 | 0.17795 | | 0.03309 |

Observation: Patients that meet the CoDEX inclusion criteria and are treated with dexamethasone that are identified by the test to be in Subphenotype A do not see as strong of an improvement in ventilator free days as patients in Subphenotype B who are treated with dexamethasone.

Hypothesis: The highest improvement in outcomes from dexamethasone therapy for ARDS patients are achieved in patients identified by the test to be in Subphenotype B.

Product use, if hypothesis is confirmed: When considering dexamethasone treatment for ARDS patients, the subphenotyping test should be run and dexamethasone should be administered to patients identified by the test to be in Subphenotype B. The subphenotyping test can be used as a prognostic to better understand the expected ventilator use in individual patients or in a pandemic situation.

### Lisofvlline and Ketoconazole (ARMA-KARMA-LARMA Study)

The dataset from the ARMA-KARMA-LARMA study was used for analysis. Interventions in the study included lisofylline and ketoconazole. Subphenotype A had a strong signal to not use lisofylline. Overall mortality for ARMA study showed Subphenotype B with 34% mortality and Subphenotype A with 25.9% mortality (Table 76A).

TABLE 76A

All patients. Chi-squared = 2.9730, df= 1, p-value = 0.0847

| | Type | Dead 90: 0 0 | Dead 90: 1 0 | Total |
|---|---|---|---|---|
| Subphenotype B | Frequency | 186 | 65 | 251 |
| | Percent | 74.1 | 25.9 | - |
| Subphenotype A | Frequency | 97 | 50 | 147 |
| | Percent | 65.99 | 34.01 | |
| Total | Frequency | 283 | 115 | 398 |

Within the subset of patients identified as lisofylline: active and lisofylline: placebo, the difference in mortality between subphenotypes was negligible, with the Subphenotype A having a 27.1% mortality, and Subphenotype B having a 28% mortality (Table 76B).

TABLE 76B

Patients administered lisofylline. Chi-squared = 0.0107, df= 1, p-value = 0.9174

| | Type | Dead 90: 0 0 | Dead 90: 1 0 | Total |
|---|---|---|---|---|
| Subphenotvpe B | Frequency | 51 | 19 | 70 |
| | Percent | 72.86 | 27.14 | - |
| Subphenotype A | Frequency | 36 | 14 | 50 |
| | Percent | 72 | 28 | - |
| Total | Frequency | 87 | 33 | 120 |

When just Subphenotype B was examined, mortality was 40% for patients who got lisofylline, and 16% for patients who received placebo (p = 0.0588) (Table 76C).

TABLE 76C

Subphenotype B patients. Chi-squared = 3.5714, df= 1, p-value = 0.0588

| Intervention | Type | Dead 90: 0 0 | Dead 90: 1 0 | Total |
|---|---|---|---|---|
| Methylprednisolone | Frequency | 15 | 10 | 25 |
| | Percent | 60 | 40 | - |
| Placebo | Frequency | 21 | 4 | 25 |
| | Percent | 84 | 16 | - |
| Total | Frequency | 36 | 14 | 50 |

There was no significant difference in mortality for patients in Subphenotype A who received lisofylline versus placebo (31.4% vs 22.9%, p = 0.4201) (Table 76D).

TABLE 76D

Subphenotype A patients. Chi-squared = 0.6502, df= 1, p-value = 0.4201

| Intervention | Type | Dead 90: 0 0 | Dead 90: 1 0 | Total |
|---|---|---|---|---|
| Methylprednisolone | Frequency | 24 | 11 | 35 |
| | Percent | 68.57 | 31.43 | - |
| Placebo | Frequency | 27 | 8 | 35 |
| | Percent | 77.14 | 22.86 | - |
| Total | Frequency | 51 | 19 | 70 |

Observation: Patients that meet the ARMA-KARMA-LARMA inclusion criteria that are identified by the test to be in Subphenotype B exhibit higher mortality when treated with lisofylline vs. placebo.

Hypothesis: Lisofylline harms ARDS patients in Subphenotype B.

Product use, if hypothesis is confirmed: When considering lisofylline treatment for ARDS patients, the subphenotyping test should be run and lisofylline should be avoided for patients identified by the test to be in Subphenotype B.

### Catheter and Fluid (FACTT Study)

The dataset from the FACTT study was used for analysis. The FACTT study involved the use of a pulmonary artery catheter (PAC) in comparison to a less invasive alternative (central venous catheter (CVC). K-means clustering was applied using Model C.4 described above in Table 70 and patients were separated into two clusters, assigned to subphenotype A and subphenotype B. Findings: Preliminary logistic regression analysis showed that subphenotype, and the interaction term of subphenotype and type of line were each significant or nearing significance in predicting 90 day mortality.

Further analysis showed the overall dataset had a high mortality phenotype (Subphenotype B) (34.2%) and a low mortality phenotype (Subphenotype A) (26.0%) (Table 77A).

TABLE 77A

All patients. Chi-squared = 5.5793, df= 1, p-value = 0.0182

| | Type | Dead 90: 0 0 | Dead 90: 1 0 | Total |
|---|---|---|---|---|
| Subphenotype A | Frequency | 299 | 105 | 404 |
| | Percent | 74.01 | 25.99 | - |
| Subphenotype B | Frequency | 194 | 101 | 295 |
| | Percent | 65.76 | 34.24 | - |
| Total | Frequency | 493 | 206 | 699 |

Among patients who received the CVC line, mortality rates were similar to the overall population (38.1% and 23.7% in the Subphenotype B and Subphenotype A, respectively) (Table 77B).

TABLE 77B

Patients receiving CVC line. Chi-squared = 8.1061, df= 1, p-value = 0.0044

| | Type | Dead 90: 0 0 | Dead 90: 1 0 | Total |
|---|---|---|---|---|
| Subphenotype A | Frequency | 151 | 47 | 198 |
| | Percent | 76.26 | 23.74 | - |
| Subphenotype B | Frequency | 86 | 53 | 139 |
| | Percent | 61.87 | 38.13 | - |
| Total | Frequency | 237 | 100 | 337 |

However, there was no difference in mortality among patients who received the PAC line; mortality was slightly lower in Subphenotype B (30.8%) and slightly higher in Subphenotype A (28.2%) (Table 77C).

TABLE 77C

Patients receiving PAC line. Chi-squared = 0.2929, df= 1, p-value = 0.5884

| | Type | Dead 90: 0 0 | Dead 90: 1 0 | Total |
|---|---|---|---|---|
| Subphenotype A | Frequency | 148 | 58 | 206 |
| | Percent | 71.84 | 28.16 | - |
| Subphenotype B | Frequency | 108 | 48 | 156 |
| | Percent | 69.23 | 30.77 | - |
| Total | Frequency | 256 | 106 | 362 |

There was not a significant interaction between fluid management strategy and a patient’s subphenotype. However, based on the findings that there is a significant interaction with PAC lines and subphenotype, the fluid management strategy was combined with the PAC line to identify interactions. In the Subphenotype B, there was no significant difference (p = 0.9346) in 90-day mortality between PAC line and liberal fluid (34.6% mortality) and the other combinations of line and fluid management (34.1% mortality).

TABLE 77D

FIG. 77D: Subphenotype B patients. Chi-squared = 0.0067, df= 1, p-value = 0.9346

| Intervention | Type | Dead 90: 0 0 | Dead 90: 1 0 | Total |
|---|---|---|---|---|
| PAC line, conservative fluid or CVC line with any fluid | Frequency | 143 | 74 | 217 |
| | Row Percent | 65.9 | 34.1 | - |
| PAC line, liberal fluid | Frequency | 51 | 27 | 78 |
| | Row Percent | 65.38 | 34.62 | - |
| Total | Frequency | 194 | 101 | 295 |

However, in Subphenotype A, mortality increased to 30.3% if a patient was treated with a PAC line and liberal fluid, whereas mortality in the remaining population was 24.6% (p = 0.2601).

TABLE 77E

FIG. 77E: Subphenotype A patients. Chi-squared = 1.2681, df= 1, p-value = 0.2601

| Intervention | Type | Dead 90: 0 0 | Dead 90: 1 0 | Total |
|---|---|---|---|---|
| PAC line, conservative fluid or CVC line with any fluid | Frequency | 230 | 75 | 305 |
| | Row Percent | 75.41 | 24.59 | - |
| PAC line, liberal fluid | Frequency | 69 | 30 | 99 |
| | Row Percent | 69.7 | 30.3 | - |
| Total | Frequency | 299 | 105 | 404 |

A Welch’s two-sample t-test also showed a difference in ventilator free days which neared significance for patients in Subphenotype A who got a PAC line and liberal fluid (13.1 ventilator free days on average) vs all other patients within Subphenotype A(14.9 ventilator free days on average). Specifically, for a t-statistic of 1.62 and 168.81 degrees of freedom, the comparison yielded a p-value of 0.10716.

Observation 1: patients who get a CVC line exhibit similar behavior to subphenotypes, with a high mortality and a low mortality subphenotype; however, mortality rates are not consistent when patients receive a PAC line.

Observation 2: Patients that meet the FACTT inclusion criteria that are identified by the test to be in Subphenotype A exhibit higher mortality when treated with PAC+ liberal fluids vs. PAC + conservative fluid, CVC + conservative fluid, or CVC + liberal fluid.

Hypothesis: PAC+liberal fluids harms ARDS patients in the Subphenotype A.

Product use, if hypothesis is confirmed: When considering PAC+liberal fluids treatment for ARDS patients, the subphenotyping test should be run and PAC+liberal fluids should be avoided for patients identified by the test to be in Subphenotype A.

### Recruitment Maneuver (ART Study)

The dataset from the ART study was used for analysis. The ART study involved administering recruitment maneuvers to patients. K-means clustering was applied using Model C.4 described above in Table 70 and patients were separated into two clusters assigned to subphenotype A and subphenotype B. Logistic regression analysis showed that subphenotype, recruitment maneuver vs standard ARDSnet guidance care, and the interaction term of subphenotype and recruitment maneuver were each significant or nearing significance in predicting 90 day mortality based on Pr(>|z|) scores.

Further chi-square analysis showed the following: Similar to previous findings, a low mortality subphenotype (31.1%) - Subphenotype A, and a high mortality subphenotype (49.6%) - Subphenotype B, were identified (Table 78A).

TABLE 78A

All patients. Chi-squared = 18.0544, df= 1, p-value = 0.0000

| | Type | Dead 90: 0 0 | Dead 90: 1 0 | Total |
|---|---|---|---|---|
| Subphenotype B | Frequency | 127 | 125 | 252 |
| | Percent | 50.4 | 49.6 | - |
| Subphenotype A | Frequency | 177 | 80 | 257 |
| | Percent | 68.67 | 31.13 | - |
| Total | Frequency | 304 | 205 | 509 |

Among the Subphenotype A, there was no difference in mortality for those who received the standards ARDSnet care (30.6%) versus those who received additional recruitment maneuver via the ART protocol (31.7%, p = 0.8477) (Table 78B).

TABLE 78B

Low mortality patients. Chi-squared = 0.0369, df= 1, p-value = 0.8477

| | Type | Dead 90: 0 0 | Dead 90: 1 0 | Total |
|---|---|---|---|---|
| ARDSnet protocol | Frequency | 93 | 41 | 134 |
| | Percent | 69.4 | 30.6 | - |
| ART protocol | Frequency | 84 | 39 | 123 |
| | Percent | 68.29 | 31.71 | - |
| Total | Frequency | 177 | 80 | 257 |

Among the Subphenotype B, patients who received recruitment maneuvers according to the ART protocol had significantly lower mortality (42.5%) than those who received the standard ARDSnet care protocol (56.8%, p = 0.0234) (Table 78C).

TABLE 78C

High mortality patients. Chi-squared = 5.1390, df= 1, p-value = 0.0234

| | Type | Dead 90: 0 0 | Dead 90: 1 0 | Total |
|---|---|---|---|---|
| ARDSnet protocol | Frequency | 54 | 71 | 125 |
| | Percent | 43.2 | 56.8 | - |
| ART protocol | Frequency | 73 | 54 | 127 |
| | Percent | 57.48 | 42.52 | - |
| Total | Frequency | 127 | 125 | 252 |

Observation 2: Patients that meet the ART inclusion criteria and that are identified by the test to be in Subphenotype B exhibit lower mortality when treated with a more aggressive recruitment maneuver protocol.

Hypothesis: recruitment maneuvers support ARDS patients in Subphenotype B.

Product use, if hypothesis is confirmed: When considering recruitment maneuver treatment for ARDS patients, the subphenotyping test should be run and recruitment maneuvers should be considered as treatment for Subphenotype B.

### Statins (eICU Dataset)

The dataset from the eICU (v1) dataset was used for analysis. The intervention of interest was statins. K-means clustering was applied using Model C.4 described above in Table 70 and patients were separated into two clusters, assigned to subphenotype A and subphenotype B. Patients in the Subphenotype A who were charted as on any statin at the time of ICU admission (6.81% mortality) may have increased survival as compared with those who had no statin during their stay (13.28% mortality) (Chi-square = 6.2409, p = 0.012). Patients who initiated a statin during their ICU stay did not see the same mortality benefit as patients on a statin at admission (Chi-square = 0.0802, p = 0.777051); in fact, their mortality rate was closer to that of patients who received no statin therapy (12.56%).

Observation: ARDS patients in the eICU dataset that are identified by the test to be in Subphenotype A and who were taking statins at the time of ICU admission exhibit lower mortality vs. those who were not taking statins at the time of ICU admission.

Hypothesis: ARDS Subphenotype A patients on statins prior to ICU admission exhibit lower mortality.

Product use, if hypothesis is confirmed: ARDS Subphenotype A patients on statins prior to ICU admission exhibit better prognosis. Patients presenting to the emergency department with pneumonia, sepsis or other ARDS risk factors should be tested for their subphenotype. If found to be in Subphenotype A with no contraindications, pre-emptive statins may be considered.

Conversely, in the Subphenotype B, statin therapy seemed to benefit patient outcomes regardless of timing of therapy initiation. Patients who received a statin at any time in their stay had a mortality rate of 26.44% whereas patients who did not receive a statin had a mortality rate of 35.46% (Chi-square = 4.8126, p = 0.028253). Mortality rates were similar whether the statin was already initiated at the time of ICU admit (27%) or initiated during the ICU stay (26%); however chi square was nonsignificant compared with patients not receiving statins, due to the smaller sample size of the subgroups.

Observation: ARDS patients in the eICU dataset that are identified by the test to be in the Subphenotype B exhibit lower mortality when receiving statins during their ICU stay vs. when not receiving statins during their ICU stay. Tables 79A-79C show characteristics of patients that were administered any of simvastatin, atorvastatin, or any statin.

Hypothesis: Subphenotype B ARDS patients exhibit lower mortality when treated with statins.

Product use, if hypothesis is confirmed: ARDS patients identified to be in Subphenotype B using the sub-phenotyping test should be treated with statins

TABLE 79A

Characteristics of Patients admitted and Simvastatin Intervention

| | | Subphenotype B | Subphenotype A | All Patients |
|---|---|---|---|---|
| Simvastatin initiated during ICU stay | Alive | 26 | 63 | 89 |
| | Dead | 9 | 6 | 15 |
| | Mortality Rate | 25.71 | 8.70 | 14.42 |
| Patients on simvastatin at time of ICU admit | Alive | 24 | 63 | 87 |
| | Dead | 7 | 4 | 11 |
| | Mortality Rate | 22.58 | 5.97 | 11.22 |
| Patients admitted with simvastatin or initiated during ICU stay | Alive | 50 | 126 | 176 |
| | Dead | 16 | 10 | 26 |
| | Mortality Rate | 24.24 | 7.35 | 12.87 |
| Patients not admitted to ICU on statin and did not receive any statin during ICU stay | Alive | 131 | 346 | 477 |
| | Dead | 54 | 56 | 110 |
| | Mortality Rate | 29.19 | 13.93 | 18.74 |

TABLE 79B

Characteristics of Patients Admitted and Atorvastatin Intervention

| | | Subphenotype B | Subphenotype A | All Patients |
|---|---|---|---|---|
| Atorvastatin initiated during ICU stay | Alive | 61 | 149 | 210 |
| | Dead | 21 | 19 | 40 |
| | Mortality Rate | 25.61 | 11.31 | 16 |
| Patients on atorvastatin at time of ICU admit | Alive | 20 | 60 | 80 |
| | Dead | 7 | 7 | 14 |
| | Mortality Rate | 25.93 | 10.45 | 14.89 |
| Patients admitted with atorvastatin or initiated during ICU stay | Alive | 81 | 209 | 290 |
| | Dead | 28 | 26 | 54 |
| | Mortality Rate | 25.69 | 11.06 | 15.70 |
| Patients not admitted to ICU on statin and did not receive any statin during ICU stay | Alive | 131 | 346 | 477 |
| | Dead | 54 | 56 | 110 |
| | Mortality Rate | 29.19 | 13.93 | 18.74 |

TABLE 79C

Characteristics of Patients Admitted and any Statin Intervention

| | | Subphenotype B | Subphenotype A | All Patients |
|---|---|---|---|---|
| Any statin initiated during ICU stay | Alive | 254 | 726 | 980 |
| | Dead | 142 | 120 | 262 |
| | Mortality Rate | 35.86 | 14.18 | 21.10 |
| Patients on any statin at time of ICU admit | Alive | 61 | 171 | 232 |
| | Dead | 19 | 14 | 33 |
| | Mortality Rate | 23.75 | 7.57 | 12.45 |
| Patients admitted with any statin or initiated during ICU stay | Alive | 315 | 897 | 1212 |
| | Dead | 161 | 134 | 295 |
| | Mortality Rate | 33.82 | 13 | 19.58 |
| Patients not admitted to ICU on statin and did not receive any statin during ICU stay | Alive | 131 | 346 | 477 |
| | Dead | 54 | 56 | 110 |
| | Mortality Rate | 29.19 | 13.93 | 18.74 |

The analysis was repeated on the eICU data, removing patients who had medical history codes which would indicate a patient had an indication for statin use prior to ICU admission. This included patients with history of angina, congestive heart failure, coronary artery bypass grafting, multiple coronary artery bypass, hypertension requiring treatment, previous acute myocardial infarction, peripheral vascular disease, previous coronary intervention procedure, stroke, and/or transient ischemic attack. Tables 80A-80C summarize the results of the analysis.

TABLE 80A

Characteristics of Patients admitted and Simvastatin Intervention in filtered eICU data

| | | Subphenotype B | Subphenotype A | All Patients |
|---|---|---|---|---|
| Simvastatin initiated during ICU stay | Alive | 1 | 5 | 6 |
| | Dead | 3 | 1 | 4 |
| | Mortality Rate | 75 | 16.67 | 40 |
| Patients on simvastatin at time of ICU admit | Alive | 7 | 10 | 17 |
| | Dead | 1 | 0 | 1 |
| | Mortality Rate | 12.5 | 0 | 5.56 |
| Patients admitted with simvastatin or initiated during ICU stay | Alive | 8 | 15 | 23 |
| | Dead | 4 | 1 | 5 |
| | Mortality Rate | 33.33 | 6.25 | 17.86 |
| Patients not admitted to ICU on statin and did not receive any statin during ICU stay | Alive | 131 | 346 | 477 |
| | Dead | 54 | 56 | 110 |
| | Mortality Rate | 29.19 | 13.93 | 18.74 |

TABLE 80B

Characteristics of Patients Admitted and Atorvastatin Intervention in filtered eICU data

| | | Subphenotype B | Subphenotype A | All Patients |
|---|---|---|---|---|
| Atorvastatin initiated during ICU stay | Alive | 8 | 34 | 42 |
| | Dead | 4 | 5 | 9 |
| | Mortality Rate | 33.33 | 12.82 | 17.65 |
| Patients on atorvastatin at time of ICU admit | Alive | 3 | 6 | 9 |
| | Dead | 2 | 1 | 3 |
| | Mortality Rate | 40 | 14.29 | 25 |
| Patients admitted with atorvastatin or initiated during ICU stay | Alive | 11 | 40 | 51 |
| | Dead | 6 | 6 | 12 |
| | Mortality Rate | 35.29 | 13.04 | 19.05 |
| Patients not admitted to ICU on statin and did not receive any statin during ICU stay | Alive | 131 | 346 | 477 |
| | Dead | 54 | 56 | 110 |
| | Mortality Rate | 29.19 | 13.93 | 18.74 |

TABLE 80C

Characteristics of Patients Admitted and any Statin Intervention in filtered eICU data

| | | Subphenotype B | Subphenotype A | All Patients |
|---|---|---|---|---|
| Any statin initiated during ICU stay | Alive | 8 | 38 | 46 |
| | Dead | 6 | 6 | 12 |
| | Mortality Rate | 42.86 | 13.64 | 20.69 |
| Patients on any statin at time of ICU admit | Alive | 14 | 22 | 36 |
| | Dead | 4 | 1 | 5 |
| | Mortality Rate | 22.22 | 435 | 12.2 |
| Patients admitted with any statin or initiated during ICU stay | Alive | 22 | 60 | 82 |
| | Dead | 10 | 7 | 17 |
| | Mortality Rate | 31.25 | 10.45 | 17.17 |
| Patients not admitted to ICU on statin and did not receive any statin during ICU stay | Alive | 131 | 346 | 477 |
| | Dead | 54 | 56 | 110 |
| | Mortality Rate | 29.19 | 13.93 | 18.74 |

The individual statins were then examined with no consideration to number of doses and minimum dose size. Using this methodology, there were several differential responses identified (bolded and underlined cells as shown below in Table 81).

TABLE 81

Differential responses with no consideration to number doses and minimum dose size

| | Subphenotype B | | | p vs no | Subphenotype A | | | p vs no | All Patients | | | p vs no |
|---|---|---|---|---|---|---|---|---|---|---|---|---|
| Treatment | Alive | Dead | Mortality | Statin | Alive | Dead | Mortality | Statin | Alive | Dead | Mortality | Statin |
| No statin | 313 | 166 | 35% | | 870 | 148 | 15% | | 1183 | 314 | 21% | |
| Any Statin | 130 | 45 | 26% | 0.03036 | 362 | 41 | 10% | 0.02897 | 492 | 86 | 15% | 0.00160 |
| Atorvastatin | 80 | 28 | 26% | 0.08148 | 209 | 26 | 11% | 0.16504 | 289 | 54 | 16% | 0.02889 |
| Simvastatin | 45 | 16 | 26% | 0.18978 | 122 | 10 | 8% | 0.02880 | 167 | 26 | 13% | 0.01439 |
| Pravastatin | 9 | 2 | 18% | 0.34550 | 20 | 4 | 17% | 0.76872 | 29 | 6 | 17% | 0.67877 |
| Rosuvastatin | 9 | 2 | 18% | 0.34550 | 23 | 2 | 8% | 0.56302 | 32 | 4 | 11% | 0.21005 |
| Lovastatin | 2 | 1 | 33% | 1.0000 | 10 | 0 | 0% | 0.37326 | 12 | 1 | 8% | 0.32390 |

### Feeding (EDEN Dataset)

This was a retrospective study in a de-identified dataset from one randomized clinical trial in patients with ARDS, entitled ‘Early Versus Delayed Enteral Feeding to Treat People with Acute Lung Injury or Acute Respiratory Distress Syndrome (EDEN)’. Patients were included in the trial in they met the American-European consensus for ARDS, including patients with a PaO2 / FiO2 ratio < 300 up to 48 hours before enrollment, and compared the use of full enteral feeding to trophic feeding.

Data was assessed for completeness and consistency. Of 1,000 patients enrolled, 777 had complete data to train and apply model B.2 as described in Example 5. The majority of the patients were male, and pneumonia was the prevailing etiology followed by sepsis and aspiration.

The primary outcome of the study was 60-day mortality. No secondary outcome was assessed.

The statistical analysis plan was pre-planned. Continuous data were presented as median (quartile 25% - quartile 75%) and compared with the Wilcoxon rank-sum test, and categorical data were presented as number and percentage and compared with Fisher exact tests.

Heterogeneity of Treatment Effect (HTE) of full enteral feeding was assessed following a Bayesian hierarchical logistic model for the primary outcome. All hierarchical models were modelled as a simple regression and shrinkage model. The hierarchical models partially pool the data and shrink the estimates in each subphenotype towards the overall estimate, with shrinkage proportional to the size of the subphenotype. While traditional subgroup analyses are at higher risk of increased type 1 error due to exaggeration of the subgroup effects, the proposed hierarchical model limits this risk through shrinkage.

For all analyses, weakly informative priors were used, aiming to encompass all plausible effect sizes. Since the sample size of the pooled dataset was expected to be large, probably the likelihood will dominate the posteriors.

All described Bayesian models were done using a Markov Chain Monte Carlo simulation with four chains. All models will consider a burn-in of 1,000 iterations, with sampling from a further 10,000 iterations for each chain. All chains were required to be free of divergent transitions and additional sampler settings (adapt delta) were tuned accordingly until this is achieved. To monitor convergence, trace plots, and the Gelman-Rubin convergence diagnostic (Rhat < 1.01) were used for all parameters.

The probability of the following odds ratios (OR) was considered as possible thresholds for the minimum clinically important treatment effect: 1) OR < 1.00; 2) OR < 0.97; and 3) OR < 0.90. These thresholds seem reasonable in view of several considerations. First, the null hypothesis in the frequentist approach is no benefit (OR = 1.00), thus the probability of any benefit (OR < 1.00) will be estimated to evaluate the equivalent hypothesis under Bayesian terms. Second, since the use of statins is a highly feasible intervention, even small effects on mortality would be sufficient to justify its use. Indeed, an OR of 0.97 would be equivalent to an estimated 440 lives saved per year in United States of America (assuming 104000 cases of ARDS annually [7], 40% of these cases meet criteria for moderate-to-severe ARDS [8], and a baseline mortality rate of 35% [8]). To expand the possible detectable effects, we also computed the posterior probabilities at a OR of 0.90, equivalent to 1456 lives saved annually in USA.

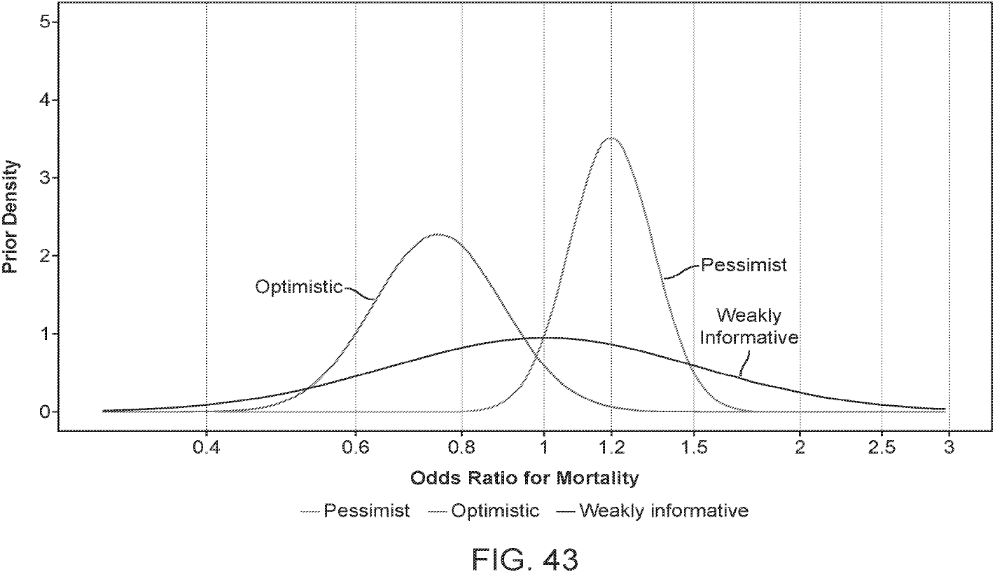

The priors were used to reflect varying degrees of beliefs for benefit or harm of use of statins. Specifically, FIG. 43 shows the treatment prior’s distributions for Bayesian re-analysis of the EDEN trial.

Intercept: The prior was a normally distributed prior with mean 0 and variance 2.25 (prior risk with a 95% probability between 5% and 95%). This prior was used for all analysis including the sensitivity analysis with optimistic and pessimistic priors.

Shrinkage parameter: The prior was a normally distributed prior with mean of 0 and variance of Ω, where Ω is the shrinkage factor having a half-normally distributed prior with variance of 1. This prior was used for all analysis including the sensitivity analysis with optimistic and pessimistic priors.

Treatment Effect - Weakly informative prior: A weakly informative prior was used to produce results essentially dependent on data from the analysis. This was a normally distributed prior with mean of 0 and standard deviation of 0.421 (variance of 0.177). In this prior, there is 90% probability of an 0.50 < OR < 2.00.

Treatment Effect - Optimistic prior: An optimistic prior will be defined to represent archetypes of prior belief that the use of statins effectively lowers mortality. This will be a normally distributed prior with mean of -0.287 and standard deviation of 0.174 (variance of 0.030). This prior distribution will be centered at an OR of 0.75 with a probability of an OR > 1.00 of 5%. This was chosen because and OR ≤ 0.75 was used to power several studies in the field of ARDS, like the ART, EXPRESS, ALVEOLI, SAILS and ROSE trials. Specifically, the SAILS trial was powered to detect an OR ≤ 0.66, however, we judged this an implausible effect size and chose a more conservative one.

Treatment Effect - Pessimistic Prior: A pessimistic prior will be defined to represent archetypes of prior belief that the use of statins increases mortality. This will be a normally distributed prior with mean of 0.183 and standard deviation of 0.113 (variance of 0.012). This prior distribution will be centered at a OR of 1.20 based on the relative risk of death found in the ART trial with a probability of OR < 1.00 of 5%. This was chosen because the ART trial reports an intervention that ultimately increased mortality in ARDS patients.

For the primary outcome, in addition to the odds ratio (OR) with 95% credible interval (CrI), the probability of the following OR was considered as possible thresholds for the minimum clinically important treatment effect: 1) OR < 1.00; 2) OR < 0.97; and 3) OR < 0.90. To understand the possible harm, the probability of harm, defined as a OR > 1.00 (null), is also reported.

All effect estimates were drawn from the median of the posterior distribution and the 95% CrI from the 95% percentiles of the distribution. Additional analyses considering pessimistic and optimistic priors were conducted as sensitivity analyses for the primary HTE analysis. All analyses were performed using the R software (R, version 4.0.2, Core Team, Vienna, Austria, 2016) with the beanz package and Stan through brms.

Baseline characteristics of the patients according to the subphenotype is described in Table 82. Overall, patients in subphenotype B had statistically significant higher severity of illness, rate of vasopressor use, heart rate, creatinine, and bilirubin, as well as lower platelets, pH, BUN and bicarbonate compared to patients in subphenotype A.

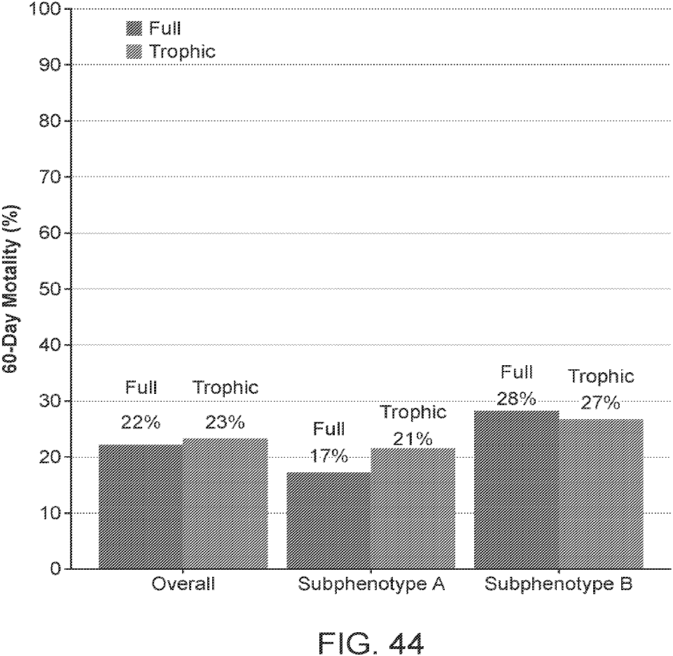

Table 83 summarizes EDEN outcomes by subphenotype and feeding intervention. 60-day mortality was higher and ventilator-free days at day 28 was lower in patients in subphenotype B. 60-day mortality was lower in patients in the full enteral feeding group in subphenotype A, and it was higher in this group in subphenotype B (Table 83). Additionally, FIG. 44 shows 60-day mortality according to subphenotype and intervention group.

TABLE 82

Baseline characteristics of the EDEN subphenotypes

| | Subphenotype A (n = 449) | Subphenotype B (n = 328) | p value |
|---|---|---|---|
| Age, year* | 53.0 (44.0 - 63.0) | 51.0 (41.0 - 62.2) | 0.183 |
| Male gender - no. (%) | 233 (51.9) | 168 (51.2) | 0.910 |
| Body mass index, kg/m² | 29.1 (24.6 - 34.5) | 28.5 (23.4 - 35.1) | 0.476 |
| Caucasian - no. (%) | 349 (81.5) | 237 (75.7) | 0.067 |
| Etiology - no. (%) | | | 0.003 |
| Pneumonia | 296 (65.9) | 217 (66.2) | |
| Sepsis | 50 (11.1) | 60 (18.3) | |
| Aspiration | 45 (10.0) | 27 (8.2) | |
| Trauma | 24 (5.3) | 5 (1.5) | |
| Other | 34 (7.6) | 19 (5.8) | |
| Prognostic scores | | | |
| APACHE III | 66.0 (54.0 - 79.0) | 84.0 (71.0 - 100.2) | < 0.001 |
| Use of vasopressor - no. (%) | 187 (41.6) | 209 (63.7) | < 0.001 |
| Vital signs | | | |
| Temperature, °C | 37.3 (36.8 - 37.8) | 37.3 (36.7 - 38.1) | 0.212 |
| Heart rate, bpm | 89 (77 - 102) | 101 (89 - 116) | < 0.001 |
| Mean arterial Pressure, mmHg | 77.0 (68.0 - 84.0) | 71.0 (66.0 - 80.0) | < 0.001 |
| SpO₂, % | 96 (94 - 98) | 95 (92 - 98) | 0.032 |
| Urine output in 24 hours, mL | 1505 (977 - 2250) | 1165 (566 - 1816) | < 0.001 |
| Laboratory tests | | | |
| Hematocrit, % | 30.0 (26.0 - 34.0) | 30.0 (26.0 - 35.0) | 0.919 |
| White blood cell count, 10⁹/L | 11.4 (7.7 - 15.5) | 12.7 (7.7 - 19.0) | 0.019 |
| Platelets, 10⁹/L | 163 (108 - 241) | 164 (103 - 227) | 0.552 |
| Creatinine, mg/dL | 1.0 (0.7 - 1.5) | 1.6 (1.0 - 2.8) | < 0.001 |
| Bilirubin, mg/dL | 0.8 (0.5 - 1.3) | 0.8 (0.5 - 1.7) | 0.128 |
| Arterial blood gas | | | |
| pH* | 7.40 (7.35 - 7.44) | 7.30 (7.24 - 7.35) | < 0.001 |
| PaO₂, mmHg | 83 (70 - 107) | 81 (67 - 107) | 0.416 |
| PaO₂ / FiO₂ | 133 (98- 193) | 101 (73- 162) | < 0.001 |
| PaCO₂, mmHg | 38 (34 - 44) | 38 (33 - 46) | 0.55 |
| Bicarbonate, mmol/L | 23.0 (21.0 - 26.0) | 18.5 (15.0 - 21.0) | < 0.001 |
| Ventilatory variables | | | |
| Tidal volume, mL | 420 (356 - 487) | 400 (350 - 450) | 0.032 |
| Per PBW, mL/kg PBW | 6.3 (6.0 - 7.5) | 6.1 (6.0 - 7.3) | 0.079 |
| Plateau pressure, cmH₂O | 23.0 (19.0 - 27.0) | 24.0 (21.0 - 28.0) | 0.004 |
| PEEP, cmH₂O | 10 (5 - 10) | 10 (8 -14) | < 0.001 |
| Respiratory rate, breaths/min | 22 (19 - 26) | 30 (25 - 35) | < 0.001 |
| FiO₂ | 0.60 (0.45 - 0.70) | 0.80 (0.60 - 1.00) | < 0.001 |

TABLE 83

Baseline characteristics and clinical outcomes according to allocation group and subphenotypes

| | Subphenotype A | | Subphenotype B | | |
|---|---|---|---|---|---|
| | Full (n = 216) | Trophic (n = 233) | Full (n = 167) | Trophic (n = 161) | p value |
| APACHE III | 66.0 (54.8 - 77.2) | 68.0 (54.0 - 81.0) | 82.0 (70.0 - 99.0) | 88.0 (73.0 - 102.0) | < 0.001 |
| PaO₂ / FiO₂ | 147.9 (109.8 -202.7) | 162.0 (114.0 -210.0) | 114.0 (85.8 -170.0) | 112.0 (85.0 -160.0) | < 0.001 |
| Ventilator-free days at day 28 | 21.0 (11.0 - 25.0) | 22.0 (0.0 - 25.0) | 15.0 (0.0 - 23.0) | 15.0 (0.0 - 22.0) | < 0.001 |
| Duration of ventilation, days | 7.0 (4.0 - 11.0) | 6.0 (3.0 - 11.0) | 8.5 (6.0 - 18.8) | 8.0 (6.0 - 18.0) | < 0.001 |
| Among survivors | 7.0 (4.0 - 11.0) | 6.0 (3.0 - 11.0) | 8.5 (6.0 - 18.8) | 8.0 (6.0 - 18.0) | < 0.001 |
| 28-day mortality - no. (%) | 31 (14.4) | 43 (18.5) | 41 (24.6) | 36 (22.4) | 0.057 |
| 60-day mortality - no. (%) | 37 (17.1) | 50 (21.5) | 47 (28.1) | 43 (26.7) | 0.038 |

Data are median (quartile 25^{th} - quartile 75^{th}) or N (%).

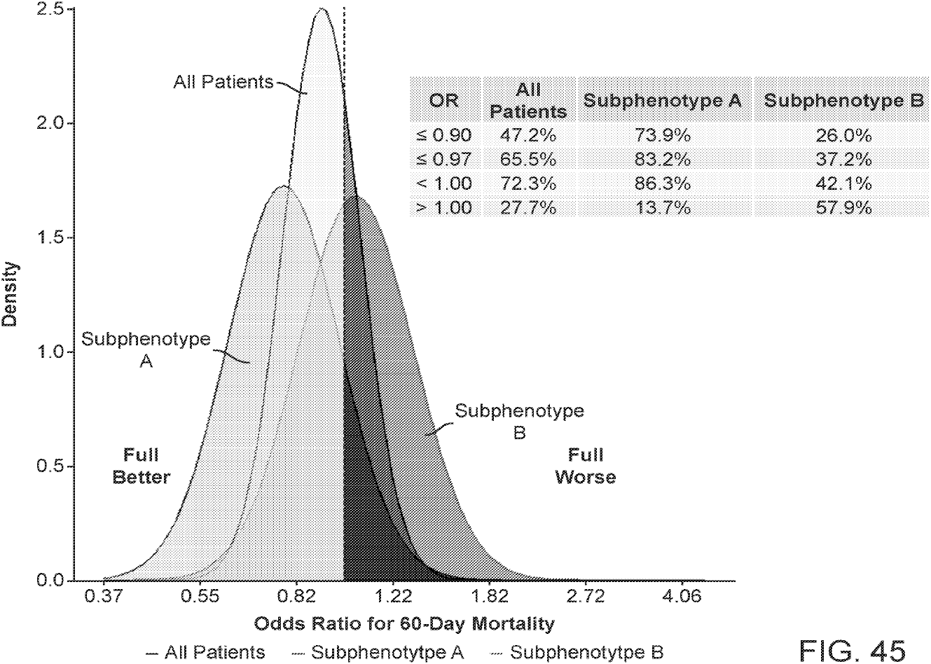

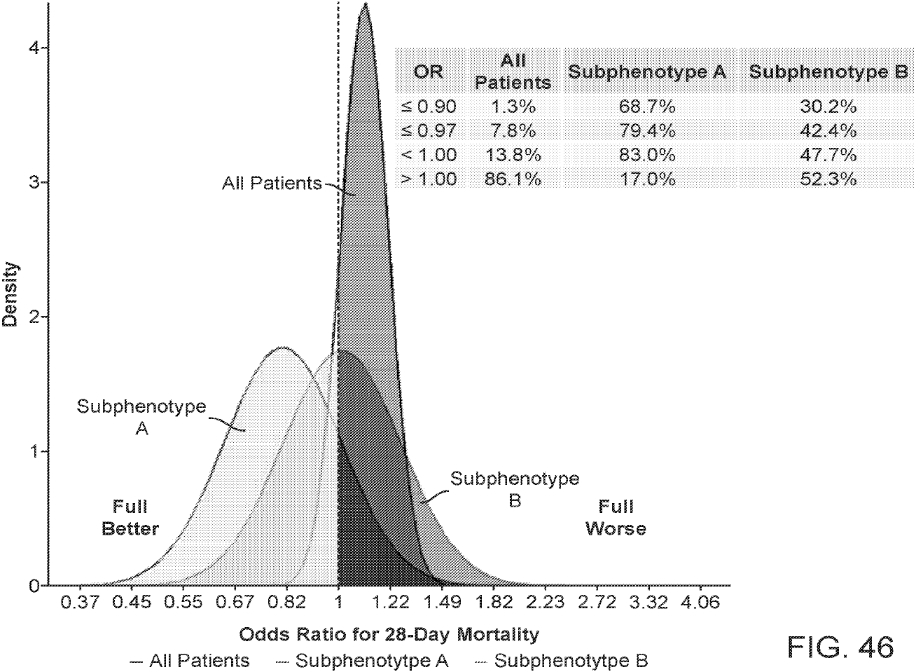

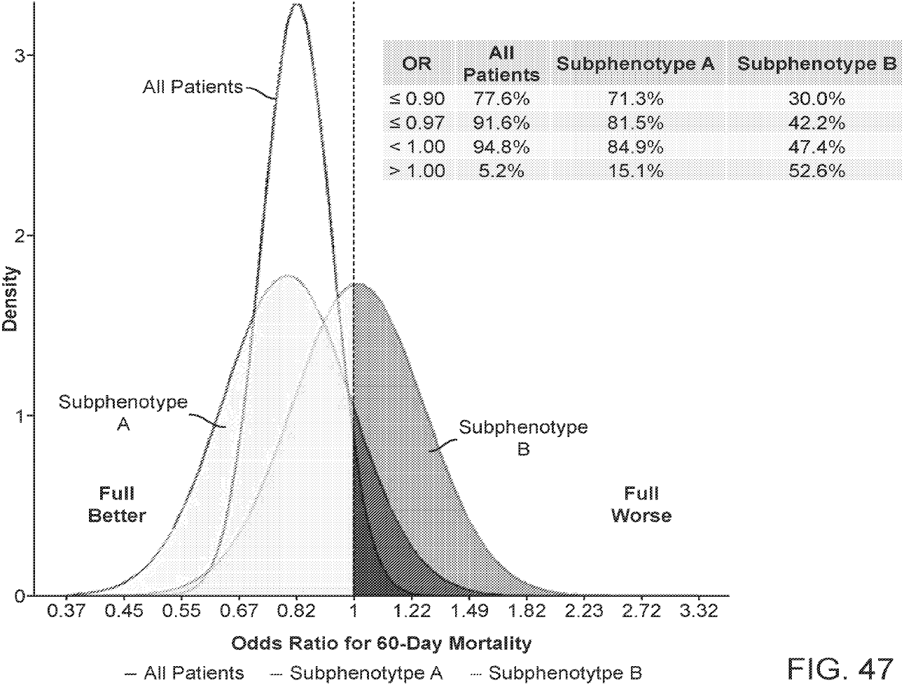

There was no difference in mortality with the use of full enteral feeding neither in subphenotype A (OR, 0.78 [95% CrI, 0.49 to 1.22], probability of benefit of 86.3%) nor in subphenotype B (OR, 1.05 [95% CrI, 0.66 to 1.67], probability of benefit of 42.1%) (Table 84). However, the probability that assignment to a full enteral feeding group results in lower OR for 60-day mortality in patients in subphenotype B (more beneficial), compared to subphenotype A, was only 18.3%. The use of different priors did not materially change these findings (Table 84). These results are further observed in FIGS. 45-47. Specifically, FIG. 45 shows heterogeneity of treatment effect of full feeding in 60-day mortality according to subphenotype, with weakly informative priors considered. Values less than 1 indicate lower mortality. FIG. 46 shows heterogeneity of treatment effect of full feeding in 60-day mortality according to subphenotype considering pessimistic priors. FIG. 47 shows heterogeneity of treatment effect of full feeding in 60-day mortality according to subphenotype considering optimistic priors.

TABLE 84

Heterogeneity of Treatment Effect with 60-day mortality as outcome

| | Odds Ratio (95% CrI) | Probability of OR < 1.00 |
|---|---|---|
| Weakly informative prior* | | |
| All patients | 0.91 (0.66 to 1.24) | 72.3% |
| Subphenotype A | 0.78 (0.49 to 1.22) | 86.3% |
| Subphenotype B | 1.05 (0.66 to 1.67) | 42.1% |
| Probability of lower OR in Subphenotype B | 18.3% | |
| Optimistic prior* | | |
| All patients | 0.82 (0.65 to 1.04) | 94.8% |
| Subphenotype A | 0.79 (0.51 to 1.22) | 84.9% |
| Subphenotype B | 1.02 (0.65 to 1.61) | 47.4% |
| Probability of lower OR in Subphenotype B | 22.3% | |
| Pessimistic prior* | | |
| All patients | 1.11 (0.92 to 1.32) | 13.8% |
| Subphenotype A | 0.81 (0.51 to 1.24) | 83.0% |
| Subphenotype B | 1.01 (0.66 to 1.60) | 47.7% |
| Probability of lower OR in Subphenotype B | 23.8% | |

CrI: credible interval; OR: odds ratio * priors described in the Online Supplement

Product use, if hypothesis confirmed: ARDS patients identified as Subphenotype A should be treated with full feeding; ARDS patients identified as Subphenotype B should be treated with full or trophic feeding.

### Example 8: Guided Neuromuscular Block Treatment in Rose Trial Patients

The preliminary analysis of ARDS subphenotypes to drive neuromuscular block treatment guidance described above in Example 1 represents preliminary findings in observational data and randomized clinical trials studying interventions other than neuromuscular block. Findings in these trials may be driven by patient severity of illness, hospital and/or study protocol, or other unknown factors.

These findings suggest the presence of a differential response, but a clinical trial of neuromuscular block would be required to show a differential response. In May 2021, data from the Reevaluation of Systemic Early Neuromuscular Blockade (ROSE) trial became publicly available. Because the trial was a controlled study of neuromuscular blockade, it allows for more accurate analysis of differential response in ARDS subphenotypes to neuromuscular blockade.

The ROSE trial enrolled 1006 ARDS patients with a PaO2/FiO2 ratio < 150 and a PEEP > 8 between January 2016 and April 2018. Data was cleaned and prepared in Python. Data elements of interest were identified across the various data tables provided by the ROSES authors and collated into a single dataframe/CSV. Data columns with text for missing values were changed to numeric, with NaN replacing text strings.

In previous work, the MAP, creatinine, heart rate, and respiratory rate used in the subphenotyper were aggregated based on the value measured closest to randomization. The ROSE trial did not provide that aggregation measure; instead the highest and lowest values in the 24 hours prior to randomization were provided for those values, which is consistent with calculation of the APACHE score. Because the most recent aggregation method was not available, the APACHE aggregation method to determine values to input to the subphenotyping algorithm. The APACHE method provides a standard midpoint for each clinical variable. For the highest and lowest value, the distance from the mean is calculated. Whichever value (highest or lowest) was furthest from the midpoint was used for input to the subphenotyper.

If the high MAP was further from the APACHE midpoint, it was used. If the low MAP was furthest from the APACHE midpoint, it was used. If the high and low value were equidistant to the midpoint, the value which would receive more APACHE points was used. In the event that high and low value were equidistant to the APACHE midpoint and had the same APACHE points, the lower MAP value was used.

All high and low heart rate values which were equidistant to the APACHE midpoint were in the zero APACHE points range (low value >= 50 bmp and high value <=99 bmp). In all cases, the higher heart rate was used.

Based on study inclusion criteria, all patients were assumed to be mechanically ventilated. This was confirmed in the SCREENING.csv data form in the field scr_intubdttm (hours from randomization to current intubation). 1005/1006 patients had a negative value, signifying intubation prior to study enrollment (one patient had a null value). Because all patients were ventilated, respiratory rates 6 - 12 and 14-24 were both considered 0 APACHE points. APACHE documentation is unclear on how to handle a respiratory rate of 13 in ventilated patients. In one patient with a low respriatory rate of 13 and high respiratory rate of 25, we made the assumption that 13 bpm would be scored as a 0 and used the higher respiratory rate as the most recent respiratory rate. 11 patients had a high and low respiratory rate between 14 - 24. For those patients, the higher respiratory rate was used.

1 patient had a high and low creatinine value that were equidistant from the APACHE midpoint. They were found to not have acute renal failure (high creatinine = 1.02, low creatinine 0.98, urine output = 1885 mL, no history of chronic dialysis). Both the high and low value fell in the 0 point range for APACHE. For that patient, the higher creatinine score was used, because higher creatinine values are typically associated with higher APACHE scores. 398 patients had equal high and low creatinine values, in which case the value from the higher creatinine field was used.

The physiologic limits identified in previous work were applied to the 1006 patients in the ROSE trial (Table 85). 3 patients had values outside of the previously identified physiologic limits. Those values were replaced with null values, which exclude the patient from being assigned a subphenotype.

TABLE 85

Patients with outlying data. R-0247 excluded for heart rate of 0, R-0659 excluded for respiratory rate of 0, and R-0962 excluded for FiO2 of 0.16

| | ID | FIO2R | ARTPHR | BICARL | BILIH | CREATR |
|---|---|---|---|---|---|---|
| 246 | R-0247 | 0.75 | 7.237 | 20.4 | 0.4 | 2 |
| 658 | R-0659 | 0.45 | 7.377 | 22.7 | 4.3 | 0.8 |
| 961 | R-0962 | 0.16 | 7.420 | 24.0 | 0.8 | 1.04 |

TABLE 85 (cont.)

| | ID | HRATER | RESPR | MEANAPR | PAO2R |
|---|---|---|---|---|---|
| 246 | R-0247 | 0 | 45 | 29 | 69.1 |
| 658 | R-0659 | 133 | 0 | 51 | 71.1 |
| 961 | R-0962 | 131 | 29 | 53.33 | 67.0 |

Table 86 shows the percentage of missing data for each of the 9 data elements used in the ARDS phenotyper. Rates of missingness were less than 7% for all elements except bilirubin, which had 27.8% missing.

TABLE 86

Missingness of ROSE trial data

| Variable | % missing |
|---|---|
| Heart Rate | 0.1% |
| Respiratory Rate | 0.2% |
| MAP | 2.1% |
| FiO2 | 0.5% |
| PaO2 | 6.2% |
| Bicarbonate | 3.5% |
| Arterial pH | 6.2% |
| Creatinine | 0.3% |
| Bilirubin | 27.8% |
| Scored Subtype | 34.7% |

Outcome data derived from study data was calculated and provided by the study authors without need for further processing. Derived outcomes included all cause mortality prior to discharge home before 90 (the primary study outcome), study hospital mortality prior to discharge alive to day 28, vent free days (to day 28), hospital free days (to day 28), and ICU free days (to day 28). The date of hospital discharge alive through 90 days and the last date of assisted breathing to day 28 were also provided.

A patient subphenotype classifier (referred as Model B.2 in Example 5) was applied to the 657 ROSE trial data patients that did not have missing data. Of those, 127 (19.3%) were identified as subphenotype A and 525 (80.7%) were assigned to subphenotype B.

The previous hypothesis of lower inflammation in subphenotype A was supported in this data by subphenotype A exhibiting a lower SOFA and APACHE score at study enrollment, lower use of vasopressors and corticosteroids at enrollment, and, in general less severe clinical manifestation, including lower temperature, heart rate, respiratory rate, creatinine, BUN, FiO2, and plateau pressure, and higher mean arterial pressure, urine output, albumin, bicarbonate, arterial pH, PaO2/FiO2. Similarly, Subphenotype A had better outcomes, with lower mortality at 28 and 90 days, and more ventilator, icu, and hospital free days at day 28.

Clinical characteristics of the ROSE population and subphenotypes A and B are shown in Table 87.

TABLE 87

Clinical characteristics of ROSES patients, according to their assigned subphenotype

| | Overall | Subphenotype A | Subphenotype B | P-Value |
|---|---|---|---|---|
| n | 1006 | 126 | 531 | |
| AGE, median [Q1,Q3] | 58.0 [46.0,66.0] | 58.5 [46.0,66.8] | 57.0 [43.5,65.0] | 0.312 |
| MALE GENDER, n (%) | 560 (55.7) | 74 (58.7) | 282 (53.1) | 0.299 |
| BMI, median [Q1,Q3] | 0.0 [0.0,0.0] | 0.0 [0.0,0.0] | 0.0 [0.0,0.0] | 0.077 |
| Etiology, n (%) | | | | |
| Aspiration | 166 (16.5) | 29 (23.0) | 93 (17.5) | 0.009 |
| Other | 49 (4.9) | 11 (8.7) | 27 (5.1) | |
| Pneumonia | 593 (58.9) | 70 (55.6) | 297 (55.9) | |
| Sepsis | 139 (13.8) | 10 (7.9) | 92 (17.3) | |
| Transfusion | 20 (2.0) | 5 (4.0) | 7 (1.3) | |
| Trauma | 39 (3.9) | 1 (0.8) | 15 (2.8) | |
| SOFA, median [Q1,Q3] | 8.0 [6.0,11.0] | 7.0 [5.0,9.0] | 10.0 [7.5,12.0] | <0.001 |
| GCS, median [Q1,Q3] | 7.0 [3.0,9.0] | 6.0 [3.0,6.0] | 6.5 [3.0,9.0] | 0.187 |
| APACHE, median [Q1,Q3] | 106.0 [85.0,128.0] | 90.0 [71.5,107.0] | 114.0 [92.0,137.0] | <0.001 |
| VASOL24, n (%) | 585 (58.2) | 52 (41.3) | 368 (69.3) | <0.001 |
| corticosteroids, n (%) | 231 (23.0) | 27 (21.4) | 127 (23.9) | 0.634 |
| sedatives, n (%) | 905 (90.0) | 120 (95.2) | 477 (89.8) | 0.085 |
| benzos, n (%) | 337 (33.5) | 43 (34.1) | 192 (36.2) | 0.111 |
| ketamines, n (%) | 52 (5.2) | 5 (4.0) | 35 (6.6) | 0.075 |
| propofol, n (%) | 723 (71.9) | 107 (84.9) | 360 (67.8) | 0.001 |
| dexmed, n (%) | 120 (11.9) | 13 (10.3) | 64 (12.1) | 0.124 |
| opioid, n (%) | 844 (83.9) | 105 (83.3) | 443 (83.4) | 0.914 |
| TEMPL, median [Q1,Q3] | 36.5 [36.1,36.9] | 36.5 [36.1,37.0] | 36.4 [36.0,36.9] | 0.355 |
| TEMPH, median [Q1,Q3] | 37.8 [37.2,38.6] | 37.4 [37.0,38.2] | 37.8 [37.2,38.8] | <0.001 |
| MEANAPL, median [Q1,Q3] | 59.0 [53.0,65.0] | 62.0 [57.2,69.0] | 58.0 [51.0,63.0] | <0.001 |
| MEANAPH, median [Q1,Q3] | 98.0 [87.0,112.0] | 100.3 [89.0,120.8] | 96.0 [85.5,112.0] | 0.012 |
| MEANAPR, median [Q1,Q3] | 60.0 [53.0,70.0] | 65.0 [59.0,119.2] | 59.0 [51.0,66.0] | <0.001 |
| HRATEL, median [Q1,Q3] | 83.0 [70.0,95.8] | 72.5 [63.2,86.0] | 86.0 [73.0,99.0] | <0.001 |
| HRATEH, median [Q1,Q3] | 121.0 [104.0,137.0] | 108.0 [93.0,121.8] | 127.0 [111.0,142.0] | <0.001 |
| HRATER, median [Q1,Q3] | 121.0 [104.0,137.0] | 108.0 [89.2,121.8] | 127.0 [111.0,142.0] | <0.001 |
| RESPL, median [Q1,Q3] | 16.0 [14.0,20.0] | 16.0 [14.0,18.0] | 17.0 [14.0,20.0] | 0.035 |
| RESPH, median [Q1,Q3] | 35.0 [29.0,41.0] | 29.0 [24.0,33.0] | 36.0 [31.0,42.0] | <0.001 |
| RESPR, median [Q1,Q3] | 35.0 [29.0,41.0] | 29.0 [24.0,33.0] | 36.0 [31.0,42.0] | <0.001 |
| URINE, median [Q1,Q3] | 942.5 [370.0,1747.5] | 1200.0 [585.0,2115.0] | 732.0 [247.5,1516.2] | <0.001 |
| HCTL, median [Q1,Q3] | 29.9 [25.0,36.4] | 31.7 [26.0,36.7] | 30.2 [25.0,37.3] | 0.324 |
| HCTH, median [Q1,Q3] | 32.2 [26.9,38.2] | 33.3 [27.9,38.4] | 33.0 [27.7,39.8] | 0.967 |
| WBCL, median [Q1,Q3] | 10.8 [5.1,16.1] | 10.9 [6.7,14.3] | 10.1 [4.1,16.1] | 0.374 |
| WBCH, median [Q1,Q3] | 12.7 [6.9,18.6] | 12.2 [8.2,16.5] | 12.7 [6.3,19.6] | 0.612 |
| PLATEL, median [Q1,Q3] | 162.0 [92.0,238.0] | 172.0 [113.2,232.8] | 154.0 [85.0,232.0] | 0.129 |
| SODIUML, median [Q1,Q3] | 137.0 [134.0,140.0] | 138.5 [135.2,142.0] | 137.0 [133.0,140.0] | <0.001 |
| SODIUMH, median [Q1,Q3] | 139.0 [136.0,142.0] | 140.0 [137.0,144.0] | 139.0 [136.0,142.0] | 0.044 |
| CREATL, median [Q1,Q3] | 1.2 [0.8,2.1] | 0.9 [0.7,1.2] | 1.5 [0.9,2.6] | <0.001 |
| CREATH, median [Q1,Q3] | 1.4 [0.9,2.5] | 1.0 [0.7,1.4] | 1.8 [1.1,3.2] | <0.001 |
| CREATR, median [Q1,Q3] | 1.4 [0.8,2.5] | 0.9 [0.7,1.4] | 1.8 [1.0,3.2] | <0.001 |
| GLUCL, median [Q1,Q3] | 122.0 [99.0,155.0] | 122.0 [100.0,155.0] | 119.0 [96.0,155.0] | 0.358 |
| GLUCH, median [Q1,Q3] | 157.0 [123.0,212.0] | 149.0 [120.8,190.5] | 165.0 [125.0,221.0] | 0.015 |
| ALBUMH, median [Q1,Q3] | 2.6 [2.2,3.1] | 2.9 [2.5,3.2] | 2.5 [2.1,3.1] | <0.001 |
| ALBUML, median [Q1,Q3] | 2.8 [2.3,3.3] | 2.9 [2.5,3.4] | 2.8 [2.3,3.3] | 0.03 |
| BILIH, median [Q1,Q3] | 0.8 [0.5,1.9] | 0.7 [0.5,1.1] | 0.9 [0.5,2.0] | 0.076 |
| BICARL, median [Q1,Q3] | 21.0 [17.0,24.6] | 26.0 [23.0,28.0] | 19.0 [16.0,22.0] | <0.001 |
| BUN, median [Q1,Q3] | 28.0 [17.0,48.0] | 21.5 [16.0,33.5] | 32.0 [19.0,53.0] | <0.001 |
| POTASL, median [Q1,Q3] | 3.9 [3.5,4.4] | 3.9 [3.6,4.2] | 3.9 [3.5,4.4] | 0.385 |
| POTASH, median [Q1,Q3] | 4.3 [3.9,4.9] | 4.1 [3.8,4.5] | 4.4 [4.0,5.0] | <0.001 |
| ARTPHR, median [Q1,Q3] | 7.33 [7.26,7.39] | 7.39 [7.36,7.43] | 7.30 [7.23,7.36] | <0.001 |
| PACO2R, median [Q1,Q3] | 42.0 [37.0,49.0] | 43.0 [39.0,49.0] | 42.0 [36.0,49.0] | 0.068 |
| PAO2R, median [Q1,Q3] | 76.0 [67.0,92.0] | 76.0 [65.5,90.8] | 77.0 [67.8,92.0] | 0.411 |
| SPO2R_abg, median [Q1,Q3] | 94.6 [92.0,97.0] | 94.0 [93.0,97.0] | 94.0 [91.2,97.0] | 0.099 |
| SPO2R, median [Q1,Q3] | 95.0 [93.0,97.0] | 95.0 [93.0,97.0] | 95.0 [92.0,97.0] | 0.411 |
| FIO2R, median [Q1,Q3] | 0.7 [0.6,0.9] | 0.6 [0.5,0.8] | 0.7 [0.6,1.0] | <0.001 |
| FIO2R_abg, median [Q1,Q3] | 0.8 [0.6,1.0] | 0.7 [0.6,0.9] | 0.8 [0.6,1.0] | 0.003 |
| PAFIL, median [Q1,Q3] | 85.0 [66.7,110.0] | 94.5 [68.1,118.8] | 81.9 [65.9,106.0] | 0.024 |
| PAFI_abg, median [Q1,Q3] | 114.0 [87.5,138.5] | 120.0 [91.5,140.9] | 112.6 [85.2,138.5] | 0.135 |
| PEEPR, median [Q1,Q3] | 12.0 [10.0,15.0] | 12.0 [10.0,15.0] | 12.0 [10.0,16.0] | 0.696 |
| TIDALR, median [Q1,Q3] | 400.0 [340.0,450.0] | 400.0 [345.0,450.0] | 400.0 [340.0,440.0] | 0.164 |
| TIDALR/PBW, median [Q1,Q3] | 6.0 [5.9,6.6] | 6.0 [5.9,6.4] | 6.0 [5.9,6.6] | 0.701 |
| TIDAL_derived, median [Q1,Q3] | 6.0 [5.9,6.6] | 6.0 [5.9,6.4] | 6.0 [5.9,6.6] | 0.691 |
| TMVNTR, median [Q1,Q3] | 10.9 [8.9,13.3] | 9.6 [8.0,11.2] | 11.3 [9.3,13.8] | <0.001 |
| PLATEAUR, median [Q1,Q3] | 25.5 [22.0,29.0] | 24.0 [21.0,27.8] | 26.0 [22.0,30.0] | 0.029 |
| vfd, median [Q1,Q3] | 0.0 [0.0,21.0] | 17.0 [0.0,22.8] | 0.0 [0.0,20.5] | 0.001 |
| hospfd28, median [Q1,Q3] | 0.0 [0.0,13.0] | 4.0 [0.0,16.0] | 0.0 [0.0,11.0] | 0.001 |
| icufd28, median [Q1,Q3] | 6.0 [0.0,18.0] | 15.0 [0.0,21.0] | 3.0 [0.0,17.0] | <0.001 |
| DEAD28, n (%) | 371 (36.9) | 35 (27.8) | 209 (39.4) | 0.021 |
| DEAD90, n (%) | 429 (42.6) | 46 (36.5) | 236 (44.4) | 0.129 |

Next, the outcomes were compared across intervention and subphenotype (Table 88).

TABLE 88

Clinical outcomes of ROSES patients, according to their assigned subphenotype and intervention group

| | Overall | Subphenotype A_Control | Subphenotype A_NMB | Subphenotype B_Control | Subphenotype B_NMB | P-Value |
|---|---|---|---|---|---|---|
| n | 1006 | 57 | 69 | 277 | 254 | |
| vfd, median [Q1,Q3] | 0.0 [0.0,21.0] | 17.0 [0.0,23.0] | 17.0 [0.0,22.0] | 0.0 [0.0,21.0] | 0.0 [0.0,19.0] | 0.007 |
| hospfd28, median [Q1,Q3] | 0.0 [0.0,13.0] | 0.0 [0.0,15.0] | 8.0 [0.0,16.0] | 0.0 [0.0,12.0] | 0.0 [0.0, 8.8] | 0.002 |
| icufd28, median [Q1,Q3] | 6.0 [0.0,18.0] | 12.0 [0.0,21.0] | 16.0 [0.0,22.0] | 4.0 [0.0,18.0] | 3.0 [0.0,16.0] | <0.001 |
| DEAD28, n (%) | 371 (36.9) | 17 (29.8) | 18 (26.1) | 112 (40.4) | 97 (38.2) | 0.097 |
| DEAD90, n (%) | 429 (42.6) | 26 (45.6) | 20 (29.0) | 121 (43.7) | 115 (45.3) | 0.099 |

Patients in subphenotype A who received no treatment (the control group) had higher mortality and fewer ventilator, ICU, and hospital free days than subphenotype A patients in the cohort who received NMB. Thus, NMB therapy can benefit patients in subphenotype A. Conversely, patients in subphenotype B did not have dramatic differences in mortality or ventilator, ICU, or hospital free days.

Further analysis of differential response was carried out using binomial regression for binary outcomes and quantile regression for continuous variables. Of note, model B.2. trained on all EDEN and FACTT and applied to ROSE showed a p value of 0.077 for 90-day mortality (the primary study outcome) interaction between subphenotype and NMB treatment (Table 89).

TABLE 89

Regression analysis to identify differential response to treatment

| Raw ROSES data | NMB | Control | p-value |
|---|---|---|---|
| n | 501 | 505 | |
| DEAD28, n (%) | 184 (36.7) | 187 (37.0) | 0.973 |
| DEAD90, n (%) | 213 (42.5) | 216 (42.8) | 0.985 |
| vfd, median (IQR) | 1.5 [0.0,21.0] | 0.0 [0.0,22.0] | 0.508 |
| hospfd28, median (IQR) | 0.0 [0.0,13.0] | 0.0 [0.0,13.0] | 0.975 |
| icufd28, median (IQR) | 6.0 [0.0,18.0] | 6.0 [0.0,19.0] | 0.535 |

| Model B.2. | Subphenotype A | | Subphenotype B | | p-value |
|---|---|---|---|---|---|
| | Control | NMB | Control | NMB | |
| n | 57 | 69 | 277 | 254 | |
| DEAD28, n (%) | 17 (29.8) | 18 (26.1) | 112 (40.4) | 97 (38.2) | 0.834 |
| DEAD90, n (%) | 26 (45.6) | 20 (29.0) | 121 (43.7) | 115 (45.3) | 0.058 |
| vfd, median (IQR) | 17.0 (0.0 -23.0) | 17.0 (0.0 -22.0) | 0.0 (0.0 -21.0) | 0.0 (0.0 -19.0) | 0.684 |
| hospfd28, median (IQR) | 0.0 (0.0 - 15.0) | 9.0 (0.0 - 16.0) | 0.0 (0.0 -12.0) | 0.0 (0.0 - 8.8) | 0.31 |
| icufd28, median (IQR) | 12.0 (0.0 -21.0) | 16.0 (0.0 -22.0) | 4.0 (0.0 -18.0) | 3.0 (0.0 -16.0) | 0.318 |

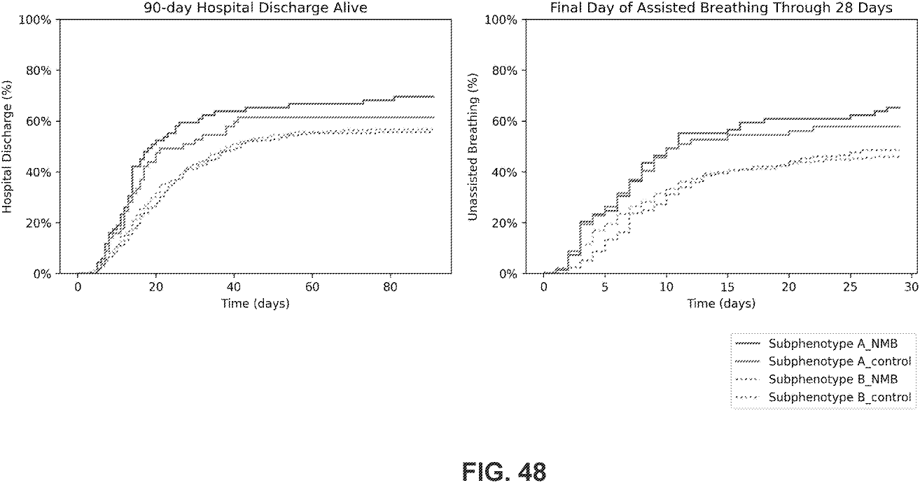

Day of hospital discharge through 90 days and final day of assisted breathing through day 28 were available. FIG. 48 depicts the percentage of patients discharged alive over time through 90 days, stratified by subphenotype and neuromuscular block intervention, and the percentage of patients reaching their final day of unassisted breathing through 28 days, stratified by subphenotype and neuromuscular block intervention. Cumulative density plots were created to show the rate of hospital discharge and unassisted breathing over time for each subphenotype/intervention arm. Both plots show consistently better outcomes in the NMB arm of subphenotype A after around 10 days.

Overall, the findings of the re-analysis of the randomized controlled ROSE trial suggest that patients in Subphenotype A benefit from neuromuscular blockade, while patients in Subphenotype B may or may not benefit from neuromuscular blockaded.

### Example 9: Summary of Guided Differential Treatments

Table 90 summarizes the guided differential treatments for ARDS patients K-means clustered in either Subphenotype A or Subphenotype B using a model (e.g., model C.4) disclosed herein.

TABLE 90

Preliminary findings on guided differential treatment for patients of high mortality risk (Subphenotype B) or low mortality risk (Subphenotype A)

| | | |
|---|---|---|
| Treatment | Subphenotype B (high mortality risk) | Subphenotype A (low mortality risk) |
| Neuromuscular blockage (NMB) | No treatment or administer NMB therapy | Administer NMB therapy |
| Positive End-Expiratory Pressure (PEEP) | High PEEP or low PEEP | Administer Low PEEP |
| Methylpredinosolone | No treatment or administer methylprednisolone | No methylprednisolone |
| Dexamethasone (in Covid-19 induced ARDS) | Administer dexamethasone | No treatment or administer dexamethasone |
| Lisofylline | No lisofylline | No treatment or administer lisofylline |
| Ketoconazole | Administer ketoconazole | No treatment or administer ketoconazole |
| Catheter and Fluid | PAC or CVC line Liberal or conservative fluid management | Do not treat with combination of PAC line and liberal fluid |
| Recruitment Maneuver | Consider recruitment maneuver | No recruitment maneuver |
| Statins | Administer statins at any time | Administer statins as early as possible, even prior to ARDs diagnosis (if no contraindications) |
| Enteral Feeding | Full Feeding or Trophic Feeding | Full Feeding |

1. A method, comprising:
obtaining or having obtained electronic health record (EHR) data for a subject exhibiting acute respiratory distress syndrome (ARDS); and
determining a classification of the subject selected from two or more subphenotypes by analyzing, using a patient subphenotype classifier, the EHR data for the subject without analyzing biomarker levels of the subject.

2. The method of claim 1, wherein the patient subphenotype classifier receives one or more input variables comprising heart rate, mean arterial pressure, and respiratory rate.

3. The method of claim 2, wherein the patient subphenotype classifier receives each of the input variables of heart rate, mean arterial pressure, and respiratory rate.

4. The method of claim 2 or 3, wherein the patient subphenotype classifier further receives one or more input variables comprising arterial pH, partial pressure of oxygen, and bicarbonate.

5. The method of claim 4, wherein the patient subphenotype classifier further receives each of the input variables comprising arterial pH, partial pressure of oxygen, and bicarbonate.

6. The method of any one of claims 2-5, wherein the patient subphenotype classifier further receives one or more input variables comprising inspirited fraction of oxygen, creatinine, and bilirubin.

7. The method of claim 6, wherein the patient subphenotype classifier further receives each of the input variables comprising inspirited fraction of oxygen, creatinine, and bilirubin.

8. The method of any one of claims 2-7, wherein the patient subphenotype classifier further receives one or more input variables comprising partial pressure of carbon dioxide, PaO₂/FiO₂, platelet count, age, gender, positive end-expiratory pressure, and tidal volume.

9. The method of claim 8, wherein the patient subphenotype classifier further receives each of the input variables comprising partial pressure of carbon dioxide, PaO₂/FiO₂, platelet count, age, gender, positive end-expiratory pressure, and tidal volume.

10. The method of any one of claims 2-9, wherein the patient subphenotype classifier further receives one or more input variables comprising body mass index, plateau pressure, minute ventilation, and vasopressor use in prior 24 hours.

11. The method of claim 10, wherein the patient subphenotype classifier further receives each of the input variables comprising body mass index, plateau pressure, minute ventilation, and vasopressor use in prior 24 hours.

12. The method of claim 1, wherein the patient subphenotype classifier comprises a subphenotyping submodel that outputs a prediction for an ARDS subphenotype.

13. The method of claim 1, wherein the patient subphenotype classifier comprises a mortality submodel that outputs a prediction of an ARDS mortality rate.

14. The method of claim 1, wherein the patient subphenotype classifier comprises:
(A) a subphenotyping submodel that outputs a prediction for an ARDS subphenotype; and
(B) a mortality submodel that outputs a prediction of an ARDS mortality rate.

15. The method of claim 14, wherein the prediction for the ARDS subphenotype outputted by the subphenotyping submodel serves as an input to the mortality submodel.

16. The method of any one of claims 12 or 14-15, wherein the subphenotyping submodel receives one or more input variables comprising the subject’s arterial pH, bicarbonate, creatinine, fraction of inspired oxygen (FiO₂), heart rate, arterial pressure, respiration rate, and partial pressure of oxygen (PaO₂).

17. The method of any one of claims 12 or 14-16, wherein the subphenotyping submodel receives each of the input variables of the subject’s arterial pH, bicarbonate, creatinine, fraction of inspired oxygen (FIO₂), heart rate, arterial pressure, respiration rate, and partial pressure of oxygen (PaO₂).

18. The method of any one of claims 12 or 14-17, wherein implementation of the subphenotyping submodel comprises implementing an unsupervised clustering algorithm.

19. The method of any one of claims 13-18, wherein the mortality submodel receives input variables comprising the subject’s gender and age.

20. The method of any one of claims 13-19, wherein the mortality submodel receives input variables comprising the subject’s bilirubin, partial pressure of carbon dioxide (PaCO₂), PaO₂/FiO₂, positive end expiratory pressure (PEEP), platelet count, and tidal volume.

21. The method of any one of claims 13-19, wherein the mortality submodel receives input variables comprising the subject’s arterial pH, bicarbonate, creatinine, fraction of inspired oxygen (FiO₂), heart rate, arterial pressure, respiration rate, and partial pressure of oxygen (PaO₂).

22. The method of any one of claims 13-19, wherein the mortality submodel receives 10 or more input variables comprising the prediction for the ARDS subphenotype outputted by the subphenotyping submodel, the subject’s gender, age, bilirubin, partial pressure of carbon dioxide (PaCO₂), PaO₂/FiO₂, positive end expiratory pressure (PEEP), platelet count, tidal volume, and BMI.

23. The method of claim 22, wherein the patient subphenotype classifier has at least one of an area under receiver-operator curve (AUROC) greater than or equal to 0.689 and an area under the precision-recall curve (AUPRC) greater than or equal to 0.650.

24. The method of any one of claims 13-19, wherein the mortality submodel receives 9 or more input variables comprising the prediction for the ARDS subphenotype outputted by the subphenotyping submodel, the subject’s gender, age, bilirubin, partial pressure of carbon dioxide (PaCO₂), PaO₂/FiO₂, positive end expiratory pressure (PEEP), platelet count, and tidal volume.

25. The method of claim 24, wherein the patient subphenotype classifier has at least one of an area under receiver-operator curve (AUROC) greater than or equal to 0.673 and an area under the precision-recall curve (AUPRC) greater than or equal to 0.668.

26. The method of any one of claims 13-19, wherein the mortality submodel receives 12 or more input variables comprising the prediction for the ARDS subphenotype outputted by the subphenotyping submodel, the subject’s gender, age, bilirubin, arterial pH, bicarbonate, creatinine, fraction of inspired oxygen (FIO₂), heart rate, arterial pressure, respiration rate, and partial pressure of oxygen (PaO₂).

27. The method of claim 26, wherein the patient subphenotype classifier has at least one of an area under receiver-operator curve (AUROC) greater than or equal to 0.658 and an area under the precision-recall curve (AUPRC) greater than or equal to 0.597.

28. The method of any one of claims 13-19, wherein the mortality submodel receives 11 or more input variables comprising the prediction for the ARDS subphenotype outputted by the subphenotyping submodel, the subject’s gender, age, arterial pH, bicarbonate, creatinine, fraction of inspired oxygen (FiO₂), heart rate, arterial pressure, respiration rate, and partial pressure of oxygen (PaO₂).

29. The method of claim 28, wherein the patient subphenotype classifier has at least one of an area under receiver-operator curve (AUROC) greater than or equal to 0.643 and an area under the precision-recall curve (AUPRC) greater than or equal to 0.532.

30. The method of any one of claims 13-29, wherein implementation of the mortality submodel comprises implementing a supervised machine learning algorithm.

31. The method of any one of claims 13-30, wherein determining the classification of the subject based on the EHR data using the patient subphenotype classifier comprises
determining that data elements of a higher rank mortality submodel are unavailable in the EHR data; and
determining that data elements of the mortality submodel are available in the EHR data.

32. The method of any one of claims 13-31, wherein determining the classification of the subject based on the EHR data using the patient subphenotype classifier comprises implementing the mortality submodel responsive to determining that data elements of the mortality submodel are available in the EHR data.

33. The method of any one of claims 14-18, wherein the mortality submodel comprises two or more sub-models that each outputs a prediction informative for determining an ARDS mortality rate.

34. The method of claim 33, wherein the first sub-model receives input variables comprising a first prediction for the ARDS subphenotype outputted by the subphenotyping submodel and the second sub-model receives input variables comprising a second prediction for the ARDS subphenotype outputted by the subphenotyping submodel.

35. The method of claim 34, wherein the first sub-model receives input variables further comprising the subject’s bilirubin.

36. The method of claim 34, wherein the second sub-model receives input variables further comprising the subject’s bilirubin, partial pressure of carbon dioxide (PaCO₂), PaO₂/FiO₂, positive end expiratory pressure (PEEP), platelet count, and tidal volume.

37. The method of any one of claims 12 or 14-32, wherein the subphenotyping submodel comprises two or more sub-models that each outputs a prediction of an ARDS subphenotype.

38. The method of claim 37, wherein implementation of the two or more sub-models comprises implementing unsupervised clustering algorithms.

39. The method of any one of claims 12 or 14-32, wherein the patient subphenotype classifier further comprises a pre-mortality model that outputs a prediction that serves as input to the mortality submodel.

40. The method of claim 39, wherein implementation of the pre-mortality model comprises implementing a supervised machine learning algorithm.

41. The method of claim 13, wherein the mortality submodel receives, as input, 8 or more input variables.

42. The method of claim 41, wherein the 8 or more input variables comprise at least the subject’s arterial pH, bicarbonate, creatinine, fraction of inspired oxygen (FiO₂), and heart rate.

43. The method of claim 41, wherein the 8 or more input variables further comprise at least the subject’s airway pressure, arterial pressure, respiration rate, and partial pressure of oxygen (PaO₂).

44. The method of claim 41, wherein the patient subphenotype classifier comprises one of a first model, a second model, a third model, and a fourth model,
wherein the first model receives, as input, 13 input variables,
wherein the second model receives, as input, 8 input variables,
wherein the third model receives, as input, 17 input variables, and
wherein the fourth model receives, as input, 13 input variables.

45. The method of claim 44, wherein the 13 input variables of the first model comprise the subject’s arterial pH, bicarbonate, creatinine, diastolic blood pressure (BP), FiO₂, heart rate, highest mean arterial pressure, lowest mean arterial pressure, potassium, highest respiratory rate, lowest respiratory rate, SPO₂, and systolic BP.

46. The method of claim 44 or 45, wherein the 13 input variables of the first model comprise the subject’s most recent arterial pH, lowest bicarbonate, most recent creatinine, most recent diastolic blood pressure (BP), most recent FiO₂, most recent heart rate, highest mean arterial pressure, lowest mean arterial pressure, most recent potassium, highest respiratory rate, lowest respiratory rate, most recent SPO₂, and most recent systolic BP.

47. The method of any one of claims 44-46, wherein the patient subphenotype classifier has at least one of an area under receiver-operator curve (AUROC) greater than or equal to 0.67 and an area under the precision-recall curve (AUPRC) greater than or equal to 0.40.

48. The method of claim 44, wherein the 8 input variables of the second model comprise the subject’s arterial pH, bicarbonate, creatinine, FiO₂, heart rate, PaO₂, mean arterial pressure, and respiratory rate.

49. The method of claim 44 or 48, wherein the 8 input variables of the second model comprise the subject’s most recent arterial pH, lowest bicarbonate, most recent creatinine, most recent FiO₂, most recent heart rate, most recent PaO₂, most recent mean arterial pressure, and most recent respiratory rate.

50. The method of any one of claims 44 or 48-49, wherein the patient subphenotype classifier has at least one of an area under receiver-operator curve (AUROC) greater than or equal to 0.69 and an area under the precision-recall curve (AUPRC) greater than or equal to 0.42.

51. The method of claim 44, wherein the 17 input variables of the third model comprise the subject’s age, arterial pH, bicarbonate, bilirubin, BMI, creatinine, FiO₂, gender, heart rate, PaCO₂, PaO₂/FiO₂, PaO₂, positive end-expiratory pressure (PEEP), platelet count, tidal volume, mean arterial pressure, and respiratory rate.

52. The method of claim 44 or 51, wherein the 17 input variables of the third model comprise the subject’s age, most recent arterial pH, lowest bicarbonate, highest bilirubin, BMI, most recent creatinine, most recent FiO₂, gender, most recent heart rate, most recent PaCO₂, lowest PaO₂/FiO₂ within 24 hours following ARDS diagnosis, most recent PaO₂, most recent positive end-expiratory pressure (PEEP), lowest platelet count, lowest tidal volume, most recent mean arterial pressure, and most recent respiratory rate.

53. The method of any one of claims 44 or 51-52, wherein the patient subphenotype classifier has at least one of an area under receiver-operator curve (AUROC) greater than or equal to 0.71 and an area under the precision-recall curve (AUPRC) greater than or equal to 0.62.

54. The method of claim 44, wherein the 13 input variables of the fourth model comprise the subject’s arterial pH, bicarbonate, BMI, creatinine, FiO₂, gender, heart rate, PaCO₂, PaO₂/FiO₂, PEEP, platelet count, mean arterial pressure, and respiratory rate.

55. The method of claim 44 or 54, wherein the 13 input variables of the fourth model comprise the subject’s most recent arterial pH, most recent bicarbonate, BMI, most recent creatinine, most recent FiO₂, gender, most recent heart rate, most recent PaCO₂, lowest PaO₂/FiO₂ within 24 hours following ARDS diagnosis, most recent PEEP, lowest platelet count, most recent mean arterial pressure, and most recent respiratory rate.

56. The method of any one of claims 44 or 54-55, wherein the patient subphenotype classifier has at least one of an area under receiver-operator curve (AUROC) greater than or equal to 0.67 and an area under the precision-recall curve (AUPRC) greater than or equal to 0.46.

57. The method of claim 1, wherein the classification of the subject is selected from three or more subphenotypes.

58. The method of claim 57, wherein the three or more subphenotypes comprise a lower risk subphenotype, a medium risk subphenotype, and a high risk subphenotype.

59. The method of claim 57 or 58, wherein the classification of the subject is selected from three by comparing a score to two threshold values.

60. The method of any one of claims 57-59, wherein the patient subphenotype classifier has at least an area under receiver-operator curve (AUROC) greater than or equal to 0.691.

61. The method of any one of claims 1-60, wherein the patient subphenotype classifier is trained using a training dataset comprising patient data from one or more clinical trial datasets.

62. The method of claim 61, wherein the one or more clinical trial datasets are any of ARMA dataset, KARMA dataset, LARMA dataset, ALVEOLI dataset, EDEN dataset, FACTT dataset, SAILS dataset, ROSE dataset, eICU-CRD dataset, and the Brazillian ART dataset.

63. The method of claim 61 or 62, wherein the patient data is derived from a sub-cohort of patients of the one or more clinical trial datasets, wherein the sub-cohort of patients are characterized by having a ratio of arterial oxygen concentration to the fraction of inspired oxygen (P/F ratio) of less than or equal to 200.

64. The method of claim 61 or 62, wherein the patient data is derived from a sub-cohort of patients of the one or more clinical trial datasets, wherein the sub-cohort of patients are characterized by having a ratio of arterial oxygen concentration to the fraction of inspired oxygen (P/F ratio) of less than or equal to 300.

65. The method of any one of claims 1-64, wherein the two or more subphenotypes comprise subphenotype A and subphenotype B that are characterized by differences in expression levels in one or more biomarkers.

66. The method of claim 65, wherein the one or more biomarkers comprise one or more of PAI-1, IL-6, IL-8, IL-10, TNFR-I, TNFR-II, ICAM-1, or von Willebrand factor.

67. The method of claim 65, wherein the one or more biomarkers comprise each of PAI-1, IL-6, IL-8, IL-10, TNFR-I, TNFR-II, ICAM-1, or von Willebrand factor.

68. A method for identifying a mortality prognosis for a subject, the method comprising:
obtaining a classification of the subject exhibiting acute respiratory distress syndrome (ARDS), the classification of the subject selected from two or more subphenotypes and determined using the method of any one of claims 1-67; and
identifying a mortality prognosis for the subject based at least in part on the classification,
wherein responsive to the classification of the subject comprising subphenotype B from the two or more subphenotypes, the mortality prognosis identified for the subject comprises high mortality risk, and
wherein responsive to the classification of the subject comprising subphenotype A from the two or more subphenotypes, the mortality prognosis identified for the subject comprises low mortality risk.

69. The method of claim 68, wherein low mortality risk comprises at least one of reduced risk of hospital mortality, reduced risk of ICU mortality, reduced risk of 28-day mortality, reduced risk of 90-day mortality, reduced risk of 180-day mortality, and reduced risk of 6-month mortality relative to high mortality risk.

70. The method of claim 68 or 69, wherein low mortality risk further comprises positive patient outcome, wherein high mortality risk further comprises negative patient outcome, and wherein positive patient outcome comprises at least one of shorter hospital length of stay, shorter ICU length of stay and more ventilator-free days relative to negative patient outcome.

71. A method for identifying a therapy recommendation for a subject, the method comprising:
obtaining a classification of a subject exhibiting acute respiratory distress syndrome (ARDS), the classification of the subject selected from two or more subphenotypes and determined using the method of any one of claims 1-67; and
identifying a therapy recommendation for the subject based at least in part on the classification,
wherein responsive to the classification of the subject comprising subphenotype B from the two or more subphenotypes, the therapy recommendation identified for the subject comprises one or more of neuromuscular blockade (NMB) therapy or no NMB therapy, high PEEP or low PEEP, no treatment or methylprednisolone, dexamethasone, no lisofylline, ketoconazole, catheter and fluid treatment, recruitment maneuver, statins, or full or trophic enteral feeding and
wherein responsive to the classification of the subject comprising subphenotype A from the two or more subphenotypes, the therapy recommendation identified for the subject comprises one or more of NMB therapy, low PEEP therapy, no methylprednisolone, no treatment or dexamethasone, no treatment or lisofylline, no treatment or ketoconazole, no combination of catheter and fluid treatment, no recruitment maneuver, statins as a preemptive therapy, or full enteral feeding.

72. A method for identifying candidate subjects to be provided a therapy, the method comprising:
for one or more subjects, obtaining a classification of the subject exhibiting acute respiratory distress syndrome (ARDS), the classification of the subject selected from two or more subphenotypes and determined using the method of any one of claims 1-67; and
determining whether the subject is a candidate subject based at least in part on the classification.

73. The method of claim 72, wherein the therapy is a neuromuscular blockade (NMB) therapy, and wherein determining whether the subject is a candidate subject comprises determining that the subject is a likely responder responsive to the classification of the subject comprising subphenotype A from the two or more subphenotypes.

74. The method of claim 72, wherein the therapy is a neuromuscular blockade (NMB) therapy, and wherein determining whether the subject is a candidate subject comprises determining that the subject is unlikely to be a responder responsive to the classification of the subject comprising subphenotype B from the two or more subphenotypes.

75. The method of claim 72, wherein the therapy is a low positive end-expiratory pressure (PEEP) treatment, and wherein determining whether the subject is a candidate subject comprises determining that the subject is likely to be a responder responsive to the classification of the subject comprising subphenotype A from the two or more subphenotypes.

76. The method of claim 72, wherein the therapy is a high positive end-expiratory pressure (PEEP) treatment, and wherein determining whether the subject is a candidate subject comprises determining that the subject is likely to be a responder responsive to the classification of the subject comprising subphenotype B from the two or more subphenotypes.

77. The method of claim 72, wherein the therapy is a corticosteroid treatment, and wherein determining whether the subject is a candidate subject comprises determining that the subject is likely to be a responder responsive to the classification of the subject comprising subphenotype B from the two or more subphenotypes.

78. The method of claim 72, wherein the therapy is a corticosteroid treatment, and wherein determining whether the subject is a candidate subject comprises determining that the subject is unlikely to be a responder responsive to the classification of the subject comprising subphenotype A from the two or more subphenotypes.

79. The method of claim 77 or 78, wherein the corticosteroid treatment is methylpredinosolone or dexamethasone.

80. The method of claim 72, wherein the therapy is a lisofylline treatment, and wherein determining whether the subject is a candidate subject comprises determining that the subject is unlikely to be a responder responsive to the classification of the subject comprising subphenotype B from the two or more subphenotypes.

81. The method of claim 72, wherein the therapy is a lisofylline treatment, and wherein determining whether the subject is a candidate subject comprises determining that the subject is likely to be a responder responsive to the classification of the subject comprising subphenotype A from the two or more subphenotypes.

82. The method of claim 72, wherein the therapy is a ketoconazole treatment, and wherein determining whether the subject is a candidate subject comprises determining that the subject is likely to be a responder responsive to the classification of the subject comprising subphenotype B from the two or more subphenotypes.

83. The method of claim 72, wherein the therapy is a pulmonary artery catheter and liberal fluid treatment, and wherein determining whether the subject is a candidate subject comprises determining that the subject is likely to be a responder responsive to the classification of the subject comprising subphenotype B from the two or more subphenotypes.

84. The method of claim 72, wherein the therapy is a pulmonary artery catheter and liberal fluid treatment, and wherein determining whether the subject is a candidate subject comprises determining that the subject is unlikely to be a responder responsive to the classification of the subject comprising subphenotype A from the two or more subphenotypes.

85. The method of claim 83 or 84, wherein the catheter and fluid treatment comprises a central venous catheter line treatment or a pulmonary artery catheter line treatment.

86. The method of claim 72, wherein the therapy is a recruitment maneuver, and wherein determining whether the subject is a candidate subject comprises determining that the subject is likely to be a responder responsive to the classification of the subject comprising subphenotype B from the two or more subphenotypes.

87. The method of claim 72, wherein the therapy is a recruitment maneuver, and wherein determining whether the subject is a candidate subject comprises determining that the subject is unlikely to be a responder responsive to the classification of the subject comprising subphenotype A from the two or more subphenotypes.

88. The method of claim 72, wherein the therapy is a statin treatment, and wherein determining whether the subject is a candidate subject comprises determining that the subject is likely to be a responder responsive to the classification of the subject comprising subphenotype B from the two or more subphenotypes.

89. The method of claim 72, wherein the therapy is a preemptive statin treatment, and wherein determining whether the subject is a candidate subject comprises determining that the subject is likely to be a responder responsive to the classification of the subject comprising subphenotype A from the two or more subphenotypes.

90. The method of claim 72, wherein the therapy is full enteral feeding, and wherein determining whether the subject is a candidate subject comprises determining that the subject is likely to be a responder responsive to the classification of the subject comprising subphenotype A from the two or more subphenotypes.

91. The method of claim 72, wherein the therapy is trophic enteral feeding, and wherein determining whether the subject is a candidate subject comprises determining that the subject is likely to be a responder responsive to the classification of the subject comprising subphenotype B from the two or more subphenotypes.

92. A non-transitory computer readable medium comprising instructions that, when executed by a processor, cause the processor to:
obtain or have obtained electronic health record (EHR) data for a subject exhibiting acute respiratory distress syndrome (ARDS); and
determine a classification of the subject selected from two or more subphenotypes by analyzing, using a patient subphenotype classifier, the EHR data for the subject without analyzing biomarker levels of the subject.

93. The non-transitory computer readable medium of claim 92, wherein the patient subphenotype classifier receives one or more input variables comprising heart rate, mean arterial pressure, and respiratory rate.

94. The non-transitory computer readable medium of claim 93, wherein the patient subphenotype classifier receives each of the input variables of heart rate, mean arterial pressure, and respiratory rate.

95. The non-transitory computer readable medium of claim 93 or 94, wherein the patient subphenotype classifier further receives one or more input variables comprising arterial pH, partial pressure of oxygen, and bicarbonate.

96. The non-transitory computer readable medium of claim 95, wherein the patient subphenotype classifier further receives each of the input variables comprising arterial pH, partial pressure of oxygen, and bicarbonate.

97. The non-transitory computer readable medium of any one of claims 93-96, wherein the patient subphenotype classifier further receives one or more input variables comprising inspirited fraction of oxygen, creatinine, and bilirubin.

98. The non-transitory computer readable medium of claim 97, wherein the patient subphenotype classifier further receives each of the input variables comprising inspirited fraction of oxygen, creatinine, and bilirubin.

99. The non-transitory computer readable medium of any one of claims 93-98, wherein the patient subphenotype classifier further receives one or more input variables comprising partial pressure of carbon dioxide, PaO₂/FiO₂, platelet count, age, gender, positive end-expiratory pressure, and tidal volume.

100. The non-transitory computer readable medium of claim 99, wherein the patient subphenotype classifier further receives each of the input variables comprising partial pressure of carbon dioxide, PaO₂/FiO₂, platelet count, age, gender, positive end-expiratory pressure, and tidal volume.

101. The non-transitory computer readable medium of any one of claims 93-100, wherein the patient subphenotype classifier further receives one or more input variables comprising body mass index, plateau pressure, minute ventilation, and vasopressor use in prior 24 hours.

102. The non-transitory computer readable medium of claim 101, wherein the patient subphenotype classifier further receives each of the input variables comprising body mass index, plateau pressure, minute ventilation, and vasopressor use in prior 24 hours.

103. The non-transitory computer readable medium of claim 93, wherein the patient subphenotype classifier comprises a subphenotyping submodel that outputs a prediction for an ARDS subphenotype.

104. The non-transitory computer readable medium of claim 93, wherein the patient subphenotype classifier comprises a mortality submodel that outputs a prediction of an ARDS mortality rate.

105. The non-transitory computer readable medium of claim 93, wherein the patient subphenotype classifier comprises:
(A) a subphenotyping submodel that outputs a prediction for an ARDS subphenotype; and
(B) a mortality submodel that outputs a prediction of an ARDS mortality rate.

106. The non-transitory computer readable medium of claim 105, wherein the prediction for the ARDS subphenotype outputted by the subphenotyping submodel serves as an input to the mortality submodel.

107. The non-transitory computer readable medium of any one of claims 103 or 105-106, wherein the subphenotyping submodel receives one or more input variables comprising the subject’s arterial pH, bicarbonate, creatinine, fraction of inspired oxygen (FiO₂), heart rate, arterial pressure, respiration rate, and partial pressure of oxygen (PaO₂).

108. The non-transitory computer readable medium of any one of claims 103 or 105-107, wherein the subphenotyping submodel receives each of the input variables of the subject’s arterial pH, bicarbonate, creatinine, fraction of inspired oxygen (FIO₂), heart rate, arterial pressure, respiration rate, and partial pressure of oxygen (PaO₂).

109. The non-transitory computer readable medium of any one of claims 103 or 105-108, wherein implementation of the subphenotyping submodel comprises implementing an unsupervised clustering algorithm.

110. The non-transitory computer readable medium of any one of claims 104-109, wherein the mortality submodel receives input variables comprising the subject’s gender and age.

111. The non-transitory computer readable medium of any one of claims 104-110, wherein the mortality submodel receives input variables comprising the subject’s bilirubin, partial pressure of carbon dioxide (PaCO₂), PaO₂/FiO₂, positive end expiratory pressure (PEEP), platelet count, and tidal volume.

112. The non-transitory computer readable medium of any one of claims 104-110, wherein the mortality submodel receives input variables comprising the subject’s arterial pH, bicarbonate, creatinine, fraction of inspired oxygen (FiO₂), heart rate, arterial pressure, respiration rate, and partial pressure of oxygen (PaO₂).

113. The non-transitory computer readable medium of any one of claims 104-110, wherein the mortality submodel receives 10 or more input variables comprising the prediction for the ARDS subphenotype outputted by the subphenotyping submodel, the subject’s gender, age, bilirubin, partial pressure of carbon dioxide (PaCO₂), PaO₂/FiO_{2,} positive end expiratory pressure (PEEP), platelet count, tidal volume, and BMI.

114. The non-transitory computer readable medium of claim 113, wherein the patient subphenotype classifier has at least one of an area under receiver-operator curve (AUROC) greater than or equal to 0.689 and an area under the precision-recall curve (AUPRC) greater than or equal to 0.650.

115. The non-transitory computer readable medium of any one of claims 104-110, wherein the mortality submodel receives 9 or more input variables comprising the prediction for the ARDS subphenotype outputted by the subphenotyping submodel, the subject’s gender, age, bilirubin, partial pressure of carbon dioxide (PaCO₂), PaO₂/FiO_{2,} positive end expiratory pressure (PEEP), platelet count, and tidal volume.

116. The non-transitory computer readable medium of claim 115, wherein the patient subphenotype classifier has at least one of an area under receiver-operator curve (AUROC) greater than or equal to 0.673 and an area under the precision-recall curve (AUPRC) greater than or equal to 0.668.

117. The non-transitory computer readable medium of any one of claims 104-110, wherein the mortality submodel receives 12 or more input variables comprising the prediction for the ARDS subphenotype outputted by the subphenotyping submodel, the subject’s gender, age, bilirubin, arterial pH, bicarbonate, creatinine, fraction of inspired oxygen (FIO₂), heart rate, arterial pressure, respiration rate, and partial pressure of oxygen (PaO₂).

118. The non-transitory computer readable medium of claim 117, wherein the patient subphenotype classifier has at least one of an area under receiver-operator curve (AUROC) greater than or equal to 0.658 and an area under the precision-recall curve (AUPRC) greater than or equal to 0.597.

119. The non-transitory computer readable medium of any one of claims 104-110, wherein the mortality submodel receives 11 or more input variables comprising the prediction for the ARDS subphenotype outputted by the subphenotyping submodel, the subject’s gender, age, arterial pH, bicarbonate, creatinine, fraction of inspired oxygen (FiO₂), heart rate, arterial pressure, respiration rate, and partial pressure of oxygen (PaO₂).

120. The non-transitory computer readable medium of claim 119, wherein the patient subphenotype classifier has at least one of an area under receiver-operator curve (AUROC) greater than or equal to 0.643 and an area under the precision-recall curve (AUPRC) greater than or equal to 0.532.

121. The non-transitory computer readable medium of any one of claims 104-120, wherein implementation of the mortality submodel comprises implementing a supervised machine learning algorithm.

122. The non-transitory computer readable medium of any one of claims 104-121, wherein the instructions that cause the processor to determine the classification of the subject based on the EHR data using the patient subphenotype classifier further comprises instructions that, when executed by the processor, cause the processor to:
determine that data elements of a higher rank mortality submodel are unavailable in the EHR data; and
determine that data elements of the mortality submodel are available in the EHR data.

123. The non-transitory computer readable medium of any one of claims 104-120, wherein the instructions that cause the processor to determine the classification of the subject based on the EHR data using the patient subphenotype classifier further comprises instructions that, when executed by the processor, cause the processor to implement the mortality submodel responsive to determining that data elements of the mortality submodel are available in the EHR data.

124. The non-transitory computer readable medium of any one of claims 105-109, wherein the mortality submodel comprises two or more sub-models that each outputs a prediction informative for determining an ARDS mortality rate.

125. The non-transitory computer readable medium of claim 124, wherein the first submodel receives input variables comprising a first prediction for the ARDS subphenotype outputted by the subphenotyping submodel and the second sub-model receives input variables comprising a second prediction for the ARDS subphenotype outputted by the subphenotyping submodel.

126. The non-transitory computer readable medium of claim 125, wherein the first submodel receives input variables further comprising the subject’s bilirubin.

127. The non-transitory computer readable medium of claim 125, wherein the second submodel receives input variables further comprising the subject’s bilirubin, partial pressure of carbon dioxide (PaCO₂), PaO₂/FiO_{2,} positive end expiratory pressure (PEEP), platelet count, and tidal volume.

128. The non-transitory computer readable medium of any one of claims 103 or 105-123, wherein the subphenotyping submodel comprises two or more sub-models that each outputs a prediction of an ARDS subphenotype.

129. The non-transitory computer readable medium of claim 128, wherein implementation of the two or more sub-models comprises implementing unsupervised clustering algorithms.

130. The non-transitory computer readable medium of any one of claims 103 or 105-123, wherein the patient subphenotype classifier further comprises a pre-mortality model that outputs a prediction that serves as input to the mortality submodel.

131. The non-transitory computer readable medium of claim 130, wherein implementation of the pre-mortality model comprises implementing a supervised machine learning algorithm.

132. The non-transitory computer readable medium of claim 104, wherein the mortality submodel receives, as input, 8 or more input variables.

133. The non-transitory computer readable medium of claim 132, wherein the 8 or more input variables comprise at least the subject’s arterial pH, bicarbonate, creatinine, fraction of inspired oxygen (FiO₂), and heart rate.

134. The non-transitory computer readable medium of claim 133, wherein the 8 or more input variables further comprise at least the subject’s airway pressure, arterial pressure, respiration rate, and partial pressure of oxygen (PaO₂).

135. The non-transitory computer readable medium of claim 132, wherein the patient subphenotype classifier comprises one of a first model, a second model, a third model, and a fourth model,
wherein the first model receives, as input, 13 input variables,
wherein the second model receives, as input, 8 input variables,
wherein the third model receives, as input, 17 input variables, and
wherein the fourth model receives, as input, 13 input variables.

136. The non-transitory computer readable medium of claim 135, wherein the 13 input variables of the first model comprise the subject’s arterial pH, bicarbonate, creatinine, diastolic blood pressure (BP), FiO₂, heart rate, highest mean arterial pressure, lowest mean arterial pressure, potassium, highest respiratory rate, lowest respiratory rate, SPO₂, and systolic BP.

137. The non-transitory computer readable medium of claim 135 or 136, wherein the 13 input variables of the first model comprise the subject’s most recent arterial pH, lowest bicarbonate, most recent creatinine, most recent diastolic blood pressure (BP), most recent FiO₂, most recent heart rate, highest mean arterial pressure, lowest mean arterial pressure, most recent potassium, highest respiratory rate, lowest respiratory rate, most recent SPO₂, and most recent systolic BP.

138. The non-transitory computer readable medium of any one of claims 135-137, wherein the patient subphenotype classifier has at least one of an area under receiver-operator curve (AUROC) greater than or equal to 0.67 and an area under the precision-recall curve (AUPRC) greater than or equal to 0.40.

139. The non-transitory computer readable medium of claim 135, wherein the 8 input variables of the second model comprise the subject’s arterial pH, bicarbonate, creatinine, FiO₂, heart rate, PaO₂, mean arterial pressure, and respiratory rate.

140. The non-transitory computer readable medium of claim 135 or 139, wherein the 8 input variables of the second model comprise the subject’s most recent arterial pH, lowest bicarbonate, most recent creatinine, most recent FiO₂, most recent heart rate, most recent PaO₂, most recent mean arterial pressure, and most recent respiratory rate.

141. The non-transitory computer readable medium of any one of claims 135 or 139-140, wherein the patient subphenotype classifier has at least one of an area under receiver-operator curve (AUROC) greater than or equal to 0.69 and an area under the precision-recall curve (AUPRC) greater than or equal to 0.42.

142. The non-transitory computer readable medium of claim 135, wherein the 17 input variables of the third model comprise the subject’s age, arterial pH, bicarbonate, bilirubin, BMI, creatinine, FiO₂, gender, heart rate, PaCO₂, PaO₂/FiO_{2,} PaO₂, positive end-expiratory pressure (PEEP), platelet count, tidal volume, mean arterial pressure, and respiratory rate.

143. The non-transitory computer readable medium of claim 135 or 142, wherein the 17 input variables of the third model comprise the subject’s age, most recent arterial pH, lowest bicarbonate, highest bilirubin, BMI, most recent creatinine, most recent FiO₂, gender, most recent heart rate, most recent PaCO₂, lowest PaO₂/FiO₂ within 24 hours following ARDS diagnosis, most recent PaO₂, most recent positive end-expiratory pressure (PEEP), lowest platelet count, lowest tidal volume, most recent mean arterial pressure, and most recent respiratory rate.

144. The non-transitory computer readable medium of any one of claims 135 or 142-143, wherein the patient subphenotype classifier has at least one of an area under receiver-operator curve (AUROC) greater than or equal to 0.71 and an area under the precision-recall curve (AUPRC) greater than or equal to 0.62.

145. The non-transitory computer readable medium of claim 135, wherein the 13 input variables of the fourth model comprise the subject’s arterial pH, bicarbonate, BMI, creatinine, FiO₂, gender, heart rate, PaCO₂, PaO₂/FiO₂, PEEP, platelet count, mean arterial pressure, and respiratory rate.

146. The non-transitory computer readable medium of claim 135 or 145, wherein the 13 input variables of the fourth model comprise the subject’s most recent arterial pH, most recent bicarbonate, BMI, most recent creatinine, most recent FiO₂, gender, most recent heart rate, most recent PaCO₂, lowest PaO₂/FiO₂ within 24 hours following ARDS diagnosis, most recent PEEP, lowest platelet count, most recent mean arterial pressure, and most recent respiratory rate.

147. The non-transitory computer readable medium of any one of claims 135 or 145-146, wherein the patient subphenotype classifier has at least one of an area under receiver-operator curve (AUROC) greater than or equal to 0.67 and an area under the precision-recall curve (AUPRC) greater than or equal to 0.46.

148. The non-transitory computer readable medium of claim 92, wherein the classification of the subject is selected from three or more subphenotypes.

149. The non-transitory computer readable medium of claim 148, wherein the three or more subphenotypes comprise a lower risk subphenotype, a medium risk subphenotype, and a high risk subphenotype.

150. The non-transitory computer readable medium of claim 148 or 149, wherein the classification of the subject is selected from three by comparing a score to two threshold values.

151. The non-transitory computer readable medium of any one of claims 148-150, wherein the patient subphenotype classifier has at least an area under receiver-operator curve (AUROC) greater than or equal to 0.691.

152. The non-transitory computer readable medium of any one of claims 92-151, wherein the patient subphenotype classifier is trained using a training dataset comprising patient data from one or more clinical trial datasets.

153. The non-transitory computer readable medium of claim 152, wherein the one or more clinical trial datasets are any of ARMA dataset, KARMA dataset, LARMA dataset, ALVEOLI dataset, EDEN dataset, FACTT dataset, SAILS dataset, ROSE dataset, eICU-CRD dataset, and the Brazillian ART dataset.

154. The non-transitory computer readable medium of claim 152 or 153, wherein the patient data is derived from a sub-cohort of patients of the one or more clinical trial datasets, wherein the sub-cohort of patients are characterized by having a ratio of arterial oxygen concentration to the fraction of inspired oxygen (P/F ratio) of less than or equal to 200.

155. The non-transitory computer readable medium of claim 152 or 153, wherein the patient data is derived from a sub-cohort of patients of the one or more clinical trial datasets, wherein the sub-cohort of patients are characterized by having a ratio of arterial oxygen concentration to the fraction of inspired oxygen (P/F ratio) of less than or equal to 300.

156. The non-transitory computer readable medium of any one of claims 92-155, wherein the two or more subphenotypes comprise subphenotype A and subphenotype B that are characterized by differences in expression levels in one or more biomarkers.

157. The non-transitory computer readable medium of claim 156, wherein the one or more biomarkers comprise one or more of PAI-1, IL-6, IL-8, IL-10, TNFR-I, TNFR-II, ICAM-1, or von Willebrand factor.

158. The non-transitory computer readable medium of claim 156, wherein the one or more biomarkers comprise each of PAI-1, IL-6, IL-8, IL-10, TNFR-I, TNFR-II, ICAM-1, or von Willebrand factor.

159. A non-transitory computer readable medium comprising instructions that, when executed by a processor, cause the processor to:
obtain a classification of the subject exhibiting acute respiratory distress syndrome (ARDS), the classification of the subject selected from two or more subphenotypes and determined using the non-transitory computer readable medium of any one of claims 92-158; and
identify a mortality prognosis for the subject based at least in part on the classification,
wherein responsive to the classification of the subject comprising subphenotype B from the two or more subphenotypes, the mortality prognosis identified for the subject comprises high mortality risk, and
wherein responsive to the classification of the subject comprising subphenotype A from the two or more subphenotypes, the mortality prognosis identified for the subject comprises low mortality risk.

160. The non-transitory computer readable medium of claim 159, wherein low mortality risk comprises at least one of reduced risk of hospital mortality, reduced risk of ICU mortality, reduced risk of 28-day mortality, reduced risk of 90-day mortality, reduced risk of 180-day mortality, and reduced risk of 6-month mortality relative to high mortality risk.

161. The non-transitory computer readable medium of claim 159 or 160, wherein low mortality risk further comprises positive patient outcome, wherein high mortality risk further comprises negative patient outcome, and wherein positive patient outcome comprises at least one of shorter hospital length of stay, shorter ICU length of stay and more ventilator-free days relative to negative patient outcome.

162. A non-transitory computer readable medium comprising instructions that, when executed by a processor, cause the processor to:
obtain a classification of a subject exhibiting acute respiratory distress syndrome (ARDS), the classification of the subject selected from two or more subphenotypes and determined using the non-transitory computer readable medium of any one of claims 92-158; and
identify a therapy recommendation for the subject based at least in part on the classification,
wherein responsive to the classification of the subject comprising subphenotype B from the two or more subphenotypes, the therapy recommendation identified for the subject comprises one or more of neuromuscular blockade (NMB) therapy or no NMB therapy, high PEEP or low PEEP, no treatment or methylprednisolone, dexamethasone, no lisofylline, ketoconazole, catheter and fluid treatment, recruitment maneuver, statins, or full or trophic enteral feeding and
wherein responsive to the classification of the subject comprising subphenotype A from the two or more subphenotypes, the therapy recommendation identified for the subject comprises one or more of NMB therapy, low PEEP therapy, no methylprednisolone, no treatment or dexamethasone, no treatment or lisofylline, no treatment or ketoconazole, no combination of catheter and fluid treatment, no recruitment maneuver, statins as a preemptive therapy, or full enteral feeding.

163. A non-transitory computer readable medium comprising instructions that, when executed by a processor, cause the processor to:
for one or more subjects, obtain a classification of the subject exhibiting acute respiratory distress syndrome (ARDS), the classification of the subject selected from two or more subphenotypes and determined using the non-transitory computer readable medium of any one of claims 92-158; and
determine whether the subject is a candidate subject based at least in part on the classification.

164. The non-transitory computer readable medium of claim 163, wherein the therapy is a neuromuscular blockade (NMB) therapy, and wherein determining whether the subject is a candidate subject comprises determining that the subject is a likely responder responsive to the classification of the subject comprising subphenotype A from the two or more subphenotypes.

165. The non-transitory computer readable medium of claim 163, wherein the therapy is a neuromuscular blockade (NMB) therapy, and wherein determining whether the subject is a candidate subject comprises determining that the subject is unlikely to be a responder responsive to the classification of the subject comprising subphenotype B from the two or more subphenotypes.

166. The non-transitory computer readable medium of claim 163, wherein the therapy is a low positive end-expiratory pressure (PEEP) treatment, and wherein determining whether the subject is a candidate subject comprises determining that the subject is likely to be a responder responsive to the classification of the subject comprising subphenotype A from the two or more subphenotypes.

167. The non-transitory computer readable medium of claim 163, wherein the therapy is a high positive end-expiratory pressure (PEEP) treatment, and wherein determining whether the subject is a candidate subject comprises determining that the subject is likely to be a responder responsive to the classification of the subject comprising subphenotype B from the two or more subphenotypes.

168. The non-transitory computer readable medium of claim 163, wherein the therapy is a corticosteroid treatment, and wherein determining whether the subject is a candidate subject comprises determining that the subject is likely to be a responder responsive to the classification of the subject comprising subphenotype B from the two or more subphenotypes.

169. The non-transitory computer readable medium of claim 163, wherein the therapy is a corticosteroid treatment, and wherein determining whether the subject is a candidate subject comprises determining that the subject is unlikely to be a responder responsive to the classification of the subject comprising subphenotype A from the two or more subphenotypes.

170. The non-transitory computer readable medium of claim 168 or 169, wherein the corticosteroid treatment is methylpredinosolone or dexamethasone.

171. The non-transitory computer readable medium of claim 163, wherein the therapy is a lisofylline treatment, and wherein determining whether the subject is a candidate subject comprises determining that the subject is unlikely to be a responder responsive to the classification of the subject comprising subphenotype B from the two or more subphenotypes.

172. The non-transitory computer readable medium of claim 163, wherein the therapy is a lisofylline treatment, and wherein determining whether the subject is a candidate subject comprises determining that the subject is likely to be a responder responsive to the classification of the subject comprising subphenotype A from the two or more subphenotypes.

173. The non-transitory computer readable medium of claim 163, wherein the therapy is a ketoconazole treatment, and wherein determining whether the subject is a candidate subject comprises determining that the subject is likely to be a responder responsive to the classification of the subject comprising subphenotype B from the two or more subphenotypes.

174. The non-transitory computer readable medium of claim 163, wherein the therapy is a pulmonary artery catheter and liberal fluid treatment, and wherein determining whether the subject is a candidate subject comprises determining that the subject is likely to be a responder responsive to the classification of the subject comprising subphenotype B from the two or more subphenotypes.

175. The non-transitory computer readable medium of claim 163, wherein the therapy is a pulmonary artery catheter and liberal fluid treatment, and wherein determining whether the subject is a candidate subject comprises determining that the subject is unlikely to be a responder responsive to the classification of the subject comprising subphenotype A from the two or more subphenotypes.

176. The non-transitory computer readable medium of claim 174 or 175, wherein the catheter and fluid treatment comprises a central venous catheter line treatment or a pulmonary artery catheter line treatment.

177. The non-transitory computer readable medium of claim 163, wherein the therapy is a recruitment maneuver, and wherein determining whether the subject is a candidate subject comprises determining that the subject is likely to be a responder responsive to the classification of the subject comprising subphenotype B from the two or more subphenotypes.

178. The non-transitory computer readable medium of claim 163, wherein the therapy is a recruitment maneuver, and wherein determining whether the subject is a candidate subject comprises determining that the subject is unlikely to be a responder responsive to the classification of the subject comprising subphenotype A from the two or more subphenotypes.

179. The non-transitory computer readable medium of claim 163, wherein the therapy is a statin treatment, and wherein determining whether the subject is a candidate subject comprises determining that the subject is likely to be a responder responsive to the classification of the subject comprising subphenotype B from the two or more subphenotypes.

180. The non-transitory computer readable medium of claim 163, wherein the therapy is a preemptive statin treatment, and wherein determining whether the subject is a candidate subject comprises determining that the subject is likely to be a responder responsive to the classification of the subject comprising subphenotype A from the two or more subphenotypes.

181. The non-transitory computer readable medium of claim 163, wherein the therapy is full enteral feeding, and wherein determining whether the subject is a candidate subject comprises determining that the subject is likely to be a responder responsive to the classification of the subject comprising subphenotype A from the two or more subphenotypes.

182. The non-transitory computer readable medium of claim 163, wherein the therapy is trophic enteral feeding, and wherein determining whether the subject is a candidate subject comprising determining that the subject is likely to be a responder responsive to the classification of the subject comprising subphenotype B from the two or more subphenotypes.

183. A system comprising:
a storage memory configured to store electronic health record (EHR) data for a subject exhibiting acute respiratory distress syndrome (ARDS); and
a processor communicatively coupled to the storage memory to determine a classification of the subject selected from two or more subphenotypes by analyzing, using a patient subphenotype classifier, the EHR data for the subject without analyzing biomarker levels of the subject.

184. The system of claim 183, wherein the patient subphenotype classifier receives one or more input variables comprising heart rate, mean arterial pressure, and respiratory rate.

185. The system of claim 184, wherein the patient subphenotype classifier receives each of the input variables of heart rate, mean arterial pressure, and respiratory rate.

186. The system of claim 184 or 185, wherein the patient subphenotype classifier further receives one or more input variables comprising arterial pH, partial pressure of oxygen, and bicarbonate.

187. The system of claim 186, wherein the patient subphenotype classifier further receives each of the input variables comprising arterial pH, partial pressure of oxygen, and bicarbonate.

188. The system of any one of claims 184-187, wherein the patient subphenotype classifier further receives one or more input variables comprising inspirited fraction of oxygen, creatinine, and bilirubin.

189. The system of claim 188, wherein the patient subphenotype classifier further receives each of the input variables comprising inspirited fraction of oxygen, creatinine, and bilirubin.

190. The system of any one of claims 184-189, wherein the patient subphenotype classifier further receives one or more input variables comprising partial pressure of carbon dioxide, PaO₂/FiO_{2,} platelet count, age, gender, positive end-expiratory pressure, and tidal volume.

191. The system of claim 190, wherein the patient subphenotype classifier further receives each of the input variables comprising partial pressure of carbon dioxide, PaO₂/FiO_{2,} platelet count, age, gender, positive end-expiratory pressure, and tidal volume.

192. The system of any one of claims 184-191, wherein the patient subphenotype classifier further receives one or more input variables comprising body mass index, plateau pressure, minute ventilation, and vasopressor use in prior 24 hours.

193. The system of claim 192, wherein the patient subphenotype classifier further receives each of the input variables comprising body mass index, plateau pressure, minute ventilation, and vasopressor use in prior 24 hours.

194. The system of claim 184, wherein the patient subphenotype classifier comprises a subphenotyping submodel that outputs a prediction for an ARDS subphenotype.

195. The system of claim 184, wherein the patient subphenotype classifier comprises a mortality submodel that outputs a prediction of an ARDS mortality rate.

196. The system of claim 184, wherein the patient subphenotype classifier comprises:
(A) a subphenotyping submodel that outputs a prediction for an ARDS subphenotype; and
(B) a mortality submodel that outputs a prediction of an ARDS mortality rate.

197. The system of claim 196, wherein the prediction for the ARDS subphenotype outputted by the subphenotyping submodel serves as an input to the mortality submodel.

198. The system of any one of claims 194 or 196-197, wherein the subphenotyping submodel receives one or more input variables comprising the subject’s arterial pH, bicarbonate, creatinine, fraction of inspired oxygen (FiO₂), heart rate, arterial pressure, respiration rate, and partial pressure of oxygen (PaO₂).

199. The system of any one of claims 194 or 196-198, wherein the subphenotyping submodel receives each of the input variables of the subject’s arterial pH, bicarbonate, creatinine, fraction of inspired oxygen (FIO₂), heart rate, arterial pressure, respiration rate, and partial pressure of oxygen (PaO₂).

200. The system of any one of claims 194 or 196-199, wherein implementation of the subphenotyping submodel comprises implementing an unsupervised clustering algorithm.

201. The system of any one of claims 195-200, wherein the mortality submodel receives input variables comprising the subject’s gender and age.

202. The system of any one of claims 195-201, wherein the mortality submodel receives input variables comprising the subject’s bilirubin, partial pressure of carbon dioxide (PaCO₂), PaO₂/FiO_{2,} positive end expiratory pressure (PEEP), platelet count, and tidal volume.

203. The system of any one of claims 195-201, wherein the mortality submodel receives input variables comprising the subject’s arterial pH, bicarbonate, creatinine, fraction of inspired oxygen (FiO₂), heart rate, arterial pressure, respiration rate, and partial pressure of oxygen (PaO₂).

204. The system of any one of claims 195-201, wherein the mortality submodel receives 10 or more input variables comprising the prediction for the ARDS subphenotype outputted by the subphenotyping submodel, the subject’s gender, age, bilirubin, partial pressure of carbon dioxide (PaCO₂), PaO₂/FiO_{2,} positive end expiratory pressure (PEEP), platelet count, tidal volume, and BMI.

205. The system of claim 204, wherein the patient subphenotype classifier has at least one of an area under receiver-operator curve (AUROC) greater than or equal to 0.689 and an area under the precision-recall curve (AUPRC) greater than or equal to 0.650.

206. The system of any one of claims 195-201, wherein the mortality submodel receives 9 or more input variables comprising the prediction for the ARDS subphenotype outputted by the subphenotyping submodel, the subject’s gender, age, bilirubin, partial pressure of carbon dioxide (PaCO₂), PaO₂/FiO_{2,} positive end expiratory pressure (PEEP), platelet count, and tidal volume.

207. The system of claim 206, wherein the patient subphenotype classifier has at least one of an area under receiver-operator curve (AUROC) greater than or equal to 0.673 and an area under the precision-recall curve (AUPRC) greater than or equal to 0.668.

208. The system of any one of claims 195-201, wherein the mortality submodel receives 12 or more input variables comprising the prediction for the ARDS subphenotype outputted by the subphenotyping submodel, the subject’s gender, age, bilirubin, arterial pH, bicarbonate, creatinine, fraction of inspired oxygen (FIO₂), heart rate, arterial pressure, respiration rate, and partial pressure of oxygen (PaO₂).

209. The system of claim 208, wherein the patient subphenotype classifier has at least one of an area under receiver-operator curve (AUROC) greater than or equal to 0.658 and an area under the precision-recall curve (AUPRC) greater than or equal to 0.597.

210. The system of any one of claims 195-201, wherein the mortality submodel receives 11 or more input variables comprising the prediction for the ARDS subphenotype outputted by the subphenotyping submodel, the subject’s gender, age, arterial pH, bicarbonate, creatinine, fraction of inspired oxygen (FiO₂), heart rate, arterial pressure, respiration rate, and partial pressure of oxygen (PaO₂).

211. The system of claim 210, wherein the patient subphenotype classifier has at least one of an area under receiver-operator curve (AUROC) greater than or equal to 0.643 and an area under the precision-recall curve (AUPRC) greater than or equal to 0.532.

212. The system of any one of claims 195-211, wherein implementation of the mortality submodel comprises implementing a supervised machine learning algorithm.

213. The system of any one of claims 195-212, wherein the instructions that cause the processor to determine the classification of the subject based on the EHR data using the patient subphenotype classifier further comprises instructions that, when executed by the processor, cause the processor to:
determine that data elements of a higher rank mortality submodel are unavailable in the EHR data; and
determine that data elements of the mortality submodel are available in the EHR data.

214. The system of any one of claims 195-211, wherein the instructions that cause the processor to determine the classification of the subject based on the EHR data using the patient subphenotype classifier further comprises instructions that, when executed by the processor, cause the processor to implement the mortality submodel responsive to determining that data elements of the mortality submodel are available in the EHR data.

215. The system of any one of claims 196-200, wherein the mortality submodel comprises two or more sub-models that each outputs a prediction informative for determining an ARDS mortality rate.

216. The system of claim 215, wherein the first sub-model receives input variables comprising a first prediction for the ARDS subphenotype outputted by the subphenotyping submodel and the second sub-model receives input variables comprising a second prediction for the ARDS subphenotype outputted by the subphenotyping submodel.

217. The system of claim 216, wherein the first sub-model receives input variables further comprising the subject’s bilirubin.

218. The system of claim 216, wherein the second sub-model receives input variables further comprising the subject’s bilirubin, partial pressure of carbon dioxide (PaCO₂), PaO₂/FiO_{2,} positive end expiratory pressure (PEEP), platelet count, and tidal volume.

219. The system of any one of claims 194 or 196-214, wherein the subphenotyping submodel comprises two or more sub-models that each outputs a prediction of an ARDS subphenotype.

220. The system of claim 219, wherein implementation of the two or more sub-models comprises implementing unsupervised clustering algorithms.

221. The system of any one of claims 194 or 196-214, wherein the patient subphenotype classifier further comprises a pre-mortality model that outputs a prediction that serves as input to the mortality submodel.

222. The system of claim 221, wherein implementation of the pre-mortality model comprises implementing a supervised machine learning algorithm.

223. The system of claim 194, wherein the mortality submodel receives, as input, 8 or more input variables.

224. The system of claim 223, wherein the 8 or more input variables comprise at least the subject’s arterial pH, bicarbonate, creatinine, fraction of inspired oxygen (FiO₂), and heart rate.

225. The system of claim 224, wherein the 8 or more input variables further comprise at least the subject’s airway pressure, arterial pressure, respiration rate, and partial pressure of oxygen (PaO₂).

226. The system of claim 223, wherein the patient subphenotype classifier comprises one of a first model, a second model, a third model, and a fourth model,
wherein the first model receives, as input, 13 input variables,
wherein the second model receives, as input, 8 input variables,
wherein the third model receives, as input, 17 input variables, and
wherein the fourth model receives, as input, 13 input variables.

227. The system of claim 226, wherein the 13 input variables of the first model comprise the subject’s arterial pH, bicarbonate, creatinine, diastolic blood pressure (BP), FiO₂, heart rate, highest mean arterial pressure, lowest mean arterial pressure, potassium, highest respiratory rate, lowest respiratory rate, SPO₂, and systolic BP.

228. The system of claim 226 or 227, wherein the 13 input variables of the first model comprise the subject’s most recent arterial pH, lowest bicarbonate, most recent creatinine, most recent diastolic blood pressure (BP), most recent FiO₂, most recent heart rate, highest mean arterial pressure, lowest mean arterial pressure, most recent potassium, highest respiratory rate, lowest respiratory rate, most recent SPO₂, and most recent systolic BP.

229. The system of any one of claims 226-228, wherein the patient subphenotype classifier has at least one of an area under receiver-operator curve (AUROC) greater than or equal to 0.67 and an area under the precision-recall curve (AUPRC) greater than or equal to 0.40.

230. The system of claim 226, wherein the 8 input variables of the second model comprise the subject’s arterial pH, bicarbonate, creatinine, FiO₂, heart rate, PaO₂, mean arterial pressure, and respiratory rate.

231. The system of claim 226 or 230, wherein the 8 input variables of the second model comprise the subject’s most recent arterial pH, lowest bicarbonate, most recent creatinine, most recent FiO₂, most recent heart rate, most recent PaO₂, most recent mean arterial pressure, and most recent respiratory rate.

232. The system of any one of claims 226 or 230-231, wherein the patient subphenotype classifier has at least one of an area under receiver-operator curve (AUROC) greater than or equal to 0.69 and an area under the precision-recall curve (AUPRC) greater than or equal to 0.42.

233. The system of claim 226, wherein the 17 input variables of the third model comprise the subject’s age, arterial pH, bicarbonate, bilirubin, BMI, creatinine, FiO₂, gender, heart rate, PaO₂, PaO₂/FiO_{2,} PaO₂, positive end-expiratory pressure (PEEP), platelet count, tidal volume, mean arterial pressure, and respiratory rate.

234. The system of claim 226 or 233, wherein the 17 input variables of the third model comprise the subject’s age, most recent arterial pH, lowest bicarbonate, highest bilirubin, BMI, most recent creatinine, most recent FiO₂, gender, most recent heart rate, most recent PaCO₂, lowest PaO₂/FiO₂ within 24 hours following ARDS diagnosis, most recent PaO₂, most recent positive end-expiratory pressure (PEEP), lowest platelet count, lowest tidal volume, most recent mean arterial pressure, and most recent respiratory rate.

235. The system of any one of claims 226 or 233-234, wherein the patient subphenotype classifier has at least one of an area under receiver-operator curve (AUROC) greater than or equal to 0.71 and an area under the precision-recall curve (AUPRC) greater than or equal to 0.62.

236. The system of claim 226, wherein the 13 input variables of the fourth model comprise the subject’s arterial pH, bicarbonate, BMI, creatinine, FiO₂, gender, heart rate, PaCO₂, PaO₂/FiO₂, PEEP, platelet count, mean arterial pressure, and respiratory rate.

237. The system of claim 226 or 236, wherein the 13 input variables of the fourth model comprise the subject’s most recent arterial pH, most recent bicarbonate, BMI, most recent creatinine, most recent FiO₂, gender, most recent heart rate, most recent PaCO₂, lowest PaO₂/FiO₂ within 24 hours following ARDS diagnosis, most recent PEEP, lowest platelet count, most recent mean arterial pressure, and most recent respiratory rate.

238. The system of any one of claims 226 or 236-237, wherein the patient subphenotype classifier has at least one of an area under receiver-operator curve (AUROC) greater than or equal to 0.67 and an area under the precision-recall curve (AUPRC) greater than or equal to 0.46.

239. The system of claim 183, wherein the classification of the subject is selected from three or more subphenotypes.

240. The system of claim 239, wherein the three or more subphenotypes comprise a lower risk subphenotype, a medium risk subphenotype, and a high risk subphenotype.

241. The system of claim 239 or 240, wherein the classification of the subject is selected from three by comparing a score to two threshold values.

242. The system of any one of claims 239-241, wherein the patient subphenotype classifier has at least an area under receiver-operator curve (AUROC) greater than or equal to 0.691.

243. The system of any one of claims 183-242, wherein the patient subphenotype classifier is trained using a training dataset comprising patient data from one or more clinical trial datasets.

244. The system of claim 243, wherein the one or more clinical trial datasets are any of ARMA dataset, KARMA dataset, LARMA dataset, ALVEOLI dataset, EDEN dataset, FACTT dataset, SAILS dataset, ROSE dataset, eICU-CRD dataset, and the Brazillian ART dataset.

245. The system of claim 243 or 244, wherein the patient data is derived from a sub-cohort of patients of the one or more clinical trial datasets, wherein the sub-cohort of patients are characterized by having a ratio of arterial oxygen concentration to the fraction of inspired oxygen (P/F ratio) of less than or equal to 200.

246. The system of claim 243 or 244, wherein the patient data is derived from a sub-cohort of patients of the one or more clinical trial datasets, wherein the sub-cohort of patients are characterized by having a ratio of arterial oxygen concentration to the fraction of inspired oxygen (P/F ratio) of less than or equal to 300.

247. The system of any one of claims 183-246, wherein the two or more subphenotypes comprise subphenotype A and subphenotype B that are characterized by differences in expression levels in one or more biomarkers.

248. The system of claim 247, wherein the one or more biomarkers comprise one or more of PAI-1, IL-6, IL-8, IL-10, TNFR-I, TNFR-II, ICAM-1, or von Willebrand factor.

249. The system of claim 247, wherein the one or more biomarkers comprise each of PAI-1, IL-6, IL-8, IL-10, TNFR-I, TNFR-II, ICAM-1, or von Willebrand factor.

250. A system comprising:
a storage memory configured to store electronic health record (EHR) data for a subject exhibiting acute respiratory distress syndrome (ARDS); and
a processor communicatively coupled to the storage memory to:
obtain a classification of the subject exhibiting acute respiratory distress syndrome (ARDS), the classification of the subject selected from two or more subphenotypes and determined using the system of any one of claims 183-249; and
identify a mortality prognosis for the subject based at least in part on the classification,
wherein responsive to the classification of the subject comprising subphenotype B from the two or more subphenotypes, the mortality prognosis identified for the subject comprises high mortality risk, and
wherein responsive to the classification of the subject comprising subphenotype A from the two or more subphenotypes, the mortality prognosis identified for the subject comprises low mortality risk.

251. The system of claim 250, wherein low mortality risk comprises at least one of reduced risk of hospital mortality, reduced risk of ICU mortality, reduced risk of 28-day mortality, reduced risk of 90-day mortality, reduced risk of 180-day mortality, and reduced risk of 6-month mortality relative to high mortality risk.

252. The system of claim 250 or 251, wherein low mortality risk further comprises positive patient outcome, wherein high mortality risk further comprises negative patient outcome, and wherein positive patient outcome comprises at least one of shorter hospital length of stay, shorter ICU length of stay and more ventilator-free days relative to negative patient outcome.

253. A system comprising:
a storage memory configured to store electronic health record (EHR) data for a subject exhibiting acute respiratory distress syndrome (ARDS); and
a processor communicatively coupled to the storage memory to:
obtain a classification of a subject exhibiting acute respiratory distress syndrome (ARDS), the classification of the subject selected from two or more subphenotypes and determined using the system of any one of claims 183-249; and
identify a therapy recommendation for the subject based at least in part on the classification,
wherein responsive to the classification of the subject comprising subphenotype B from the two or more subphenotypes, the therapy recommendation identified for the subject comprises one or more of neuromuscular blockade (NMB) therapy or no NMB therapy, high PEEP or low PEEP, no treatment or methylprednisolone, dexamethasone, no lisofylline, ketoconazole, catheter and fluid treatment, recruitment maneuver, statins, or full or trophic enteral feeding and
wherein responsive to the classification of the subject comprising subphenotype A from the two or more subphenotypes, the therapy recommendation identified for the subject comprises one or more of NMB therapy, low PEEP therapy, no methylprednisolone, no treatment or dexamethasone, no treatment or lisofylline, no treatment or ketoconazole, no combination of catheter and fluid treatment, no recruitment maneuver, statins as a preemptive therapy, or full enteral feeding.

254. A system comprising:
a storage memory configured to store electronic health record (EHR) data for a subject exhibiting acute respiratory distress syndrome (ARDS); and
a processor communicatively coupled to the storage memory to:
for one or more subjects, obtain a classification of the subject exhibiting acute respiratory distress syndrome (ARDS), the classification of the subject selected from two or more subphenotypes and determined using the system of any one of claims 183-249; and
determine whether the subject is a candidate subject based at least in part on the classification.

255. The system of claim 254, wherein the therapy is a neuromuscular blockade (NMB) therapy, and wherein determining whether the subject is a candidate subject comprises determining that the subject is a likely responder responsive to the classification of the subject comprising subphenotype A from the two or more subphenotypes.

256. The system of claim 254, wherein the therapy is a neuromuscular blockade (NMB) therapy, and wherein determining whether the subject is a candidate subject comprises determining that the subject is unlikely to be a responder responsive to the classification of the subject comprising subphenotype B from the two or more subphenotypes.

257. The system of claim 254, wherein the therapy is a low positive end-expiratory pressure (PEEP) treatment, and wherein determining whether the subject is a candidate subject comprises determining that the subject is likely to be a responder responsive to the classification of the subject comprising subphenotype A from the two or more subphenotypes.

258. The system of claim 254, wherein the therapy is a high positive end-expiratory pressure (PEEP) treatment, and wherein determining whether the subject is a candidate subject comprises determining that the subject is likely to be a responder responsive to the classification of the subject comprising subphenotype B from the two or more subphenotypes.

259. The system of claim 254, wherein the therapy is a corticosteroid treatment, and wherein determining whether the subject is a candidate subject comprises determining that the subject is likely to be a responder responsive to the classification of the subject comprising subphenotype B from the two or more subphenotypes.

260. The system of claim 254, wherein the therapy is a corticosteroid treatment, and wherein determining whether the subject is a candidate subject comprises determining that the subject is unlikely to be a responder responsive to the classification of the subject comprising subphenotype A from the two or more subphenotypes.

261. The system of claim 259 or 260, wherein the corticosteroid treatment is methylpredinosolone or dexamethasone.

262. The system of claim 254, wherein the therapy is a lisofylline treatment, and wherein determining whether the subject is a candidate subject comprises determining that the subject is unlikely to be a responder responsive to the classification of the subject comprising subphenotype B from the two or more subphenotypes.

263. The system of claim 254, wherein the therapy is a lisofylline treatment, and wherein determining whether the subject is a candidate subject comprises determining that the subject is likely to be a responder responsive to the classification of the subject comprising subphenotype A from the two or more subphenotypes.

264. The system of claim 254, wherein the therapy is a ketoconazole treatment, and wherein determining whether the subject is a candidate subject comprises determining that the subject is likely to be a responder responsive to the classification of the subject comprising subphenotype B from the two or more subphenotypes.

265. The system of claim 254, wherein the therapy is a pulmonary artery catheter and liberal fluid treatment, and wherein determining whether the subject is a candidate subject comprises determining that the subject is likely to be a responder responsive to the classification of the subject comprising subphenotype B from the two or more subphenotypes.

266. The system of claim 254, wherein the therapy is a pulmonary artery catheter and liberal fluid treatment, and wherein determining whether the subject is a candidate subject comprises determining that the subject is unlikely to be a responder responsive to the classification of the subject comprising subphenotype A from the two or more subphenotypes.

267. The system of claim 265 or 266, wherein the catheter and fluid treatment comprises a central venous catheter line treatment or a pulmonary artery catheter line treatment.

268. The system of claim 254, wherein the therapy is a recruitment maneuver, and wherein determining whether the subject is a candidate subject comprises determining that the subject is likely to be a responder responsive to the classification of the subject comprising subphenotype B from the two or more subphenotypes.

269. The system of claim 254, wherein the therapy is a recruitment maneuver, and wherein determining whether the subject is a candidate subject comprises determining that the subject is unlikely to be a responder responsive to the classification of the subject comprising subphenotype A from the two or more subphenotypes.

270. The system of claim 254, wherein the therapy is a statin treatment, and wherein determining whether the subject is a candidate subject comprises determining that the subject is likely to be a responder responsive to the classification of the subject comprising subphenotype B from the two or more subphenotypes.

271. The system of claim 254, wherein the therapy is a preemptive statin treatment, and wherein determining whether the subject is a candidate subject comprises determining that the subject is likely to be a responder responsive to the classification of the subject comprising subphenotype A from the two or more subphenotypes.

272. The system of claim 254, wherein the therapy is a full enteral feeding, and wherein determining whether the subject is a candidate subject comprises determining that the subject is likely to be a responder responsive to the classification of the subject comprising subphenotype A from the two or more subphenotypes.

273. The system of claim 254, wherein the therapy is a trophic enteral feeding, and wherein determining whether the subject is a candidate subject comprises determining that the subject is likely to be a responder responsive to the classification of the subject comprising subphenotype B from the two or more subphenotypes.
